(19) United States
(12) Patent Application Publication
(10) Pub. No.: US 2022/0110933 A1
(43) Pub. Date: Apr. 14, 2022

(54) METHODS OF TREATING DISEASE WITH MAGL INHIBITORS

(71) Applicant: H. Lundbeck A/S, Valby (DK)

(72) Inventor: Channing Rodney BEALS, San Diego, CA (US)

(21) Appl. No.: 17/425,124

(22) PCT Filed: Jan. 24, 2020
(86) PCT No.: PCT/US2020/015083

§ 371 Date: Jul. 22, 2021

Related U.S. Application Data
(60) Provisional application No. 62/796,941, filed on Jan. 25, 2019.

(51) Int. Cl. A61K 31/496; A61P 25/14; A61K 31/495; A61K 31/5377; A61K 31/4709; A61K 31/4545; A61K 31/55; A61K 31/537; A61K 31/438; A61K 31/4985

(52) U.S. Cl. CPC A61K 31/496; A61P 25/14; A61K 31/495; A61K 31/5377; A61K 31/4985; A61K 31/4545; A61K 31/55; A61K 31/537; A61K 31/438; A61K 31/4709

(57) ABSTRACT

Provided herein are methods for the treatment of disease with monoacylglycerol lipase (MAGL) inhibitors.

### CROSS-REFERENCE

This application claims benefit of U.S. Provisional Application No. 62/796,941, filed on Jan. 25, 2019, which is herein incorporated by reference in its entirety.

### BACKGROUND

Monoacylglycerol lipase (MAGL) is an enzyme responsible for hydrolyzing endocannabinoids such as 2-AG (2-arachidonoylglycerol), an arachidonate based lipid, in the nervous system.

### BRIEF SUMMARY OF THE INVENTION

This disclosure provides, for example, methods for treating dyskinesia with compounds and pharmaceutical compositions which are modulators of MAGL. The disclosure also provides for the use of disclosed compounds as medicaments and/or in the manufacture of medicaments for the inhibition of MAGL in warm-blooded animals such as humans.

In some embodiments is a method for treating dyskinesia in a patient, comprising administering to the patient in need thereof a therapeutically effective amount of a compound of Formula (I′):

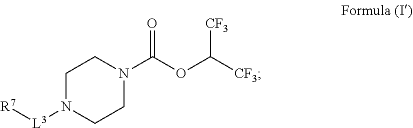

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is optionally substituted with one or two substituents independently selected from C₁₋₆ haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

In some embodiments is method for treating dyskinesia with a compound of Formula (I′), wherein the compound of Formula (I′) is a compound of Formula (III):

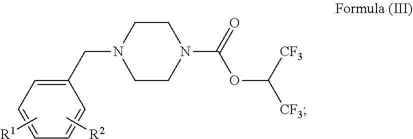

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

In some embodiments is method for treating dyskinesia with a compound of Formula (I′) or (III), wherein R⁵ and R⁶, together with the nitrogen to which they are attached, form a 4-6 membered saturated monocyclic heterocycle, wherein the 4-6 membered saturated monocyclic heterocycle is substituted with one substituent selected from C₁₋₆haloalkyl, —C(O)OR⁹, and —NR⁹SO₂R⁸; and

the 4-6 membered saturated monocyclic heterocycle optionally contains an additional 0, N, or S. In some embodiments is method for treating dyskinesia with a compound of Formula (I′) or (III), wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine, piperidine, and morpholine. In some embodiments is method for treating dyskinesia with a compound of Formula (I′) or (III), wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine and piperidine. In some embodiments is method for treating dyskinesia with a compound of Formula (I′), wherein R⁵ and R⁶, together with the nitrogen to which they are attached, form an unsubstituted 4-6 membered saturated monocyclic heterocycle. In some embodiments is method for treating dyskinesia with a compound of Formula (I′), wherein R⁵ and R⁶, together with the nitrogen to which they are attached, form an unsubstituted 4-6 membered saturated monocyclic heterocycle, wherein the 4-6 membered saturated monocyclic heterocycle is selected from pyrrolidine, piperidine, and morpholine. In some embodiments is method for treating dyskinesia with a compound of Formula (I′) or (III), wherein R⁵ and R⁶, together with the nitrogen to which they are attached, form a 7-8 membered bridged heterocyclic ring optionally substituted with one or two substituents independently selected from halogen, oxo, and C₁₋₆alkyl. In some embodiments is method for treating dyskinesia with a compound of Formula (I′) or (III), wherein R⁵ and R⁶, together with the nitrogen to which they are attached, form an unsubstituted 7-8 membered bridged heterocyclic ring. In some embodiments is method for treating dyskinesia with a compound of Formula (I′) or (III), wherein R¹ is halogen, —SF₅, or optionally substituted C₁₋₆alkyl optionally substituted by halogen. In some embodiments is method for treating dyskinesia with a compound of Formula (I′) or (III), wherein R¹ is halogen. In some embodiments is method for treating dyskinesia with a compound of Formula (I′) or (III), wherein R¹ is C₁₋₆alkyl optionally substituted by halogen. In some embodiments is method for treating dyskinesia with a compound of Formula (I′) or (III), wherein R¹ is —CF₃.

In some embodiments is a method for treating dyskinesia in a patient, comprising administering to the patient in need thereof a therapeutically effective amount of a compound of Formula (I):

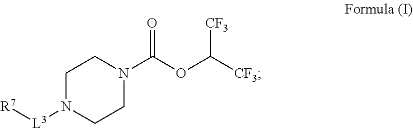

wherein:
L³ is a bond, —CH₂—, —S(O)₂—, or —C(O)—;
R⁷ is phenyl; wherein R⁷ is optionally substituted by one, two, or three moieties independently selected from R^{h};
R^{a} and R^{b} are independently selected, for each occurrence, from the group consisting of hydrogen and C₁₋₃alkyl; wherein C₁₋₃alkyl is optionally substituted by one or more substituents selected from halogen, cyano, oxo, hydroxyl, heterocycle, and phenyl; or R^{a} and R^{b}, when they occur together with the nitrogen to which they are attached, form a 4-6 membered saturated heterocyclic ring, which may have an additional heteroatom selected from O, S, and N, or a spirocyclic ring selected from 8-oxa-2-azaspiro[4.5]decane and 2,8-diazaspiro[4.5]decane, wherein the 4-6 membered saturated heterocyclic ring or the spirocyclic ring are optionally substituted by one or more substituents selected from the group consisting of halogen, cyano, oxo, C₁₋₆ alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl;
R^{c} is selected from the group consisting of halogen, hydroxyl, C₁₋₆alkyl (optionally substituted by one, two, or three halogens), and C₁₋₆alkoxy (optionally substituted by one, two, or three halogens); and
R^{h} is selected from the group consisting of: halogen, phenyl (optionally substituted by one, two, or three moieties each independently selected from R^{c}), hydroxyl, cyano, C₁₋₆alkyl (optionally substituted by one, two or three halogens), C₁₋₆alkoxy (optionally substituted by one, two or three halogens), R^{a}R^{b}N—, R^{a}—C(O)NR^{a}—, R^{a}R^{b}N—SO₂—, R^{a}R^{b}N—C(O)—, R^{a}—S(O)_{w}— (wherein w is 0, 1 or 2), R^{a}—SO₂—NR^{b}—, and heteroaryl (optionally substituted by one, two or three moieties each independently selected from R^{c});
   or a pharmaceutically acceptable salt or solvate thereof.

In some embodiments is method for treating dyskinesia with a compound of Formula (I), wherein L³ is a —CH₂—. In some embodiments is method for treating dyskinesia with a compound of Formula (I), wherein L³ is a —CH₂—; and R^{h} is selected from the group consisting of: halogen, phenyl (optionally substituted by one, two, or three moieties each independently selected from halogen, methyl, ethyl, propyl, t-butyl, and CF₃), C₁₋₆alkyl (optionally substituted by one, two or three halogens), C₁₋₆alkoxy (optionally substituted by one, two or three halogens), R^{a}R^{b}N—, R^{a}R^{b}N—C(O)—, and heteroaryl (optionally substituted by one, two or three moieties each independently selected from C₁₋₆alkyl or halogen). In some embodiments is method for treating dyskinesia with a compound of Formula (I), wherein L³ is a —CH₂—; and R^{h} is selected from the group consisting of: halogen, C₁₋₆alkyl (optionally substituted by one, two or three halogens), C₁₋₆alkoxy (optionally substituted by one, two or three halogens), and R^{a}R^{b}N—. In some embodiments is method for treating dyskinesia with a compound of Formula (I), wherein R⁷ is substituted by two moieties independently selected from R^{h}. In some embodiments is method for treating dyskinesia with a compound of Formula (I), wherein L³ is a —CH₂—; and R⁷ is substituted by R^{a}R^{b}N— and a moiety selected from the group consisting of: halogen, C₁₋₆alkyl (optionally substituted by one, two or three halogens), and C₁₋₆alkoxy (optionally substituted by one, two or three halogens). In some embodiments is method for treating dyskinesia with a compound of Formula (I), wherein R^{a} and R^{b}, together with the nitrogen to which they are attached, form a 4-6 membered saturated heterocyclic ring, which may have an additional heteroatom selected from O, S, and N, and the 4-6 membered saturated heterocyclic ring is optionally substituted by one or more substituents selected from the group consisting of halogen, cyano, oxo, C₁₋₆alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl. In some embodiments is method for treating dyskinesia with a compound of Formula (I), wherein the 4-6 membered saturated heterocyclic ring is selected from azetidine, pyrrolidine, piperidine, piperazine, and morpholine, and the 4-6 membered saturated heterocyclic ring is optionally substituted by one or more substituents selected from the group consisting of halogen, cyano, oxo, C₁₋₆alkyl, —S(O)_{w}—C₁₋₆alkyl (where w is 0, 1 or 2), hydroxyl, —C(O)—C₁₋₆alkyl, —NH₂, and —NH—C(O)—C₁₋₆ alkyl. In some embodiments is method for treating dyskinesia with a compound of Formula (I), wherein the 4-6 membered saturated heterocyclic ring is pyrrolidine. In some embodiments is method for treating dyskinesia with a compound of Formula (I), wherein the 4-6 membered saturated heterocyclic ring is morpholine. In some embodiments is method for treating dyskinesia with a compound of Formula (I), wherein the 4-6 membered saturated heterocyclic ring is piperidine. In some embodiments is method for treating dyskinesia with a compound of Formula (I), wherein L³ is a —CH₂—;

and R^{h} is selected from the group consisting of: halogen, phenyl (optionally substituted by one, two, or three moieties each independently selected from halogen, methyl, ethyl, propyl, t-butyl, and CF₃), C₁₋₆alkyl (optionally substituted by one, two or three halogens), C₁₋₆alkoxy (optionally substituted by one, two or three halogens), and heteroaryl (optionally substituted by one, two or three moieties each independently selected from C₁₋₆alkyl or halogen). In some embodiments is method for treating dyskinesia with a compound of Formula (I), wherein R⁷ is substituted by two moieties independently selected from R^{h}. In some embodiments is method for treating dyskinesia with a compound of Formula (I), wherein L³ is a —CH₂—; and R^{h} is selected from the group consisting of: halogen, C₁₋₆alkyl (optionally substituted by one, two or three halogens), C₁₋₆alkoxy (optionally substituted by one, two or three halogens), and R^{a}R^{b}N—. In some embodiments is method for treating dyskinesia with a compound of Formula (I), wherein R⁷ is substituted by two moieties independently selected from R^{h}.

In some embodiments is a method for treating dyskinesia in a patient, comprising administering to the patient in need thereof a therapeutically effective amount of a compound of Formula (II):

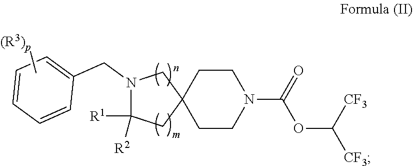

wherein:
R¹ is H or C₁₋₆alkyl;
R² is H or C₁₋₆alkyl;
each R³ is independently selected from C₁₋₆alkyl, C₂₋₆alkenyl, C₂₋₆alkynyl, halogen, —CN, C₁₋₆haloalkyl, C₁₋₆aminoalkyl, heterocycloalkyl, —C₁₋₆alkyl(heterocycloalkyl), heteroaryl, —SF₅, —NR⁵R⁶, —OR⁷, —CO₂R⁸, —C(O)R⁸, and —C(O)NR⁸R⁹, wherein heterocycloalkyl and —C₁₋₆alkyl(heterocycloalkyl) are optionally substituted with one or two R⁴; or two adjacent R³ form a heterocycloalkyl ring optionally substituted with one, two, or three R⁴;
each R⁴ is independently selected from C₁₋₆alkyl, C₁₋₆haloalkyl, C₃₋₈cycloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
each R⁵ and R⁶ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl; or R⁵ and R⁶, together with the nitrogen to which they are attached, form a heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl, wherein heterocycloalkyl, aryl, and heteroaryl are optionally substituted with one or two groups selected from oxo, C₁₋₆alkyl, C₁₋₆haloalkyl, CO₂H, and C(O)NH₂;
each R⁸ and R⁹ is independently selected from H, C₁₋₆alkyl, C₁₋₆haloalkyl, C₃₋₈cycloalkyl, aryl, and heteroaryl; or R⁸ and R⁹, together with the nitrogen to which they are attached, form a heterocycloalkyl ring optionally substituted with one or two groups selected from C₁₋₆alkyl, C₁₋₆haloalkyl, CO₂H, and C(O)NH₂;
each R¹⁰ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
p is 0, 1, 2, 3, 4, or 5;
n is 0 or 1; and
m is 1 or 2; provided that when n is 0, then m is 2; and when n is 1, then m is 1;
or a pharmaceutically acceptable salt or solvate thereof.

In some embodiments is method for treating dyskinesia with a compound of Formula (II), wherein each R³ is independently selected from C₁₋₆alkyl, C₂₋₆alkynyl, halogen, —CN, C₁₋₆haloalkyl, heterocycloalkyl, —C₁₋₆alkyl(heterocycloalkyl), heteroaryl, —SF₅, —NR⁵R⁶, —OR⁷, —CO₂R⁸, and —C(O)NR⁸R⁹. In some embodiments is method for treating dyskinesia with a compound of Formula (II), wherein R¹ and R² are both H. In some embodiments is method for treating dyskinesia with a compound of Formula (II), wherein each R³ is independently selected from halogen, C₁₋₆haloalkyl, —NR⁵R⁶, and —OR⁷. In some embodiments is method for treating dyskinesia with a compound of Formula (II), wherein R⁵ and R⁶, together with the nitrogen to which they are attached, form a heterocycloalkyl ring optionally substituted with one or two R¹⁰ independently selected from C₁₋₆alkyl, C₃₋₈cycloalkyl, C₁₋₆haloalkyl, halogen, —CO₂R⁸, —C(O)R⁸, —C(O)NR⁸R⁹, —SO₂R⁸, —NR⁹C(O)R⁸, and —NR⁹SO₂R⁸. In some embodiments is method for treating dyskinesia with a compound of Formula (II), wherein R⁵ and R⁶, together with the nitrogen to which they are attached, form a heterocycloalkyl ring substituted with one or two R¹⁰ independently selected from C₁₋₆alkyl and —CO₂H. In some embodiments is method for treating dyskinesia with a compound of Formula (II), wherein R⁵ and R⁶, together with the nitrogen to which they are attached, form an unsubstituted heterocycloalkyl ring. In some embodiments is method for treating dyskinesia with a compound of Formula (II), wherein R⁵ and R⁶, together with the nitrogen to which they are attached, form a heterocycloalkyl ring optionally substituted with one, two, or three R¹⁰ selected from:

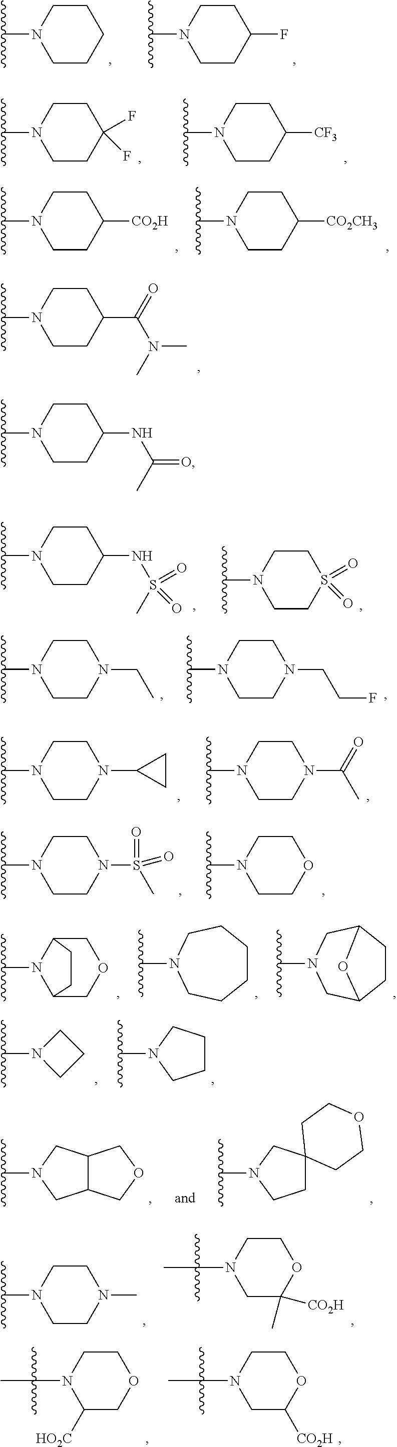

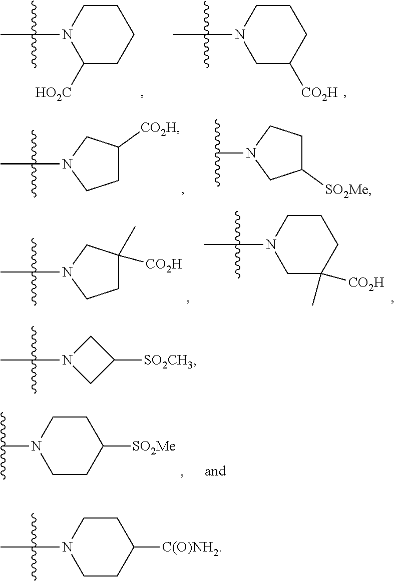

In some embodiments is method for treating dyskinesia with a compound of Formula (II), wherein p is 1 or 2. In some embodiments is method for treating dyskinesia with a compound of Formula (II), wherein n is 0 and m is 2.

In some embodiments is a method for treating dyskinesia in a patient, comprising administering to the patient in need thereof a therapeutically effective amount of a compound of Formula (III):

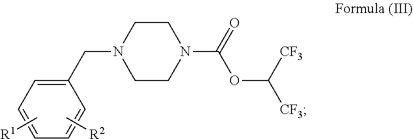

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

In some embodiments is method for treating dyskinesia with a compound of Formula (III), wherein R¹ is halogen, —SF₅, or optionally substituted C₁₋₆alkyl optionally substituted by halogen. In some embodiments of the methods for treating dyskinesia with a compound of Formula (III), R¹ is halogen. In some embodiments of the methods for treating dyskinesia with a compound of Formula (III), R¹ is C₁₋₆alkyl optionally substituted by halogen. In some embodiments of the methods for treating dyskinesia with a compound of Formula (III), R¹ is —CF₃. In some embodiments is method for treating dyskinesia with a compound of Formula (III), wherein R⁵ and R⁶, together with the nitrogen to which they are attached, form a 4-6 membered saturated monocyclic heterocycle, wherein the 4-6 membered saturated monocyclic heterocycle is substituted with one substituent selected from C₁₋₆haloalkyl, —C(O)OR⁹, and —NR⁹SO₂R⁸; and the 4-6 membered saturated monocyclic heterocycle optionally contains an additional O, N, or S. In some embodiments is method for treating dyskinesia with a compound of Formula (III), wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine, piperidine, and morpholine. In some embodiments is method for treating dyskinesia with a compound of Formula (III), wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine and piperidine. In some embodiments is method for treating dyskinesia with a compound of Formula (III), wherein R⁵ and R⁶, together with the nitrogen to which they are attached, form a 7-8 membered bridged heterocyclic ring optionally substituted with one or two substituents independently selected from halogen, oxo, and C₁₋₆alkyl. In some embodiments is method for treating dyskinesia with a compound of Formula (III), wherein R⁵ and R⁶, together with the nitrogen to which they are attached, form an unsubstituted 7-8 membered bridged heterocyclic ring.

In some embodiments is a method for treating dyskinesia in a patient, comprising administering to the patient in need thereof a therapeutically effective amount of a compound of Formula (IV):

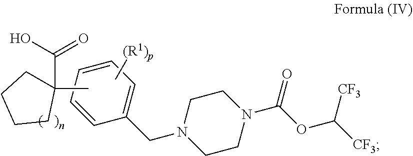

wherein:
each R¹ is independently halogen, C₁₋₆alkyl, C₁₋₆haloalkyl, C₁₋₆alkoxy, C₁₋₆haloalkoxy, C₃₋₈cycloalkyl, —OH, —CN, or —SF₅;
n is 1 or 2; and
p is 0, 1, 2, 3, or 4;
   or a pharmaceutically acceptable salt or solvate thereof.

In some embodiments is method for treating dyskinesia with a compound of Formula (IV), wherein n is 1.

In some embodiments is a method for treating dyskinesia in a patient, comprising administering to the patient in need thereof a therapeutically effective amount of a compound of Formula (V)

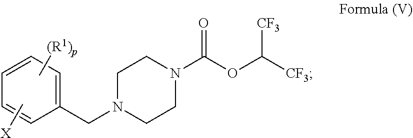

wherein:
X is —N(R²)(R³), —C₁₋₆alkyl-N(R⁴)(R⁵), —C(O)N(R⁴)(R⁵),

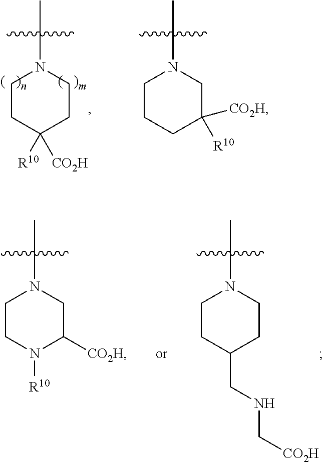

each R¹ is independently halogen, C₁₋₆alkyl, C₁₋₆haloalkyl, C₁₋₆alkoxy, C₁₋₆haloalkoxy, C₃₋₈cycloalkyl, —OH, —CN, or —SF₅;
R² and R³, together with the nitrogen to which they are attached, form
   (i) a C₂-C₈heterocycloalkyl; or
   (ii) a C₂-C₈heteroaryl;
wherein the C₂-C₈heterocycloalkyl or the C₂-C₈heteroaryl is substituted with one R⁶ and optionally substituted with one or two additional substituents selected from halogen, C₁₋₆alkyl, C₁₋₆haloalkyl, and C₁₋₆alkoxy;
R⁴ and R⁵, together with the nitrogen to which they are attached, form
   (i) a C₂-C₈heterocycloalkyl; or
   (ii) a C₂-C₈heteroaryl;
wherein the C₂-C₈heterocycloalkyl or the C₂-C₈heteroaryl is substituted with one R⁷ and optionally substituted with one or two additional substituents selected from halogen, C₁₋₆alkyl, C₁₋₆haloalkyl, and C₁₋₆alkoxy;
R⁶ is —C₁₋₆alkyl-CO₂H or —N(R⁸)—C₁₋₆alkyl-CO₂H;
R⁷ is —CO₂H, —C₁₋₆alkyl-CO₂H, or —N(R⁹)—C₁₋₆alkyl-CO₂H;
R⁸ is H or C₁₋₆alkyl;
R⁹ is H or C₁₋₆alkyl;
R¹⁰ is C₁₋₆alkyl;
m is 0, 1, or 2;
n is 0 or 1; and
p is 0, 1, 2, 3, or 4;
   or a pharmaceutically acceptable salt or solvate thereof.

In some embodiments is method for treating dyskinesia with a compound of Formula (V), wherein X is

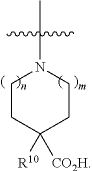

In some embodiments is method for treating dyskinesia with a compound of Formula (V), wherein m is 1 and n is 1. In some embodiments is method for treating dyskinesia with a compound of Formula (V), wherein X is —N(R²)(R³). In some embodiments is method for treating dyskinesia with a compound of Formula (V), wherein R² and R³, together with the nitrogen to which they are attached, form a C₂-C₈heterocycloalkyl substituted with one R⁶. In some embodiments is method for treating dyskinesia with a compound of Formula (V), wherein R² and R³, together with the nitrogen to which they are attached, form a C₂-C₈heterocycloalkyl selected from:

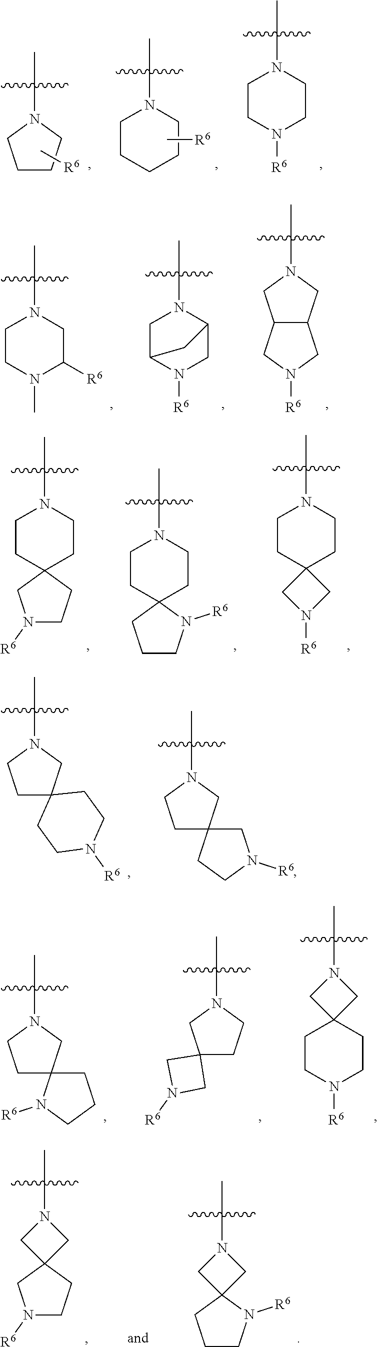

In some embodiments is method for treating dyskinesia with a compound of Formula (V), wherein R⁶ is —C₁₋₆alkyl-CO₂H.

In some embodiments is method for treating dyskinesia with a compound of Formula (IV) or (V), wherein each R¹ is independently halogen. In some embodiments is method for treating dyskinesia with a compound of Formula (IV) or (V), wherein each R¹ is independently C₁₋₆haloalkyl. In some embodiments is method for treating dyskinesia with a compound of Formula (IV) or (V), wherein each R¹ is independently C₁₋₆alkyl. In some embodiments is method for treating dyskinesia with a compound of Formula (IV) or (V), wherein p is 1.

In some embodiments is a method for treating dyskinesia in a patient, comprising administering to the patient in need thereof a therapeutically effective amount of a compound of Formula (VI):

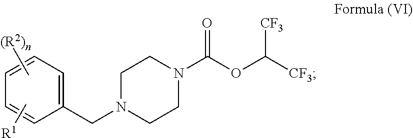

wherein:
R¹ is —N(R³)(R⁵) or —NH(R⁴);
each R² is independently selected from halogen, C₁₋₆alkyl, —CN, C₁₋₆haloalkyl, and —OR⁶;
R³ is —CH₂CO₂H, —CH₂CH₂CO₂H, or —CH(CH₃)CO₂H;
R⁴ is —(CH₂)ₘ—CO₂H;
R⁵ is H or C₁₋₃alkyl;
each R⁶ is independently selected from H, C₁₋₆alkyl, and C₁₋₆haloalkyl;
n is 0, 1, 2, 3, or 4; and
m is 3;
or a pharmaceutically acceptable salt or solvate thereof.

In some embodiments is method for treating dyskinesia with a compound of Formula (VI), wherein R¹ is —N(R³)(R⁵). In some embodiments is method for treating dyskinesia with a compound of Formula (VI), wherein R⁵ is H. In some embodiments is method for treating dyskinesia with a compound of Formula (VI), wherein R¹ is —NH(R⁴). In some embodiments is method for treating dyskinesia with a compound of Formula (VI), wherein each R² is independently selected from halogen, C₁₋₆alkyl, and C₁₋₆ haloalkyl. In some embodiments is method for treating dyskinesia with a compound of Formula (VI), wherein n is 1.

In some embodiments is a method for treating dyskinesia in a patient, comprising administering to the patient in need thereof a therapeutically effective amount of a compound of Formula (VII):

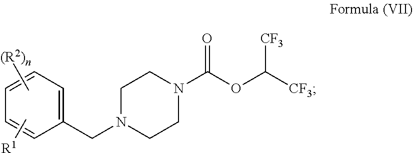

wherein:
R¹ is —R¹⁴, —OR³, —SR⁴, —S(O)₂R⁴, or —C≡C—(CR⁶R)—R⁸;
each R² is independently selected from C₁₋₆alkyl, halogen, —CN, C₁₋₆haloalkyl, —C₁₋₆alkyl(heterocycloalkyl), —OR¹⁷, and —C(O)NR¹⁸R¹⁹;
R³ is —(CR⁶R⁷)ₘ—R⁸, —(CR⁶R⁷)ₚ—Y—(CR⁶R⁷)_{q}—R⁸, or —(CR⁶R⁷)ₜ—C₃₋₆cycloalkyl-R⁸;
R⁴ is —(CR⁶R⁷)ₘ—R^{8′}, —(CR⁶R⁷)—C(O)OH, or —(CR⁶R⁷)ₚ—Y—(CR⁶R⁷)_{q}—R⁸;
Y is —O— or —N(R²²)—;
each R⁶ and R⁷ is each independently selected from H, F, and C₁₋₆alkyl; or R⁶ and R⁷, together with the carbon to which they are attached, form a C₃₋₆cycloalkyl ring;
R⁸ is —C(O)OR⁹, —C(O)R¹⁰, or —C(O)O—(CR¹²R¹³)—OC(O)R¹¹;
R^{8′} is —C(O)OR^{9′}, —C(O)R^{10′}, or —C(O)O—(CR¹²R¹³)—OC(O)R¹¹;
R⁹ is H or C₁₋₆alkyl;
R^{9′} is C₁₋₆alkyl;
R¹⁰ is C₁₋₆alkyl or —NHSO₂R²¹;
R^{10′} is C₂₋₆alkyl or —NHSO₂R²¹;
R¹¹ is C₁₋₆alkyl or C₁₋₆alkoxy;
R¹² and R¹³ is each independently H or C₁₋₆alkyl;
R¹⁴ is —(CR¹⁵R¹⁶)ₘ—R⁸ or —(CR⁶R⁷)ₚ—Y—(CR⁶R⁷)_{q}—R⁸;
each R¹⁵ and R¹⁶ is each independently selected from H, F, and C₁₋₆alkyl;
each R¹⁷ is independently selected from H, C₁₋₆alkyl, C₁₋₆haloalkyl, and C₃₋₆cycloalkyl;
each R¹⁸ and R¹⁹ is each independently selected from H, C₁₋₆alkyl, C₃₋₆cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl or C₃₋₆cycloalkyl;
R²² is H, C₁₋₆alkyl, or —SO₂R²³;
R²³ is C₁₋₆alkyl;
m is 1, 2, 3 or 4;
n is 0, 1, 2, 3, or 4;
p is 2, 3, or 4;
q is 1, 2, or 3;
t is 0, 1, or 2; and
v is 3 or 4;
   or a pharmaceutically acceptable salt or solvate thereof.

In some embodiments is method for treating dyskinesia with a compound of Formula (VII), wherein R¹ is —OR³. In some embodiments is method for treating dyskinesia with a compound of Formula (VII), wherein R³ is —(CR⁶R⁷)ₘ—R⁸. In some embodiments is method for treating dyskinesia with a compound of Formula (VII), wherein m is 1, 2, or 3. In some embodiments is method for treating dyskinesia with a compound of Formula (VII), wherein each R⁶ and R⁷ is each independently selected from H and C₁₋₆alkyl, or R⁶ and R⁷, together with the carbon to which they are attached, form a C₃₋₆cycloalkyl ring. In some embodiments is method for treating dyskinesia with a compound of Formula (VII), wherein R⁸ is —C(O)OR⁹. In some embodiments is method for treating dyskinesia with a compound of Formula (VII), wherein R⁹ is H. In some embodiments is method for treating dyskinesia with a compound of Formula (VII), wherein each R² is independently selected from C₁₋₆alkyl, halogen, and C₁₋₆haloalkyl. In some embodiments is method for treating dyskinesia with a compound of Formula (VII), wherein n is 2. In some embodiments is method for treating dyskinesia with a compound of Formula (VII), wherein n is 1.

In some embodiments is a method for treating dyskinesia in a patient, comprising administering to the patient in need thereof a therapeutically effective amount of a compound of Formula (VIII):

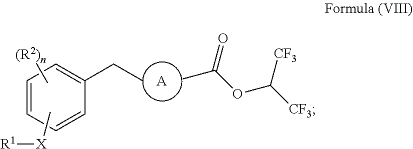

wherein:

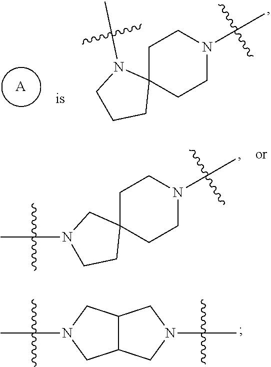

X is —O—, —S—, —SO₂—, —N(R³)—, or —CH₂—;
Y is —O— or —N(R⁷)—;
R¹ is —(CR⁴R⁵)ₘ—R⁶, —(CR⁴R⁵)ₚ—Y—(CR⁴R⁵)_{q}—R⁶, or —(CR⁴R⁵)ₜ—C₃₋₆cycloalkyl-R⁶;
each R² is independently selected from halogen, —CN, C₁₋₆alkyl, C₁₋₆haloalkyl, —C₁₋₆alkyl(heterocycloalkyl), —OR¹⁷, and —C(O)NR¹⁸R¹⁹;
R³ is H or C₁₋₆alkyl;
each R⁴ and R⁵ is each independently selected from H, F, and C₁₋₆alkyl; or R⁴ and R⁵, together with the carbon to which they are attached, form a C₃₋₆cycloalkyl ring;
R⁶ is —CO₂R⁹, —C(O)R¹⁰, or —C(O)O—(CR¹²R¹³)—OC(O)R¹¹;
R⁷ is H, C₁₋₆alkyl, or —SO₂R⁸;
R⁸ is C₁₋₆alkyl;
R⁹ is H or C₁₋₆alkyl;
R¹⁰ is C₁₋₆alkyl or —NHSO₂R²¹;
R¹¹ is C₁₋₆alkyl or C₁₋₆alkoxy;
R¹² and R¹³ is each independently H or C₁₋₆alkyl;
each R¹⁷ is independently selected from H, C₁₋₆alkyl, C₁₋₆haloalkyl, aminoalkyl, cycloalkyl, —C₁₋₆alkyl(heterocycloalkyl), —C₁₋₆alkyl-C(O)(heterocycloalkyl), optionally substituted heterocycloalkyl, optionally substituted aryl, and optionally substituted heteroaryl;
each R¹⁸ and R¹⁹ is independently selected from H, C₁₋₆alkyl, C₁₋₆haloalkyl, cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl;
m is 1, 2, 3 or 4;
n is 0, 1, 2, 3, or 4;
p is 2, 3, or 4;
q is 1, 2, or 3; and
t is 0, 1, or 2;
   or a pharmaceutically acceptable salt or solvate thereof.

In some embodiments is method for treating dyskinesia with a compound of Formula (VIII), wherein R¹ is —(CR⁴R⁵)ₘ—R⁶. In some embodiments is method for treating dyskinesia with a compound of Formula (VIII), wherein each R⁴ and R⁵ is each independently selected from H and C₁₋₆alkyl. In some embodiments is method for treating dyskinesia with a compound of Formula (VIII), wherein each R⁴ and R⁵ is H. In some embodiments is method for treating dyskinesia with a compound of Formula (VIII), wherein R⁶ is —CO₂R⁹. In some embodiments is method for treating dyskinesia with a compound of Formula (VIII), wherein R⁹ is H. In some embodiments is method for treating dyskinesia with a compound of Formula (VIII), wherein R⁶ is —C(O)R¹⁰. In some embodiments is method for treating dyskinesia with a compound of Formula (VIII), wherein R¹⁰ is —NHSO₂R²¹. In some embodiments is method for treating dyskinesia with a compound of Formula (VIII), wherein X is —O—. In some embodiments is method for treating dyskinesia with a compound of Formula (VIII), wherein X is —N(R³)—. In some embodiments is method for treating dyskinesia with a compound of Formula (VIII), wherein

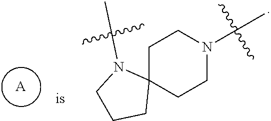

In some embodiments is method for treating dyskinesia with a compound of Formula (VIII), wherein

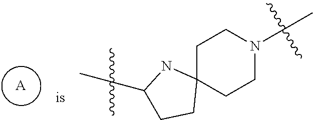

In some embodiments is method for treating dyskinesia with a compound of Formula (VIII), wherein each R² is independently selected from halogen, C₁₋₆alkyl, and C₁₋₆ haloalkyl. In some embodiments is method for treating dyskinesia with a compound of Formula (VIII), wherein n is 1.

In some embodiments is a method for treating dyskinesia in a patient, comprising administering to the patient in need thereof a therapeutically effective amount of a compound of Formula (IX):

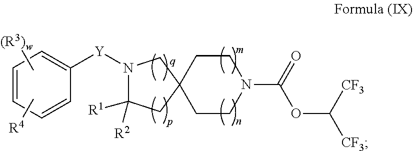

wherein:
Y is —CH₂— or —C(O)—;
R¹ is H or C₁₋₆alkyl;
R² is H or C₁₋₆alkyl;
each R³ is independently selected from C₁₋₆alkyl, halogen, —CN, C₁₋₆haloalkyl, —SF₅, and —OR⁷;
R⁴ is selected from —C≡C—C₁₋₆alkyl-CO₂H and —C₃₋₈cycloalkyl-CO₂H;
each R⁷ is independently selected from H, C₁₋₆alkyl, C₁₋₆haloalkyl, C₁₋₆aminoalkyl, C₃₋₈cycloalkyl, and —C₁₋₆alkyl-C₃₋₈cycloalkyl;
w is 0, 1, 2, 3, or 4;
n is 0 or 1;
m is 0 or 1;
p is 0, 1, or 2; and
q is 0, 1, or 2; provided that when q is 0, then p is 2;
   or a pharmaceutically acceptable salt or solvate thereof.

In some embodiments is method for treating dyskinesia with a compound of Formula (IX), wherein R⁴ is —C₃₋₈cycloalkyl-CO₂H. In some embodiments is method for treating dyskinesia with a compound of Formula (IX), wherein R⁴ is

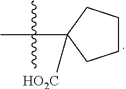 [SMILES: *C1(C(=O)O)CCCC1]

In some embodiments is method for treating dyskinesia with a compound of Formula (IX), wherein R⁴ is —C≡C—C₁₋₆alkyl-CO₂H. In some embodiments is method for treating dyskinesia with a compound of Formula (IX), wherein R⁴ is

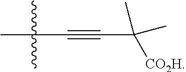 [SMILES: *C#CC(C)(C)C(=O)O]

In some embodiments is method for treating dyskinesia with a compound of Formula (IX), wherein Y is —CH₂—. In some embodiments is method for treating dyskinesia with a compound of Formula (IX), wherein R¹ and R² are both H. In some embodiments is method for treating dyskinesia with a compound of Formula (IX), wherein each R³ is independently selected from halogen and C₁₋₆haloalkyl. In some embodiments is method for treating dyskinesia with a compound of Formula (IX), wherein w is 1. In some embodiments is method for treating dyskinesia with a compound of Formula (IX), wherein m is 1, n is 1, q is 0, and p is 2.

In some embodiments is a method for treating dyskinesia in a patient, comprising administering to the patient in need thereof a therapeutically effective amount of a compound of Formula (X):

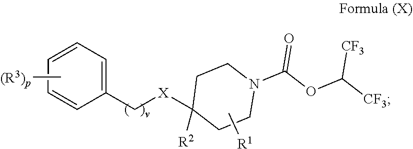

wherein:
X is —O— or —N(R¹¹)—;
R¹ is H or C₁₋₆alkyl;
R² is C₁₋₆alkyl;
each R³ is independently selected from C₁₋₆alkyl, C₂₋₆alkenyl, C₂₋₆alkynyl, —C≡C—C₁₋₆ alkyl-CO₂H, halogen, —CN, C₁₋₆haloalkyl, C₁₋₆aminoalkyl, C₃₋₈cycloalkyl, —C₁₋₆alkyl(C₂₋₉heterocycloalkyl), C₁₋₉heteroaryl, —SF₅, —NR⁵R⁶, —OR⁷, —CO₂R⁸, and —C(O)NR⁸R⁹, wherein C₃₋₈cycloalkyl, —C₁₋₆alkyl(C₂₋₉heterocycloalkyl), and C₁₋₉ heteroaryl are optionally substituted with one or two R⁴; or two adjacent R³ form a C₂₋₉heterocycloalkyl ring, wherein the C₂₋₉heterocycloalkyl ring is optionally substituted with one, two, or three R⁴;
each R⁴ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
each R⁵ and R⁶ is independently selected from H, C₁₋₆alkyl, C₁₋₆haloalkyl, C₁₋₆ aminoalkyl, C₃₋₈cycloalkyl, —C₁₋₆alkyl(C₂₋₉heterocycloalkyl), —C₁₋₆alkyl-C(O)(C₂₋₉heterocycloalkyl), C₂₋₉heterocycloalkyl, C₆₋₁₀aryl, and C₁₋₉heteroaryl; or R⁵ and R⁶, together with the nitrogen to which they are attached, form a C₂₋₉ heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(C₂₋₉heterocycloalkyl), —C₁₋₆alkyl-C(O)(C₂₋₉ heterocycloalkyl), —C₁₋₆alkyl-CO₂H, C₂₋₉heterocycloalkyl, C₆₋₁₀aryl, and C₁₋₉ heteroaryl, wherein C₂₋₉heterocycloalkyl, C₆₋₁₀aryl, and C₁₋₉heteroaryl are optionally substituted with one or two groups selected from oxo, C₁₋₆alkyl, C₁₋₆ haloalkyl, CO₂H, and CO₂NH₂;
each R⁸ and R⁹ is independently selected from H, C₁₋₆alkyl, C₁₋₆haloalkyl, C₃₋₈cycloalkyl, C₆₋₁₀aryl, and C₁₋₉heteroaryl; or R⁸ and R⁹, together with the nitrogen to which they are attached, form a C₂₋₉heterocycloalkyl ring optionally substituted with one or two groups selected from C₁₋₆alkyl, C₁₋₆haloalkyl, CO₂H, and CO₂NH₂;
each R¹⁰ is independently selected from halogen, C₁₋₆alkyl, C₁₋₆haloalkyl, C₃₋₈cycloalkyl, oxo, —CN, —CO₂R⁸, —C(O)R⁸, —C(O)NR⁸R⁹, —SO₂R⁸, —NR⁹C(O)R⁸, and —NR⁹SO₂R⁸;
R¹¹ is H, C₁₋₆alkyl, —C(O)—C₁₋₆alkyl, or —CH₂CO₂H;
p is 0, 1, 2, 3, 4, or 5; and
v is 0 or 1;
   or a pharmaceutically acceptable salt or solvate thereof.

In some embodiments is method for treating dyskinesia with a compound of Formula (X), wherein each R³ is independently selected from halogen, C₁₋₆haloalkyl, —NR⁵R⁶, and —OR⁷. In some embodiments is method for treating dyskinesia with a compound of Formula (X), wherein R⁵ and R⁶, together with the nitrogen to which they are attached, form a C₂₋₉heterocycloalkyl ring optionally substituted with one, two, or three R¹⁰. In some embodiments is method for treating dyskinesia with a compound of Formula (X), wherein R⁵ and R⁶, together with the nitrogen to which they are attached, form a C₂₋₉heterocycloalkyl ring substituted with one or two R¹⁰ independently selected from C₁₋₆alkyl and —CO₂H. In some embodiments is method for treating dyskinesia with a compound of Formula (X), wherein R⁵ and R⁶, together with the nitrogen to which they are attached, form an unsubstituted C₂₋₉heterocycloalkyl ring. In some embodiments is method for treating dyskinesia with a compound of Formula (X), wherein R⁵ and R⁶, together with the nitrogen to which they are attached, form a C₂₋₉heterocycloalkyl ring selected from:

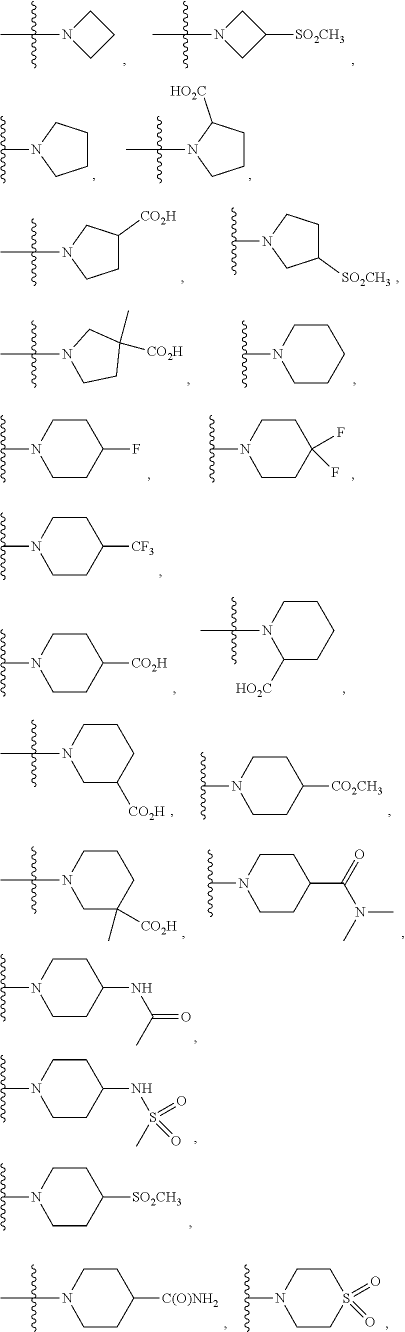

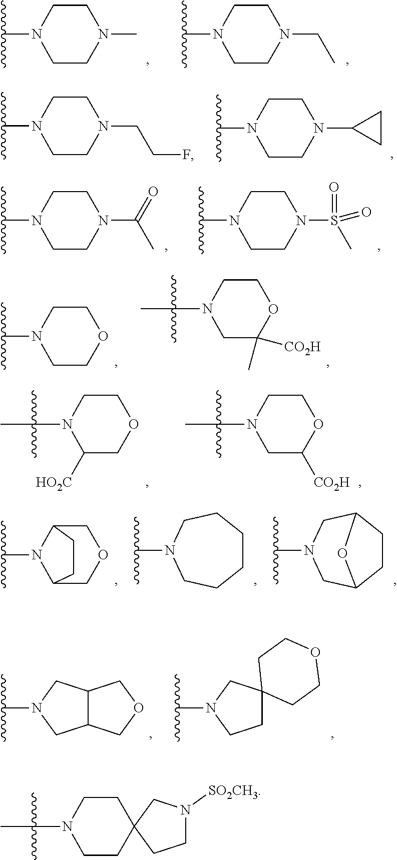

In some embodiments is a method for treating dyskinesia in a patient, comprising administering to the patient in need thereof a therapeutically effective amount of a compound of Formula (XI):

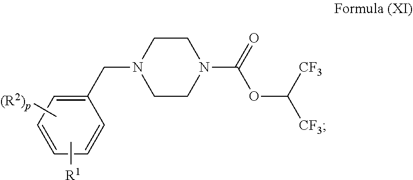

wherein:
R¹ is selected from

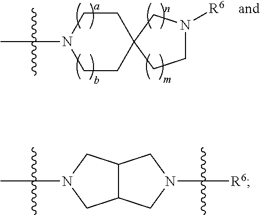

each R² is independently selected from C₁₋₆alkyl, halogen, —CN, C₁₋₆haloalkyl, C₃₋₈cycloalkyl, —SF₅, —OR³, and —C(O)NR⁴R⁵;
each R³ is independently selected from H, C₁₋₆alkyl, C₁₋₆haloalkyl, C₃₋₈cycloalkyl, and —C₁₋₆alkyl-C₃₋₈cycloalkyl;
each R⁴ and R⁵ is independently selected from H, C₁₋₆alkyl, and C₃₋₈cycloalkyl;
R⁶ is selected from C₁₋₆alkyl, —C(O)—C₁₋₆alkyl, and —S(O)₂—C₁₋₆alkyl;
a is 0 or 1;
b is 0 or 1;
m is 0, 1, or 2;
n is 0, 1, or 2; provided that when n is 0, then m is 2; and
p is 0, 1, 2, 3, or 4;
   or a pharmaceutically acceptable salt or solvate thereof.

In some embodiments is method for treating dyskinesia with a compound of Formula (XI), wherein R¹ is

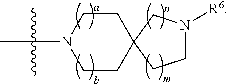

In some embodiments is method for treating dyskinesia with a compound of Formula (XI), wherein R⁶ is —C(O)—C₁₋₆alkyl. In some embodiments is method for treating dyskinesia with a compound of Formula (XI), wherein R⁶ is —S(O)₂—C₁₋₆alkyl. In some embodiments is method for treating dyskinesia with a compound of Formula (XI), wherein each R³ is independently selected from halogen and C₁₋₆haloalkyl. In some embodiments is method for treating dyskinesia with a compound of Formula (XI), wherein p is 1.

In some embodiments is a method for treating dyskinesia in a patient, comprising administering to the patient in need thereof a therapeutically effective amount of a compound of Formula (XII):

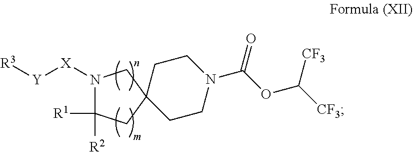

wherein:
X is —CH₂— or —C(O)—;
Y is a bond, C₁₋₆alkyl, C₁₋₆haloalkyl, or C₃₋₈cycloalkyl;
R¹ is H or C₁₋₆alkyl;
R² is H or C₁₋₆alkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, —CH₂CO₂R⁶, and —CH₂C(O)N(H)SO₂R⁸; wherein C₂₋₉heterocycloalkyl, —C₁₋₆alkyl(C₂₋₉heterocycloalkyl), phenyl, and C₁₋₉ heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl;
each R⁸ is independently selected from C₁₋₆alkyl, C₁₋₆haloalkyl, and C₃₋₈cycloalkyl;
n is 0 or 1; and
m is 1 or 2; provided that when n is 0, then m is 2; and when n is 1, then m is 1;
   or a pharmaceutically acceptable salt or solvate thereof.

In some embodiments is method for treating dyskinesia with a compound of Formula (XII), wherein Y is a bond. In some embodiments is method for treating dyskinesia with a compound of Formula (XII), wherein R¹ and R² are both H. In some embodiments is method for treating dyskinesia with a compound of Formula (XII), wherein X is —CH₂—. In some embodiments is method for treating dyskinesia with a compound of Formula (XII), wherein X is —C(O)—. In some embodiments is method for treating dyskinesia with a compound of Formula (XII), wherein n is 0 and m is 2.

In some embodiments is a method for treating dyskinesia in a patient, comprising administering to the patient in need thereof a therapeutically effective amount of a compound of Formula (XIII):

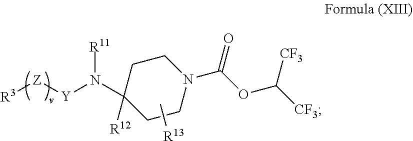

wherein:
Y is —CH₂— or —C(O)—;
Z is C₃₋₆cycloalkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, and —CH₂CO₂R⁶; wherein C₂₋₉heterocycloalkyl, —C₁₋₆alkyl(C₂₋₉heterocycloalkyl), phenyl, and C₁₋₉heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl;
R¹¹ is H, C₁₋₆alkyl, or —C₁₋₆alkyl-O—C₁₋₆alkyl;
R¹² is C₁₋₆alkyl;
R¹³ is H or C₁₋₆alkyl; and
v is 0 or 1;
   or a pharmaceutically acceptable salt or solvate thereof.

In some embodiments is method for treating dyskinesia with a compound of Formula (XIII), wherein R¹³ is H. In some embodiments is method for treating dyskinesia with a compound of Formula (XIII), wherein v is 0. In some embodiments is method for treating dyskinesia with a compound of Formula (XIII), wherein R¹ is Y is —C(O)—.

In some embodiments is method for treating dyskinesia with a compound of Formula (XII) or (XIII), wherein R³ is a 5-membered heteroaryl ring substituted with one, two, or three R⁴. In some embodiments is method for treating dyskinesia with a compound of Formula (XII) or (XIII), wherein R³ is a 5-membered heteroaryl ring substituted with two or three R⁴, wherein two adjacent R⁴ form a 6-membered heterocycloalkyl ring optionally substituted with one or two R⁵. In some embodiments is method for treating dyskinesia with a compound of Formula (XII) or (XIII), wherein R³ is a 5-membered heteroaryl ring substituted with two adjacent R⁴, wherein the two adjacent R⁴ form an unsubstituted 6-membered heterocycloalkyl ring. In some embodiments is method for treating dyskinesia with a compound of Formula (XII) or (XIII), wherein R³ is a 5-membered heteroaryl ring substituted with two adjacent R⁴, wherein the two adjacent R⁴ form a 6-membered heterocycloalkyl ring substituted with one R⁵. In some embodiments is method for treating dyskinesia with a compound of Formula (XII) or (XIII), wherein R⁵ is selected from C₁₋₆alkyl, C₁₋₆heteroalkyl, C₃₋₈cycloalkyl, —C₁₋₆alkyl(C₃₋₈cycloalkyl), C₂₋₉heterocycloalkyl, and —CH₂CO₂H. In some embodiments is method for treating dyskinesia with a compound of Formula (XII) or (XIII), wherein:

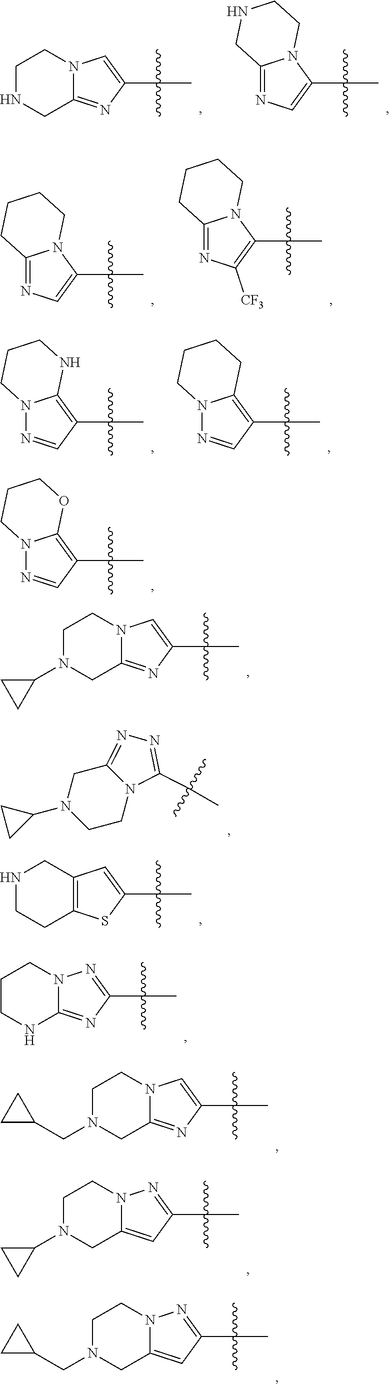

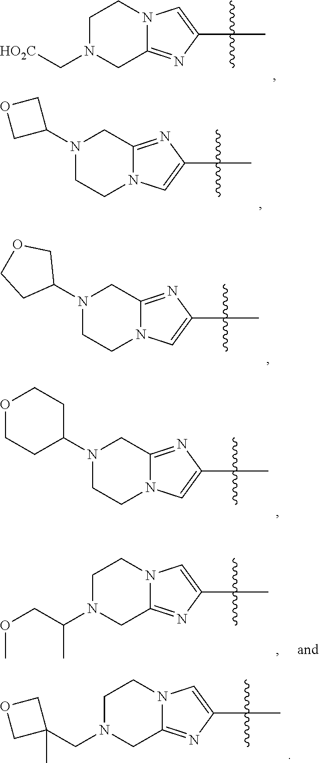

In some embodiments is a method for treating dyskinesia in a patient, comprising administering to the patient in need thereof a therapeutically effective amount of a compound of Formula (XIV):

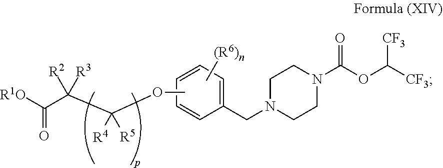

wherein:
R¹ is H or C₁₋₆alkyl;
R² is C₁₋₆alkyl;
R³ is H or C₁₋₆alkyl;
R⁴ and R⁵ are independently selected from H and C₁₋₆alkyl;
each R⁶ is independently selected from C₁₋₆alkyl, halogen, —CN, C₁₋₆haloalkyl, —OR⁷, —C(O)NR⁸R⁹, C₃₋₆cycloalkyl, C₂₋₉heterocycloalkyl, —C₁₋₆alkyl(C₂₋₉heterocycloalkyl), and C₂₋₉heteroaryl, wherein C₃₋₆cycloalkyl, C₂₋₉heterocycloalkyl, —C₁₋₆alkyl(C₂₋₉heterocycloalkyl), and C₂₋₉heteroaryl are optionally substituted with one, two, or three groups independently selected from halogen, C₁₋₆alkyl, C₁₋₆haloalkyl, and C₁₋₆alkoxy;
each R⁷ is independently selected from H, C₁₋₆alkyl, C₁₋₆haloalkyl, and C₃₋₆cycloalkyl;
each R⁸ and R⁹ is each independently selected from H, C₁₋₆alkyl, C₃₋₆cycloalkyl, aryl, and heteroaryl; or R⁸ and R⁹, together with the nitrogen to which they are attached, form a heterocycloalkyl ring optionally substituted with one, two, or three R¹⁰;
each R¹⁰ is independently selected from halogen, C₁₋₆alkyl, C₁₋₆haloalkyl, oxo, —CN, and C₃₋₆cycloalkyl;
n is 0, 1, 2, 3, or 4; and
p is 0 or 1;
   or a pharmaceutically acceptable salt or solvate thereof.

In some embodiments is method for treating dyskinesia with a compound of Formula (XIV), wherein p is 0. In some embodiments is method for treating dyskinesia with a compound of Formula (XIV), wherein p is 1. In some embodiments is method for treating dyskinesia with a compound of Formula (XIV), wherein R⁴ and R⁵ are H In some embodiments is method for treating dyskinesia with a compound of Formula (XIV), wherein R³ is C₁₋₆alkyl. In some embodiments is method for treating dyskinesia with a compound of Formula (XIV), wherein each R⁶ is independently selected from C₁₋₆ alkyl, halogen, —CN, C₁₋₆haloalkyl, —OR⁷, C₃₋₆cycloalkyl, C₂₋₉heterocycloalkyl, and C₂₋₉heteroaryl, wherein C₃₋₆cycloalkyl, C₂₋₉heterocycloalkyl, and C₂₋₉heteroaryl are optionally substituted with one or two groups independently selected from halogen, C₁₋₆alkyl, C₁₋₆haloalkyl, and C₁₋₆alkoxy. In some embodiments is method for treating dyskinesia with a compound of Formula (XIV), wherein each R⁶ is independently selected from C₁₋₆alkyl, halogen, —CN, and C₁₋₆haloalkyl. In some embodiments is method for treating dyskinesia with a compound of Formula (XIV), wherein n is 1 or 2.

In some embodiments is a method for treating dyskinesia in a patient, comprising administering to the patient in need thereof a therapeutically effective amount of a compound of Formula (XV):

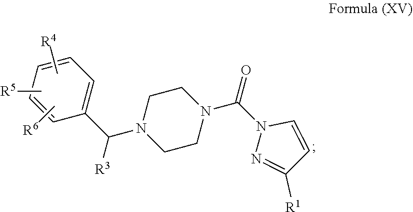

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₆alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring; and
R¹⁵ is optionally substituted C₁₋₆alkyl;
   or a pharmaceutically acceptable salt or solvate thereof.

In some embodiments is a method for treating dyskinesia in a patient, comprising administering to the patient in need thereof a therapeutically effective amount of a compound of Formula (XVI):

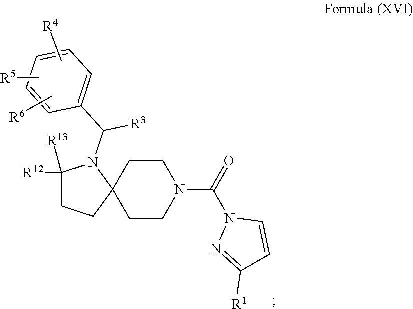

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₆alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring;
R¹² is H or C₁₋₆alkyl;
R¹³ is H or C₁₋₆alkyl; and
R¹⁵ is optionally substituted C₁₋₆alkyl;
   or a pharmaceutically acceptable salt or solvate thereof.

In some embodiments is method for treating dyskinesia with a compound of Formula (XVI), wherein R¹² and R¹³ are H.

In some embodiments is method for treating dyskinesia with a compound of Formula (XV) or (XVI), wherein R⁴ is optionally substituted heterocycloalkyl. In some embodiments is method for treating dyskinesia with a compound of Formula (XV) or (XVI), wherein R⁴ is heterocycloalkyl optionally substituted with one or more groups selected from halogen, hydroxy, C₁₋₆alkyl, —C₁₋₆alkyl-OH, C₁₋₆fluoroalkyl, C₃₋₆cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl. In some embodiments is method for treating dyskinesia with a compound of Formula (XV) or (XVI), wherein R⁴ is optionally substituted heterocycloalkyl and the heterocycloalkyl is a 4-6 membered monocyclic heterocycloalkyl, a 8-9 membered bicyclic heterocycloalkyl, a 7-8 membered bridged heterocycloalkyl, a 5,5 fused heterocycloalkyl, or an 8-11 membered spirocyclic heterocycloalkyl. In some embodiments is method for treating dyskinesia with a compound of Formula (XV) or (XVI), wherein R⁴ is

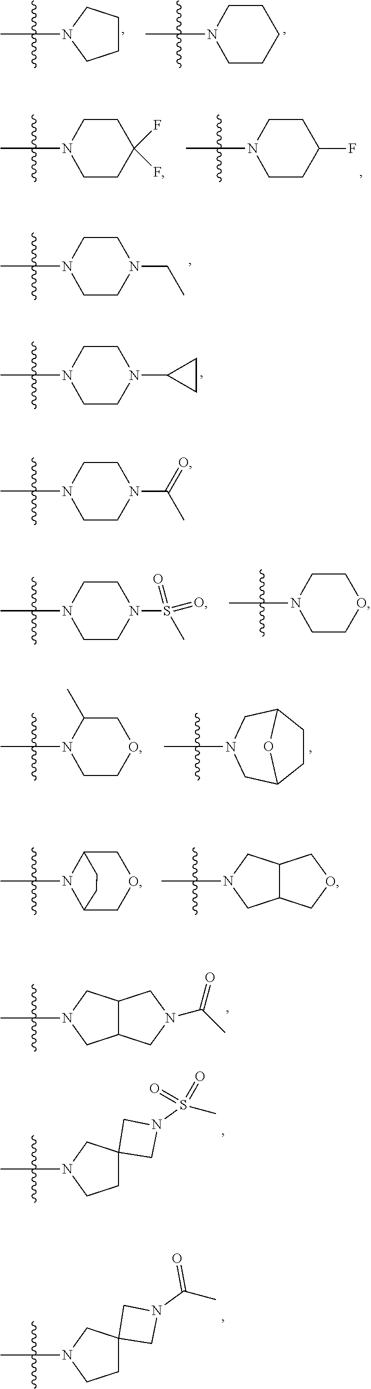

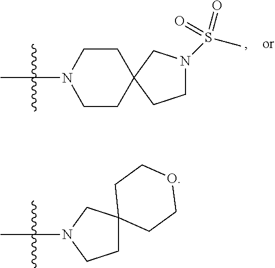 [SMILES: *N1CCC2(CC1)CCN(S(C)(=O)=O)C2 ; *N1CCC2(CCOCC2)C1]

In some embodiments is method for treating dyskinesia with a compound of Formula (XV) or (XVI), wherein R⁴ is

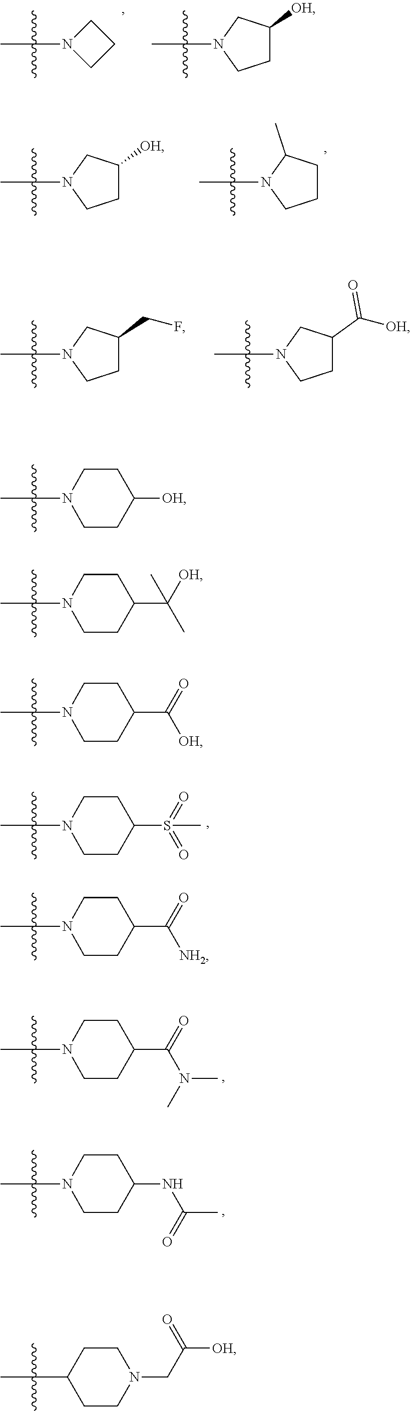

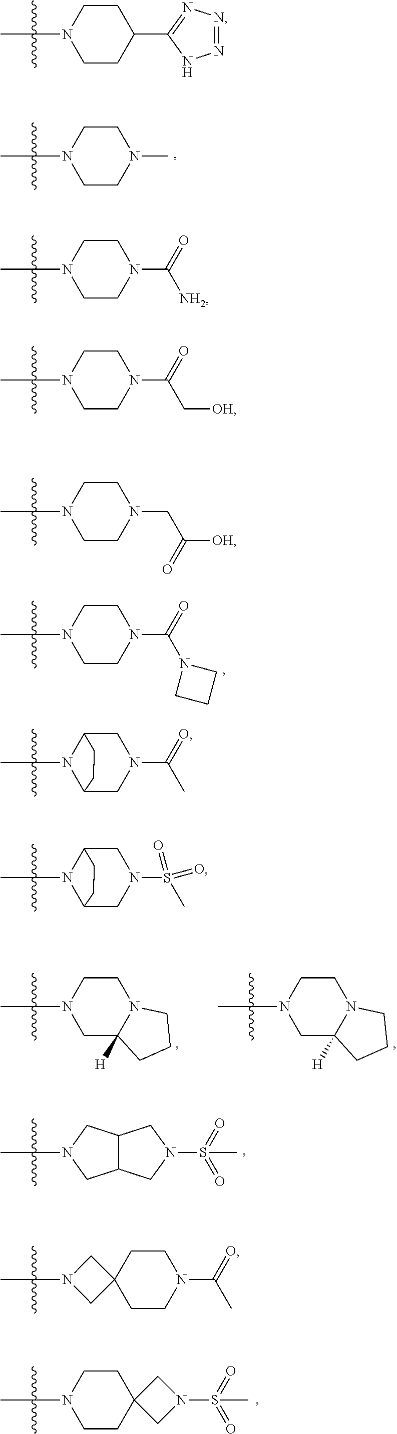

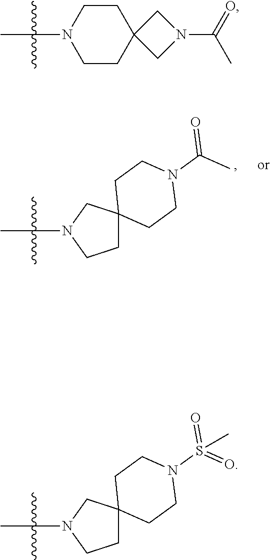 [SMILES: *N1CCC2(CC1)CN(C(C)=O)C2 ; *N1CCC2(CCN(C(C)=O)CC2)C1 ; *N1CCC2(CCN(S(C)(=O)=O)CC2)C1]

In some embodiments is method for treating dyskinesia with a compound of Formula (XV) or (XVI), wherein R⁴ is halogen. In some embodiments is method for treating dyskinesia with a compound of Formula (XV) or (XVI), wherein R⁴ is C₁₋₆haloalkyl. In some embodiments is method for treating dyskinesia with a compound of Formula (XV) or (XVI), wherein R⁵ is halogen. In some embodiments is method for treating dyskinesia with a compound of Formula (XV) or (XVI), wherein R⁵ is C₁₋₆haloalkyl. In some embodiments is method for treating dyskinesia with a compound of Formula (XV) or (XVI), wherein R⁵ is C₁₋₆ alkyl. In some embodiments is method for treating dyskinesia with a compound of Formula (XV) or (XVI), wherein R⁶ is H. In some embodiments is method for treating dyskinesia with a compound of Formula (XV) or (XVI), wherein R³ is H. In some embodiments is method for treating dyskinesia with a compound of Formula (XV) or (XVI), wherein R¹ is —N(R²)C(O)R¹⁵. In some embodiments is method for treating dyskinesia with a compound of Formula (XV) or (XVI), wherein R¹ is —N(H)SO₂R¹⁵. In some embodiments is method for treating dyskinesia with a compound of Formula (XV) or (XVI), wherein R¹⁵ is unsubstituted C₁₋₆alkyl.

In some embodiments is a compound of Formula (XVII):

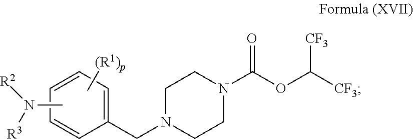

wherein:
each R¹ is independently halogen, C₁₋₆alkyl, C₁₋₆haloalkyl, C₁₋₆alkoxy, C₁₋₆haloalkoxy, C₃₋₈cycloalkyl, —OH, or —CN;
R² and R³, together with the carbon to which they are attached, form
   (i) a C₂-C₇heterocycloalkyl; or
   (ii) a C₂-C₉heteroaryl;
wherein the C₂-C₇heterocycloalkyl or the C₂-C₉heteroaryl is substituted with one R⁴ and optionally substituted with one or two additional substituents selected from halogen, C₁₋₆alkyl, C₁₋₆haloalkyl, and C₁₋₆alkoxy;
R⁴ is —CO₂H or —C₁₋₆alkyl-CO₂H; and
p is 0, 1, 2, 3, or 4;
   or a pharmaceutically acceptable salt or solvate thereof.

In some embodiments is method for treating dyskinesia with a compound of Formula (XVII), R² and R³, together with the carbon to which they are attached, form a C₂-C₇heterocycloalkyl substituted with one R⁴ and optionally substituted with one or two additional substituents selected from halogen, C₁₋₆alkyl, C₁₋₆haloalkyl, and C₁₋₆alkoxy. In some embodiments is method for treating dyskinesia with a compound of Formula (XVII), R⁴ is —CO₂H. In some embodiments is method for treating dyskinesia with a compound of Formula (XVII), R⁴ is —C₁₋₆alkyl-CO₂H. In some embodiments is method for treating dyskinesia with a compound of Formula (XVII), each R¹ is independently selected from halogen, C₁₋₆alkyl, and C₁₋₆haloalkyl. In some embodiments is method for treating dyskinesia with a compound of Formula (XVII), p is 1 or 2. In some embodiments is method for treating dyskinesia with a compound of Formula (XVII), p is 2. In some embodiments is method for treating dyskinesia with a compound of Formula (XVII), p is 1.

In some embodiments of the methods described herein for treating dyskinesia with a compound of Formula (I)-(XVII), the dyskinesia is levodopa-induced dyskinesia.

### BRIEF DESCRIPTION OF THE FIGURES

FIG. 1A depicts dyskinesia in MPTP-lesioned cynomolgus macaques dosed with amantadine (AMT) (10 mg/kg, p.o.) or vehicle following L-DOPA administration.

FIG. 1B depicts dyskinesia in MPTP-lesioned cynomolgus macaques dosed with amantadine (AMT) (10 mg/kg, p.o.) or vehicle following L-DOPA administration.

FIG. 1C depicts dyskinesia in MPTP-lesioned cynomolgus macaques dosed with Compound 21 (3, 10, and 30 mg/kg, p.o.) or vehicle following L-DOPA administration.

FIG. 1D depicts dyskinesia in MPTP-lesioned cynomolgus macaques dosed with Compound 21 (3, 10, and 30 mg/kg, p.o.) or vehicle following L-DOPA administration.

FIG. 1E depicts Parkinson disability in MPTP-lesioned cynomolgus macaques dosed with amantadine (AMT) (10 mg/kg, p.o.) or vehicle following L-DOPA administration.

FIG. 1F depicts Parkinson disability in MPTP-lesioned cynomolgus macaques dosed with Compound 21 (3, 10, and 30 mg/kg, p.o.) or vehicle following L-DOPA administration.

### DETAILED DESCRIPTION OF THE INVENTION

Dyskinesia is a type of hyperkinetic movement disorder. In Parkinson's disease, dyskinesia develops in response to long-term levodopa use and affects 90% of patients within approximately 10 years of treatment. Dyskinesia is characterized by involuntary, abnormal, purposeless movements and can be quite debilitating and disruptive to the patient. Dyskinesia can be broken down into subsets of hyperkinetic movements including chorea characterized by frequent, brief, unpredictable, purposeless movements flowing from body part to body part and dystonia which consists of intermittent muscle contractions causing abnormal, repetitive movements and postures. The clinical manifestation of dyskinesia can be categorized by the temporal occurrence after administration as peak-dose dyskinesias, biphasic dyskinesia and OFF dyskinesias.

Dyskinesia and hyperkinetic movements are also associated with other neurological disorders including tardive dyskinesia, Huntington's diseases, restless legs syndrome, tremor, traumatic brain injury and stroke.

This disclosure is directed, at least in part, to a method for treating dyskinesia with a MAGL inhibitor. In some embodiments described is a method for treating dyskinesia with a compound of Formula (I)-(XVII) described herein.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In certain embodiments, an alkyl comprises one to eight carbon atoms (e.g., C₁-C₆ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{a}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl or heteroarylalkyl.

“Alkoxy” refers to a radical bonded through an oxygen atom of the formula —O— alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In certain embodiments, an alkenyl comprises two to six carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{a}, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl, or heteroarylalkyl.

“Alkynyl” refers to a straight or branched hydrocarbon chain radical group consisting solely of carbon and hydrogen atoms, containing at least one carbon-carbon triple bond, and having from two to twelve carbon atoms. In certain embodiments, an alkenyl comprises two to eight carbon atoms. In certain embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is hydrogen, alkyl, fluoroalkyl, cycloalkyl, aryl, aralkyl, heterocycloalkyl, heteroaryl or heteroarylalkyl, and each R^{f} is independently alkyl, fluoroalkyl, cycloalkyl, aryl, aralkyl, heterocycloalkyl, heteroaryl, or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, and each R^{f} is independently alkyl, fluoroalkyl, carbocyclyl, carbocyclylalkyl, aryl, aralkyl, heterocyclyl, heterocyclylalkyl, heteroaryl or heteroarylalkyl.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR a, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R-aryl where R is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated, (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. In certain embodiments, a cycloalkyl comprises three to eight carbon atoms (e.g., C₃-C₈ cycloalkyl). In other embodiments, a cycloalkyl comprises three to seven carbon atoms (e.g., C₃-C₇ cycloalkyl). In other embodiments, a cycloalkyl comprises three to six carbon atoms (e.g., C₃-C₆ cycloalkyl). In other embodiments, a cycloalkyl comprises three to five carbon atoms (e.g., C₃-C₅ cycloalkyl). In other embodiments, a cycloalkyl comprises three to four carbon atoms (e.g., C₃-C₄ cycloalkyl). An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR a, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which includes fused or bridged ring systems. The heteroatoms in the heterocyclyl radical are optionally oxidized. One or more nitrogen atoms, if present, are optionally quaternized. The heterocyclyl radical is partially or fully saturated. In some embodiments, the heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR a, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated. The terms “heterocyclyl”, “heterocycle”, and “heterocycloalkyl” are used interchangeably.

“Heterocyclylalkyl” refers to a radical of the formula —R^{c}— heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{e}-heterocyclyl where R^{e} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR a, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O-heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{e}-heteroaryl, where R^{e} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

In some embodiments, he compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

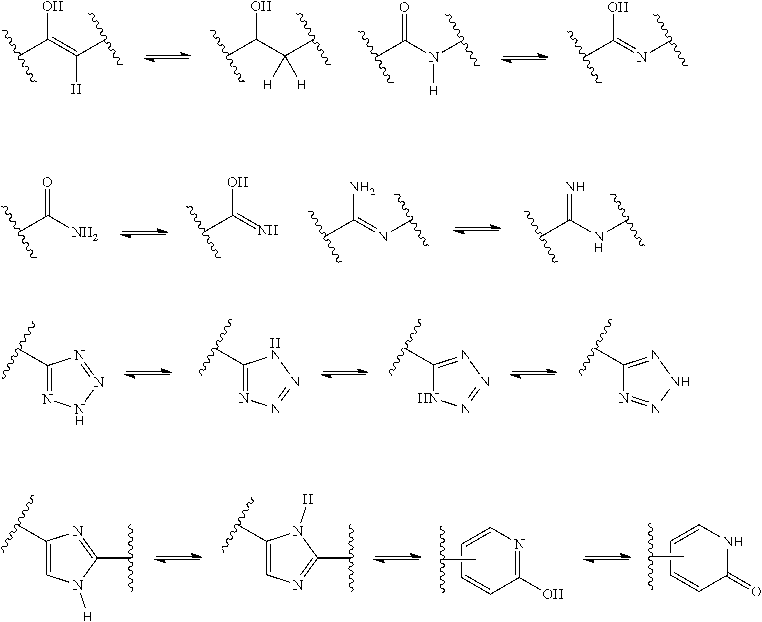

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

### Embodiments of the Invention

In the following, embodiments of the invention are disclosed. The first embodiment is denoted E1, the second embodiment E2 and so forth.

In a first embodiment E1 the present invention relates to a method of treating a disease with a compound of Formula (I).

E1: A method for treating dyskinesia in a patient in need thereof, comprising administering to the patient a therapeutically effective amount of a compound of Formula (I):

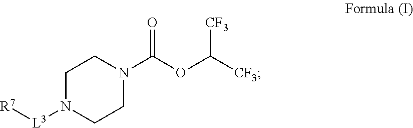

wherein:
L³ is a bond, —CH₂—, —S(O)₂—, or —C(O)—;
R⁷ is phenyl; wherein R⁷ is optionally substituted by one, two, or three moieties independently selected from R^{h};
R^{a} and R^{b} are independently selected, for each occurrence, from the group consisting of hydrogen and C₁₋₃alkyl; wherein C₁₋₃alkyl is optionally substituted by one or more substituents selected from halogen, cyano, oxo, hydroxyl, heterocycle, and phenyl;
or R^{a} and R^{b}, when they occur together with the nitrogen to which they are attached, form a 4-6 membered saturated heterocyclic ring, which may have an additional heteroatom selected from O, S, and N, or a spirocyclic ring selected from 8-oxa-2-azaspiro[4.5]decane and 2,8-diazaspiro[4.5]decane, wherein the 4-6 membered saturated heterocyclic ring or the spirocyclic ring are optionally substituted by one or more substituents selected from the group consisting of halogen, cyano, oxo, C₁₋₆ alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl;
R^{c} is selected from the group consisting of halogen, hydroxyl, C₁₋₆alkyl (optionally substituted by one, two, or three halogens), and C₁₋₆alkoxy (optionally substituted by one, two, or three halogens); and
R^{h} is selected from the group consisting of: halogen, phenyl (optionally substituted by one, two, or three moieties each independently selected from R^{c}), hydroxyl, cyano, C₁₋₆alkyl (optionally substituted by one, two or three halogens), C₁₋₆alkoxy (optionally substituted by one, two or three halogens), R^{a}R^{b}N—, R^{a}—C(O)NR^{a}—, R^{a}R^{b}N—SO₂—, R^{a}R^{b}N—C(O)—, R^{a}—S(O)_{w}— (wherein w is 0, 1 or 2), R^{a}—SO₂—NR^{b}—, and heteroaryl (optionally substituted by one, two or three moieties each independently selected from R^{c});
   or a pharmaceutically acceptable salt or solvate thereof.

E2: The method of embodiment 1, wherein L³ is a —CH₂—.

E3: The method of embodiment 1, wherein L³ is a —CH₂—; and R^{h} is selected from the group consisting of: halogen, phenyl (optionally substituted by one, two, or three moieties each independently selected from halogen, methyl, ethyl, propyl, t-butyl, and CF₃), C₁₋₆alkyl (optionally substituted by one, two or three halogens), C₁₋₆alkoxy (optionally substituted by one, two or three halogens), R^{a}R^{b}N—, R^{a}R^{b}N—C(O)—, and heteroaryl (optionally substituted by one, two or three moieties each independently selected from C₁₋₆alkyl or halogen).

E4: The method of embodiment 1, wherein L³ is a —CH₂—; and R^{h} is selected from the group consisting of: halogen, C₁₋₆alkyl (optionally substituted by one, two or three halogens), C₁₋₆alkoxy (optionally substituted by one, two or three halogens), and R^{a}R^{b}N—.

E5: The method of embodiment 1, wherein R⁷ is substituted by two moieties independently selected from R^{h}.

E6: The method of embodiment 1, wherein L³ is a —CH₂—; and R⁷ is substituted by R^{a}R^{b}N— and a moiety selected from the group consisting of: halogen, C₁₋₆alkyl (optionally substituted by one, two or three halogens), and C₁₋₆alkoxy (optionally substituted by one, two or three halogens).

E7: The method of embodiment 6, wherein R^{a} and R^{b}, together with the nitrogen to which they are attached, form a 4-6 membered saturated heterocyclic ring, which may have an additional heteroatom selected from O, S, and N, and the 4-6 membered saturated heterocyclic ring is optionally substituted by one or more substituents selected from the group consisting of halogen, cyano, oxo, C₁₋₆alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl.

E8: The method of embodiment 7, wherein the 4-6 membered saturated heterocyclic ring is selected from azetidine, pyrrolidine, piperidine, piperazine, and morpholine, and the 4-6 membered saturated heterocyclic ring is optionally substituted by one or more substituents selected from the group consisting of halogen, cyano, oxo, C₁₋₆alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl.

E9: The method of embodiment 7, wherein the 4-6 membered saturated heterocyclic ring is pyrrolidine.

E10: The method of embodiment 7, wherein the 4-6 membered saturated heterocyclic ring is morpholine.

E11: The method of embodiment 7, wherein the 4-6 membered saturated heterocyclic ring is piperidine.

E12: The method of embodiment 1, wherein L³ is a —CH₂—; and R^{h} is selected from the group consisting of: halogen, phenyl (optionally substituted by one, two, or three moieties each independently selected from halogen, methyl, ethyl, propyl, t-butyl, and CF₃), C₁₋₆alkyl (optionally substituted by one, two or three halogens), C₁₋₆alkoxy (optionally substituted by one, two or three halogens), and heteroaryl (optionally substituted by one, two or three moieties each independently selected from C₁₋₆alkyl or halogen).

E13: The method of embodiment 12, wherein R⁷ is substituted by two moieties independently selected from R^{h}.

E14: The method of embodiment 1, wherein L³ is a —CH₂—; and R^{h} is selected from the group consisting of: halogen, C₁₋₆alkyl (optionally substituted by one, two or three halogens), C₁₋₆alkoxy (optionally substituted by one, two or three halogens), and R^{a}R^{b}N—.

E15: The method of embodiment 14, wherein R⁷ is substituted by two moieties independently selected from R^{h}.

E16: The method of embodiment 1, wherein the compound of Formula (I) is selected from:

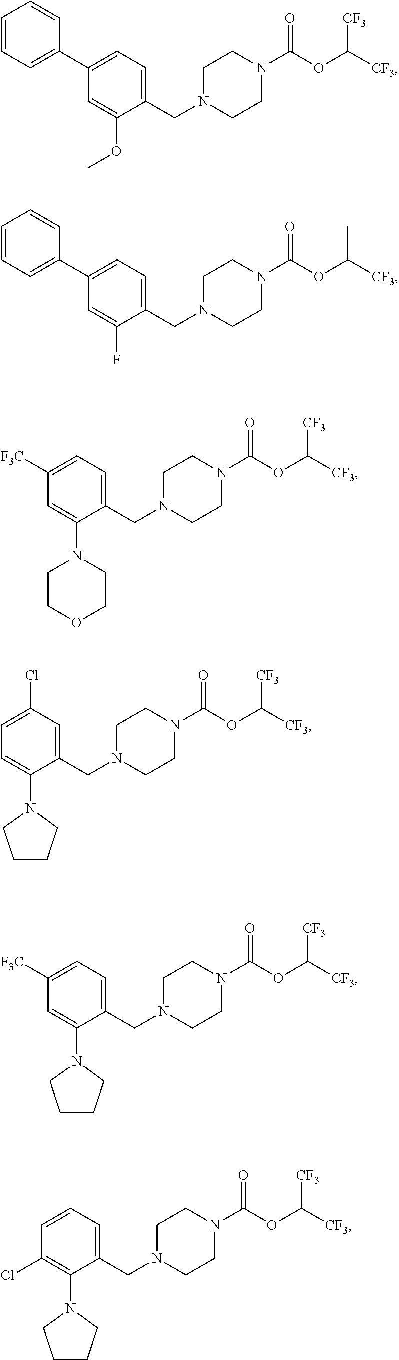 [SMILES: COc1cc(-c2ccccc2)ccc1CN1CCN(C(=O)OC(C(F)(F)F)C(F)(F)F)CC1 ; CC(OC(=O)N1CCN(Cc2ccc(-c3ccccc3)cc2F)CC1)C(F)(F)F ; O=C(OC(C(F)(F)F)C(F)(F)F)N1CCN(Cc2ccc(C(F)(F)F)cc2N2CCOCC2)CC1 ; O=C(OC(C(F)(F)F)C(F)(F)F)N1CCN(Cc2cc(Cl)ccc2N2CCCC2)CC1 ; O=C(OC(C(F)(F)F)C(F)(F)F)N1CCN(Cc2ccc(C(F)(F)F)cc2N2CCCC2)CC1 ; O=C(OC(C(F)(F)F)C(F)(F)F)N1CCN(Cc2cccc(Cl)c2N2CCCC2)CC1]

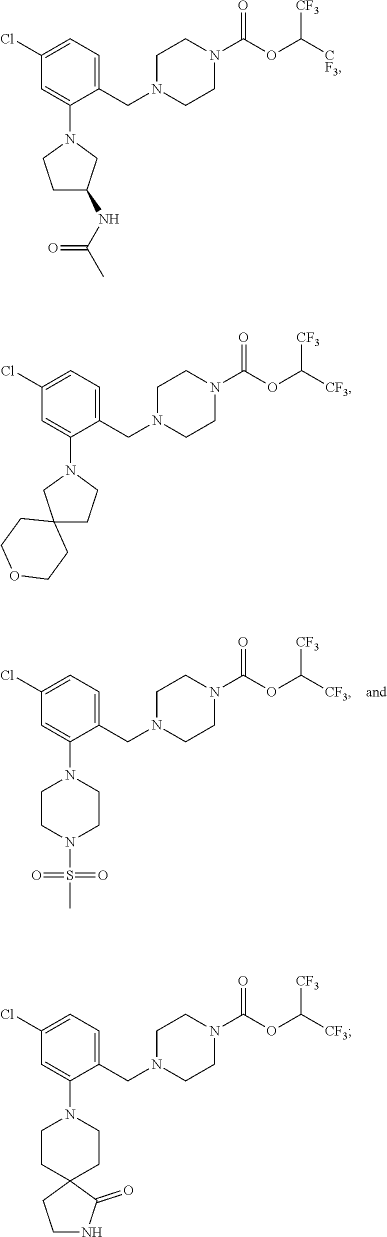 [SMILES: CC(=O)N[C@H]1CCN(c2cc(Cl)ccc2CN2CCN(C(=O)OC(C(F)(F)F)C(F)(F)F)CC2)C1 ; O=C(OC(C(F)(F)F)C(F)(F)F)N1CCN(Cc2ccc(Cl)cc2N2CCC3(CCOCC3)C2)CC1 ; CS(=O)(=O)N1CCN(c2cc(Cl)ccc2CN2CCN(C(=O)OC(C(F)(F)F)C(F)(F)F)CC2)CC1 ; "and" ; O=C(OC(C(F)(F)F)C(F)(F)F)N1CCN(Cc2ccc(Cl)cc2N2CCC3(CCNC3=O)CC2)CC1]

or a pharmaceutically acceptable salt or solvate thereof.

E17: A method for treating dyskinesia in a patient in need thereof, comprising administering to the patient a therapeutically effective amount of a compound of Formula (II):

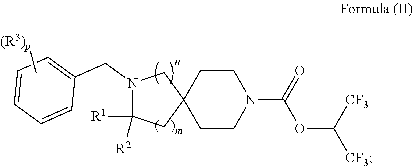

wherein:
R¹ is H or C₁₋₆alkyl;
R² is H or C₁₋₆alkyl;
each R³ is independently selected from C₁₋₆alkyl, C₂₋₆alkenyl, C₂₋₆alkynyl, halogen, —CN, C₁₋₆haloalkyl, C₁₋₆aminoalkyl, heterocycloalkyl, —C₁₋₆alkyl(heterocycloalkyl), heteroaryl, —SF₅, —NR⁵R⁶, —OR⁷, —CO₂R⁸, —C(O)R⁸, and —C(O)NR⁸R⁹, wherein heterocycloalkyl and —C₁₋₆alkyl(heterocycloalkyl) are optionally substituted with one or two R⁴; or two adjacent R³ form a heterocycloalkyl ring optionally substituted with one, two, or three R⁴;
each R⁴ is independently selected from C₁₋₆alkyl, C₁₋₆haloalkyl, C₃₋₈cycloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
each R⁵ and R⁶ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl; or R⁵ and R⁶, together with the nitrogen to which they are attached, form a heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl, wherein heterocycloalkyl, aryl, and heteroaryl are optionally substituted with one or two groups selected from oxo, C₁₋₆alkyl, C₁₋₆haloalkyl, CO₂H, and C(O)NH₂;
each R⁸ and R⁹ is independently selected from H, C₁₋₆alkyl, C₁₋₆haloalkyl, C₃₋₈cycloalkyl, aryl, and heteroaryl; or R⁸ and R⁹, together with the nitrogen to which they are attached, form a heterocycloalkyl ring optionally substituted with one or two groups selected from C₁₋₆alkyl, C₁₋₆haloalkyl, CO₂H, and C(O)NH₂;
each R¹⁰ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
p is 0, 1, 2, 3, 4, or 5;
n is 0 or 1; and
m is 1 or 2; provided that when n is 0, then m is 2; and when n is 1, then m is 1;
   or a pharmaceutically acceptable salt or solvate thereof.

E18: The method of embodiment 17, wherein each R³ is independently selected from C₁₋₆alkyl, C₂₋₆alkynyl, halogen, —CN, C₁₋₆haloalkyl, heterocycloalkyl, —C₁₋₆ alkyl(heterocycloalkyl), heteroaryl, —SF₅, —NR⁵R⁶, —OR⁷, —CO₂R⁸, and —C(O)NR⁸R⁹.

E19: The method of embodiment 17 or embodiment 18, wherein R¹ and R² are both H.

E20: The method of any one of embodiments 17-19, wherein each R³ is independently selected from halogen, C₁₋₆haloalkyl, —NR⁵R⁶, and —OR⁷.

E21: The method of any one of embodiments 17-20, wherein R⁵ and R⁶, together with the nitrogen to which they are attached, form a heterocycloalkyl ring optionally substituted with one or two R¹⁰ independently selected from C₁₋₆alkyl, C₃₋₈cycloalkyl, C₁₋₆ haloalkyl, halogen, —CO₂R⁸, —C(O)R⁸, —C(O)NR⁸R⁹, —SO₂R⁸, —NR⁹C(O)R⁸, and —NR⁹SO₂R⁸.

E22: The method of embodiment 21, wherein R⁵ and R⁶, together with the nitrogen to which they are attached, form a heterocycloalkyl ring substituted with one or two R¹⁰ independently selected from C₁₋₆alkyl and —CO₂H.

E23: The method of embodiment 21, wherein R⁵ and R⁶, together with the nitrogen to which they are attached, form an unsubstituted heterocycloalkyl ring.

E24: The method of any one of embodiments 17-20, wherein R⁵ and R⁶, together with the nitrogen to which they are attached, form a heterocycloalkyl ring optionally substituted with one, two, or three R¹⁰ selected from:

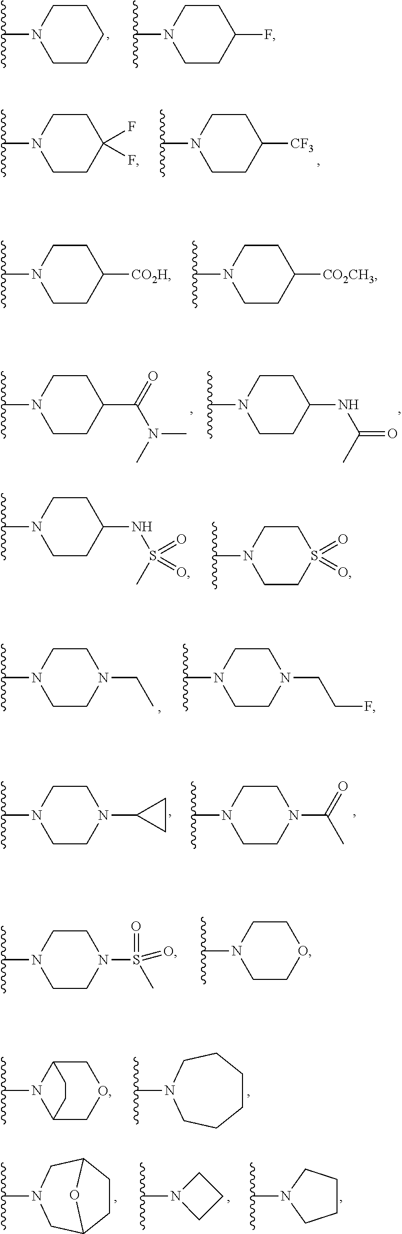

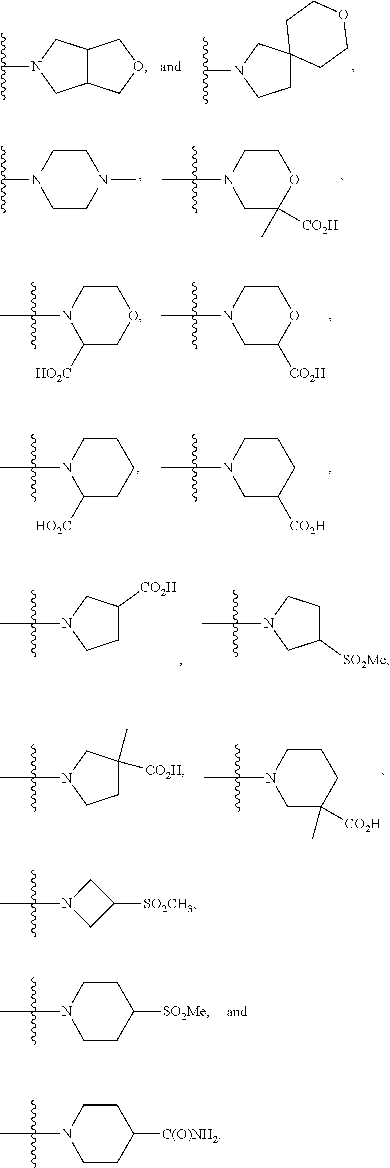

E25: The method of any one of embodiments 17-24, wherein p is 1 or 2.

E26: The method of any one of embodiments 17-25, wherein n is 0 and m is 2.

E27: The method of embodiment 17, wherein the compound of Formula (II) is selected from:

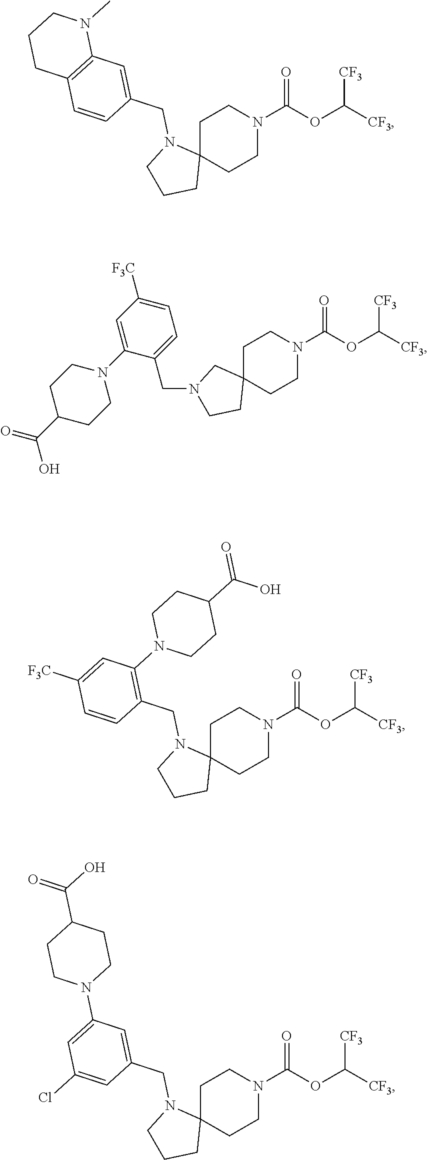 [SMILES: CN1CCCc2ccc(CN3CCCC34CCN(C(=O)OC(C(F)(F)F)C(F)(F)F)CC4)cc21 ; O=C(O)C1CCN(c2cc(C(F)(F)F)ccc2CN2CCC3(CCN(C(=O)OC(C(F)(F)F)C(F)(F)F)CC3)C2)CC1 ; O=C(O)C1CCN(c2cc(C(F)(F)F)ccc2CN2CCCC23CCN(C(=O)OC(C(F)(F)F)C(F)(F)F)CC3)CC1 ; O=C(O)C1CCN(c2cc(Cl)cc(CN3CCCC34CCN(C(=O)OC(C(F)(F)F)C(F)(F)F)CC4)c2)CC1]

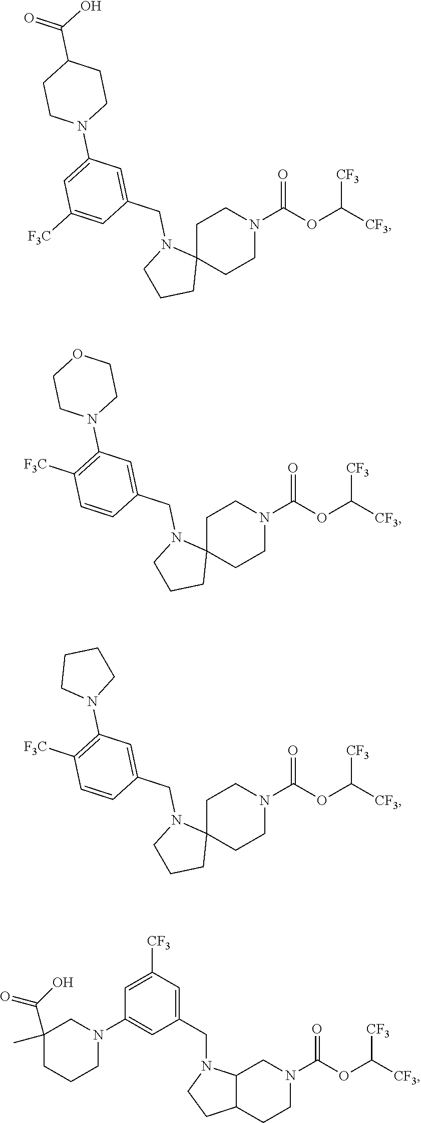 [SMILES: O=C(O)C1CCN(c2cc(CN3CCCC34CCN(C(=O)OC(C(F)(F)F)C(F)(F)F)CC4)cc(C(F)(F)F)c2)CC1 ; O=C(OC(C(F)(F)F)C(F)(F)F)N1CCC2(CCCN2Cc2ccc(C(F)(F)F)c(N3CCOCC3)c2)CC1 ; O=C(OC(C(F)(F)F)C(F)(F)F)N1CCC2(CCCN2Cc2ccc(C(F)(F)F)c(N3CCCC3)c2)CC1 ; CC1(C(=O)O)CCCN(c2cc(CN3CCC4CCN(C(=O)OC(C(F)(F)F)C(F)(F)F)CC43)cc(C(F)(F)F)c2)C1]

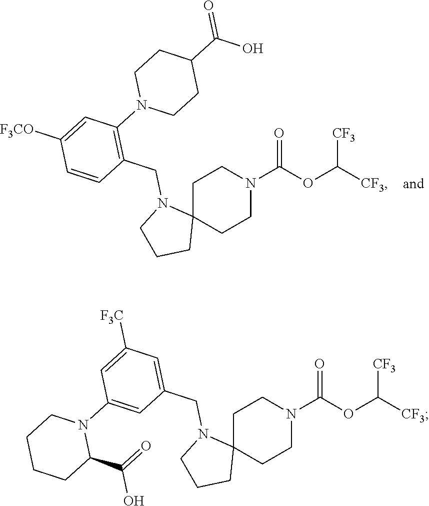 [SMILES: O=C(O)C1CCN(c2cc(OC(F)(F)F)ccc2CN2CCCC23CCN(C(=O)OC(C(F)(F)F)C(F)(F)F)CC3)CC1 ; "and" ; O=C(O)[C@H]1CCCCN1c1cc(CN2CCCC23CCN(C(=O)OC(C(F)(F)F)C(F)(F)F)CC3)cc(C(F)(F)F)c1]

or a pharmaceutically acceptable salt or solvate thereof.

E28: A method for treating dyskinesia in a patient in need thereof, comprising administering to the patient a therapeutically effective amount of a compound of Formula (III):

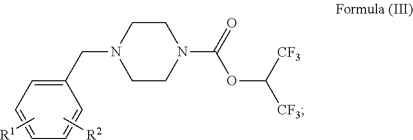

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

E29: The method of embodiment 28, wherein R¹ is halogen, —SF₅, or optionally substituted C₁₋₆alkyl optionally substituted by halogen.

E30: The method of embodiment 28 or embodiment 29, wherein R⁵ and R⁶, together with the nitrogen to which they are attached, form a 4-6 membered saturated monocyclic heterocycle, wherein the 4-6 membered saturated monocyclic heterocycle is substituted with one substituent selected from C₁₋₆haloalkyl, —C(O)OR⁹, and —NR⁹SO₂R⁸; and the 4-6 membered saturated monocyclic heterocycle optionally contains an additional O, N, or S.

E31: The method of embodiment 30, wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine, piperidine, and morpholine.

E32: The method of embodiment 28 or embodiment 29, wherein R⁵ and R⁶, together with the nitrogen to which they are attached, form a 7-8 membered bridged heterocyclic ring optionally substituted with one or two substituents independently selected from halogen, oxo, and C₁₋₆alkyl.

E33: The method of embodiment 28, wherein the compound of Formula (III) is selected from:

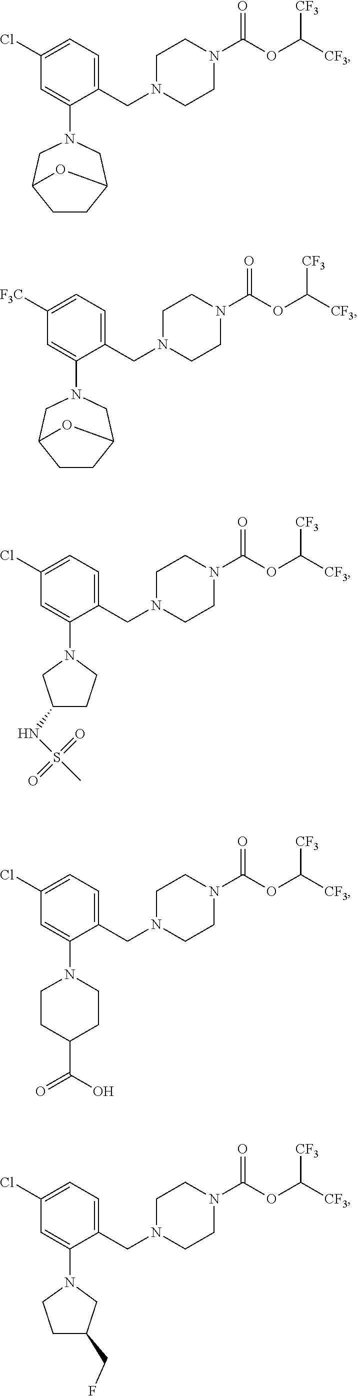 [SMILES: O=C(OC(C(F)(F)F)C(F)(F)F)N1CCN(Cc2ccc(Cl)cc2N2CC3CCC(C2)O3)CC1 ; O=C(OC(C(F)(F)F)C(F)(F)F)N1CCN(Cc2ccc(C(F)(F)F)cc2N2CC3CCC(C2)O3)CC1 ; CS(=O)(=O)N[C@H]1CCN(c2cc(Cl)ccc2CN2CCN(C(=O)OC(C(F)(F)F)C(F)(F)F)CC2)C1 ; O=C(O)C1CCN(c2cc(Cl)ccc2CN2CCN(C(=O)OC(C(F)(F)F)C(F)(F)F)CC2)CC1 ; O=C(OC(C(F)(F)F)C(F)(F)F)N1CCN(Cc2ccc(Cl)cc2N2CC[C@H](CF)C2)CC1]

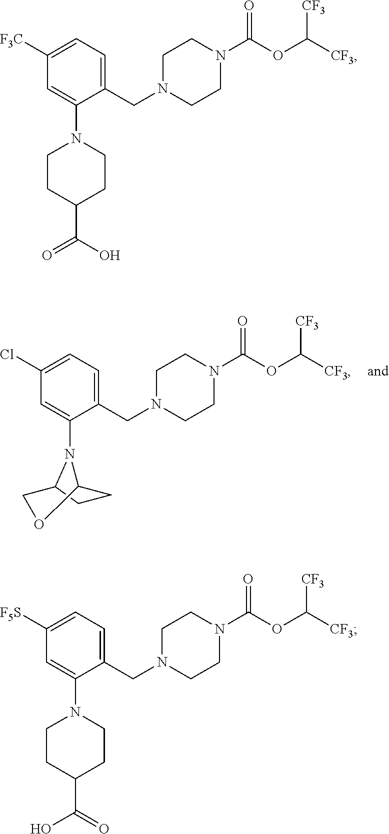

or a pharmaceutically acceptable salt or solvate thereof.

E34: A method for treating dyskinesia in a patient in need thereof, comprising administering to the patient a therapeutically effective amount of a compound of Formula (IV):

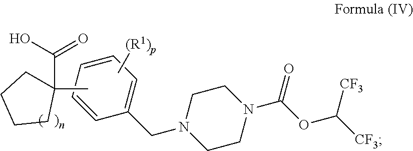

wherein:
each R¹ is independently halogen, C₁₋₆alkyl, C₁₋₆haloalkyl, C₁₋₆alkoxy, C₁₋₆haloalkoxy, C₃₋₈cycloalkyl, —OH, —CN, or —SF₅;
n is 1 or 2; and
p is 0, 1, 2, 3, or 4;
   or a pharmaceutically acceptable salt or solvate thereof.

E35: The method of embodiment 34, wherein n is 1.

E36: A method for treating dyskinesia in a patient in need thereof, comprising administering to the patient a therapeutically effective amount of a compound of Formula (V):

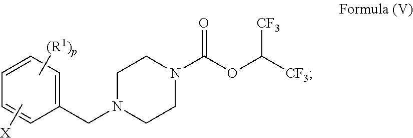

wherein:
X is —N(R²)(R³), —C₁₋₆alkyl-N(R⁴)(R⁵), —C(O)N(R⁴)(R⁵),

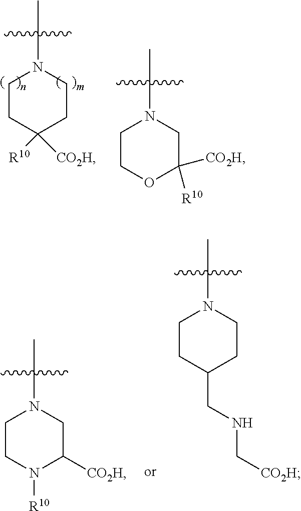

each R¹ is independently halogen, C₁₋₆alkyl, C₁₋₆haloalkyl, C₁₋₆alkoxy, C₁₋₆ haloalkoxy, C₃₋₈cycloalkyl, —OH, —CN, or —SF₅;
R² and R³, together with the nitrogen to which they are attached, form
   (i) a C₂-C₈heterocycloalkyl; or
   (ii) a C₂-C₈heteroaryl;
wherein the C₂-C₈heterocycloalkyl or the C₂-C₈heteroaryl is substituted with one R⁶ and optionally substituted with one or two additional substituents selected from halogen, C₁₋₆alkyl, C₁₋₆haloalkyl, and C₁₋₆alkoxy;
R⁴ and R⁵, together with the nitrogen to which they are attached, form
   (i) a C₂-C₈heterocycloalkyl; or
   (ii) a C₂-C₈heteroaryl;
wherein the C₂-C₈heterocycloalkyl or the C₂-C₈heteroaryl is substituted with one R⁷ and optionally substituted with one or two additional substituents selected from halogen, C₁₋₆alkyl, C₁₋₆haloalkyl, and C₁₋₆alkoxy;
R⁶ is —C₁₋₆alkyl-CO₂H or —N(R⁸)—C₁₋₆alkyl-CO₂H;
R⁷ is —CO₂H, —C₁₋₆alkyl-CO₂H, or —N(R⁹)—C₁₋₆alkyl-CO₂H;
R⁸ is H or C₁₋₆alkyl;
R⁹ is H or C₁₋₆alkyl;
R¹⁰ is C₁₋₆alkyl;
m is 0, 1, or 2;
n is 0 or 1; and
p is 0, 1, 2, 3, or 4;
   or a pharmaceutically acceptable salt or solvate thereof.

E37: The method of embodiment 36, wherein X is

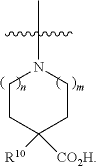

E38: The method of embodiment 37, wherein m is 1 and n is 1.

E39: The method of embodiment 36, wherein X is —N(R²)(R³).

E40: The method of embodiment 39, wherein R² and R³, together with the nitrogen to which they are attached, form a C₂-C₈heterocycloalkyl substituted with one R⁶.

E41: The method of embodiment 40, wherein R² and R³, together with the nitrogen to which they are attached, form a C₂-C₈heterocycloalkyl selected from:

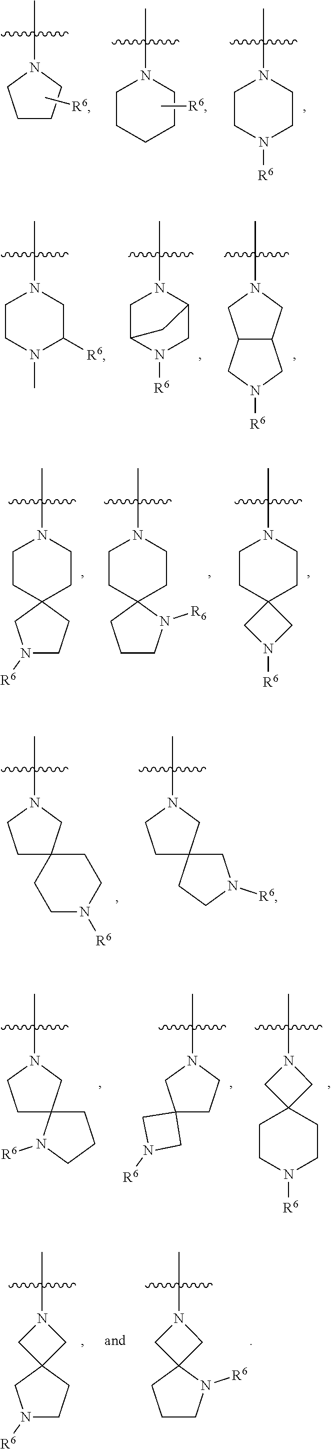

E42: The method of embodiment 40 or embodiment 41, wherein R⁶ is —C₁₋₆alkyl-CO₂H.

E43: The method of any one of embodiments 34-42, wherein each R¹ is independently halogen.

E44: The method of any one of embodiments 34-42, wherein each R¹ is independently C₁₋₆haloalkyl.

E45: The method of any one of embodiments 34-42, wherein each R¹ is independently C₁₋₆alkyl.

E46: The method of any one of embodiments 34-45, wherein p is 1.

E47: The method of embodiment 34, wherein the compound of Formula (IV) is selected from:

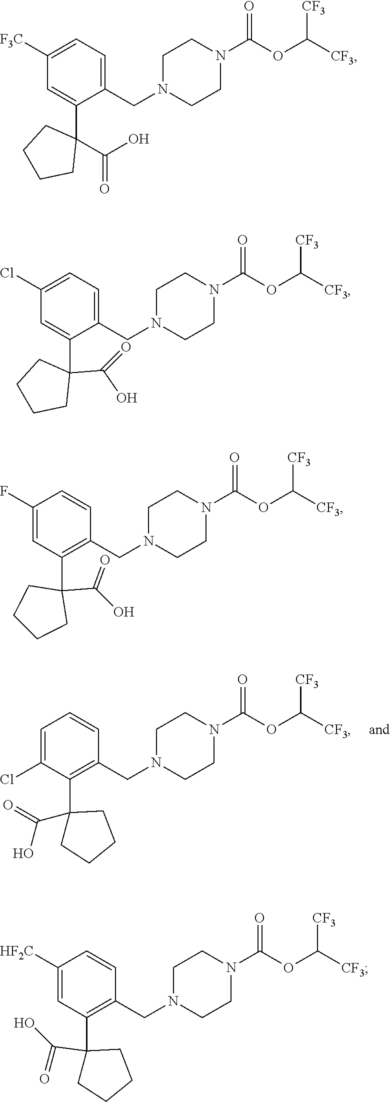

or a pharmaceutically acceptable salt or solvate thereof.

E48: The method of embodiment 36, wherein the compound of Formula (V) is selected from:

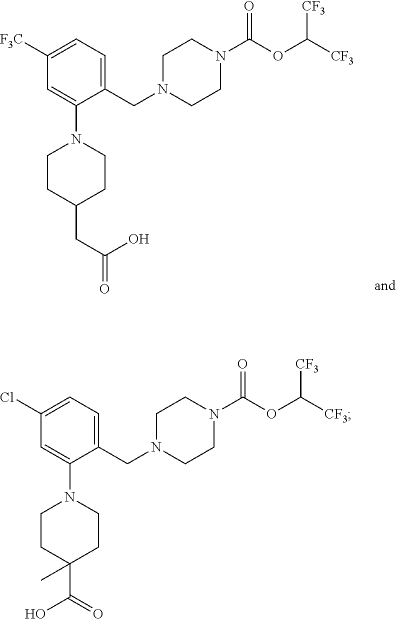 [SMILES: O=C(O)CC1CCN(c2cc(C(F)(F)F)ccc2CN2CCN(C(=O)OC(C(F)(F)F)C(F)(F)F)CC2)CC1 ; CC1(C(=O)O)CCN(c2cc(Cl)ccc2CN2CCN(C(=O)OC(C(F)(F)F)C(F)(F)F)CC2)CC1]

or a pharmaceutically acceptable salt or solvate thereof.

E49: A method for treating dyskinesia in a patient in need thereof, comprising administering to the patient a therapeutically effective amount of a compound of Formula (VI):

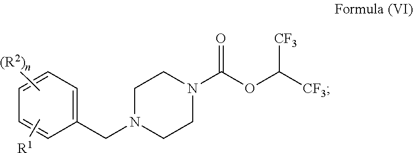

wherein:
R¹ is —N(R³)(R⁵) or —NH(R⁴);
each R² is independently selected from halogen, C₁₋₆alkyl, —CN, C₁₋₆haloalkyl, and —OR⁶;
R³ is —CH₂CO₂H, —CH₂CH₂CO₂H, or —CH(CH₃)CO₂H;
R⁴ is —(CH₂)ₘ—CO₂H;
R⁵ is H or C₁₋₃alkyl;
each R⁶ is independently selected from H, C₁₋₆alkyl, and C₁₋₆haloalkyl;
n is 0, 1, 2, 3, or 4; and
m is 3;
   or a pharmaceutically acceptable salt or solvate thereof.

E50: The method of embodiment 49, wherein R¹ is —N(R³)(R⁵).

E51: The method of embodiment 50, wherein R⁵ is H.

E52: The method of embodiment 49, wherein R¹ is —NH(R⁴).

E53: The method of any one of embodiments 49-52, wherein each R² is independently selected from halogen, C₁₋₆alkyl, and C₁₋₆haloalkyl.

E54: The method of any one of embodiments 49-53, wherein n is 1.

E55: The method of embodiment 49, wherein the compound of Formula (VI) is selected from:

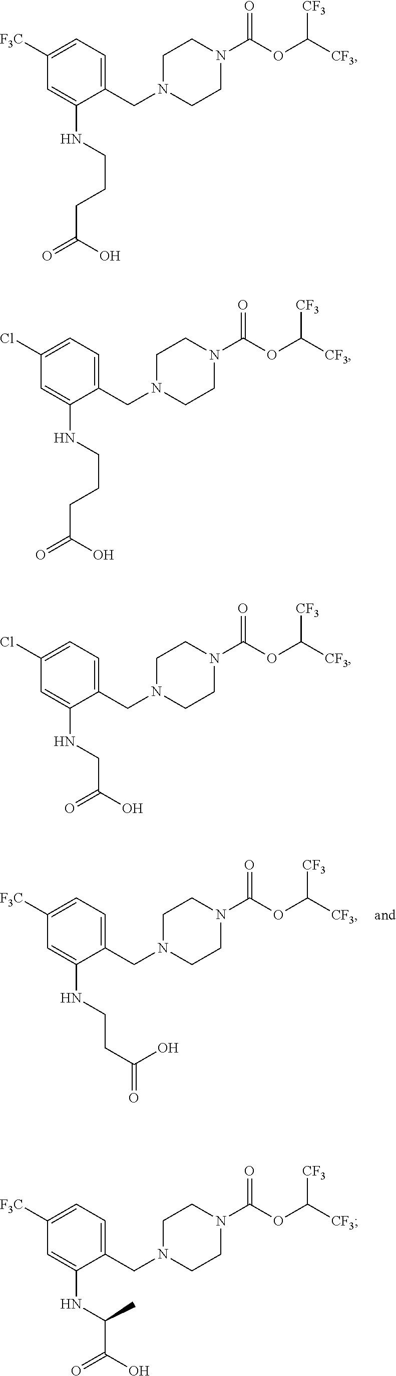 [SMILES: O=C(O)CCCNc1cc(C(F)(F)F)ccc1CN1CCN(C(=O)OC(C(F)(F)F)C(F)(F)F)CC1 ; O=C(O)CCCNc1cc(Cl)ccc1CN1CCN(C(=O)OC(C(F)(F)F)C(F)(F)F)CC1 ; O=C(O)CNc1cc(Cl)ccc1CN1CCN(C(=O)OC(C(F)(F)F)C(F)(F)F)CC1 ; O=C(O)CCNc1cc(C(F)(F)F)ccc1CN1CCN(C(=O)OC(C(F)(F)F)C(F)(F)F)CC1 ; "and" ; C[C@H](Nc1cc(C(F)(F)F)ccc1CN1CCN(C(=O)OC(C(F)(F)F)C(F)(F)F)CC1)C(=O)O]

or a pharmaceutically acceptable salt or solvate thereof.

E56: A method for treating dyskinesia in a patient in need thereof, comprising administering to the patient a therapeutically effective amount of a compound of Formula (VII):

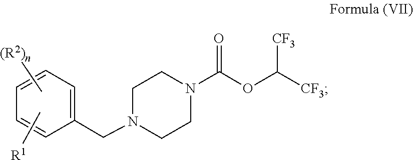

wherein:
R¹ is —R¹⁴, —OR³, —SR⁴, —S(O)₂R⁴, or —C≡C—(CR⁶R)—R⁸;
each R² is independently selected from C₁₋₆alkyl, halogen, —CN, C₁₋₆haloalkyl, —C₁₋₆alkyl(heterocycloalkyl), —OR¹⁷, and —C(O)NR¹⁸R¹⁹;
R³ is —(CR⁶R⁷)ₘ—R⁸, —(CR⁶R⁷)ₚ—Y—(CR⁶R⁷)_{q}—R⁸, or —(CR⁶R⁷)ₜ—C₃₋₆cycloalkyl-R⁸;
R⁴ is —(CR⁶R⁷)ₘ—R⁸, —(CR⁶R⁷)—C(O)OH, or —(CR⁶R⁷)ₚ—Y—(CR⁶R⁷)_{q}—R⁸;
Y is —O— or —N(R²²)—;
each R⁶ and R⁷ is each independently selected from H, F, and C₁₋₆alkyl; or R⁶ and R⁷, together with the carbon to which they are attached, form a C₃₋₆cycloalkyl ring;
R⁸ is —C(O)OR⁹, —C(O)R¹⁰, or —C(O)O—(CR¹²R¹³)—OC(O)R¹¹;
R^{8′} is —C(O)OR^{9′}, —C(O)R^{10′}, or —C(O)O—(CR¹²R¹³)—OC(O)R¹¹;
R⁹ is H or C₁₋₆alkyl;
R^{9′} is C₁₋₆alkyl;
R¹⁰ is C₁₋₆alkyl or —NHSO₂R²¹;
R^{10′} is C₂₋₆alkyl or —NHSO₂R²¹;
R¹¹ is C₁₋₆alkyl or C₁₋₆alkoxy;
R¹² and R¹³ is each independently H or C₁₋₆alkyl;
R¹⁴ is —(CR¹⁵R¹⁶)ₘ—R⁸ or —(CR⁶R⁷)ₚ—Y—(CR⁶R⁷)_{q}—R⁸;
each R¹⁵ and R¹⁶ is each independently selected from H, F, and C₁₋₆alkyl;
each R¹⁷ is independently selected from H, C₁₋₆alkyl, C₁₋₆haloalkyl, and C₃₋₆cycloalkyl;
each R¹⁸ and R¹⁹ is each independently selected from H, C₁₋₆alkyl, C₃₋₆cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl or C₃₋₆cycloalkyl;
R²² is H, C₁₋₆alkyl, or —SO₂R²³;
R²³ is C₁₋₆alkyl;
m is 1, 2, 3 or 4;
n is 0, 1, 2, 3, or 4;
p is 2, 3, or 4;
q is 1, 2, or 3;
t is 0, 1, or 2; and
v is 3 or 4;
   or a pharmaceutically acceptable salt or solvate thereof.

E57: The method of embodiment 56, wherein R¹ is —OR³.

E58: The method of embodiment 57, wherein R³ is —(CR⁶R⁷)ₘ—R⁸.

E59: The method of embodiment 58, wherein m is 1, 2, or 3.

E60: The method of embodiment 59, wherein each R⁶ and R⁷ is each independently selected from H and C₁₋₆alkyl, or R⁶ and R⁷, together with the carbon to which they are attached, form a C₃₋₆cycloalkyl ring.

E61: The method of embodiment 60, wherein R⁸ is —C(O)OR⁹.

E62: The method of embodiment 61, wherein R⁹ is H.

E63: The method of any one of embodiments 56-62, wherein each R² is independently selected from C₁₋₆alkyl, halogen, and C₁₋₆haloalkyl.

E64: The method of embodiment 63, wherein n is 2.

E65: The method of embodiment 63, wherein n is 1.

E66: The method of embodiment 56, wherein the compound of Formula (VII) is selected from:

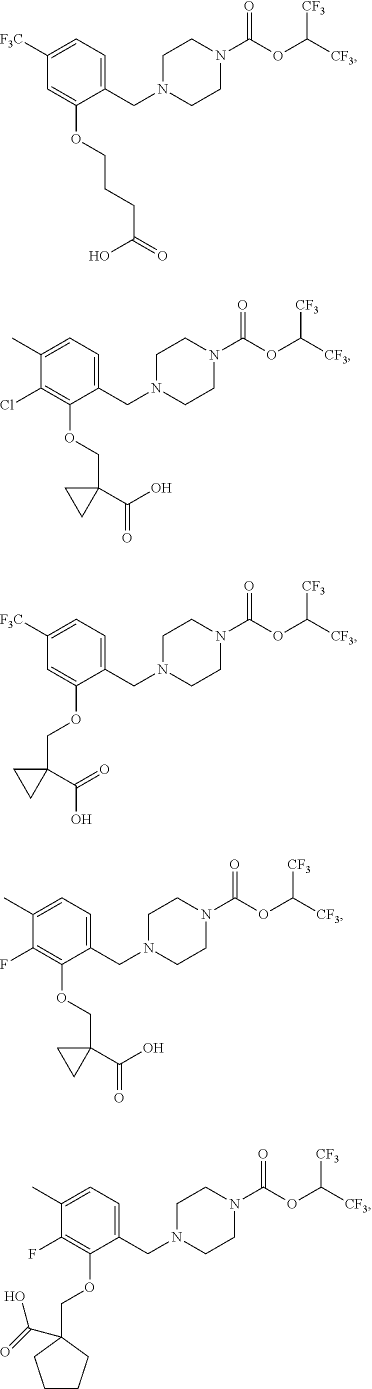 [SMILES: O=C(O)CCCOc1cc(C(F)(F)F)ccc1CN1CCN(C(=O)OC(C(F)(F)F)C(F)(F)F)CC1 ; Cc1ccc(CN2CCN(C(=O)OC(C(F)(F)F)C(F)(F)F)CC2)c(OCC2(C(=O)O)CC2)c1Cl ; O=C(OC(C(F)(F)F)C(F)(F)F)N1CCN(Cc2ccc(C(F)(F)F)cc2OCC2(C(=O)O)CC2)CC1 ; Cc1ccc(CN2CCN(C(=O)OC(C(F)(F)F)C(F)(F)F)CC2)c(OCC2(C(=O)O)CC2)c1F ; Cc1ccc(CN2CCN(C(=O)OC(C(F)(F)F)C(F)(F)F)CC2)c(OCC2(C(=O)O)CCCC2)c1F]

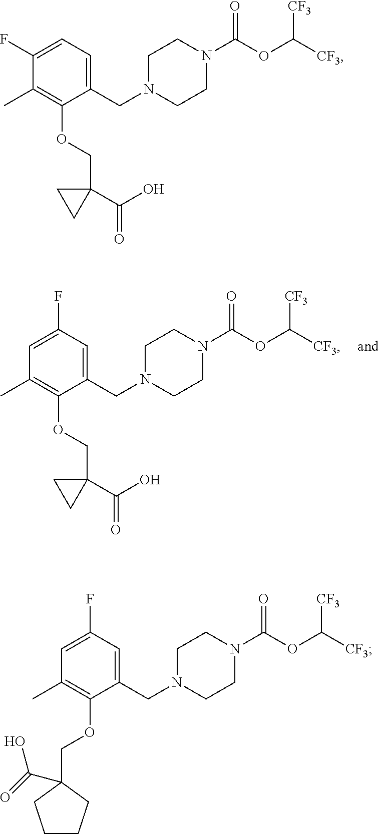 [SMILES: Cc1c(F)ccc(CN2CCN(C(=O)OC(C(F)(F)F)C(F)(F)F)CC2)c1OCC1(C(=O)O)CC1 ; Cc1cc(F)cc(CN2CCN(C(=O)OC(C(F)(F)F)C(F)(F)F)CC2)c1OCC1(C(=O)O)CC1 ; "and" ; Cc1cc(F)cc(CN2CCN(C(=O)OC(C(F)(F)F)C(F)(F)F)CC2)c1OCC1(C(=O)O)CCCC1]

or a pharmaceutically acceptable salt or solvate thereof.

E67: A method for treating dyskinesia in a patient in need thereof, comprising administering to the patient a therapeutically effective amount of a compound of Formula (VIII):

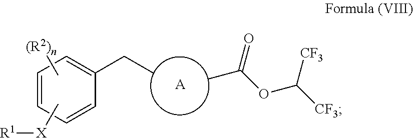

wherein:

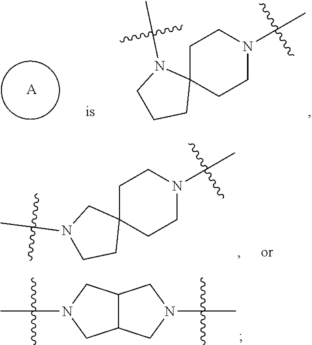

X is —O—, —S—, —SO₂—, —N(R³)—, or —CH₂—;
Y is —O— or —N(R⁷)—;
R¹ is —(CR⁴R⁵)ₘ—R⁶, —(CR⁴R⁵)ₚ—Y—(CR⁴R⁵)_{q}—R⁶, or —(CR⁴R⁵)ₜ—C₃₋₆cycloalkyl-R⁶;
each R² is independently selected from halogen, —CN, C₁₋₆alkyl, C₁₋₆haloalkyl, —C₁₋₆alkyl(heterocycloalkyl), —OR¹⁷, and —C(O)NR¹⁸R¹⁹;
R³ is H or C₁₋₆alkyl;
each R⁴ and R⁵ is each independently selected from H, F, and C₁₋₆alkyl; or R⁴ and R⁵, together with the carbon to which they are attached, form a C₃₋₆cycloalkyl ring;
R⁶ is —CO₂R⁹, —C(O)R¹⁰, or —C(O)O—(CR¹²R¹³)—OC(O)R¹¹;
R⁷ is H, C₁₋₆alkyl, or —SO₂R⁸;
R⁸ is C₁₋₆alkyl;
R⁹ is H or C₁₋₆alkyl;
R¹⁰ is C₁₋₆alkyl or —NHSO₂R²¹;
R¹¹ is C₁₋₆alkyl or C₁₋₆alkoxy;
R¹² and R¹³ is each independently H or C₁₋₆alkyl;
each R¹⁷ is independently selected from H, C₁₋₆alkyl, C₁₋₆haloalkyl, aminoalkyl, cycloalkyl, —C₁₋₆alkyl(heterocycloalkyl), —C₁₋₆alkyl-C(O)(heterocycloalkyl), optionally substituted heterocycloalkyl, optionally substituted aryl, and optionally substituted heteroaryl;
each R¹⁸ and R¹⁹ is independently selected from H, C₁₋₆alkyl, C₁₋₆haloalkyl, cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl;
m is 1, 2, 3 or 4;
n is 0, 1, 2, 3, or 4;
p is 2, 3, or 4;
q is 1, 2, or 3; and
t is 0, 1, or 2;
   or a pharmaceutically acceptable salt or solvate thereof.

E68: The method of embodiment 67, wherein R¹ is —(CR⁴R⁵)ₘ—R⁶.

E69: The method of embodiment 67 or embodiment 68, wherein each R⁴ and R⁵ is each independently selected from H and C₁₋₆alkyl.

E70: The method of any one of embodiments 67-69, wherein each R⁴ and R⁵ is H.

E71: The method of any one of embodiments 67-70, wherein R⁶ is —CO₂R⁹.

E72: The method of any one of embodiments 67-71, wherein R⁹ is H.

E73: The method of any one of embodiments 67-70, wherein R⁶ is —C(O)R¹⁰.

E74: The method of embodiment 73, wherein R¹⁰ is —NHSO₂R²¹.

E75: The method of any one of embodiments 67-74, wherein X is —O—.

E76: The method of any one of embodiments 67-74, wherein X is —N(R³)—.

E77: The method of any one of embodiments 67-76, wherein

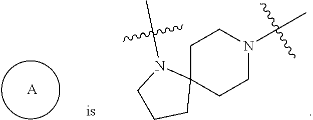

E78: The method of any one of embodiments 67-76, wherein

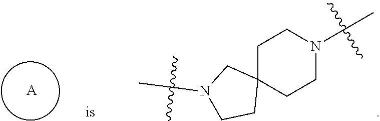

E79: The method of any one of embodiments 67-78, wherein each R² is independently selected from halogen, C₁₋₆alkyl, and C₁₋₆haloalkyl.

E80: The method of any one of embodiments 67-79, wherein n is 1.

E81: The method of embodiment 67, wherein the compound of Formula (VIII) is selected from:

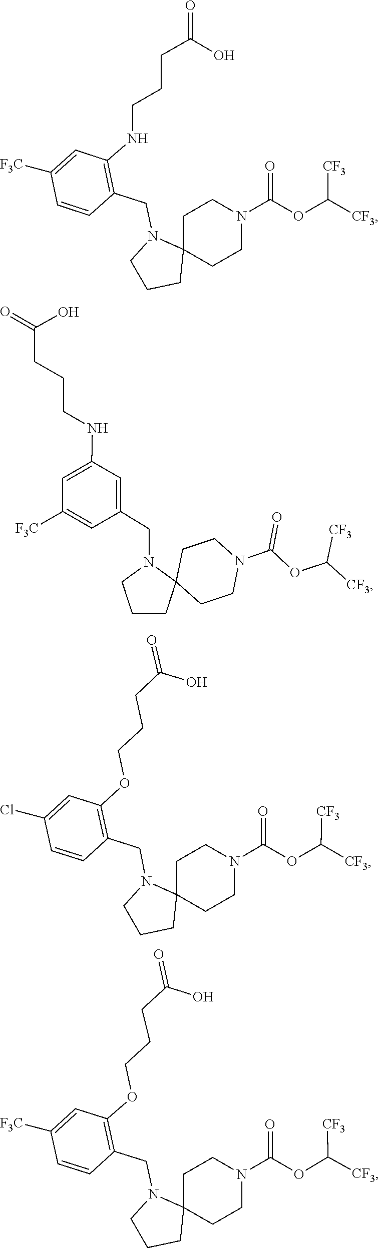

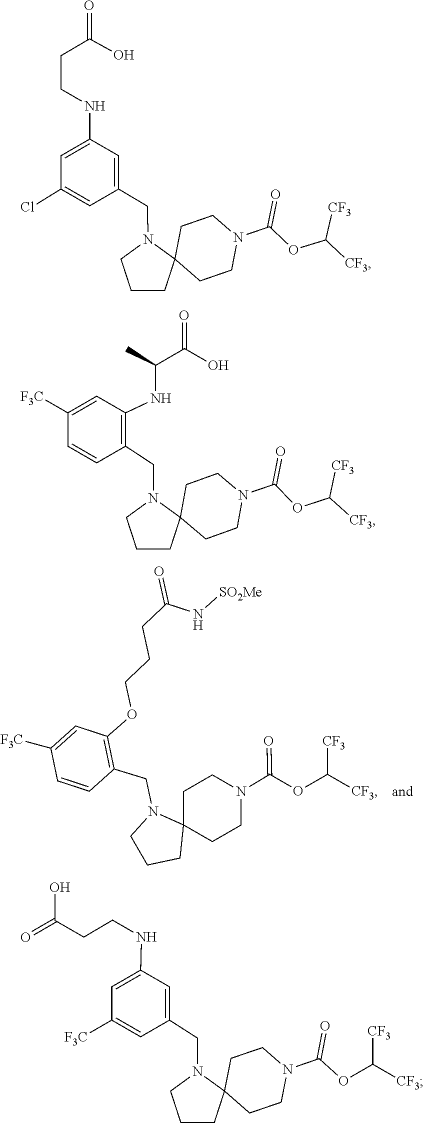

or a pharmaceutically acceptable salt or solvate thereof.

E82: A method for treating dyskinesia in a patient in need thereof, comprising administering to the patient a therapeutically effective amount of a compound of Formula (IX):

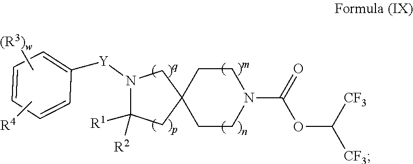

wherein:
Y is —CH₂— or —C(O)—;
R¹ is H or C₁₋₆alkyl;
R² is H or C₁₋₆alkyl;
each R³ is independently selected from C₁₋₆alkyl, halogen, —CN, C₁₋₆haloalkyl, —SF₅, and —OR⁷;
R⁴ is selected from —C≡C—C₁₋₆alkyl-CO₂H and —C₃₋₈cycloalkyl-CO₂H;
each R⁷ is independently selected from H, C₁₋₆alkyl, C₁₋₆haloalkyl, C₁₋₆aminoalkyl, C₃₋₈cycloalkyl, and —C₁₋₆alkyl-C₃₋₈cycloalkyl;
w is 0, 1, 2, 3, or 4;
n is 0 or 1;
m is 0 or 1;
p is 0, 1, or 2; and
q is 0, 1, or 2; provided that when q is 0, then p is 2;
   or a pharmaceutically acceptable salt or solvate thereof.

E83: The method of embodiment 82, wherein R⁴ is —C₃₋₈cycloalkyl-CO₂H.

E84: The method of embodiment 83, wherein R⁴ is

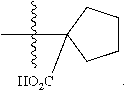 [SMILES: *C1(C(=O)O)CCCC1]

85: The method of embodiment 82, wherein R⁴ is —C≡C—C₁₋₆alkyl-CO₂H.

E86: The method of embodiment 85, wherein R⁴ is

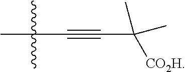 [SMILES: *C#CC(C)(C)C(=O)O]

E87: The method of any one of embodiments 82-86, wherein Y is —CH₂—.

E88: The method of any one of embodiments 82-87, wherein R¹ and R² are both H.

E89: The method of any one of embodiments 82-88, wherein each R³ is independently selected from halogen and C₁₋₆haloalkyl.

E90: The method of any one of embodiments 82-89, wherein w is 1.

E91: The method of any one of embodiments 82-90, wherein m is 1, n is 1, q is 0, and p is 2.

E92: The method of embodiment 82, wherein the compound of Formula (IX) is selected from:

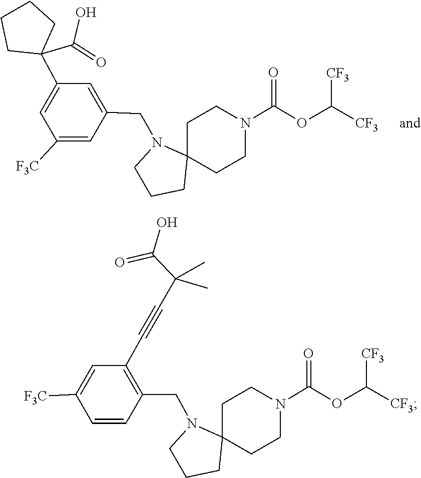

or a pharmaceutically acceptable salt or solvate thereof.

E93: A method for treating dyskinesia in a patient in need thereof, comprising administering to the patient a therapeutically effective amount of a compound of Formula (X):

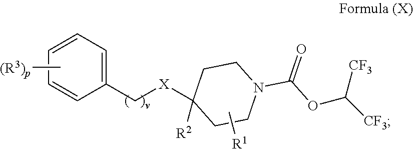

wherein:
X is —O— or —N(R¹¹)—;
R¹ is H or C₁₋₆alkyl;
R² is C₁₋₆alkyl;
each R³ is independently selected from C₁₋₆alkyl, C₂₋₆alkenyl, C₂₋₆alkynyl, —C≡C—C₁₋₆ alkyl-CO₂H, halogen, —CN, C₁₋₆haloalkyl, C₁₋₆aminoalkyl, C₃₋₈cycloalkyl, —C₁₋₆alkyl(C₂₋₉heterocycloalkyl), C₁₋₉heteroaryl, —SF₅, —NR⁵R⁶, —OR⁷, —CO₂R⁸, and —C(O)NR⁸R⁹, wherein C₃₋₈cycloalkyl, —C₁₋₆alkyl(C₂₋₉heterocycloalkyl), and C₁₋₉ heteroaryl are optionally substituted with one or two R⁴; or two adjacent R³ form a C₂₋₉heterocycloalkyl ring, wherein the C₂₋₉heterocycloalkyl ring is optionally substituted with one, two, or three R⁴;
each R⁴ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
each R⁵ and R⁶ is independently selected from H, C₁₋₆alkyl, C₁₋₆haloalkyl, C₁₋₆ aminoalkyl, C₃₋₈cycloalkyl, —C₁₋₆alkyl(C₂₋₉heterocycloalkyl), —C₁₋₆alkyl-C(O)(C₂₋₉heterocycloalkyl), C₂₋₉heterocycloalkyl, C₆₋₁₀aryl, and C₁₋₉heteroaryl; or R⁵ and R⁶, together with the nitrogen to which they are attached, form a C₂₋₉ heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(C₂₋₉heterocycloalkyl), —C₁₋₆alkyl-C(O)(C₂₋₉ heterocycloalkyl), —C₁₋₆alkyl-CO₂H, C₂₋₉heterocycloalkyl, C₆₋₁₀ aryl, and C₁₋₉ heteroaryl, wherein C₂₋₉heterocycloalkyl, C₆₋₁₀aryl, and C₁₋₉heteroaryl are optionally substituted with one or two groups selected from oxo, C₁₋₆alkyl, C₁₋₆ haloalkyl, CO₂H, and CO₂NH₂;
each R⁸ and R⁹ is independently selected from H, C₁₋₆alkyl, C₁₋₆haloalkyl, C₃₋₈cycloalkyl, C₆₋₁₀aryl, and C₁₋₉heteroaryl; or R⁸ and R⁹, together with the nitrogen to which they are attached, form a C₂₋₉heterocycloalkyl ring optionally substituted with one or two groups selected from C₁₋₆alkyl, C₁₋₆haloalkyl, CO₂H, and CO₂NH₂;
each R¹⁰ is independently selected from halogen, C₁₋₆alkyl, C₁₋₆haloalkyl, C₃₋₈cycloalkyl, oxo, —CN, —CO₂R⁸, —C(O)R⁸, —C(O)NR⁸R⁹, —SO₂R⁸, —NR⁹C(O)R⁸, and —NR⁹SO₂R⁸;
R¹¹ is H, C₁₋₆alkyl, —C(O)—C₁₋₆alkyl, or —CH₂CO₂H;
p is 0, 1, 2, 3, 4, or 5; and
v is 0 or 1;
   or a pharmaceutically acceptable salt or solvate thereof.

E94: The method of embodiment 93, wherein each R³ is independently selected from halogen, C₁₋₆haloalkyl, —NR⁵R⁶, and —OR⁷.

E95: The method of embodiment 93 or embodiment 94, wherein R⁵ and R⁶, together with the nitrogen to which they are attached, form a C₂₋₉heterocycloalkyl ring optionally substituted with one, two, or three R¹⁰.

E96: The method of embodiment 95, wherein R⁵ and R⁶, together with the nitrogen to which they are attached, form a C₂₋₉heterocycloalkyl ring substituted with one or two R¹⁰ independently selected from C₁₋₆alkyl and —CO₂H.

E97: The method of embodiment 95, wherein R⁵ and R⁶, together with the nitrogen to which they are attached, form an unsubstituted C₂₋₉heterocycloalkyl ring.

E98: The method of embodiment 93 or embodiment 94, wherein R⁵ and R⁶, together with the nitrogen to which they are attached, form a C₂₋₉heterocycloalkyl ring selected from:

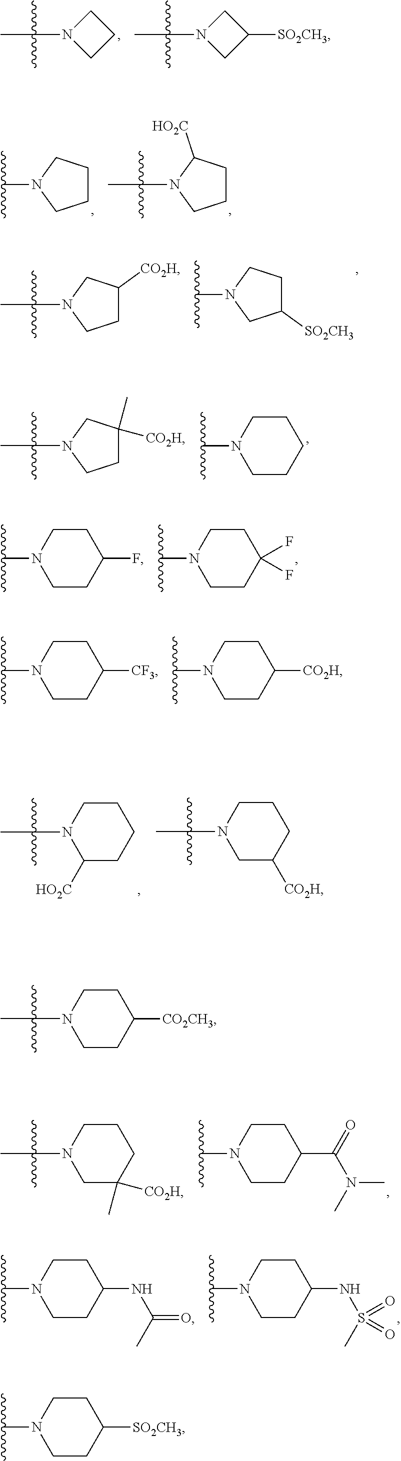

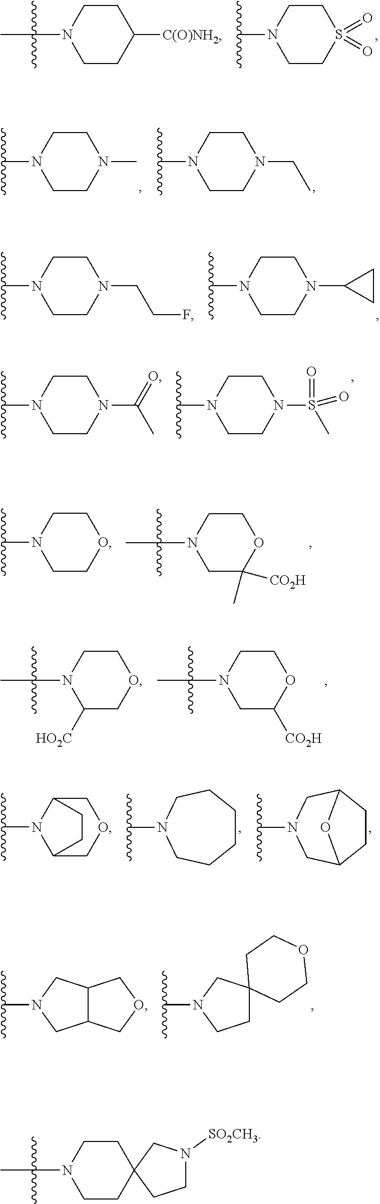

E99: The method of embodiment 93, wherein the compound of Formula (X) is selected from:

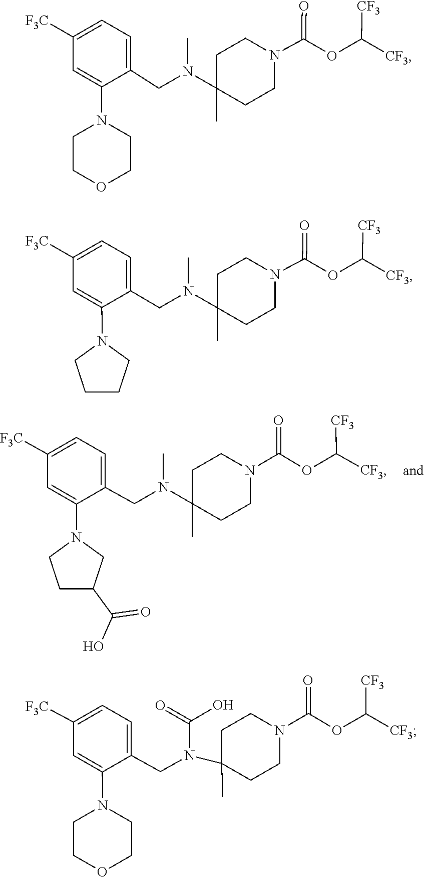

or a pharmaceutically acceptable salt or solvate thereof.

E100: A method for treating dyskinesia in a patient in need thereof, comprising administering to the patient a therapeutically effective amount of a compound of Formula (XI)

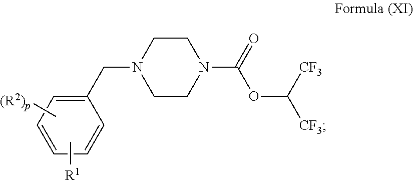

wherein:
R¹ is selected from

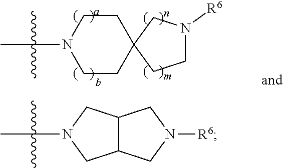

each R² is independently selected from C₁₋₆alkyl, halogen, —CN, C₁₋₆haloalkyl, C₃₋₈cycloalkyl, —SF₅, —OR³, and —C(O)NR⁴R⁵;
each R³ is independently selected from H, C₁₋₆alkyl, C₁₋₆haloalkyl, C₃₋₈cycloalkyl, and —C₁₋₆alkyl-C₃₋₈cycloalkyl;
each R⁴ and R⁵ is independently selected from H, C₁₋₆alkyl, and C₃₋₈cycloalkyl; R⁶ is selected from C₁₋₆alkyl, —C(O)—C₁₋₆alkyl, and —S(O)₂—C₁₋₆alkyl;
a is 0 or 1;
b is 0 or 1;
m is 0, 1, or 2;
n is 0, 1, or 2; provided that when n is 0, then m is 2; and
p is 0, 1, 2, 3, or 4;
   or a pharmaceutically acceptable salt or solvate thereof.

E101: The method of embodiment 100, wherein R¹ is

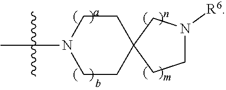

E102: The method of embodiment 100 or embodiment 101, wherein R⁶ is —C(O)—C₁₋₆alkyl.

E103: The method of embodiment 100 or embodiment 101, wherein R⁶ is —S(O)₂—C₁₋₆alkyl.

E104: The method of any one of embodiments 100-103, wherein each R³ is independently selected from halogen and C₁₋₆haloalkyl.

E105: The method of any one of embodiments 100-104, wherein p is 1.

E106: The method of embodiment 100, wherein the compound of Formula (XI) is selected from:

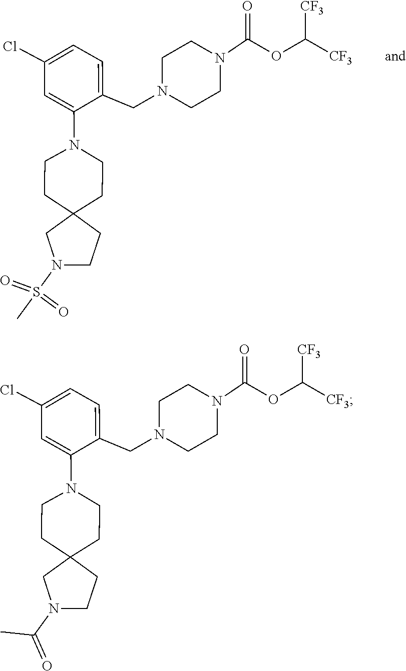 [SMILES: CS(=O)(=O)N1CCC2(CCN(c3cc(Cl)ccc3CN3CCN(C(=O)OC(C(F)(F)F)C(F)(F)F)CC3)CC2)C1 ; "and" ; CC(=O)N1CCC2(CCN(c3cc(Cl)ccc3CN3CCN(C(=O)OC(C(F)(F)F)C(F)(F)F)CC3)CC2)C1]

or a pharmaceutically acceptable salt or solvate thereof.

E107: A method for treating dyskinesia in a patient in need thereof, comprising administering to the patient a therapeutically effective amount of a compound of Formula (XII):

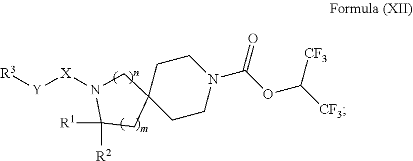

wherein:
X is —CH₂— or —C(O)—;
Y is a bond, C₁₋₆alkyl, C₁₋₆haloalkyl, or C₃₋₈cycloalkyl;
R¹ is H or C₁₋₆alkyl;
R² is H or C₁₋₆alkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, —CH₂CO₂R⁶, and —CH₂C(O)N(H)SO₂R⁸; wherein C₂₋₉heterocycloalkyl, —C₁₋₆alkyl(C₂₋₉heterocycloalkyl), phenyl, and C₁₋₉ heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl;
each R⁸ is independently selected from C₁₋₆alkyl, C₁₋₆haloalkyl, and C₃₋₈cycloalkyl;
n is 0 or 1; and
m is 1 or 2; provided that when n is 0, then m is 2; and when n is 1, then m is 1;
   or a pharmaceutically acceptable salt or solvate thereof.

E108: The method of embodiment 107, wherein Y is a bond.

E109: The method of embodiment 107 or embodiment 108, wherein R¹ and R² are both H.

E110: The method of any one of embodiments 107-109, wherein X is —CH₂—.

E111: The method of any one of embodiments 107-109, wherein X is —C(O)—.

E112: The method of any one of embodiments 107-111, wherein n is 0 and m is 2.

E113: A method for treating dyskinesia in a patient in need thereof, comprising administering to the patient a therapeutically effective amount of a compound of Formula (XIII):

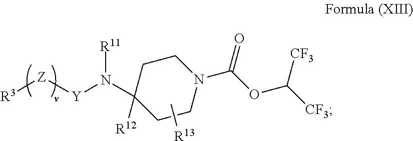

wherein:
Y is —CH₂— or —C(O)—;
Z is C₃₋₆cycloalkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, and —CH₂CO₂R⁶; wherein C₂₋₉heterocycloalkyl, —C₁₋₆alkyl(C₂₋₉heterocycloalkyl), phenyl, and C₁₋₉heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl;
R¹¹ is H, C₁₋₆alkyl, or —C₁₋₆alkyl-O—C₁₋₆alkyl;
R¹² is C₁₋₆alkyl;
R¹³ is H or C₁₋₆alkyl; and
v is 0 or 1;
   or a pharmaceutically acceptable salt or solvate thereof.

E114: The method of embodiment 113, wherein R¹³ is H.

E115: The method of embodiment 113 or embodiment 114, wherein v is 0.

E116: The method of any one of embodiments 113-115, wherein Y is —C(O)—.

E117: The method of any one of embodiments 107-116, wherein R³ is a 5-membered heteroaryl ring substituted with one, two, or three R⁴.

E118: The method of embodiment 117, wherein R³ is a 5-membered heteroaryl ring substituted with two or three R⁴, wherein two adjacent R⁴ form a 6-membered heterocycloalkyl ring optionally substituted with one or two R⁵.

E119: The method of embodiment 118, wherein R³ is a 5-membered heteroaryl ring substituted with two adjacent R⁴, wherein the two adjacent R⁴ form an unsubstituted 6-membered heterocycloalkyl ring.

E120: The method of embodiment 118, wherein R³ is a 5-membered heteroaryl ring substituted with two adjacent R⁴, wherein the two adjacent R⁴ form a 6-membered heterocycloalkyl ring substituted with one R⁵.

E121: The method of embodiment 120, wherein R⁵ is selected from C₁₋₆alkyl, C₁₋₆heteroalkyl, C₃₋₈cycloalkyl, —C₁₋₆alkyl(C₃₋₈cycloalkyl), C₂₋₉heterocycloalkyl, and —CH₂CO₂H.

E122: The method of any one of embodiments 107-116, wherein R³ is selected from:

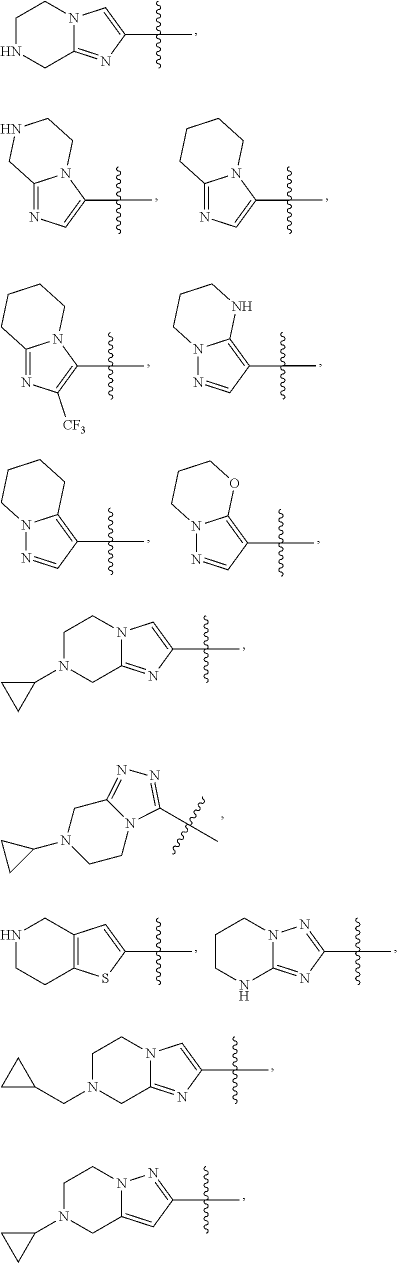

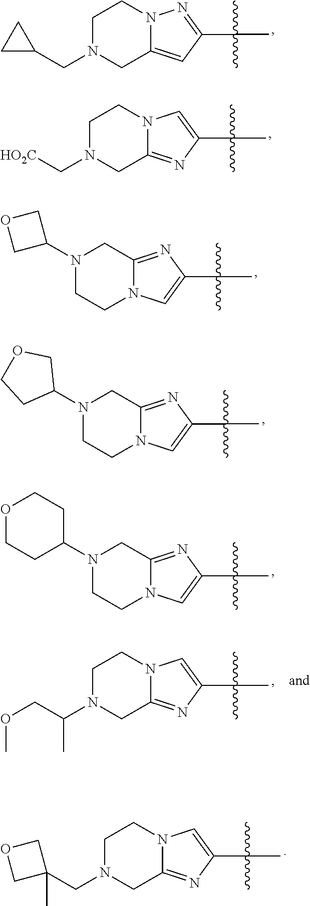

E123: The method of embodiment 107, wherein the compound of Formula (XII) is:

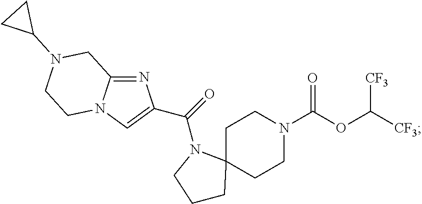 [SMILES: O=C(OC(C(F)(F)F)C(F)(F)F)N1CCC2(CCCN2C(=O)c2cn3c(n2)CN(C2CC2)CC3)CC1]

or a pharmaceutically acceptable salt or solvate thereof.

E124: The method of embodiment 113, wherein the compound of Formula (XIII) is selected from:

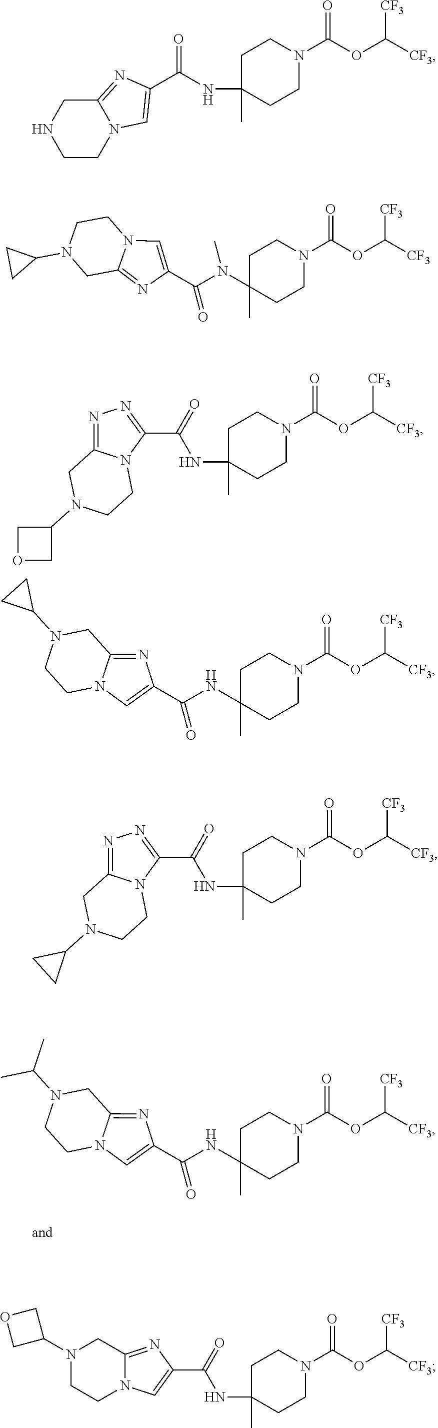

or a pharmaceutically acceptable salt or solvate thereof.

E125: A method for treating dyskinesia in a patient in need thereof, comprising administering to the patient a therapeutically effective amount of a compound of Formula (XIV):

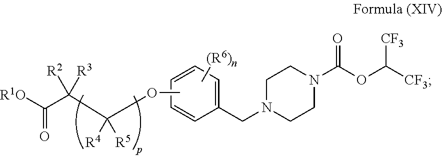

wherein:
R¹ is H or C₁₋₆alkyl;
R² is C₁₋₆alkyl;
R³ is H or C₁₋₆alkyl;
R⁴ and R⁵ are independently selected from H and C₁₋₆alkyl;
each R⁶ is independently selected from C₁₋₆alkyl, halogen, —CN, C₁₋₆haloalkyl, —OR⁷, —C(O)NR⁸R⁹, C₃₋₆cycloalkyl, C₂₋₉heterocycloalkyl, —C₁₋₆alkyl(C₂₋₉heterocycloalkyl), and C₂₋₉heteroaryl, wherein C₃₋₆cycloalkyl, C₂₋₉heterocycloalkyl, —C₁₋₆alkyl(C₂₋₉heterocycloalkyl), and C₂₋₉heteroaryl are optionally substituted with one, two, or three groups independently selected from halogen, C₁₋₆alkyl, C₁₋₆haloalkyl, and C₁₋₆alkoxy;
each R⁷ is independently selected from H, C₁₋₆alkyl, C₁₋₆haloalkyl, and C₃₋₆cycloalkyl;
each R⁸ and R⁹ is each independently selected from H, C₁₋₆alkyl, C₃₋₆cycloalkyl, aryl, and heteroaryl; or R⁸ and R⁹, together with the nitrogen to which they are attached, form a heterocycloalkyl ring optionally substituted with one, two, or three R¹⁰;
each R¹⁰ is independently selected from halogen, C₁₋₆alkyl, C₁₋₆haloalkyl, oxo, —CN, and C₃₋₆cycloalkyl;
n is 0, 1, 2, 3, or 4; and
p is 0 or 1;
   or a pharmaceutically acceptable salt or solvate thereof.

E126: The method of embodiment 125, wherein p is 0.

E127: The method of embodiment 125, wherein p is 1.

E128: The method of any one of embodiments 125-127, wherein R⁴ and R⁵ are H.

E129: The method of any one of embodiments 125-128, wherein R³ is C₁₋₆alkyl.

E130: The method of any one of embodiments 125-129, wherein each R⁶ is independently selected from C₁₋₆alkyl, halogen, —CN, C₁₋₆haloalkyl, —OR⁷, C₃₋₆cycloalkyl, C₂₋₉heterocycloalkyl, and C₂₋₉heteroaryl, wherein C₃₋₆cycloalkyl, C₂₋₉heterocycloalkyl, and C₂₋₉heteroaryl are optionally substituted with one or two groups independently selected from halogen, C₁₋₆alkyl, C₁₋₆haloalkyl, and C₁₋₆alkoxy.

E131: The method of any one of embodiments 125-130, wherein each R⁶ is independently selected from C₁₋₆alkyl, halogen, —CN, and C₁₋₆haloalkyl.

E132: The method of any one of embodiments 125-131, wherein n is 1 or 2.

E133: The method of embodiment 125, wherein the compound of Formula (XIV) is selected from:

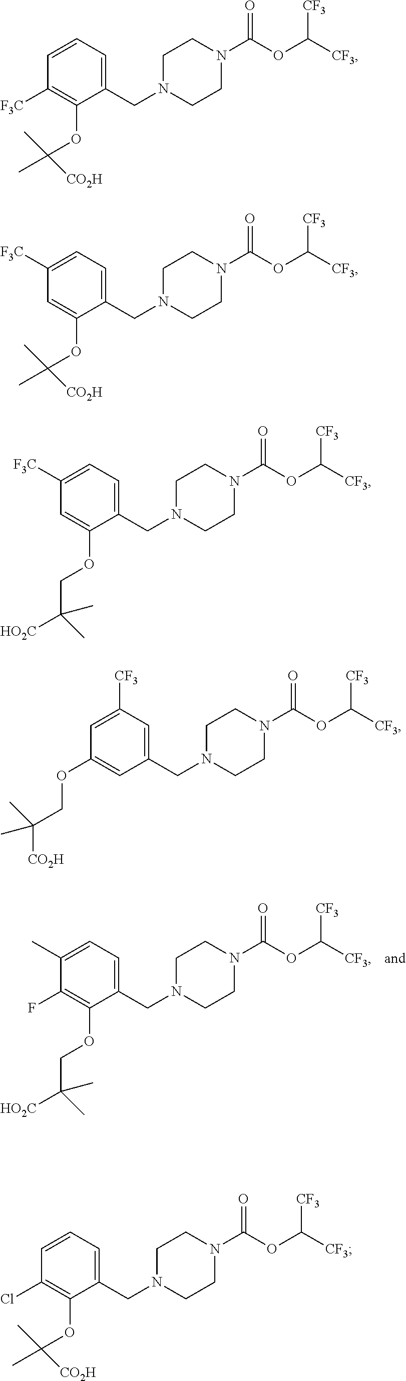 [SMILES: CC(C)(Oc1c(CN2CCN(C(=O)OC(C(F)(F)F)C(F)(F)F)CC2)cccc1C(F)(F)F)C(=O)O ; CC(C)(Oc1cc(C(F)(F)F)ccc1CN1CCN(C(=O)OC(C(F)(F)F)C(F)(F)F)CC1)C(=O)O ; CC(C)(COc1cc(C(F)(F)F)ccc1CN1CCN(C(=O)OC(C(F)(F)F)C(F)(F)F)CC1)C(=O)O ; CC(C)(COc1cc(CN2CCN(C(=O)OC(C(F)(F)F)C(F)(F)F)CC2)cc(C(F)(F)F)c1)C(=O)O ; Cc1ccc(CN2CCN(C(=O)OC(C(F)(F)F)C(F)(F)F)CC2)c(OCC(C)(C)C(=O)O)c1F ; CC(C)(Oc1c(Cl)cccc1CN1CCN(C(=O)OC(C(F)(F)F)C(F)(F)F)CC1)C(=O)O]

or a pharmaceutically acceptable salt or solvate thereof.

E134: A method for treating dyskinesia in a patient in need thereof, comprising administering to the patient a therapeutically effective amount of a compound of Formula (XV):

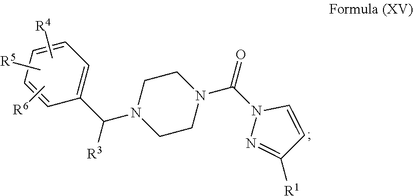

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₆alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring; and
R¹⁵ is optionally substituted C₁₋₆alkyl;
   or a pharmaceutically acceptable salt or solvate thereof.

E135: A method for treating dyskinesia in a patient in need thereof, comprising administering to the patient a therapeutically effective amount of a compound of Formula (XVI):

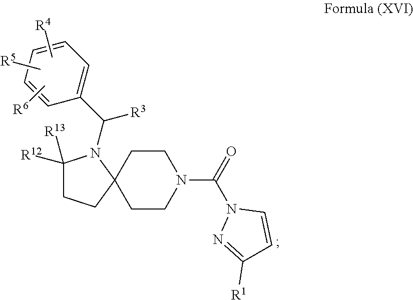

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₃alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring;
R¹² is H or C₁₋₆alkyl;
R¹³ is H or C₁₋₆alkyl; and
R¹⁵ is optionally substituted C₁₋₆alkyl;
   or a pharmaceutically acceptable salt or solvate thereof.

E136: The method of embodiment 135, wherein R¹² and R¹³ are H.

E137: The method of any one of embodiments 134-136, wherein R⁴ is optionally substituted heterocycloalkyl.

E138: The method of any one of embodiments 134-137, wherein R⁴ is heterocycloalkyl optionally substituted with one or more groups selected from halogen, hydroxy, C₁₋₆alkyl, —C₁₋₆alkyl-OH, C₁₋₆fluoroalkyl, C₃₋₆cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl.

E139: The method of any one of embodiments 134-138, wherein R⁴ is optionally substituted heterocycloalkyl and the heterocycloalkyl is a 4-6 membered monocyclic heterocycloalkyl, a 8-9 membered bicyclic heterocycloalkyl, a 7-8 membered bridged heterocycloalkyl, a 5,5 fused heterocycloalkyl, or an 8-11 membered spirocyclic heterocycloalkyl.

E140: The method of any one of embodiments 134-136, wherein R⁴ is

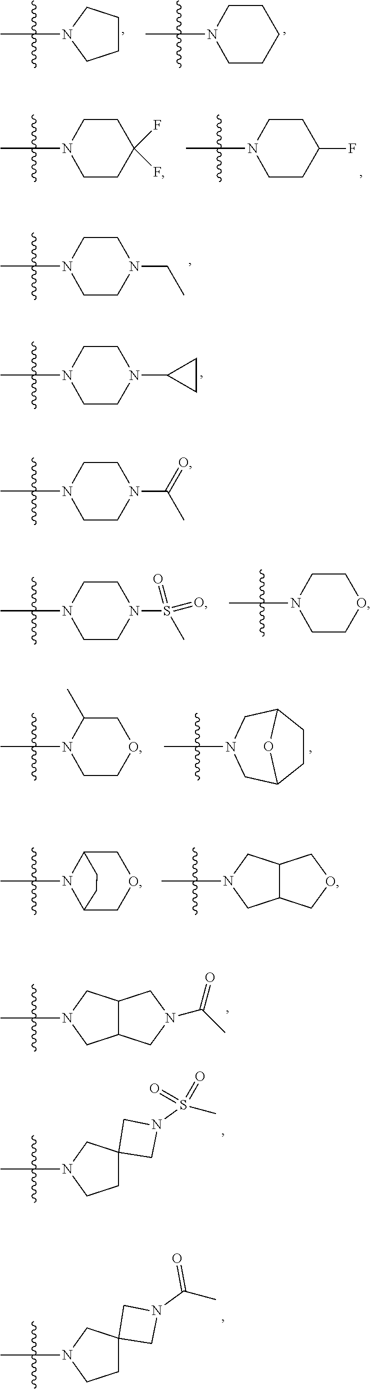

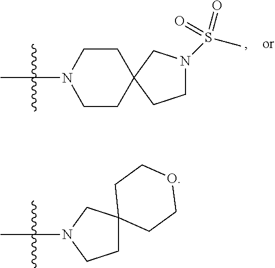 [SMILES: *N1CCC2(CC1)CCN(S(C)(=O)=O)C2 ; *N1CCC2(CCOCC2)C1]

E141: The method of any one of embodiments 134-136, wherein R⁴ is

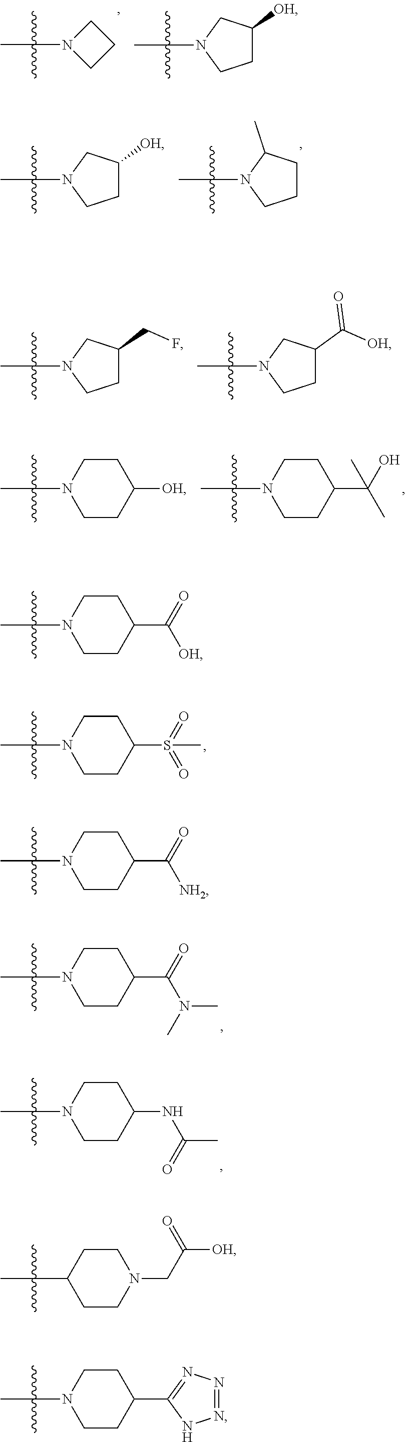

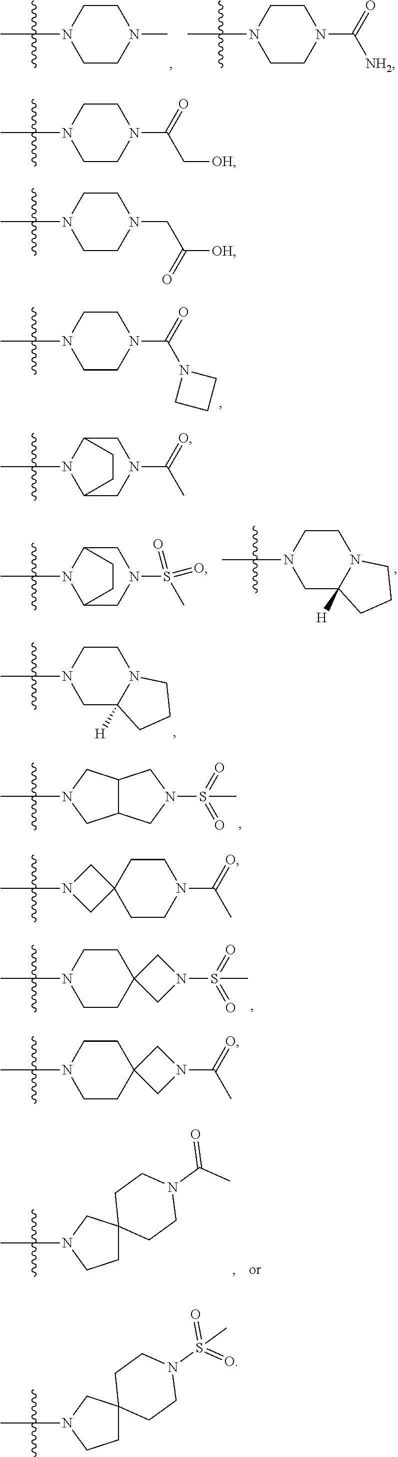

E142: The method of any one of embodiments 134-136, wherein R⁴ is halogen.

E143: The method of any one of embodiments 134-136, wherein R⁴ is C₁₋₆haloalkyl.

E144: The method of any one of embodiments 134-143, wherein R⁵ is halogen.

E145: The method of any one of embodiments 134-143, wherein R⁵ is C₁₋₆haloalkyl.

E146: The method of any one of embodiments 134-143, wherein R⁵ is C₁₋₆alkyl.

E147: The method of any one of embodiments 134-146, wherein R⁶ is H.

E148: The method of any one of embodiments 134-146, wherein R³ is H.

E149: The method of any one of embodiments 134-148, wherein R¹ is —N(R²)C(O)R¹⁵.

E150: The method of any one of embodiments 134-148, wherein R¹ is —N(H)SO₂R¹⁵.

E151: The method of any one of embodiments 134-150, wherein R¹⁵ is unsubstituted C₁₋₆alkyl.

E152: The method of embodiment 134, wherein the compound of Formula (XV) is selected from:

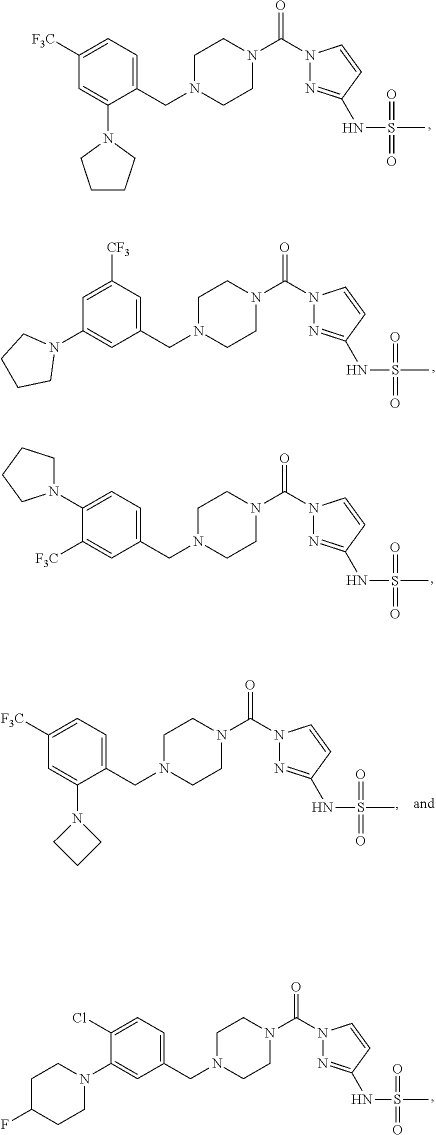 [SMILES: CS(=O)(=O)Nc1ccn(C(=O)N2CCN(Cc3ccc(C(F)(F)F)cc3N3CCCC3)CC2)n1 ; CS(=O)(=O)Nc1ccn(C(=O)N2CCN(Cc3cc(N4CCCC4)cc(C(F)(F)F)c3)CC2)n1 ; CS(=O)(=O)Nc1ccn(C(=O)N2CCN(Cc3ccc(N4CCCC4)c(C(F)(F)F)c3)CC2)n1 ; CS(=O)(=O)Nc1ccn(C(=O)N2CCN(Cc3ccc(C(F)(F)F)cc3N3CCC3)CC2)n1 ; CS(=O)(=O)Nc1ccn(C(=O)N2CCN(Cc3ccc(Cl)c(N4CCC(F)CC4)c3)CC2)n1]

or a pharmaceutically acceptable salt or solvate thereof.

E153: The method of embodiment 135, wherein the compound of Formula (XVI) is selected from:

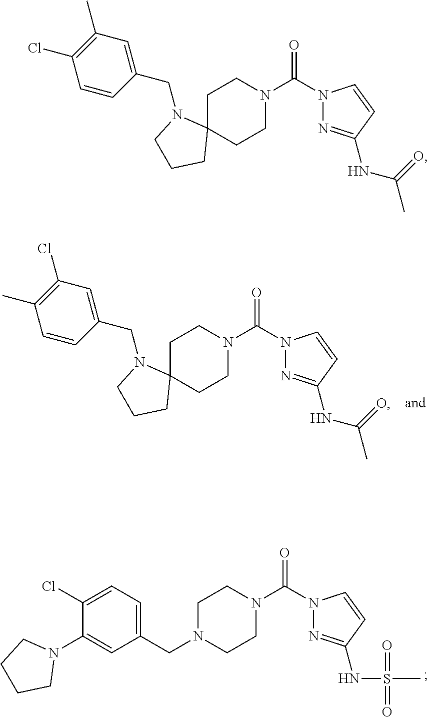 [SMILES: CC(=O)Nc1ccn(C(=O)N2CCC3(CCCN3Cc3ccc(Cl)c(C)c3)CC2)n1 ; CC(=O)Nc1ccn(C(=O)N2CCC3(CCCN3Cc3ccc(C)c(Cl)c3)CC2)n1 ; CS(=O)(=O)Nc1ccn(C(=O)N2CCN(Cc3ccc(Cl)c(N4CCCC4)c3)CC2)n1]

or a pharmaceutically acceptable salt or solvate thereof.

E154: A method for treating dyskinesia in a patient in need thereof, comprising administering to the patient a therapeutically effective amount of a compound of Formula (XVII):

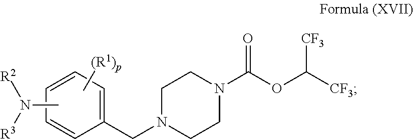

wherein:
each R¹ is independently selected from halogen, C₁₋₆alkyl, C₁₋₆haloalkyl, C₁₋₆alkoxy, C₁₋₆haloalkoxy, C₃₋₈cycloalkyl, —OH, and —CN;
R² and R³, together with the carbon to which they are attached, form
   (i) a C₂-C₇heterocycloalkyl; or
   (ii) a C₂-C₉heteroaryl;
wherein the C₂-C₇heterocycloalkyl or the C₂-C₉heteroaryl is substituted with one R⁴ and optionally substituted with one or two additional substituents selected from halogen, C₁₋₆alkyl, C₁₋₆haloalkyl, and C₁₋₆alkoxy;
R⁴ is —CO₂H or —C₁₋₆alkyl-CO₂H; and
p is 0, 1, 2, 3, or 4;
   or a pharmaceutically acceptable salt or solvate thereof.

E155: The method of embodiment 154, wherein R² and R³, together with the carbon to which they are attached, form a C₂-C₇heterocycloalkyl substituted with one R⁴ and optionally substituted with one or two additional substituents selected from halogen, C₁₋₆alkyl, C₁₋₆haloalkyl, and C₁₋₆alkoxy.

E156: The method of embodiment 154 or embodiment 155, wherein R⁴ is —CO₂H.

E157: The method of embodiment 154 or embodiment 155, wherein R⁴ is —C₁₋₆alkyl-CO₂H.

E158: The method of any one of embodiments 154-157, wherein each R¹ is independently selected from halogen, C₁₋₆alkyl, and C₁₋₆haloalkyl.

E159: The method of any one of embodiments 154-158, wherein p is 1 or 2.

E160: The method of any one of embodiments 154-159, wherein p is 2.

E161: The method of any one of embodiments 154-159, wherein p is 1.

E162: The method of embodiment 153, wherein the compound of Formula (XVII) is selected from:

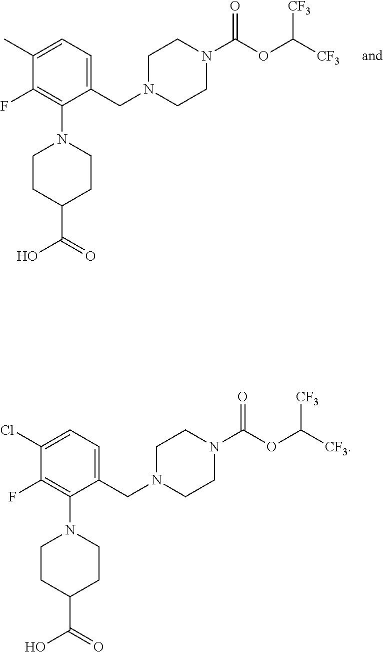 [SMILES: Cc1ccc(CN2CCN(C(=O)OC(C(F)(F)F)C(F)(F)F)CC2)c(N2CCC(C(=O)O)CC2)c1F ; O=C(O)C1CCN(c2c(CN3CCN(C(=O)OC(C(F)(F)F)C(F)(F)F)CC3)ccc(Cl)c2F)CC1]

E163: A method for treating dyskinesia in a patient in need thereof, comprising administering to the patient a therapeutically effective amount of a compound of Formula (I′):

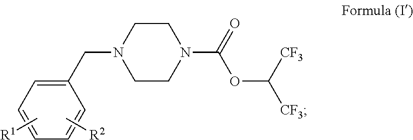

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is optionally substituted with one or two substituents independently selected from C₁₋₆ haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

E164: The method of embodiment 163, wherein the compound of Formula (I′) is a compound of Formula (III):

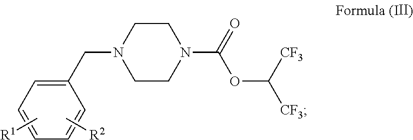

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

E165: The method of embodiment 163 or embodiment 164, wherein R⁵ and R⁶, together with the nitrogen to which they are attached, form a 4-6 membered saturated monocyclic heterocycle, wherein the 4-6 membered saturated monocyclic heterocycle is substituted with one substituent selected from C₁₋₆haloalkyl, —C(O)OR⁹, and —NR⁹SO₂R⁸; and the 4-6 membered saturated monocyclic heterocycle optionally contains an additional O, N, or S.

E166: The method of embodiment 165, wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine, piperidine, and morpholine.

E167: The method of embodiment 166, wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine and piperidine.

E168: The method of embodiment 163, wherein R⁵ and R⁶, together with the nitrogen to which they are attached, form an unsubstituted 4-6 membered saturated monocyclic heterocycle.

E169: The method of embodiment 168, wherein R⁵ and R⁶, together with the nitrogen to which they are attached, form an unsubstituted 4-6 membered saturated monocyclic heterocycle, wherein the 4-6 membered saturated monocyclic heterocycle is selected from pyrrolidine, piperidine, and morpholine.

E170: The method of embodiment 163 or embodiment 164, wherein R⁵ and R⁶, together with the nitrogen to which they are attached, form a 7-8 membered bridged heterocyclic ring optionally substituted with one or two substituents independently selected from halogen, oxo, and C₁₋₆alkyl.

E171: The method of embodiment 170, wherein R⁵ and R⁶, together with the nitrogen to which they are attached, form an unsubstituted 7-8 membered bridged heterocyclic ring.

E172: The method of any one of embodiments 163-171, wherein R¹ is halogen, —SF₅, or optionally substituted C₁₋₆alkyl optionally substituted by halogen.

E173: The method of any one of embodiments 163-172, wherein R¹ is halogen.

E174: The method of any one of embodiments 163-172, wherein R¹ is C₁₋₆alkyl optionally substituted by halogen.

E175: The method of embodiment 174, wherein R¹ is —CF₃.

E176: The method of embodiment 163, wherein the compound is selected from:

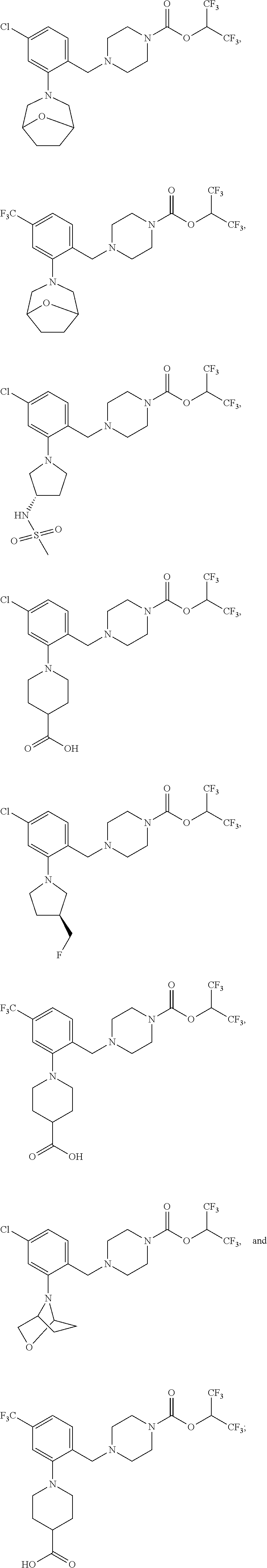

or a pharmaceutically acceptable salt or solvate thereof.

E177: The method of embodiment 163, wherein the compound is:

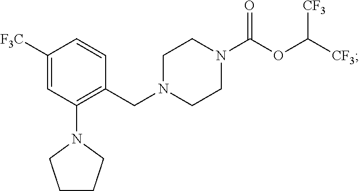 [SMILES: O=C(OC(C(F)(F)F)C(F)(F)F)N1CCN(Cc2ccc(C(F)(F)F)cc2N2CCCC2)CC1]

or a pharmaceutically acceptable salt or solvate thereof.

E178: The method of any one of embodiments 1-177, wherein the dyskinesia is levodopa-induced dyskinesia.

### Methods

In some embodiments disclosed herein are methods of modulating the activity of MAGL. Contemplated methods, for example, comprise exposing said enzyme to a compound described herein. The ability of compounds described herein to modulate or inhibit MAGL is evaluated by procedures known in the art and/or described herein. Another aspect of this disclosure provides methods of treating a disease associated with expression or activity of MAGL in a patient.

Compounds described herein are modulators of MAGL. In some embodiments, these compounds and pharmaceutical compositions comprising these compounds, are useful for the treatment of dyskinesia. In some embodiments, these compounds and pharmaceutical compositions comprising these compounds, are useful for the treatment of levadopa-induced dyskinesia.

In some embodiments is a method for treating dyskinesia in a patient in need thereof, comprising administering to the patient a therapeutically effective amount of a compound of Formula (I′):

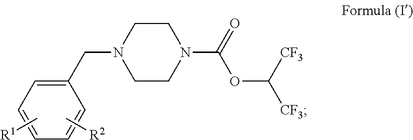

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (i) a 4-6 membered saturated monocyclic heterocycle; or
   (ii) a 7-8 membered bridged heterocyclic ring optionally containing an additional O, N, or S;
   wherein the 4-6 membered saturated monocyclic heterocycle is optionally substituted with one or two substituents independently selected from C₁₋₆ haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

In some embodiments is a method for treating dyskinesia in a patient in need thereof, comprising administering to the patient a therapeutically effective amount of a compound of Formula (I′), wherein the dyskinesia is levodopa-induced dyskinesia.

In some embodiments of the methods for treating dyskinesia with a compound of Formula (I′), R⁵ and R⁶, together with the nitrogen to which they are attached, form a 4-6 membered saturated monocyclic heterocycle substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S. In some embodiments of the methods for treating dyskinesia with a compound of Formula (I′), R⁵ and R⁶, together with the nitrogen to which they are attached, form a 4-6 membered saturated monocyclic heterocycle substituted with one or two substituents independently selected from C₁₋₆haloalkyl, —C(O)OR⁹, and —NR⁹SO₂R⁸; wherein the 4-6 membered saturated monocyclic heterocycle is selected from pyrrolidine, piperidine, and morpholine. In some embodiments of the methods for treating dyskinesia with a compound of Formula (I), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is selected from pyrrolidine, piperidine, and morpholine. In some embodiments of the methods for treating dyskinesia with a compound of Formula (I), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is selected from pyrrolidine and piperidine. In some embodiments of the methods for treating dyskinesia with a compound of Formula (I′), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is pyrrolidine. In some embodiments of the methods for treating dyskinesia with a compound of Formula (I), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is piperidine. In some embodiments of the methods for treating dyskinesia with a compound of Formula (I′), R⁵ and R⁶, together with the nitrogen to which they are attached, form a 4-6 membered saturated monocyclic heterocycle substituted with one substituent selected from C₁₋₆ haloalkyl, —C(O)OR⁹, and —NR⁹SO₂R⁸; wherein the 4-6 membered saturated monocyclic heterocycle is morpholine.

In some embodiments of the methods for treating dyskinesia with a compound of Formula (I′), R⁵ and R⁶, together with the nitrogen to which they are attached, form an unsubstituted 4-6 membered saturated monocyclic heterocycle. In some embodiments of the methods for treating dyskinesia with a compound of Formula (I′), R⁵ and R⁶, together with the nitrogen to which they are attached, form an unsubstituted 4-6 membered saturated monocyclic heterocycle, wherein the 4-6 membered saturated monocyclic heterocycle is selected from pyrrolidine, piperidine, and morpholine. In some embodiments of the methods for treating dyskinesia with a compound of Formula (I′), R⁵ and R⁶, together with the nitrogen to which they are attached, form an unsubstituted 4-6 membered saturated monocyclic heterocycle, wherein the 4-6 membered saturated monocyclic heterocycle is pyrrolidine. In some embodiments of the methods for treating dyskinesia with a compound of Formula (I′), R⁵ and R⁶, together with the nitrogen to which they are attached, form an unsubstituted 4-6 membered saturated monocyclic heterocycle, wherein the 4-6 membered saturated monocyclic heterocycle is piperidine. In some embodiments of the methods for treating dyskinesia with a compound of Formula (I′), R⁵ and R⁶, together with the nitrogen to which they are attached, form an unsubstituted 4-6 membered saturated monocyclic heterocycle, wherein the 4-6 membered saturated monocyclic heterocycle is morpholine.

In some embodiments of the methods for treating dyskinesia with a compound of Formula (I′), R⁵ and R⁶, together with the nitrogen to which they are attached, form a 7-8 membered bridged heterocyclic ring is optionally substituted with one or two substituents independently selected from halogen, oxo, and C₁₋₆alkyl. In some embodiments of the methods for treating dyskinesia with a compound of Formula (I′), R⁵ and R⁶, together with the nitrogen to which they are attached, form an unsubstituted 7-8 membered bridged heterocyclic ring. In some embodiments of the methods for treating dyskinesia with a compound of Formula (I), R⁵ and R⁶, together with the nitrogen to which they are attached, form a 7-8 membered bridged heterocyclic ring is substituted with one or two substituents independently selected from halogen, oxo, and C₁₋₆alkyl. In some embodiments of the methods for treating dyskinesia with a compound of Formula (I′), R⁵ and R⁶, together with the nitrogen to which they are attached, form a 7-8 membered bridged heterocyclic ring is substituted with one substituent selected from halogen, oxo, and C₁₋₆alkyl.

In some embodiments of the methods for treating dyskinesia with a compound of Formula (I′), R¹ is halogen, —OR³, —SF₅, or C₁₋₆alkyl optionally substituted by halogen. In some embodiments of the methods for treating dyskinesia with a compound of Formula (I′), R¹ is halogen, —CH₃, —CF₃, —OCH₃, or —OCF₃. In some embodiments of the methods for treating dyskinesia with a compound of Formula (I′), R¹ is halogen, —SF₅, or C₁₋₆alkyl optionally substituted by halogen. In some embodiments of the methods for treating dyskinesia with a compound of Formula (I′), R¹ is halogen. In some embodiments of the methods for treating dyskinesia with a compound of Formula (I′), R¹ is —Cl. In some embodiments of the methods for treating dyskinesia with a compound of Formula (I′), R¹ is C₁₋₆alkyl optionally substituted by halogen. In some embodiments of the methods for treating dyskinesia with a compound of Formula (I′), R¹ is —CH₃. In some embodiments of the methods for treating dyskinesia with a compound of Formula (I′), R¹ is —CF₃. In some embodiments of the methods for treating dyskinesia with a compound of Formula (I′), R¹ is —SF₅. In some embodiments of the methods for treating dyskinesia with a compound of Formula (I′), R¹ is —OCH₃. In some embodiments of the methods for treating dyskinesia with a compound of Formula (I′), R¹ is —OCF₃.

In some embodiments of the methods for treating dyskinesia with a compound of Formula (I′), the compound is selected from:

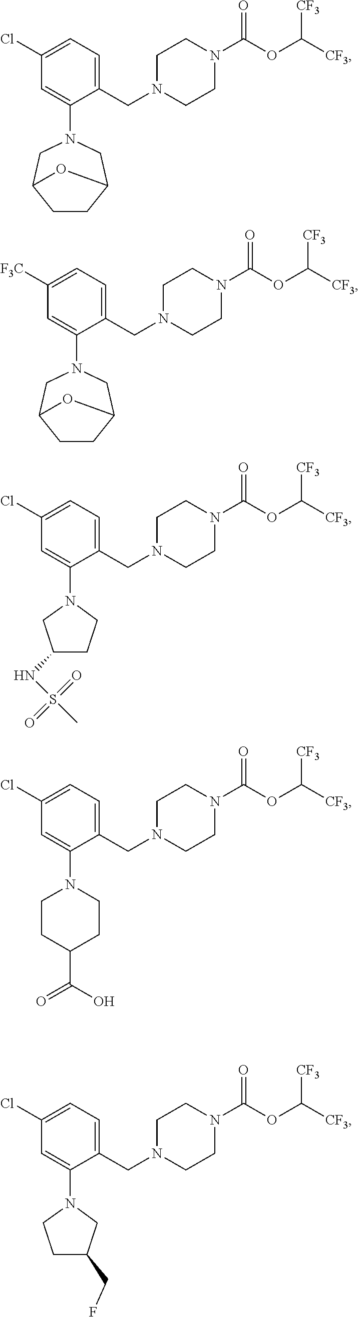 [SMILES: O=C(OC(C(F)(F)F)C(F)(F)F)N1CCN(Cc2ccc(Cl)cc2N2CC3CCC(C2)O3)CC1 ; O=C(OC(C(F)(F)F)C(F)(F)F)N1CCN(Cc2ccc(C(F)(F)F)cc2N2CC3CCC(C2)O3)CC1 ; CS(=O)(=O)N[C@H]1CCN(c2cc(Cl)ccc2CN2CCN(C(=O)OC(C(F)(F)F)C(F)(F)F)CC2)C1 ; O=C(O)C1CCN(c2cc(Cl)ccc2CN2CCN(C(=O)OC(C(F)(F)F)C(F)(F)F)CC2)CC1 ; O=C(OC(C(F)(F)F)C(F)(F)F)N1CCN(Cc2ccc(Cl)cc2N2CC[C@H](CF)C2)CC1]

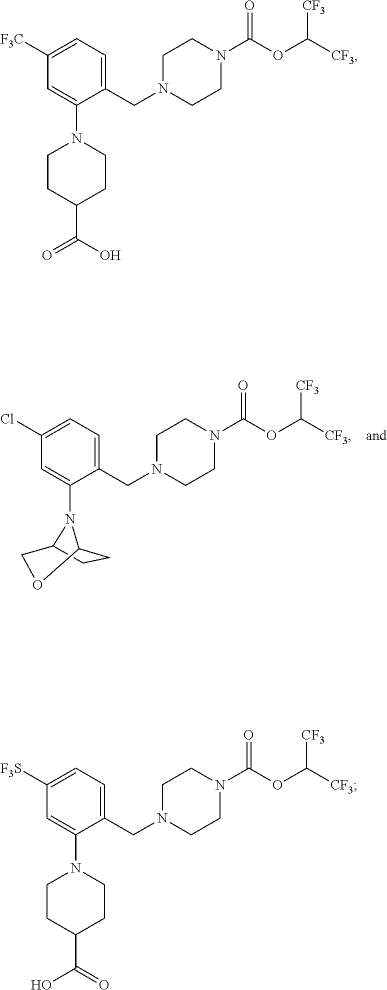

or a pharmaceutically acceptable salt or solvate thereof.

In some embodiments of the methods for treating dyskinesia with a compound of Formula (I′), the compound is:

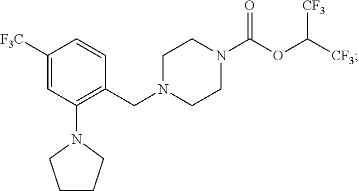 [SMILES: O=C(OC(C(F)(F)F)C(F)(F)F)N1CCN(Cc2ccc(C(F)(F)F)cc2N2CCCC2)CC1]

or a pharmaceutically acceptable salt or solvate thereof.

In some embodiments is a method for treating dyskinesia in a patient in need thereof, comprising administering to the patient a therapeutically effective amount of a compound of Formula (I):

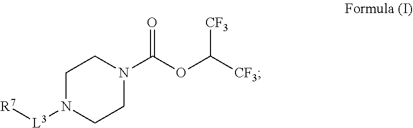

wherein:
L³ is a bond, —CH₂—, —S(O)₂—, or —C(O)—;
R⁷ is phenyl; wherein R⁷ is optionally substituted by one, two, or three moieties independently selected from R^{h};
R^{a} and R^{b} are independently selected, for each occurrence, from the group consisting of hydrogen and C₁₋₃alkyl; wherein C₁₋₃alkyl is optionally substituted by one or more substituents selected from halogen, cyano, oxo, hydroxyl, heterocycle, and phenyl;
or R^{a} and R^{b}, when they occur together with the nitrogen to which they are attached, form a 4-6 membered saturated heterocyclic ring, which may have an additional heteroatom selected from O, S, and N, or a spirocyclic ring selected from 8-oxa-2-azaspiro[4.5]decane and 2,8-diazaspiro[4.5]decane, wherein the 4-6 membered saturated heterocyclic ring or the spirocyclic ring are optionally substituted by one or more substituents selected from the group consisting of halogen, cyano, oxo, C₁₋₆ alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl;
R^{c} is selected from the group consisting of halogen, hydroxyl, C₁₋₆alkyl (optionally substituted by one, two, or three halogens), and C₁₋₆alkoxy (optionally substituted by one, two, or three halogens); and
R^{h} is selected from the group consisting of: halogen, phenyl (optionally substituted by one, two, or three moieties each independently selected from R^{c}), hydroxyl, cyano, C₁₋₆alkyl (optionally substituted by one, two or three halogens), C₁₋₆alkoxy (optionally substituted by one, two or three halogens), R^{a}R^{b}N—, R^{a}—C(O)NR^{a}—, R^{a}R^{b}N—SO₂—, R^{a}R^{b}N—C(O)—, R^{a}—S(O)_{w}— (wherein w is 0, 1 or 2), R^{a}—SO₂—NR^{b}—, and heteroaryl (optionally substituted by one, two or three moieties each independently selected from R^{c});
   or a pharmaceutically acceptable salt or solvate thereof.

In some embodiments is a method for treating dyskinesia in a patient in need thereof, comprising administering to the patient a therapeutically effective amount of a compound of Formula (I), wherein the dyskinesia is levodopa-induced dyskinesia.

In some embodiments of the methods for treating dyskinesia with a compound of Formula (I), L³ is —CH₂—. In some embodiments of the methods for treating dyskinesia with a compound of Formula (I), L³ is a bond. In some embodiments of the methods for treating dyskinesia with a compound of Formula (I), L³ is —S(O)₂—. In some embodiments of the methods for treating dyskinesia with a compound of Formula (I), L³ is —C(O)—.

In some embodiments of the methods for treating dyskinesia with a compound of Formula (I), R⁷ is phenyl optionally substituted by one or two moieties independently selected from R^{h}. In some embodiments of the methods for treating dyskinesia with a compound of Formula (I), R⁷ is phenyl optionally substituted by one or two R^{h} moieties independently selected from the group consisting of halogen, phenyl (optionally substituted by one, two, or three moieties each independently selected from halogen, methyl, ethyl, propyl, t-butyl, and CF₃), C₁₋₆alkyl (optionally substituted by one, two or three halogens), C₁₋₆alkoxy (optionally substituted by one, two or three halogens), R^{a}R^{b}N—, R^{a}R^{b}N—C(O)—, and heteroaryl (optionally substituted by one, two or three moieties each independently selected from C₁₋₆alkyl or halogen). In some embodiments of the methods for treating dyskinesia with a compound of Formula (I), R⁷ is phenyl optionally substituted by one or two R^{h} moieties independently selected from the group consisting of halogen, C₁₋₆alkyl (optionally substituted by one, two or three halogens), C₁₋₆alkoxy (optionally substituted by one, two or three halogens), and R^{a}R^{b}N—.

In some embodiments of the methods for treating dyskinesia with a compound of Formula (I), L³ is —CH₂— and R⁷ is substituted by R^{a}R^{b}N— and a moiety selected from the group consisting of: halogen, C₁₋₆alkyl (optionally substituted by one, two or three halogens), and C₁₋₆alkoxy (optionally substituted by one, two or three halogens). In some embodiments of the methods for treating dyskinesia with a compound of Formula (I), R^{a} and R^{b}, together with the nitrogen to which they are attached, form a 4-6 membered saturated heterocyclic ring, which may have an additional heteroatom selected from 0, S, and N, and the 4-6 membered saturated heterocyclic ring is optionally substituted by one or more substituents selected from the group consisting of halogen, cyano, oxo, C₁₋₆ alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl. In some embodiments of the methods for treating dyskinesia with a compound of Formula (I), the 4-6 membered saturated heterocyclic ring is selected from azetidine, pyrrolidine, piperidine, piperazine, and morpholine, and the 4-6 membered saturated heterocyclic ring is optionally substituted by one or more substituents selected from the group consisting of halogen, cyano, oxo, C₁₋₆alkyl, —S(O)_{w}—C₁₋₆alkyl (where w is 0, 1 or 2), hydroxyl, —C(O)—C₁₋₆alkyl, —NH₂, and —NH—C(O)—C₁₋₆alkyl. In some embodiments of the methods for treating dyskinesia with a compound of Formula (I), the 4-6 membered saturated heterocyclic ring is pyrrolidine. In some embodiments of the methods for treating dyskinesia with a compound of Formula (I), the 4-6 membered saturated heterocyclic ring is piperidine. In some embodiments of the methods for treating dyskinesia with a compound of Formula (I), the 4-6 membered saturated heterocyclic ring is morpholine.

In some embodiments of the methods for treating dyskinesia with a compound of Formula (I), the compound is selected from:

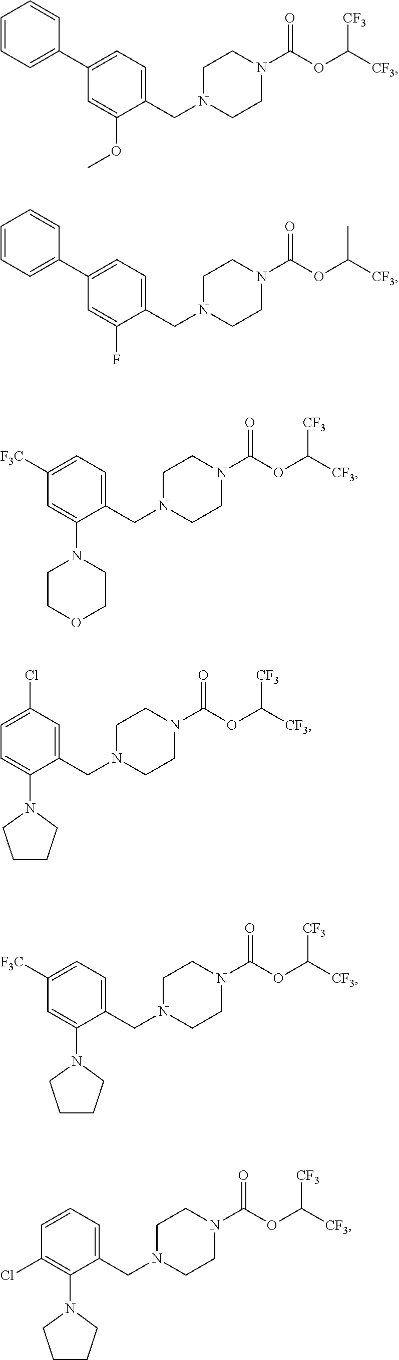 [SMILES: COc1cc(-c2ccccc2)ccc1CN1CCN(C(=O)OC(C(F)(F)F)C(F)(F)F)CC1 ; CC(OC(=O)N1CCN(Cc2ccc(-c3ccccc3)cc2F)CC1)C(F)(F)F ; O=C(OC(C(F)(F)F)C(F)(F)F)N1CCN(Cc2ccc(C(F)(F)F)cc2N2CCOCC2)CC1 ; O=C(OC(C(F)(F)F)C(F)(F)F)N1CCN(Cc2cc(Cl)ccc2N2CCCC2)CC1 ; O=C(OC(C(F)(F)F)C(F)(F)F)N1CCN(Cc2ccc(C(F)(F)F)cc2N2CCCC2)CC1 ; O=C(OC(C(F)(F)F)C(F)(F)F)N1CCN(Cc2cccc(Cl)c2N2CCCC2)CC1]

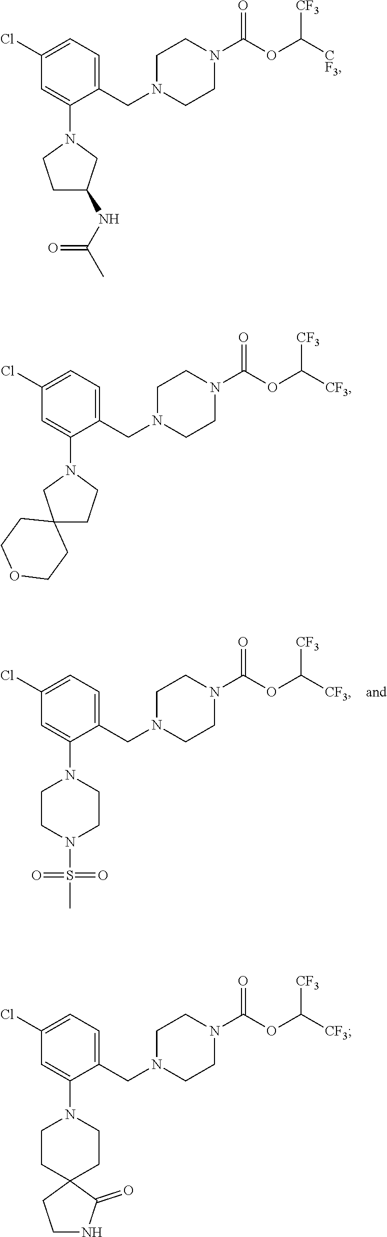 [SMILES: CC(=O)N[C@H]1CCN(c2cc(Cl)ccc2CN2CCN(C(=O)OC(C(F)(F)F)C(F)(F)F)CC2)C1 ; O=C(OC(C(F)(F)F)C(F)(F)F)N1CCN(Cc2ccc(Cl)cc2N2CCC3(CCOCC3)C2)CC1 ; CS(=O)(=O)N1CCN(c2cc(Cl)ccc2CN2CCN(C(=O)OC(C(F)(F)F)C(F)(F)F)CC2)CC1 ; "and" ; O=C(OC(C(F)(F)F)C(F)(F)F)N1CCN(Cc2ccc(Cl)cc2N2CCC3(CCNC3=O)CC2)CC1]

or a pharmaceutically acceptable salt or solvate thereof.

In some embodiments is a method for treating dyskinesia in a patient in need thereof, comprising administering to the patient a therapeutically effective amount of a compound of Formula (II):

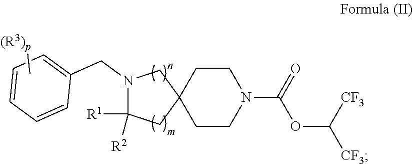

wherein:
R¹ is H or C₁₋₆alkyl;
R² is H or C₁₋₆alkyl;
each R³ is independently selected from C₁₋₆alkyl, C₂₋₆alkenyl, C₂₋₆alkynyl, halogen, —CN, C₁₋₆haloalkyl, C₁₋₆aminoalkyl, heterocycloalkyl, —C₁₋₆alkyl(heterocycloalkyl), heteroaryl, —SF₅, —NR⁵R⁶, —OR⁷, —CO₂R⁸, —C(O)R⁸, and —C(O)NR⁸R⁹, wherein heterocycloalkyl and —C₁₋₆alkyl(heterocycloalkyl) are optionally substituted with one or two R⁴; or two adjacent R³ form a heterocycloalkyl ring optionally substituted with one, two, or three R⁴;
each R⁴ is independently selected from C₁₋₆alkyl, C₁₋₆haloalkyl, C₃₋₈cycloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸; each R⁵ and R⁶ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl; or R⁵ and R⁶, together with the nitrogen to which they are attached, form a heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(heterocycloalkyl), —C₁₋₆alkyl-C(O)(heterocycloalkyl), heterocycloalkyl, aryl, and heteroaryl, wherein heterocycloalkyl, aryl, and heteroaryl are optionally substituted with one or two groups selected from oxo, C₁₋₆alkyl, C₁₋₆haloalkyl, CO₂H, and C(O)NH₂;
each R⁸ and R⁹ is independently selected from H, C₁₋₆alkyl, C₁₋₆haloalkyl, C₃₋₈cycloalkyl, aryl, and heteroaryl; or R⁸ and R⁹, together with the nitrogen to which they are attached, form a heterocycloalkyl ring optionally substituted with one or two groups selected from C₁₋₆alkyl, C₁₋₆haloalkyl, CO₂H, and C(O)NH₂;
each R¹⁰ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸;
p is 0, 1, 2, 3, 4, or 5;
n is 0 or 1; and
m is 1 or 2; provided that when n is 0, then m is 2; and when n is 1, then m is 1;
   or a pharmaceutically acceptable salt or solvate thereof.

In some embodiments is a method for treating dyskinesia in a patient in need thereof, comprising administering to the patient a therapeutically effective amount of a compound of Formula (II), wherein the dyskinesia is levodopa-induced dyskinesia.

In some embodiments of the methods for treating dyskinesia with a compound of Formula (II), n is 0 and m is 2. In some embodiments of the methods for treating dyskinesia with a compound of Formula (II), n is 1 and m is 1.

In some embodiments of the methods for treating dyskinesia with a compound of Formula (II), R¹ is H. In some embodiments of the methods for treating dyskinesia with a compound of Formula (II), R² is H. In some embodiments of the methods for treating dyskinesia with a compound of Formula (II), wherein R¹ and R² are both H.

In some embodiments of the methods for treating dyskinesia with a compound of Formula (II), p is 0, 1, 2, or 3. In some embodiments of the methods for treating dyskinesia with a compound of Formula (II), p is 0. In some embodiments of the methods for treating dyskinesia with a compound of Formula (II), p is 1 or 2. In some embodiments of the methods for treating dyskinesia with a compound of Formula (II), p is 1. In some embodiments of the methods for treating dyskinesia with a compound of Formula (II), p is 2. some embodiments of the methods for treating dyskinesia with a compound of Formula (II), p is 3.

In some embodiments of the methods for treating dyskinesia with a compound of Formula (II), p is 1 and R³ is selected from C₁₋₆alkyl, halogen, C₁₋₆ haloalkyl, —C₁₋₆alkyl(heterocycloalkyl), —NR⁵R⁶, —OR⁷, —CO₂R⁸, and —C(O)NR⁸R⁹. In some embodiments of the methods for treating dyskinesia with a compound of Formula (II), p is 1 and R³ is selected from halogen, C₁₋₆haloalkyl, —NR⁵R⁶, and —OR⁷.

In some embodiments of the methods for treating dyskinesia with a compound of Formula (II), p is 1 and R³ is —NR⁵R⁶. In some embodiments of the methods for treating dyskinesia with a compound of Formula (II), R³ is —NR⁵R⁶, and R⁵ and R⁶, together with the nitrogen to which they are attached, form a heterocycloalkyl ring optionally substituted with one, two, or three R¹⁰. In some embodiments of the methods for treating dyskinesia with a compound of Formula (II), p is 1, R³ is —NR⁵R⁶, and R⁵ and R⁶, together with the nitrogen to which they are attached, form an unsubstituted heterocycloalkyl ring. In some embodiments of the methods for treating dyskinesia with a compound of Formula (II), p is 1, R³ is —NR⁵R⁶, and R⁵ and R⁶, together with the nitrogen to which they are attached, form a heterocycloalkyl ring substituted with one or two R¹⁰ independently selected from C₁₋₆alkyl, cycloalkyl, C₁₋₆ haloalkyl, halogen, —CO₂R⁸, —C(O)R⁸, —C(O)NR⁸R⁹, —SO₂R⁸, —NR⁹C(O)R⁸, and —NR⁹SO₂R⁸.

In some embodiments of the methods for treating dyskinesia with a compound of Formula (II), p is 2 and each R³ is independently selected from C₁₋₆alkyl, halogen, C₁₋₆haloalkyl, —C₁₋₆alkyl(heterocycloalkyl), —NR⁵R⁶, —OR⁷, —CO₂R⁸, and —C(O)NR⁸R⁹. In some embodiments of the methods for treating dyskinesia with a compound of Formula (II), p is 2, one R³ is halogen, and one R³ is —OR⁷. In some embodiments of the methods for treating dyskinesia with a compound of Formula (II), p is 2, one R³ is —Cl, one R³ is —OR⁷, and R⁷ is C₁₋₆alkyl. In some embodiments of the methods for treating dyskinesia with a compound of Formula (II), p is 2, one R³ is —Cl, one R³ is —OR⁷, and R⁷ is —C₁₋₆alkyl(heterocycloalkyl). In some embodiments of the methods for treating dyskinesia with a compound of Formula (II), p is 2, one R³ is halogen, and one R³ is —NR⁵R⁶. In some embodiments of the methods for treating dyskinesia with a compound of Formula (II), p is 2, one R³ is halogen, one R³ is —NR⁵R⁶, and R⁵ and R⁶, together with the nitrogen to which they are attached, form an unsubstituted heterocycloalkyl ring. In some embodiments of the methods for treating dyskinesia with a compound of Formula (II), p is 2, one R³ is halogen, one R³ is —NR⁵R⁶, and R⁵ and R⁶, together with the nitrogen to which they are attached, form a heterocycloalkyl ring substituted with one or two R¹⁰ independently selected from C₁₋₆ alkyl, cycloalkyl, C₁₋₆haloalkyl, halogen, —CO₂R⁸, —C(O)R⁸, —C(O)NR⁸R⁹, —SO₂R⁸, —NR⁹C(O)R⁸, and —NR⁹SO₂R⁸. In some embodiments of the methods for treating dyskinesia with a compound of Formula (II), p is 2, one R³ is —Cl, and one R³ is —NR⁵R⁶.

In some embodiments of the methods for treating dyskinesia with a compound of Formula (II), p is 2, one R³ is —Cl, one R³ is —NR⁵R⁶, and R⁵ and R⁶, together with the nitrogen to which they are attached, form an unsubstituted heterocycloalkyl ring. In some embodiments of the methods for treating dyskinesia with a compound of Formula (II), p is 2, one R³ is —Cl, one R³ is —NR⁵R⁶, and R⁵ and R⁶, together with the nitrogen to which they are attached, form a heterocycloalkyl ring substituted with one or two R¹⁰ independently selected from C₁₋₆alkyl, cycloalkyl, C₁₋₆haloalkyl, halogen, —CO₂R⁸, —C(O)R⁸, —C(O)NR⁸R⁹, —SO₂R⁸, —NR⁹C(O)R⁸, and —NR⁹SO₂R⁸. In some embodiments of the methods for treating dyskinesia with a compound of Formula (II), p is 2, one R³ is C₁₋₆ haloalkyl, and one R³ is —NR⁵R⁶. In some embodiments of the methods for treating dyskinesia with a compound of Formula (II), p is 2, one R³ is C₁₋₆haloalkyl, one R³ is —NR⁵R⁶, and R⁵ and R⁶, together with the nitrogen to which they are attached, form an unsubstituted heterocycloalkyl ring. In some embodiments of the methods for treating dyskinesia with a compound of Formula (II), p is 2, one R³ is C₁₋₆haloalkyl, one R³ is —NR⁵R⁶, and R⁵ and R⁶, together with the nitrogen to which they are attached, form a heterocycloalkyl ring substituted with one or two R¹⁰ independently selected from C₁₋₆ alkyl, cycloalkyl, C₁₋₆haloalkyl, halogen, —CO₂R⁸, —C(O)R⁸, —C(O)NR⁸R⁹, —SO₂R⁸, —NR⁹C(O)R⁸, and —NR⁹SO₂R⁸. In some embodiments of the methods for treating dyskinesia with a compound of Formula (II), p is 2, one R³ is —CF₃, and one R³ is —NR⁵R⁶. In some embodiments of the methods for treating dyskinesia with a compound of Formula (II), p is 2, one R³ is —CF₃, one R³ is —NR⁵R⁶, and R⁵ and R⁶, together with the nitrogen to which they are attached, form an unsubstituted heterocycloalkyl ring. In some embodiments of the methods for treating dyskinesia with a compound of Formula (II), p is 2, one R³ is —CF₃, one R³ is —NR⁵R⁶, and R⁵ and R⁶, together with the nitrogen to which they are attached, form a heterocycloalkyl ring substituted with one or two R¹⁰ independently selected from C₁₋₆alkyl, cycloalkyl, C₁₋₆haloalkyl, halogen, —CO₂R⁸, —C(O)R⁸, —C(O)NR⁸R⁹, —SO₂R⁸, —NR⁹C(O)R⁸, and —NR⁹SO₂R⁸.

In some embodiments of the methods for treating dyskinesia with a compound of Formula (II), p is 2, one R³ is C₁₋₆alkyl, halogen, C₁₋₆haloalkyl, —C₁₋₆ alkyl(heterocycloalkyl), —OR⁷, —CO₂R⁸, or —C(O)NR⁸R⁹, and one R³ is —NR⁵R⁶, wherein R⁵ and R⁶, together with the nitrogen to which they are attached, form a heterocycloalkyl ring selected from:

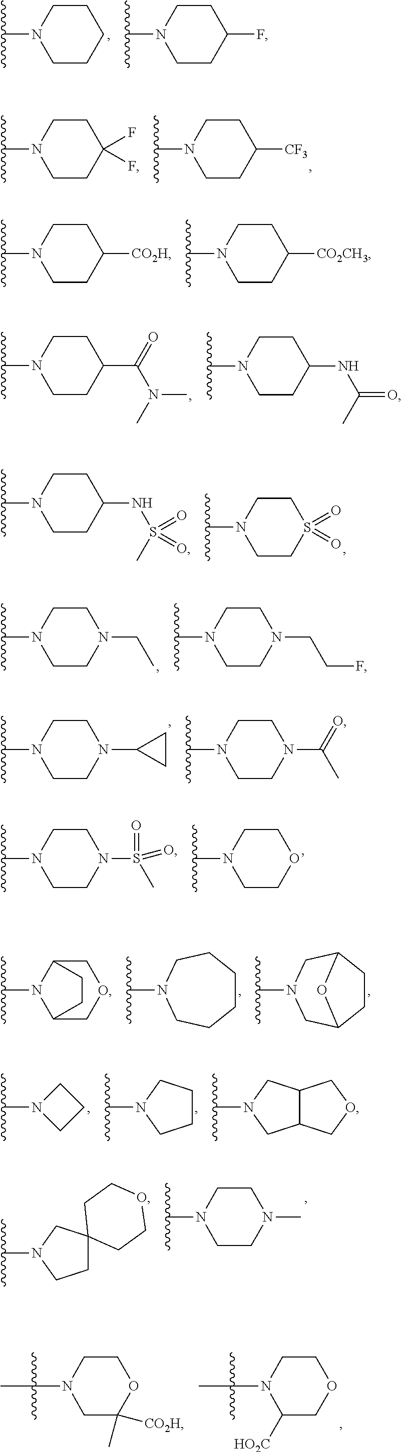

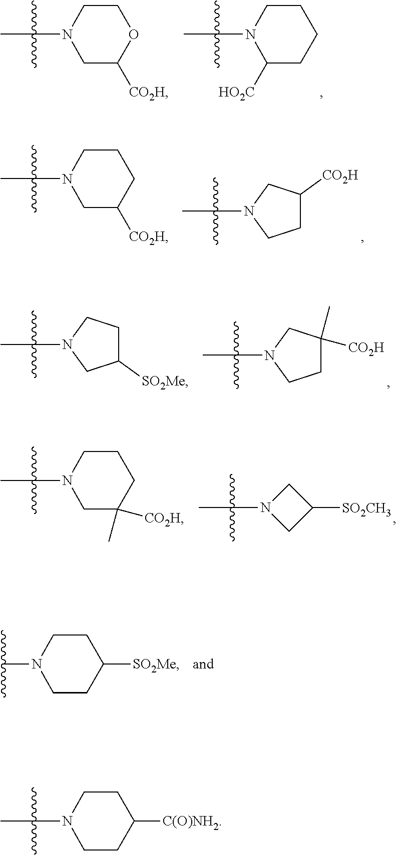

In some embodiments of the methods for treating dyskinesia with a compound of Formula (II), the compound is selected from:

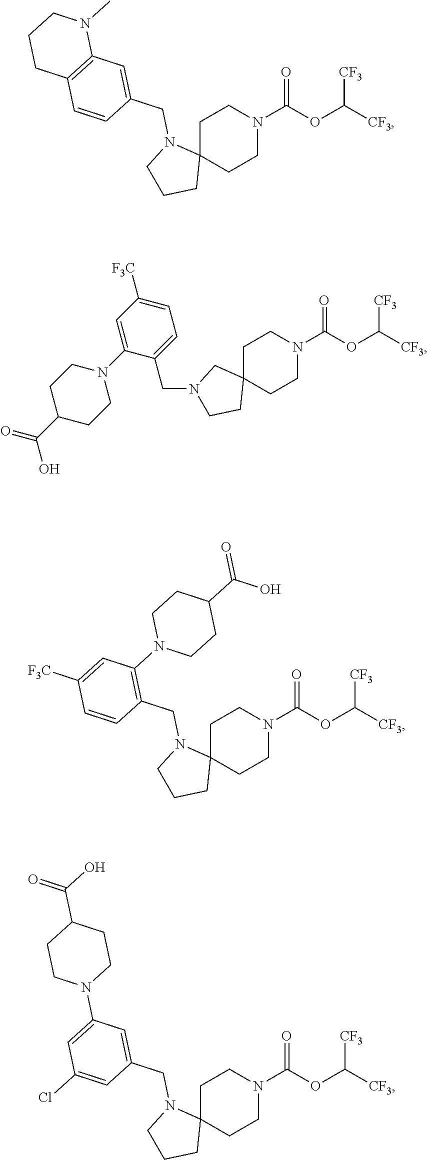 [SMILES: CN1CCCc2ccc(CN3CCCC34CCN(C(=O)OC(C(F)(F)F)C(F)(F)F)CC4)cc21 ; O=C(O)C1CCN(c2cc(C(F)(F)F)ccc2CN2CCC3(CCN(C(=O)OC(C(F)(F)F)C(F)(F)F)CC3)C2)CC1 ; O=C(O)C1CCN(c2cc(C(F)(F)F)ccc2CN2CCCC23CCN(C(=O)OC(C(F)(F)F)C(F)(F)F)CC3)CC1 ; O=C(O)C1CCN(c2cc(Cl)cc(CN3CCCC34CCN(C(=O)OC(C(F)(F)F)C(F)(F)F)CC4)c2)CC1]

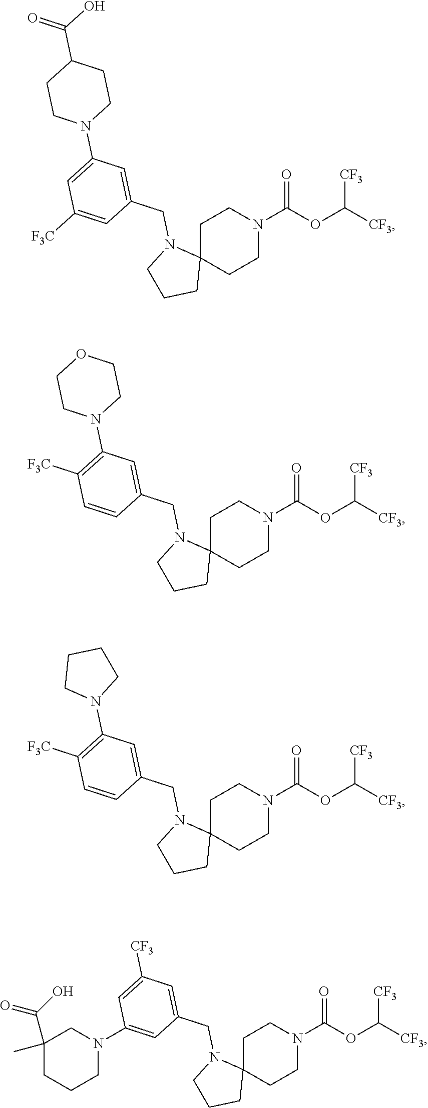 [SMILES: O=C(O)C1CCN(c2cc(CN3CCCC34CCN(C(=O)OC(C(F)(F)F)C(F)(F)F)CC4)cc(C(F)(F)F)c2)CC1 ; O=C(OC(C(F)(F)F)C(F)(F)F)N1CCC2(CCCN2Cc2ccc(C(F)(F)F)c(N3CCOCC3)c2)CC1 ; O=C(OC(C(F)(F)F)C(F)(F)F)N1CCC2(CCCN2Cc2ccc(C(F)(F)F)c(N3CCCC3)c2)CC1 ; CC1(C(=O)O)CCCN(c2cc(CN3CCCC34CCN(C(=O)OC(C(F)(F)F)C(F)(F)F)CC4)cc(C(F)(F)F)c2)C1]

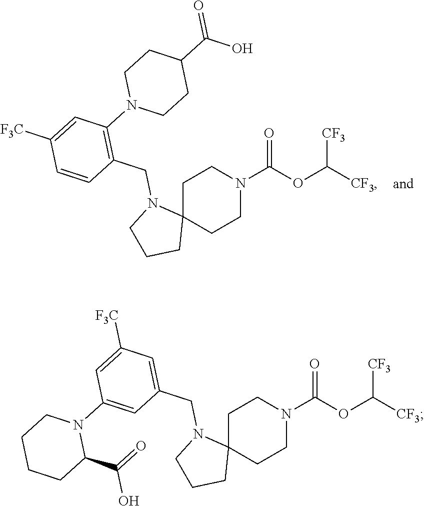 [SMILES: O=C(O)C1CCN(c2cc(C(F)(F)F)ccc2CN2CCCC23CCN(C(=O)OC(C(F)(F)F)C(F)(F)F)CC3)CC1 ; "and" ; O=C(O)[C@H]1CCCCN1c1cc(CN2CCCC23CCN(C(=O)OC(C(F)(F)F)C(F)(F)F)CC3)cc(C(F)(F)F)c1]

or a pharmaceutically acceptable salt or solvate thereof.

In some embodiments is a method for treating dyskinesia in a patient in need thereof, comprising administering to the patient a therapeutically effective amount of a compound of Formula (III):

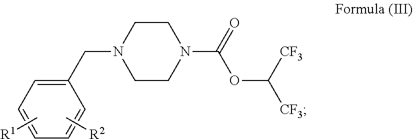

wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
   (iii) a 4-6 membered saturated monocyclic heterocycle; or
   (iv) a 7-8 membered bridged heterocyclic ring optionally containing an additional O, N, or S;
   wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
   the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
   or a pharmaceutically acceptable salt or solvate thereof.

In some embodiments is a method for treating dyskinesia in a patient in need thereof, comprising administering to the patient a therapeutically effective amount of a compound of Formula (III), wherein the dyskinesia is levodopa-induced dyskinesia.

In some embodiments of the methods for treating dyskinesia with a compound of Formula (III), R⁵ and R⁶, together with the nitrogen to which they are attached, form a 4-6 membered saturated monocyclic heterocycle substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S. In some embodiments of the methods for treating dyskinesia with a compound of Formula (III), R⁵ and R⁶, together with the nitrogen to which they are attached, form a 4-6 membered saturated monocyclic heterocycle substituted with one or two substituents independently selected from C₁₋₆haloalkyl, —C(O)OR⁹, and —NR⁹SO₂R⁸; wherein the 4-6 membered saturated monocyclic heterocycle is selected from pyrrolidine, piperidine, and morpholine. In some embodiments of the methods for treating dyskinesia with a compound of Formula (III), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is selected from pyrrolidine, piperidine, and morpholine. In some embodiments of the methods for treating dyskinesia with a compound of Formula (III), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is selected from pyrrolidine and piperidine. In some embodiments of the methods for treating dyskinesia with a compound of Formula (III), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is pyrrolidine. In some embodiments of the methods for treating dyskinesia with a compound of Formula (III), R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸; wherein the 4-6 membered saturated monocyclic heterocycle is piperidine. In some embodiments of the methods for treating dyskinesia with a compound of Formula (III), R⁵ and R⁶, together with the nitrogen to which they are attached, form a 4-6 membered saturated monocyclic heterocycle substituted with one substituent selected from C₁₋₆ haloalkyl, —C(O)OR⁹, and —NR⁹SO₂R⁸; wherein the 4-6 membered saturated monocyclic heterocycle is morpholine.

In some embodiments of the methods for treating dyskinesia with a compound of Formula (III), R⁵ and R⁶, together with the nitrogen to which they are attached, form a 7-8 membered bridged heterocyclic ring is optionally substituted with one or two substituents independently selected from halogen, oxo, and C₁₋₆alkyl. In some embodiments of the methods for treating dyskinesia with a compound of Formula (III), R⁵ and R⁶, together with the nitrogen to which they are attached, form an unsubstituted 7-8 membered bridged heterocyclic ring. In some embodiments of the methods for treating dyskinesia with a compound of Formula (III), R⁵ and R⁶, together with the nitrogen to which they are attached, form a 7-8 membered bridged heterocyclic ring is substituted with one or two substituents independently selected from halogen, oxo, and C₁₋₆alkyl. In some embodiments of the methods for treating dyskinesia with a compound of Formula (III), R⁵ and R⁶, together with the nitrogen to which they are attached, form a 7-8 membered bridged heterocyclic ring is substituted with one substituent selected from halogen, oxo, and C₁₋₆alkyl.

In some embodiments of the methods for treating dyskinesia with a compound of Formula (III), R¹ is halogen, —OR³, —SF₅, or C₁₋₆alkyl optionally substituted by halogen. In some embodiments of the methods for treating dyskinesia with a compound of Formula (III), R¹ is halogen, —CH₃, —CF₃, —OCH₃, or —OCF₃. In some embodiments of the methods for treating dyskinesia with a compound of Formula (III), R¹ is halogen, —SF₅, or C₁₋₆alkyl optionally substituted by halogen. In some embodiments of the methods for treating dyskinesia with a compound of Formula (III), R¹ is halogen. In some embodiments of the methods for treating dyskinesia with a compound of Formula (III), R¹ is —Cl. In some embodiments of the methods for treating dyskinesia with a compound of Formula (III), R¹ is C₁₋₆alkyl optionally substituted by halogen. In some embodiments of the methods for treating dyskinesia with a compound of Formula (III), R¹ is —CH₃. In some embodiments of the methods for treating dyskinesia with a compound of Formula (III), R¹ is —CF₃. In some embodiments of the methods for treating dyskinesia with a compound of Formula (III), R¹ is —SF₅. In some embodiments of the methods for treating dyskinesia with a compound of Formula (III), R¹ is —OCH₃. In some embodiments of the methods for treating dyskinesia with a compound of Formula (III), R¹ is —OCF₃.

In some embodiments of the methods for treating dyskinesia with a compound of Formula (III), the compound is selected from:

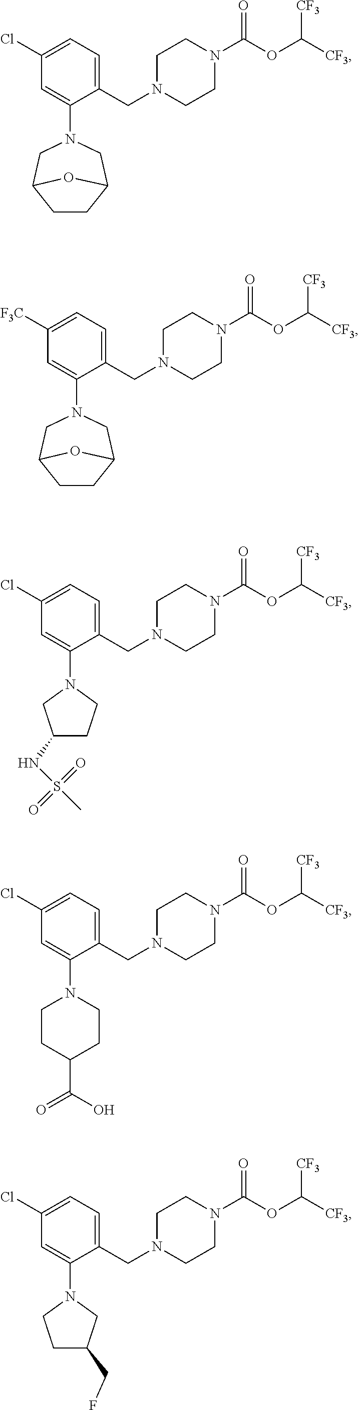 [SMILES: O=C(OC(C(F)(F)F)C(F)(F)F)N1CCN(Cc2ccc(Cl)cc2N2CC3CCC(C2)O3)CC1 ; O=C(OC(C(F)(F)F)C(F)(F)F)N1CCN(Cc2ccc(C(F)(F)F)cc2N2CC3CCC(C2)O3)CC1 ; CS(=O)(=O)N[C@H]1CCN(c2cc(Cl)ccc2CN2CCN(C(=O)OC(C(F)(F)F)C(F)(F)F)CC2)C1 ; O=C(O)C1CCN(c2cc(Cl)ccc2CN2CCN(C(=O)OC(C(F)(F)F)C(F)(F)F)CC2)CC1 ; O=C(OC(C(F)(F)F)C(F)(F)F)N1CCN(Cc2ccc(Cl)cc2N2CC[C@H](CF)C2)CC1]

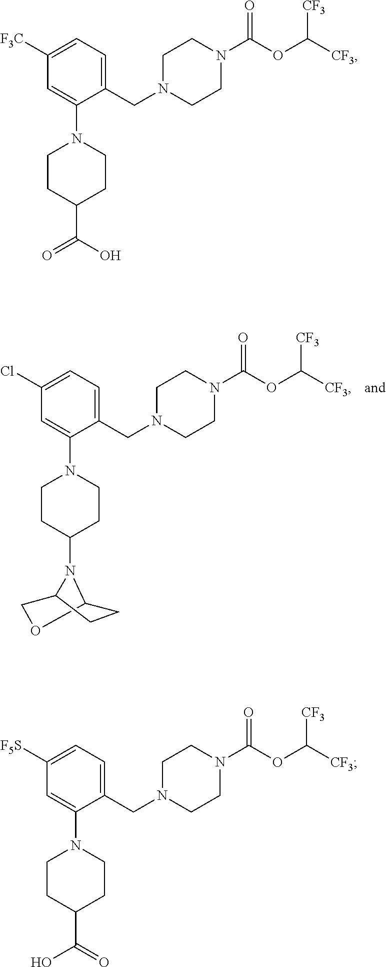

or a pharmaceutically acceptable salt or solvate thereof.

In some embodiments is a method for treating dyskinesia in a patient in need thereof, comprising administering to the patient a therapeutically effective amount of a compound of Formula (IV):

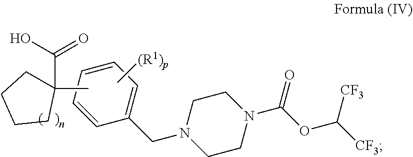

wherein:
each R¹ is independently halogen, C₁₋₆alkyl, C₁₋₆haloalkyl, C₁₋₆alkoxy, C₁₋₆haloalkoxy, C₃₋₈cycloalkyl, —OH, —CN, or —SF₅;
n is 1 or 2; and
p is 0, 1, 2, 3, or 4;
   or a pharmaceutically acceptable salt or solvate thereof.

In some embodiments is a method for treating dyskinesia in a patient in need thereof, comprising administering to the patient a therapeutically effective amount of a compound of Formula (IV), wherein the dyskinesia is levodopa-induced dyskinesia.

In some embodiments of the methods for treating dyskinesia with a compound of Formula (IV), n is 1. In some embodiments of the methods for treating dyskinesia with a compound of Formula (IV), n is 2.

In some embodiments of the methods for treating dyskinesia with a compound of Formula (IV), p is 0, 1, or 2. In some embodiments of the methods for treating dyskinesia with a compound of Formula (IV), p is 0 or 1. In some embodiments of the methods for treating dyskinesia with a compound of Formula (IV), p is 0. In some embodiments of the methods for treating dyskinesia with a compound of Formula (IV), p is 1. In some embodiments of the methods for treating dyskinesia with a compound of Formula (IV), p is 2.

In some embodiments of the methods for treating dyskinesia with a compound of Formula (IV), p is 1 and R¹ is halogen, C₁₋₆alkyl, or C₁₋₆haloalkyl. In some embodiments of the methods for treating dyskinesia with a compound of Formula (IV), p is 1 and R¹ is halogen. In some embodiments of the methods for treating dyskinesia with a compound of Formula (IV), p is 1 and R¹ is —Cl. In some embodiments of the methods for treating dyskinesia with a compound of Formula (IV), p is 1 and R¹ is —F. In some embodiments of the methods for treating dyskinesia with a compound of Formula (IV), p is 1 and R¹ is C₁₋₆alkyl. In some embodiments of the methods for treating dyskinesia with a compound of Formula (IV), p is 1 and R¹ is —CH₃. In some embodiments of the methods for treating dyskinesia with a compound of Formula (IV), p is 1 and R¹ is C₁₋₆haloalkyl. In some embodiments of the methods for treating dyskinesia with a compound of Formula (IV), p is 1 and R¹ is —CF₃.

In some embodiments of the methods for treating dyskinesia with a compound of Formula (IV), the compound is selected from:

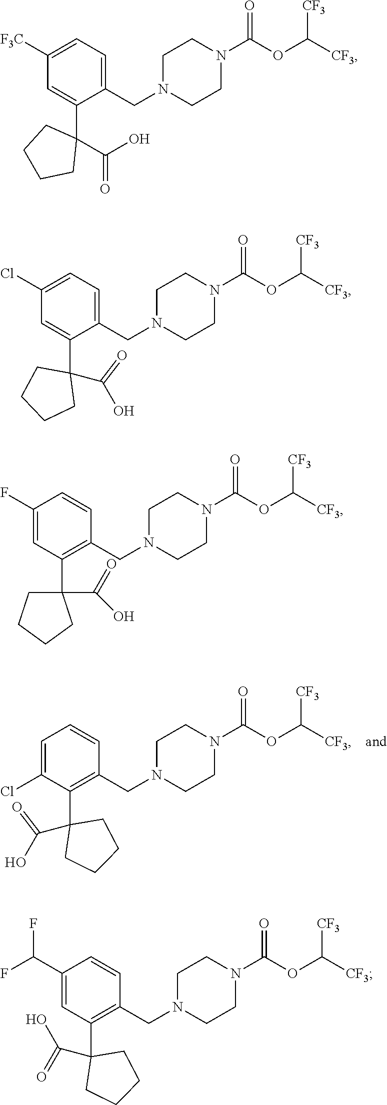 [SMILES: O=C(OC(C(F)(F)F)C(F)(F)F)N1CCN(Cc2ccc(C(F)(F)F)cc2C2(C(=O)O)CCCC2)CC1 ; O=C(OC(C(F)(F)F)C(F)(F)F)N1CCN(Cc2ccc(Cl)cc2C2(C(=O)O)CCCC2)CC1 ; O=C(OC(C(F)(F)F)C(F)(F)F)N1CCN(Cc2ccc(F)cc2C2(C(=O)O)CCCC2)CC1 ; O=C(OC(C(F)(F)F)C(F)(F)F)N1CCN(Cc2cccc(Cl)c2C2(C(=O)O)CCCC2)CC1 ; O=C(OC(C(F)(F)F)C(F)(F)F)N1CCN(Cc2ccc(C(F)F)cc2C2(C(=O)O)CCCC2)CC1]

or a pharmaceutically acceptable salt or solvate thereof.

In some embodiments is a method for treating dyskinesia in a patient in need thereof, comprising administering to the patient a therapeutically effective amount of a compound of Formula (V):

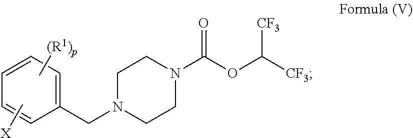

wherein:
X is —N(R²)(R³), —C₁₋₆alkyl-N(R⁴)(R⁵), —C(O)N(R⁴)(R⁵)

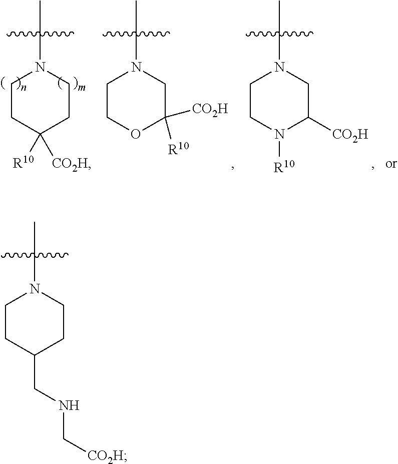

each R¹ is independently halogen, C₁₋₆alkyl, C₁₋₆haloalkyl, C₁₋₆alkoxy, C₁₋₆haloalkoxy, C₃₋₈cycloalkyl, —OH, —CN, or —SF₅;
R² and R³, together with the nitrogen to which they are attached, form
   (iii) a C₂-C₈heterocycloalkyl; or
   (iv) a C₂-C₈heteroaryl;
wherein the C₂-C₈heterocycloalkyl or the C₂-C₈heteroaryl is substituted with one R⁶ and optionally substituted with one or two additional substituents selected from halogen, C₁₋₆alkyl, C₁₋₆haloalkyl, and C₁₋₆alkoxy;
R⁴ and R⁵, together with the nitrogen to which they are attached, form
   (iii) a C₂-C₈heterocycloalkyl; or
   (iv) a C₂-C₈heteroaryl;
wherein the C₂-C₈heterocycloalkyl or the C₂-C₈heteroaryl is substituted with one R⁷ and optionally substituted with one or two additional substituents selected from halogen, C₁₋₆alkyl, C₁₋₆haloalkyl, and C₁₋₆alkoxy;
R⁶ is —C₁₋₆alkyl-CO₂H or —N(R⁸)—C₁₋₆alkyl-CO₂H;
R⁷ is —CO₂H, —C₁₋₆alkyl-CO₂H, or —N(R⁹)—C₁₋₆alkyl-CO₂H;
R⁸ is H or C₁₋₆alkyl;
R⁹ is H or C₁₋₆alkyl;
R¹⁰ is C₁₋₆alkyl;
m is 0, 1, or 2;
n is 0 or 1; and
p is 0, 1, 2, 3, or 4;
   or a pharmaceutically acceptable salt or solvate thereof.

In some embodiments is a method for treating dyskinesia in a patient in need thereof, comprising administering to the patient a therapeutically effective amount of a compound of Formula (V), wherein the dyskinesia is levodopa-induced dyskinesia.

In some embodiments of the methods for treating dyskinesia with a compound of Formula (V), X is

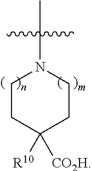

In some embodiments of the methods for treating dyskinesia with a compound of Formula (V), X is

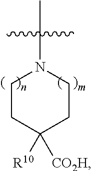

m is 1, and n is 1. In some embodiments of the methods for treating dyskinesia with a compound of Formula (V), X is

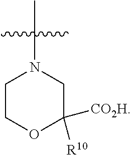

In some embodiments of the methods for treating dyskinesia with a compound of Formula (V), X is

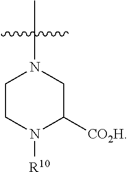

In some embodiments of the methods for treating dyskinesia with a compound of Formula (V), R¹⁰ is —CH₃. In some embodiments of the methods for treating dyskinesia with a compound of Formula (V), X is

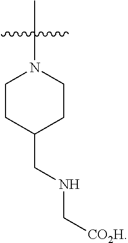 [SMILES: *N1CCC(CNCC(=O)O)CC1]

In some embodiments of the methods for treating dyskinesia with a compound of Formula (V), X is —N(R²)(R³). In some embodiments of the methods for treating dyskinesia with a compound of Formula (V), X is —N(R²)(R³) and R² and R³, together with the nitrogen to which they are attached, form a C₂-C₈heterocycloalkyl substituted with one R⁶. In some embodiments of the methods for treating dyskinesia with a compound of Formula (V), X is —N(R²)(R³) and R² and R³, together with the nitrogen to which they are attached, form a C₂-C₈heterocycloalkyl selected from

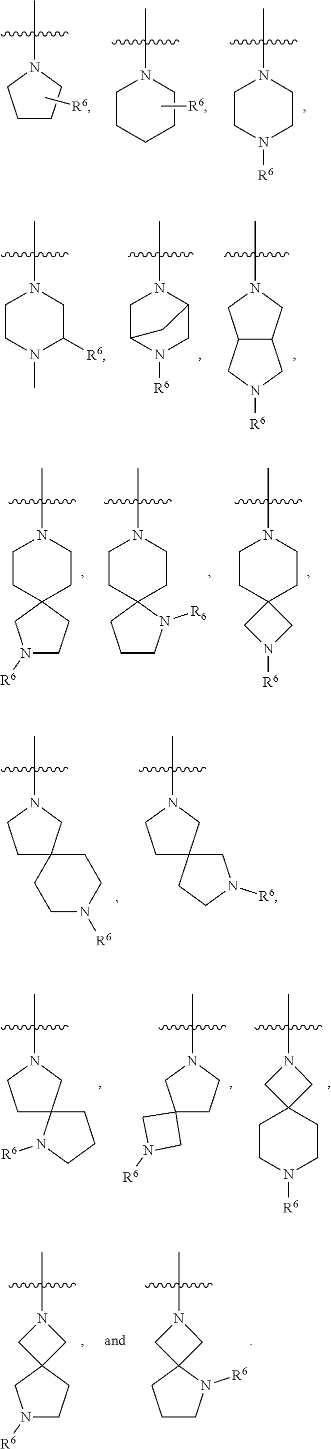

In some embodiments of the methods for treating dyskinesia with a compound of Formula (V), p is 0, 1, or 2. In some embodiments of the methods for treating dyskinesia with a compound of Formula (V), p is 0 or 1. In some embodiments of the methods for treating dyskinesia with a compound of Formula (V), p is 0. In some embodiments of the methods for treating dyskinesia with a compound of Formula (V), p is 1. In some embodiments of the methods for treating dyskinesia with a compound of Formula (V), p is 2.

In some embodiments of the methods for treating dyskinesia with a compound of Formula (V), p is 1 and R¹ is halogen, C₁₋₆alkyl, or C₁₋₆haloalkyl. In some embodiments of the methods for treating dyskinesia with a compound of Formula (V), p is 1 and R¹ is halogen. In some embodiments of the methods for treating dyskinesia with a compound of Formula (V), p is 1 and R¹ is —Cl. In some embodiments of the methods for treating dyskinesia with a compound of Formula (V), p is 1 and R¹ is —F. In some embodiments of the methods for treating dyskinesia with a compound of Formula (V), p is 1 and R¹ is C₁₋₆alkyl. In some embodiments of the methods for treating dyskinesia with a compound of Formula (V), p is 1 and R¹ is —CH₃. In some embodiments of the methods for treating dyskinesia with a compound of Formula (V), p is 1 and R¹ is C₁₋₆haloalkyl. In some embodiments of the methods for treating dyskinesia with a compound of Formula (V), p is 1 and R¹ is —CF₃.

In some embodiments of the methods for treating dyskinesia with a compound of Formula (V), the compound is selected from:

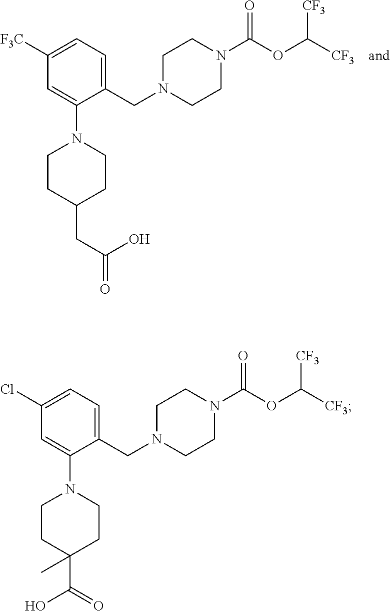 [SMILES: O=C(O)CC1CCN(c2cc(C(F)(F)F)ccc2CN2CCN(C(=O)OC(C(F)(F)F)C(F)(F)F)CC2)CC1 ; CC1(C(=O)O)CCN(c2cc(Cl)ccc2CN2CCN(C(=O)OC(C(F)(F)F)C(F)(F)F)CC2)CC1]

or a pharmaceutically acceptable salt or solvate thereof.

In some embodiments is a method for treating dyskinesia in a patient in need thereof, comprising administering to the patient a therapeutically effective amount of a compound of Formula (VI):

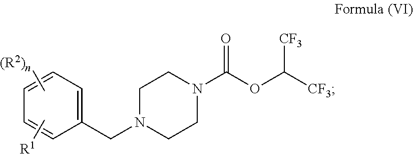

wherein:
R¹ is —N(R³)(R⁵) or —NH(R⁴);
each R² is independently selected from halogen, C₁₋₆alkyl, —CN, C₁₋₆haloalkyl, and —OR⁶;
R³ is —CH₂CO₂H, —CH₂CH₂CO₂H, or —CH(CH₃)CO₂H;
R⁴ is —(CH₂)ₘ—CO₂H;
R⁵ is H or C₁₋₃alkyl;
each R⁶ is independently selected from H, C₁₋₆alkyl, and C₁₋₆haloalkyl;
n is 0, 1, 2, 3, or 4; and
m is 3;
   or a pharmaceutically acceptable salt or solvate thereof.

In some embodiments is a method for treating dyskinesia in a patient in need thereof, comprising administering to the patient a therapeutically effective amount of a compound of Formula (VI), wherein the dyskinesia is levodopa-induced dyskinesia.

In some embodiments of the methods for treating dyskinesia with a compound of Formula (VI), wherein R¹ is —N(R³)(R⁵). In some embodiments of the methods for treating dyskinesia with a compound of Formula (VI), wherein R¹ is —N(R³)(R⁵) and R⁵ is H. In some embodiments of the methods for treating dyskinesia with a compound of Formula (VI), wherein R¹ is —N(R³)(R⁵), R⁵ is H, and R³ is —CH₂CO₂H. In some embodiments of the methods for treating dyskinesia with a compound of Formula (VI), wherein R¹ is —N(R³)(R⁵), R⁵ is H, and R³ is —CH₂CH₂CO₂H. In some embodiments of the methods for treating dyskinesia with a compound of Formula (VI), wherein R¹ is —N(R³)(R⁵), R⁵ is H, and R³ is —CH(CH₃)CO₂H.

In some embodiments of the methods for treating dyskinesia with a compound of Formula (VI), wherein R¹ is —N(R³)(R⁵) and R⁵ is C₁₋₃alkyl. In some embodiments of the methods for treating dyskinesia with a compound of Formula (VI), wherein R¹ is —N(R³)(R⁵), R⁵ is C₁₋₃alkyl, and R³ is —CH₂CO₂H. In some embodiments of the methods for treating dyskinesia with a compound of Formula (VI), wherein R¹ is —N(R³)(R⁵), R⁵ is C₁₋₃alkyl, and R³ is —CH₂CH₂CO₂H. In some embodiments of the methods for treating dyskinesia with a compound of Formula (VI), wherein R¹ is —N(R³)(R⁵), R⁵ is C₁₋₃alkyl, and R³ is —CH(CH₃)CO₂H.

In some embodiments of the methods for treating dyskinesia with a compound of Formula (VI), wherein R¹ is —NH(CH₂)₃CO₂H.

In some embodiments of the methods for treating dyskinesia with a compound of Formula (VI), wherein n is 0, 1, or 2. In some embodiments of the methods for treating dyskinesia with a compound of Formula (VI), wherein n is 0 or 1. In some embodiments of the methods for treating dyskinesia with a compound of Formula (VI), wherein n is 0. In some embodiments of the methods for treating dyskinesia with a compound of Formula (VI), wherein n is 1. In some embodiments of the methods for treating dyskinesia with a compound of Formula (VI), wherein n is 2.

In some embodiments of the methods for treating dyskinesia with a compound of Formula (VI), n is 1 and R² is halogen, C₁₋₆alkyl, C₁₋₆haloalkyl, or —OR⁶. In some embodiments of the methods for treating dyskinesia with a compound of Formula (VI), n is 1 and R² is halogen, C₁₋₆alkyl, or C₁₋₆haloalkyl. In some embodiments of the methods for treating dyskinesia with a compound of Formula (VI), n is 1 and R² is halogen. In some embodiments of the methods for treating dyskinesia with a compound of Formula (VI), n is 1 and R² is —Cl. In some embodiments of the methods for treating dyskinesia with a compound of Formula (VI), n is 1 and R² is —F. In some embodiments of the methods for treating dyskinesia with a compound of Formula (VI), n is 1 and R² is C₁₋₆ alkyl. In some embodiments of the methods for treating dyskinesia with a compound of Formula (VI), n is 1 and R² is —CH₃. In some embodiments of the methods for treating dyskinesia with a compound of Formula (VI), n is 1 and R² is C₁₋₆haloalkyl. In some embodiments of the methods for treating dyskinesia with a compound of Formula (VI), n is 1 and R² is —CF₃.

In some embodiments of the methods for treating dyskinesia with a compound of Formula (VI), the compound is selected from:

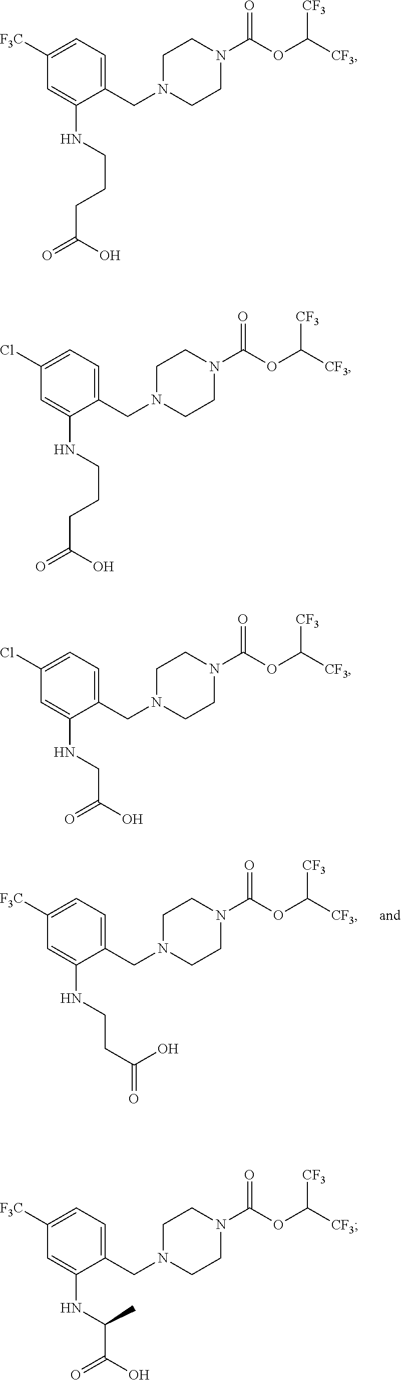 [SMILES: O=C(O)CCCNc1cc(C(F)(F)F)ccc1CN1CCN(C(=O)OC(C(F)(F)F)C(F)(F)F)CC1 ; O=C(O)CCCNc1cc(Cl)ccc1CN1CCN(C(=O)OC(C(F)(F)F)C(F)(F)F)CC1 ; O=C(O)CNc1cc(Cl)ccc1CN1CCN(C(=O)OC(C(F)(F)F)C(F)(F)F)CC1 ; O=C(O)CCNc1cc(C(F)(F)F)ccc1CN1CCN(C(=O)OC(C(F)(F)F)C(F)(F)F)CC1 ; C[C@H](Nc1cc(C(F)(F)F)ccc1CN1CCN(C(=O)OC(C(F)(F)F)C(F)(F)F)CC1)C(=O)O]

or a pharmaceutically acceptable salt or solvate thereof.

In some embodiments is a method for treating dyskinesia in a patient in need thereof, comprising administering to the patient a therapeutically effective amount of a compound of Formula (VII):

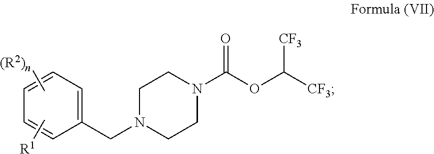

wherein:
R¹ is —R¹⁴, —OR³, —SR⁴, —S(O)₂₄, or —C≡C—(CR⁶R⁷)—R⁸;
each R² is independently selected from C₁₋₆alkyl, halogen, —CN, C₁₋₆haloalkyl, —C₁₋₆alkyl(heterocycloalkyl), —OR¹⁷, and —C(O)NR¹⁸R¹⁹;
R³ is —(CR⁶R⁷)ₘ—R⁸, —(CR⁶R⁷)ₚ—Y—(CR⁶R⁷)_{q}—R⁸, or —(CR⁶R⁷)ₜ—C₃₋₆cycloalkyl-R⁸;
R⁴ is —(CR⁶R⁷)ₘ—R⁸, —(CR⁶R⁷)—C(O)OH, or —(CR⁶R⁷)ₚ—Y—(CR⁶R⁷)_{q}—R⁸;
Y is —O— or —N(R²²)—;
each R⁶ and R⁷ is each independently selected from H, F, and C₁₋₆alkyl; or R⁶ and R⁷, together with the carbon to which they are attached, form a C₃₋₆cycloalkyl ring;
R⁸ is —C(O)OR⁹, —C(O)R¹⁰, or —C(O)O—(CR¹²R¹³)—OC(O)R¹¹;
R^{8′} is —C(O)OR^{9′}, —C(O)R^{10′}, or —C(O)O—(CR¹²R¹³)—OC(O)R¹¹;
R⁹ is H or C₁₋₆alkyl;
R^{9′} is C₁₋₆alkyl;
R¹⁰ is C₁₋₆alkyl or —NHSO₂R²¹;
R^{10′} is C₂₋₆alkyl or —NHSO₂R²¹;
R¹¹ is C₁₋₆alkyl or C₁₋₆alkoxy;
R¹² and R¹³ is each independently H or C₁₋₆alkyl;
R¹⁴ is —(CR¹⁵R¹⁶)ₘ—R⁸ or —(CR⁶R⁷)ₚ—Y—(CR⁶R⁷)_{q}—R⁸;
each R¹⁵ and R¹⁶ is each independently selected from H, F, and C₁₋₆alkyl; each R¹⁷ is independently selected from H, C₁₋₆alkyl, C₁₋₆haloalkyl, and C₃₋₆cycloalkyl;
each R¹⁸ and R¹⁹ is each independently selected from H, C₁₋₆alkyl, C₃₋₆cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl or C₃₋₆cycloalkyl;
R²² is H, C₁₋₆alkyl, or —SO₂R²³.
R²³ is C₁₋₆alkyl;
m is 1, 2, 3 or 4;
n is 0, 1, 2, 3, or 4;
p is 2, 3, or 4;
q is 1, 2, or 3;
t is 0, 1, or 2; and
v is 3 or 4;
   or a pharmaceutically acceptable salt or solvate thereof.

In some embodiments is a method for treating dyskinesia in a patient in need thereof, comprising administering to the patient a therapeutically effective amount of a compound of Formula (VII), wherein the dyskinesia is levodopa-induced dyskinesia.

In some embodiments of the methods for treating dyskinesia with a compound of Formula (VII), R¹ is —OR³. In some embodiments of the methods for treating dyskinesia with a compound of Formula (VII), R¹ is —OR³ and R³ is —(CR⁶R⁷)ₘ—R⁸. In some embodiments of the methods for treating dyskinesia with a compound of Formula (VII), R⁸ is —C(O)OR⁹. In some embodiments of the methods for treating dyskinesia with a compound of Formula (VII), R⁸ is —C(O)OR⁹ and R⁹ is H. In some embodiments of the methods for treating dyskinesia with a compound of Formula (VII), R⁸ is —C(O)OR⁹ and R⁹ is C₁₋₆alkyl. In some embodiments of the methods for treating dyskinesia with a compound of Formula (VII), R⁸ is —C(O)OR⁹ and R⁹ is —CH₃. In some embodiments of the methods for treating dyskinesia with a compound of Formula (VII), R⁸ is —C(O)OR⁹ and R⁹ is —CH₂CH₃. In some embodiments of the methods for treating dyskinesia with a compound of Formula (VII), R⁸ is —C(O)R¹⁰. In some embodiments of the methods for treating dyskinesia with a compound of Formula (VII), R⁸ is —C(O)R¹⁰ and R¹⁰ is —NHSO₂R²¹. In some embodiments of the methods for treating dyskinesia with a compound of Formula (VII), R⁸ is —C(O)R¹⁰, R¹⁰ is —NHSO₂R²¹, and R²¹ is C₁₋₆alkyl. In some embodiments of the methods for treating dyskinesia with a compound of Formula (VII), R⁸ is —C(O)R¹⁰, R¹⁰ is —NHSO₂R²¹, and R²¹ is C₃₋₆cycloalkyl. In some embodiments of the methods for treating dyskinesia with a compound of Formula (VII), R⁸ is —C(O)O—(CR¹²R¹³)—OC(O)R¹¹. In some embodiments of the methods for treating dyskinesia with a compound of Formula (VII), R⁸ is —C(O)O—(CR¹²R¹³)—OC(O)R¹¹ and R¹¹ is C₁₋₆alkyl. In some embodiments of the methods for treating dyskinesia with a compound of Formula (VII), R⁸ is —C(O)O—(CR¹²R¹³)—OC(O)R¹¹ and R¹¹ is C₁₋₆alkoxy. In some embodiments of the methods for treating dyskinesia with a compound of Formula (VII), R¹ is —OR³ and R³ is —CH₂C(O)OH. In some embodiments of the methods for treating dyskinesia with a compound of Formula (VII), R¹ is —OR³ and R³ is —CH₂CH₂C(O)OH. In some embodiments of the methods for treating dyskinesia with a compound of Formula (VII), R¹ is —OR³ and R³ is

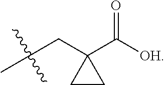 [SMILES: *CC1(C(=O)O)CC1]

In some embodiments of the methods for treating dyskinesia with a compound of Formula (VII), R¹ is —OR³ and R³ is

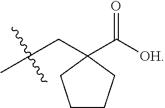 [SMILES: *CC1(C(=O)O)CCCC1]

In some embodiments of the methods for treating dyskinesia with a compound of Formula (VII), R¹ is —OR³ and R³ is —CH₂CH₂CH₂C(O)OH. In some embodiments of the methods for treating dyskinesia with a compound of Formula (VII), R¹ is —OR³ and R³ is —CH₂CH(CH₃)CH₂C(O)OH. In some embodiments of the methods for treating dyskinesia with a compound of Formula (VII), R¹ is —OR³ and R³ is —CH₂CH₂C(O)OCH₃. In some embodiments of the methods for treating dyskinesia with a compound of Formula (VII), R¹ is —OR³ and R³ is —CH₂CH₂C(O)OCH₂CH₃. In some embodiments of the methods for treating dyskinesia with a compound of Formula (VII), R¹ is —OR³ and R³ is —CH₂CH₂C(O)OC(CH₃)₃. In some embodiments of the methods for treating dyskinesia with a compound of Formula (VII), R¹ is —OR³ and R³ is —CH₂CH₂CH₂C(O)OCH₃. In some embodiments of the methods for treating dyskinesia with a compound of Formula (VII), R¹ is —OR³ and R³ is —CH₂CH₂CH₂C(O)OCH₂CH₃. In some embodiments of the methods for treating dyskinesia with a compound of Formula (VII), R¹ is —OR³ and R³ is —CH₂CH₂CH₂C(O)OC(CH₃)₃.

In some embodiments of the methods for treating dyskinesia with a compound of Formula (VII), R¹ is —OR³ and R³ is —(CR⁶R⁷)ₜ—C₃₋₆cycloalkyl-R⁸. In some embodiments of the methods for treating dyskinesia with a compound of Formula (VII), R¹ is —OR³, R³ is —(CR⁶R⁷)ₜ—C₃₋₆cycloalkyl-R⁸, and t is 0. In some embodiments of the methods for treating dyskinesia with a compound of Formula (VII), R¹ is —OR³, R³ is —(CR⁶R⁷)ₜ—C₃₋₆cycloalkyl-R⁸, and t is 1. In some embodiments of the methods for treating dyskinesia with a compound of Formula (VII), R¹ is —OR³, R³ is —(CR⁶R⁷)ₜ—C₃₋₆ cycloalkyl-R⁸, and t is 2. In some embodiments of the methods for treating dyskinesia with a compound of Formula (VII), R¹ is —OR³ and R³ is -cyclopropyl-C(O)OH. In some embodiments of the methods for treating dyskinesia with a compound of Formula (VII), R¹ is —OR³ and R³ is -cyclobutyl-C(O)OH. In some embodiments of the methods for treating dyskinesia with a compound of Formula (VII), R¹ is —OR³ and R³ is -cyclopentyl-C(O)OH.

In some embodiments of the methods for treating dyskinesia with a compound of Formula (VII), R¹ is —R¹⁴. In some embodiments of the methods for treating dyskinesia with a compound of Formula (VII), R¹ is —R¹⁴ and R¹⁴ is —(CR¹⁵R¹⁶)ₘ—R⁸. In some embodiments of the methods for treating dyskinesia with a compound of Formula (VII), R¹⁴ is —(CR¹⁵R¹⁶)ₘ—R⁸ and m is 1. In some embodiments of the methods for treating dyskinesia with a compound of Formula (VII), R¹⁴ is —(CR¹⁵R¹⁶)ₘ—R⁸ and m is 2. In some embodiments of the methods for treating dyskinesia with a compound of Formula (VII), R¹⁴ is —(CR¹⁵R¹⁶)ₘ—R⁸ and m is 3. In some embodiments of the methods for treating dyskinesia with a compound of Formula (VII), R¹⁴ is —(CR¹⁵R¹⁶)ₘ—R⁸ and m is 4. In some embodiments of the methods for treating dyskinesia with a compound of Formula (VII), R⁸ is —C(O)OR⁹. In some embodiments of the methods for treating dyskinesia with a compound of Formula (VII), R⁸ is —C(O)OR⁹ and R⁹ is H. In some embodiments of the methods for treating dyskinesia with a compound of Formula (VII), R⁸ is —C(O)OR⁹ and R⁹ is C₁₋₆alkyl. In some embodiments of the methods for treating dyskinesia with a compound of Formula (VII), R⁸ is —C(O)OR⁹ and R⁹ is —CH₃. In some embodiments of the methods for treating dyskinesia with a compound of Formula (VII), R⁸ is —C(O)OR⁹ and R⁹ is —CH₂CH₃. In some embodiments of the methods for treating dyskinesia with a compound of Formula (VII), R⁸ is —C(O)R¹⁰. In some embodiments of the methods for treating dyskinesia with a compound of Formula (VII), R⁸ is —C(O)R¹⁰ and R¹⁰ is —NHSO₂R²¹. In some embodiments of the methods for treating dyskinesia with a compound of Formula (VII), R⁸ is —C(O)R¹⁰, R¹⁰ is —NHSO₂R²¹, and R²¹ is C₁₋₆alkyl. In some embodiments of the methods for treating dyskinesia with a compound of Formula (VII), R⁸ is —C(O)R¹⁰, R¹⁰ is —NHSO₂R²¹, and R²¹ is C₃₋₆cycloalkyl. In some embodiments of the methods for treating dyskinesia with a compound of Formula (VII), R⁸ is —C(O)O—(CR¹²R¹³)—OC(O)R¹¹. In some embodiments of the methods for treating dyskinesia with a compound of Formula (VII), R⁸ is —C(O)O—(CR¹²R¹³)—OC(O)R¹¹ and R¹¹ is C₁₋₆alkyl. In some embodiments of the methods for treating dyskinesia with a compound of Formula (VII), R⁸ is —C(O)O—(CR¹²R¹³)—OC(O)R¹¹ and R¹¹ is C₁₋₆alkoxy. In some embodiments of the methods for treating dyskinesia with a compound of Formula (VII), R¹ is —R¹⁴ and R¹⁴ is —CH₂C(O)OH. In some embodiments of the methods for treating dyskinesia with a compound of Formula (VII), R¹ is —R¹⁴ and R¹⁴ is —CH₂CH₂C(O)OH. In some embodiments of the methods for treating dyskinesia with a compound of Formula (VII), R¹ is —R¹⁴ and R¹⁴ is —CH₂CH₂CH₂C(O)OH. In some embodiments of the methods for treating dyskinesia with a compound of Formula (VII), R¹ is —R¹⁴ and R¹⁴ is —CH₂CH₂CH₂CH₂C(O)OH. In some embodiments of the methods for treating dyskinesia with a compound of Formula (VII), R¹ is —R¹⁴ and R¹⁴ is —CH₂CH(CH₃)CH₂C(O)OH. In some embodiments of the methods for treating dyskinesia with a compound of Formula (VII), R¹ is —R¹⁴ and R¹⁴ is —CH₂CH₂C(O)OCH₃. In some embodiments of the methods for treating dyskinesia with a compound of Formula (VII), R¹ is —R¹⁴ and R¹⁴ is —CH₂CH₂C(O)OCH₂CH₃. In some embodiments of the methods for treating dyskinesia with a compound of Formula (VII), R¹ is —R¹⁴ and R¹⁴ is —CH₂CH₂C(O)OC(CH₃)₃. In some embodiments of the methods for treating dyskinesia with a compound of Formula (VII), R¹ is —R¹⁴ and R¹⁴ is —CH₂CH₂CH₂C(O)OCH₃. In some embodiments of the methods for treating dyskinesia with a compound of Formula (VII), R¹ is —R¹⁴ and R¹⁴ is —CH₂CH₂CH₂C(O)OCH₂CH₃. In some embodiments of the methods for treating dyskinesia with a compound of Formula (VII), R¹ is —R¹⁴ and R¹⁴ is —CH₂CH₂CH₂C(O)OC(CH₃)₃. In some embodiments of the methods for treating dyskinesia with a compound of Formula (VII), R¹ is —R¹⁴ and R¹⁴ is —CH₂CH₂CH₂CH₂C(O)OCH₃. In some embodiments of the methods for treating dyskinesia with a compound of Formula (VII), R¹ is —R¹⁴ and R¹⁴ is —CH₂CH₂CH₂CH₂C(O)OCH₂CH₃. In some embodiments of the methods for treating dyskinesia with a compound of Formula (VII), R¹ is —R¹⁴ and R¹⁴ is —CH₂CH₂CH₂CH₂C(O)OC(CH₃)₃.

In some embodiments of the methods for treating dyskinesia with a compound of Formula (VII), n is 0, 1, or 2. In some embodiments of the methods for treating dyskinesia with a compound of Formula (VII), n is 1 or 2. In some embodiments of the methods for treating dyskinesia with a compound of Formula (VII), n is 0 or 1. In some embodiments of the methods for treating dyskinesia with a compound of Formula (VII), n is 0. In some embodiments of the methods for treating dyskinesia with a compound of Formula (VII), n is 1. In some embodiments of the methods for treating dyskinesia with a compound of Formula (VII), n is 2.

In some embodiments of the methods for treating dyskinesia with a compound of Formula (VII), n is 1 and R² is halogen, C₁₋₆alkyl, C₁₋₆haloalkyl, or —OR¹⁷. In some embodiments of the methods for treating dyskinesia with a compound of Formula (VII), n is 1 and R² is independently selected from C₁₋₆alkyl, halogen, —CN, or C₁₋₆haloalkyl. In some embodiments of the methods for treating dyskinesia with a compound of Formula (VII), n is 1 and R² is halogen, C₁₋₆alkyl, or C₁₋₆haloalkyl. In some embodiments of the methods for treating dyskinesia with a compound of Formula (VII), n is 1 and R² is halogen. In some embodiments of the methods for treating dyskinesia with a compound of Formula (VII), n is 1 and R² is —Cl. In some embodiments of the methods for treating dyskinesia with a compound of Formula (VII), n is 1 and R² is —F. In some embodiments of the methods for treating dyskinesia with a compound of Formula (VII), n is 1 and R² is C₁₋₆alkyl. In some embodiments of the methods for treating dyskinesia with a compound of Formula (VII), n is 1 and R² is —CH₃. In some embodiments of the methods for treating dyskinesia with a compound of Formula (VII), n is 1 and R² is C₁₋₆haloalkyl. In some embodiments of the methods for treating dyskinesia with a compound of Formula (VII), n is 1 and R² is —CF₃.

In some embodiments of the methods for treating dyskinesia with a compound of Formula (VII), the compound is selected from:

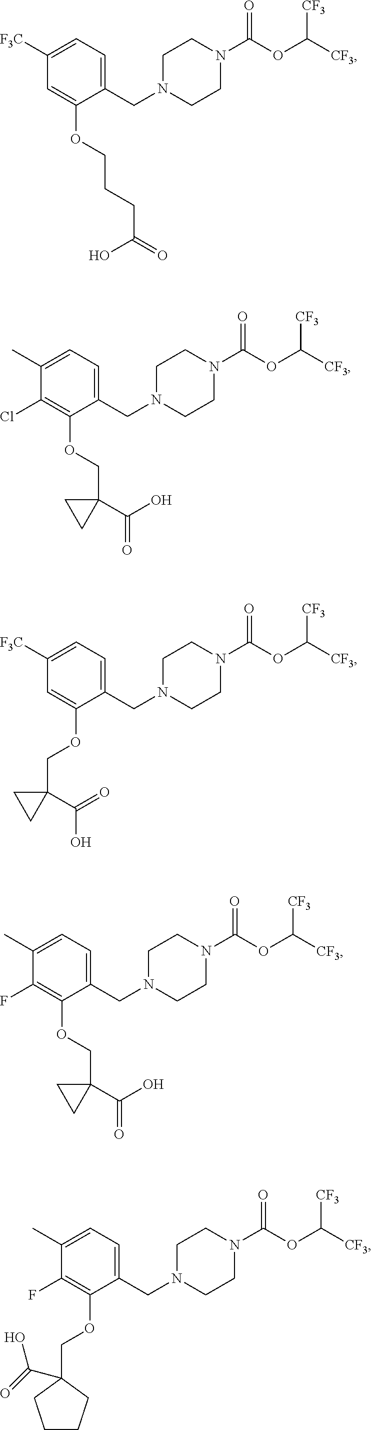 [SMILES: O=C(O)CCCOc1cc(C(F)(F)F)ccc1CN1CCN(C(=O)OC(C(F)(F)F)C(F)(F)F)CC1 ; Cc1ccc(CN2CCN(C(=O)OC(C(F)(F)F)C(F)(F)F)CC2)c(OCC2(C(=O)O)CC2)c1Cl ; O=C(OC(C(F)(F)F)C(F)(F)F)N1CCN(Cc2ccc(C(F)(F)F)cc2OCC2(C(=O)O)CC2)CC1 ; Cc1ccc(CN2CCN(C(=O)OC(C(F)(F)F)C(F)(F)F)CC2)c(OCC2(C(=O)O)CC2)c1F ; Cc1ccc(CN2CCN(C(=O)OC(C(F)(F)F)C(F)(F)F)CC2)c(OCC2(C(=O)O)CCCC2)c1F]

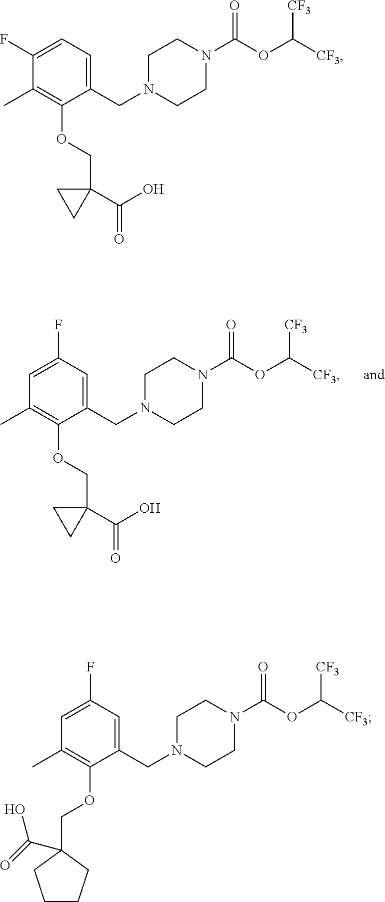 [SMILES: Cc1c(F)ccc(CN2CCN(C(=O)OC(C(F)(F)F)C(F)(F)F)CC2)c1OCC1(C(=O)O)CC1 ; Cc1cc(F)cc(CN2CCN(C(=O)OC(C(F)(F)F)C(F)(F)F)CC2)c1OCC1(C(=O)O)CC1 ; Cc1cc(F)cc(CN2CCN(C(=O)OC(C(F)(F)F)C(F)(F)F)CC2)c1OCC1(C(=O)O)CCCC1]

or a pharmaceutically acceptable salt or solvate thereof.

In some embodiments is a method for treating dyskinesia in a patient in need thereof, comprising administering to the patient a therapeutically effective amount of a compound of Formula (VIII):

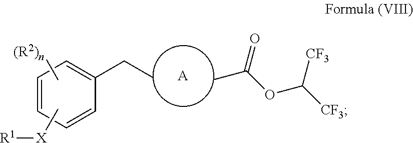

wherein:

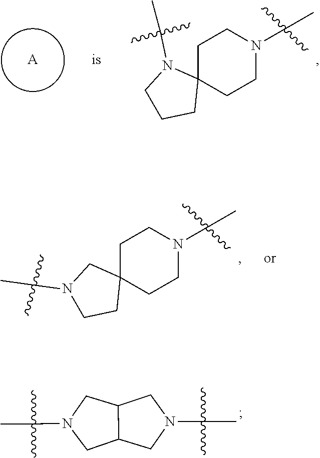

X is —O—, —S—, —SO₂—, —N(R³)—, or —CH₂—;
Y is —O— or —N(R⁷)—;
R¹ is —(CR⁴R⁵)ₘ—R⁶, —(CR⁴R⁵)ₚ—Y—(CR⁴R⁵)_{q}—R⁶, or —(CR⁴R⁵)ₜ—C₃₋₆cycloalkyl-R⁶;
each R² is independently selected from halogen, —CN, C₁₋₆alkyl, C₁₋₆haloalkyl, —C₁₋₆alkyl(heterocycloalkyl), —OR¹⁷, and —C(O)NR¹⁸R¹⁹;
R³ is H or C₁₋₆alkyl;
each R⁴ and R⁵ is each independently selected from H, F, and C₁₋₆alkyl; or R⁴ and R⁵, together with the carbon to which they are attached, form a C₃₋₆cycloalkyl ring;
R⁶ is —CO₂R⁹, —C(O)R¹⁰, or —C(O)O—(CR¹²R¹³)—OC(O)R¹¹;
R⁷ is H, C₁₋₆alkyl, or —SO₂R⁸;
R⁸ is C₁₋₆alkyl;
R⁹ is H or C₁₋₆alkyl;
R¹⁰ is C₁₋₆alkyl or —NHSO₂R²¹;
R¹¹ is C₁₋₆alkyl or C₁₋₆alkoxy;
R¹² and R¹³ is each independently H or C₁₋₆alkyl;
each R¹⁷ is independently selected from H, C₁₋₆alkyl, C₁₋₆haloalkyl, aminoalkyl, cycloalkyl, —C₁₋₆alkyl(heterocycloalkyl), —C₁₋₆alkyl-C(O)(heterocycloalkyl), optionally substituted heterocycloalkyl, optionally substituted aryl, and optionally substituted heteroaryl;
each R¹⁸ and R¹⁹ is independently selected from H, C₁₋₆alkyl, C₁₋₆haloalkyl, cycloalkyl, aryl, and heteroaryl; or R¹⁸ and R¹⁹, together with the nitrogen to which they are attached, form a heterocycloalkyl ring optionally substituted with one, two, or three R²⁰;
each R²⁰ is independently selected from halogen, C₁₋₆alkyl, C₁₋₆haloalkyl, oxo, —CN, and C₃₋₆cycloalkyl;
R²¹ is C₁₋₆alkyl;
m is 1, 2, 3 or 4;
n is 0, 1, 2, 3, or 4;
p is 2, 3, or 4;
q is 1, 2, or 3; and
t is 0, 1, or 2;
   or a pharmaceutically acceptable salt or solvate thereof.

In some embodiments is a method for treating dyskinesia in a patient in need thereof, comprising administering to the patient a therapeutically effective amount of a compound of Formula (VIII), wherein the dyskinesia is levodopa-induced dyskinesia.

In some embodiments of the methods for treating dyskinesia with a compound of Formula (VIII),

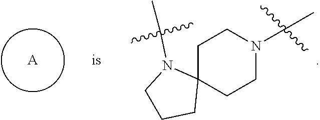

In some embodiments of the methods for treating dyskinesia with a compound of Formula (VIII),

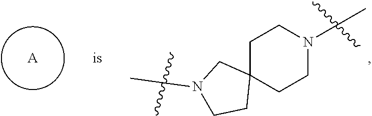

In some embodiments of the methods for treating dyskinesia with a compound of Formula (VIII),

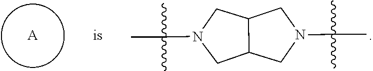

In some embodiments of the methods for treating dyskinesia with a compound of Formula (VIII), X is —O—. In some embodiments of the methods for treating dyskinesia with a compound of Formula (VIII), X is —N(R³)—. In some embodiments of the methods for treating dyskinesia with a compound of Formula (VIII), X is —N(H)—. In some embodiments of the methods for treating dyskinesia with a compound of Formula (VIII), X is —N(CH₃)—. In some embodiments of the methods for treating dyskinesia with a compound of Formula (VIII), X is —N(CH₂CH₃)—.

In some embodiments of the methods for treating dyskinesia with a compound of Formula (VIII), R¹ is —(CR⁴R⁵)ₘ—R⁶. In some embodiments of the methods for treating dyskinesia with a compound of Formula (VIII), R¹ is —(CR⁴R⁵)ₘ—R⁶ and R⁶ is —CO₂R⁹. In some embodiments of the methods for treating dyskinesia with a compound of Formula (VIII), R¹ is —(CR⁴R⁵)ₘ—R⁶ and R⁶ is —CO₂H. In some embodiments of the methods for treating dyskinesia with a compound of Formula (VIII), R¹ is —(CR⁴R⁵)ₘ—R⁶ and R⁶ is —CO₂CH₃. In some embodiments of the methods for treating dyskinesia with a compound of Formula (VIII), R¹ is —(CR⁴R⁵)ₘ—R⁶ and R⁶ is —CO₂CH₂CH₃. In some embodiments of the methods for treating dyskinesia with a compound of Formula (VIII), R¹ is —(CR⁴R⁵)ₘ—R⁶ and R⁶ is —C(O)R¹⁰.

In some embodiments of the methods for treating dyskinesia with a compound of Formula (VIII), R¹ is —(CR⁴R⁵)ₘ—R⁶ and R⁶ is —C(O)NHSO₂CH₃. In some embodiments of the methods for treating dyskinesia with a compound of Formula (VIII), R¹ is —(CR⁴R⁵)ₘ—R⁶ and m is 1. In some embodiments of the methods for treating dyskinesia with a compound of Formula (VIII), R¹ is —(CR⁴R⁵)ₘ—R⁶ and m is 2. In some embodiments of the methods for treating dyskinesia with a compound of Formula (VIII), R¹ is —(CR⁴R⁵)ₘ—R⁶ and m is 3. In some embodiments of the methods for treating dyskinesia with a compound of Formula (VIII), R¹ is —(CR⁴R⁵)ₘ—R⁶ and m is 4.

In some embodiments of the methods for treating dyskinesia with a compound of Formula (VIII), R¹ is —(CR⁴R⁵)ₘ—R⁶ and each R⁴ and R⁵ is each independently selected from H and C₁₋₆alkyl. In some embodiments of the methods for treating dyskinesia with a compound of Formula (VIII), R¹ is —(CR⁴R⁵)ₘ—R⁶ and each R⁴ and R⁵ is H.

In some embodiments of the methods for treating dyskinesia with a compound of Formula (VIII), R¹ is —(CR⁴R⁵)ₘ—R⁶, R⁶ is —CO₂H, m is 1, and R⁴ and R⁵ are H. In some embodiments of the methods for treating dyskinesia with a compound of Formula (VIII), R¹ is —(CR⁴R⁵)ₘ—R⁶, R⁶ is —CO₂H, m is 2, and each R⁴ and R⁵ is H. In some embodiments of the methods for treating dyskinesia with a compound of Formula (VIII), R¹ is —(CR⁴R⁵)ₘ—R⁶, R⁶ is —CO₂H, m is 3, and each R⁴ and R⁵ is H. In some embodiments of the methods for treating dyskinesia with a compound of Formula (VIII), R¹ is —(CR⁴R⁵)ₘ—R⁶, R⁶ is —CO₂H, m is 4, and each R⁴ and R⁵ is H.

In some embodiments of the methods for treating dyskinesia with a compound of Formula (VIII), R¹ is —(CR⁴R⁵)ₜ—C₃₋₆cycloalkyl-R⁶. In some embodiments of the methods for treating dyskinesia with a compound of Formula (VIII), R¹ is —(CR⁴R⁵)ₜ-cyclopropyl-R⁶. In some embodiments of the methods for treating dyskinesia with a compound of Formula (VIII), R¹ is —(CR⁴R⁵)ₜ-cyclobutyl-R⁶. In some embodiments of the methods for treating dyskinesia with a compound of Formula (VIII), R¹ is —(CR⁴R⁵)ₜ-cyclopentyl-R⁶. In some embodiments of the methods for treating dyskinesia with a compound of Formula (VIII), R¹ is —(CR⁴R⁵)ₜ-cyclohexyl-R⁶. In some embodiments of the methods for treating dyskinesia with a compound of Formula (VIII), R¹ is —(CR⁴R⁵)ₜ—C₃₋₆cycloalkyl-R⁶ and R⁶ is —CO₂R⁹. In some embodiments of the methods for treating dyskinesia with a compound of Formula (VIII), R¹ is —(CR⁴R⁵)ₜ—C₃₋₆cycloalkyl-R⁶ and R⁶ is —CO₂H. In some embodiments of the methods for treating dyskinesia with a compound of Formula (VIII), R¹ is —(CR⁴R⁵)ₜ—C₃₋₆cycloalkyl-R⁶ and R⁶ is —CO₂CH₃. In some embodiments of the methods for treating dyskinesia with a compound of Formula (VIII), R¹ is —(CR⁴R⁵)ₜ—C₃₋₆cycloalkyl-R⁶ and R⁶ is —CO₂CH₂CH₃. In some embodiments of the methods for treating dyskinesia with a compound of Formula (VIII), R¹ is —(CR⁴R⁵)ₜ—C₃₋₆ cycloalkyl-R⁶ and t is 0. In some embodiments of the methods for treating dyskinesia with a compound of Formula (VIII), R¹ is —(CR⁴R⁵)ₜ—C₃₋₆cycloalkyl-R⁶ and t is 1. In some embodiments of the methods for treating dyskinesia with a compound of Formula (VIII), R¹ is —(CR⁴R⁵)ₜ—C₃₋₆cycloalkyl-R⁶ and t is 2.

In some embodiments of the methods for treating dyskinesia with a compound of Formula (VIII), R¹ is —(CR⁴R⁵)ₜ—C₃₋₆cycloalkyl-R⁶, R⁶ is —CO₂H, t is 0, and R⁴ and R⁵ are H. In some embodiments of the methods for treating dyskinesia with a compound of Formula (VIII), R¹ is —(CR⁴R⁵)ₜ—C₃₋₆cycloalkyl-R⁶, R⁶ is —CO₂H, t is 1, and each R⁴ and R⁵ is H. In some embodiments of the methods for treating dyskinesia with a compound of Formula (VIII), R¹ is —(CR⁴R⁵)ₜ—C₃₋₆cycloalkyl-R⁶, R⁶ is —CO₂H, t is 2, and each R⁴ and R⁵ is H.

In some embodiments of the methods for treating dyskinesia with a compound of Formula (VIII), —X—R¹ is —OCH₂C(O)OH. In some embodiments of the methods for treating dyskinesia with a compound of Formula (VIII), —X—R¹ is —N(H)CH₂C(O)OH. In some embodiments of the methods for treating dyskinesia with a compound of Formula (VIII), —X—R¹ is —OCH(CH₃)C(O)OH. In some embodiments of the methods for treating dyskinesia with a compound of Formula (VIII), —X—R¹ is —N(H)CH(CH₃)C(O)OH. In some embodiments of the methods for treating dyskinesia with a compound of Formula (VIII), —X—R¹ is —OCH₂CH₂C(O)OH. In some embodiments of the methods for treating dyskinesia with a compound of Formula (VIII), —X—R¹ is —N(H)CH₂CH₂C(O)OH. In some embodiments of the methods for treating dyskinesia with a compound of Formula (VIII), —X—R¹ is —OCH₂CH₂CH₂C(O)OH. In some embodiments of the methods for treating dyskinesia with a compound of Formula (VIII), —X—R¹ is —N(H)CH₂CH₂CH₂C(O)OH. In some embodiments of the methods for treating dyskinesia with a compound of Formula (VIII), —X—R¹ is —OCH₂CH₂C(CH₃)₂C(O)OH. In some embodiments of the methods for treating dyskinesia with a compound of Formula (VIII), —X—R¹ is —N(H)CH₂CH₂C(CH₃)₂C(O)OH.

In some embodiments of the methods for treating dyskinesia with a compound of Formula (VIII), —X—R¹ is —O-cyclopropyl-C(O)OH. In some embodiments of the methods for treating dyskinesia with a compound of Formula (VIII), —X—R¹ is —N(H)— cyclopropyl-C(O)OH. In some embodiments of the methods for treating dyskinesia with a compound of Formula (VIII), —X—R¹ is —O-cyclobutyl-C(O)OH. In some embodiments of the methods for treating dyskinesia with a compound of Formula (VIII), —X—R¹ is —N(H)-cyclobutyl-C(O)OH.

In some embodiments of the methods for treating dyskinesia with a compound of Formula (VIII), n is 0, 1, or 2. In some embodiments of the methods for treating dyskinesia with a compound of Formula (VIII), n is 1 or 2. In some embodiments of the methods for treating dyskinesia with a compound of Formula (VIII), n is 0 or 1. In some embodiments of the methods for treating dyskinesia with a compound of Formula (VIII), n is 0. In some embodiments of the methods for treating dyskinesia with a compound of Formula (VIII), n is 1. In some embodiments of the methods for treating dyskinesia with a compound of Formula (VIII), n is 2.

In some embodiments of the methods for treating dyskinesia with a compound of Formula (VIII), n is 1 and R² is halogen, C₁₋₆alkyl, C₁₋₆haloalkyl, or —OR¹⁷. In some embodiments of the methods for treating dyskinesia with a compound of Formula (VIII), n is 1 and R² is halogen, C₁₋₆alkyl, or C₁₋₆haloalkyl. In some embodiments of the methods for treating dyskinesia with a compound of Formula (VIII), n is 1 and R² is halogen. In some embodiments of the methods for treating dyskinesia with a compound of Formula (VIII), n is 1 and R² is —Cl. In some embodiments of the methods for treating dyskinesia with a compound of Formula (VIII), n is 1 and R² is —F. In some embodiments of the methods for treating dyskinesia with a compound of Formula (VIII), n is 1 and R² is C₁₋₆alkyl. In some embodiments of the methods for treating dyskinesia with a compound of Formula (VIII), n is 1 and R² is —CH₃. In some embodiments of the methods for treating dyskinesia with a compound of Formula (VIII), n is 1 and R² is C₁₋₆ haloalkyl. In some embodiments of the methods for treating dyskinesia with a compound of Formula (VIII), n is 1 and R² is —CF₃.

In some embodiments of the methods for treating dyskinesia with a compound of Formula (VIII), the compound is selected from:

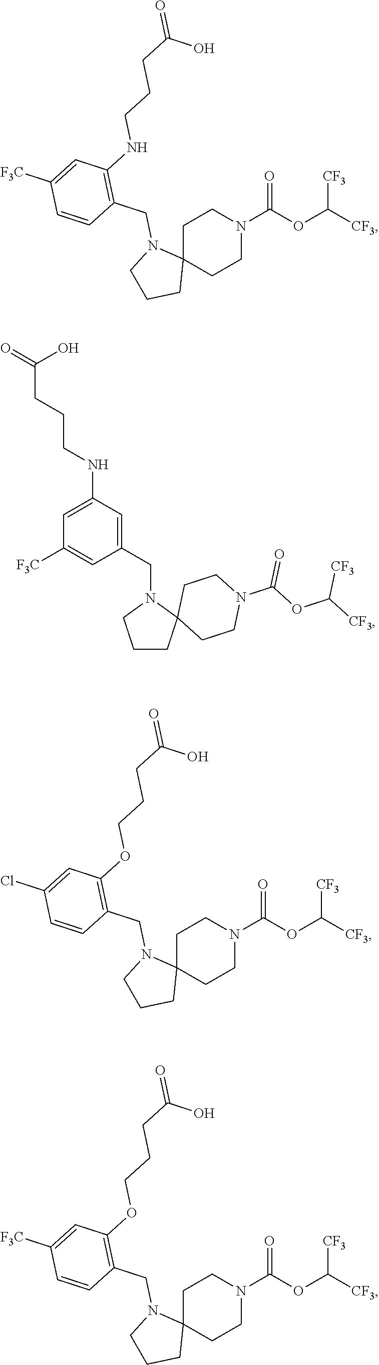 [SMILES: O=C(O)CCCNc1cc(C(F)(F)F)ccc1CN1CCCC12CCN(C(=O)OC(C(F)(F)F)C(F)(F)F)CC2 ; O=C(O)CCCNc1cc(CN2CCCC23CCN(C(=O)OC(C(F)(F)F)C(F)(F)F)CC3)cc(C(F)(F)F)c1 ; O=C(O)CCCOc1cc(Cl)ccc1CN1CCCC12CCN(C(=O)OC(C(F)(F)F)C(F)(F)F)CC2 ; O=C(O)CCCOc1cc(C(F)(F)F)ccc1CN1CCCC12CCN(C(=O)OC(C(F)(F)F)C(F)(F)F)CC2]

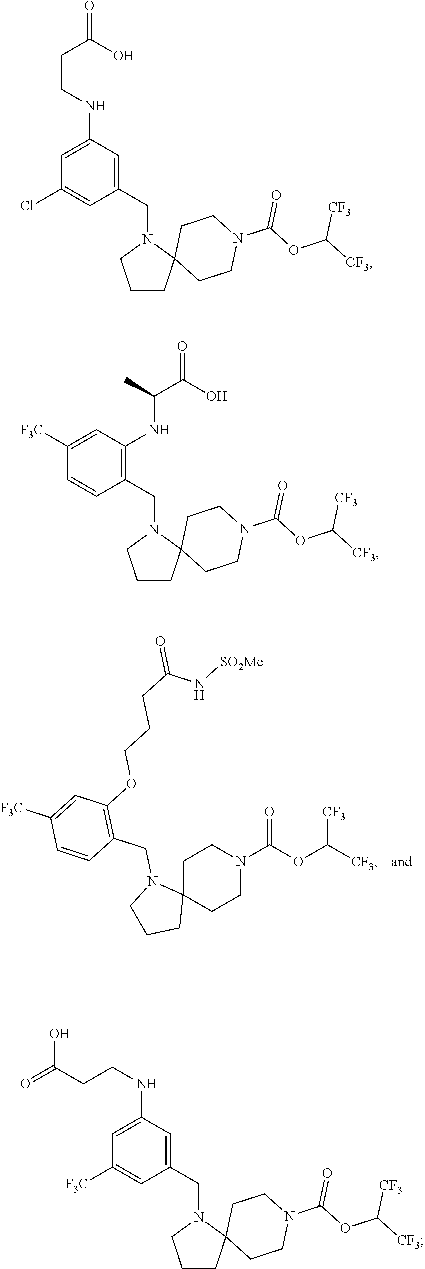 [SMILES: O=C(O)CCNc1cc(Cl)cc(CN2CCCC23CCN(C(=O)OC(C(F)(F)F)C(F)(F)F)CC3)c1 ; C[C@H](Nc1cc(C(F)(F)F)ccc1CN1CCCC12CCN(C(=O)OC(C(F)(F)F)C(F)(F)F)CC2)C(=O)O ; CS(=O)(=O)NC(=O)CCCOc1cc(C(F)(F)F)ccc1CN1CCCC12CCN(C(=O)OC(C(F)(F)F)C(F)(F)F)CC2 ; O=C(O)CCNc1cc(CN2CCCC23CCN(C(=O)OC(C(F)(F)F)C(F)(F)F)CC3)cc(C(F)(F)F)c1]

or a pharmaceutically acceptable salt or solvate thereof.

In some embodiments is a method for treating dyskinesia in a patient in need thereof, comprising administering to the patient a therapeutically effective amount of a compound of Formula (IX):

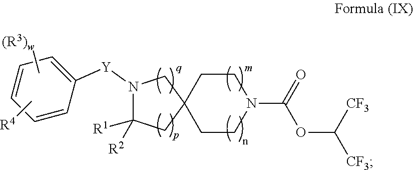

wherein:
Y is —CH₂— or —C(O)—;
R¹ is H or C₁₋₆alkyl;
R² is H or C₁₋₆alkyl;
each R³ is independently selected from C₁₋₆alkyl, halogen, —CN, C₁₋₆haloalkyl, —SF₅, and —OR⁷;
R⁴ is selected from —C≡C—C₁₋₆alkyl-CO₂H and —C₃₋₈cycloalkyl-CO₂H;
each R⁷ is independently selected from H, C₁₋₆alkyl, C₁₋₆haloalkyl, C₁₋₆aminoalkyl, C₃₋₈cycloalkyl, and —C₁₋₆alkyl-C₃₋₈cycloalkyl;
w is 0, 1, 2, 3, or 4;
n is 0 or 1;
m is 0 or 1;
p is 0, 1, or 2; and
q is 0, 1, or 2; provided that when q is 0, then p is 2;
   or a pharmaceutically acceptable salt or solvate thereof.

In some embodiments is a method for treating dyskinesia in a patient in need thereof, comprising administering to the patient a therapeutically effective amount of a compound of Formula (IX), wherein the dyskinesia is levodopa-induced dyskinesia.

In some embodiments of the methods for treating dyskinesia with a compound of Formula (IX), m is 0, n is 0, p is 1, and q is 2. In some embodiments of the methods for treating dyskinesia with a compound of Formula (IX), m is 0, n is 1, p is 1, and q is 1.

In some embodiments of the methods for treating dyskinesia with a compound of Formula (IX), m is 1, n is 0, p is 1, and q is 1. In some embodiments of the methods for treating dyskinesia with a compound of Formula (IX), m is 1, n is 1, p is 0, and q is 1. In some embodiments of the methods for treating dyskinesia with a compound of Formula (IX), m is 1, n is 1, p is 1, and q is 1. In another embodiment is a compound of Formula (IX), m is 1, n is 1, p is 2 and q is 0.

In some embodiments of the methods for treating dyskinesia with a compound of Formula (IX), Y is —CH₂—. In some embodiments of the methods for treating dyskinesia with a compound of Formula (IX), Y is —C(O)—.

In some embodiments of the methods for treating dyskinesia with a compound of Formula (IX), R¹ is H. In some embodiments of the methods for treating dyskinesia with a compound of Formula (IX), R² is H. In some embodiments of the methods for treating dyskinesia with a compound of Formula (IX), R¹ and R² are both H.

In some embodiments of the methods for treating dyskinesia with a compound of Formula (IX), w is 0, 1, or 2. In some embodiments of the methods for treating dyskinesia with a compound of Formula (IX), w is 0 or 1. In some embodiments of the methods for treating dyskinesia with a compound of Formula (IX), w is 1 or 2. In some embodiments of the methods for treating dyskinesia with a compound of Formula (IX), w is 0. In some embodiments of the methods for treating dyskinesia with a compound of Formula (IX), w is 1. In some embodiments of the methods for treating dyskinesia with a compound of Formula (IX), w is 2.

In some embodiments of the methods for treating dyskinesia with a compound of Formula (IX), R⁴ is —C≡C—C₁₋₆alkyl-CO₂H. In some embodiments of the methods for treating dyskinesia with a compound of Formula (IX), R⁴ is —C₃₋₈cycloalkyl-CO₂H. In some embodiments of the methods for treating dyskinesia with a compound of Formula (IX), R⁴ is

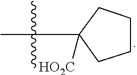 [SMILES: *C1(C(=O)O)CCCC1]

In some embodiments of the methods for treating dyskinesia with a compound of Formula (IX), R⁴ is —C≡C—C₁₋₆alkyl-CO₂H, w is 1, and R³ is selected from C₁₋₆alkyl, halogen, C₁₋₆haloalkyl, —SF₅, and —OR⁷. In some embodiments of the methods for treating dyskinesia with a compound of Formula (IX), R⁴ is —C≡C—C₁₋₆alkyl-CO₂H, w is 1, and R³ is selected from halogen, C₁₋₆haloalkyl, and —OR⁷. In some embodiments of the methods for treating dyskinesia with a compound of Formula (IX), R⁴ is —C≡C—C₁₋₆alkyl-CO₂H, w is 1, and R³ is selected from halogen and C₁₋₆haloalkyl. In some embodiments of the methods for treating dyskinesia with a compound of Formula (IX), R⁴ is —C≡C—C₁₋₆alkyl-CO₂H, w is 1, and R³ is C₁₋₆alkyl. In some embodiments of the methods for treating dyskinesia with a compound of Formula (IX), R⁴ is —C≡C—C₁₋₆ alkyl-CO₂H, w is 1, and R³ is —CH₃. In some embodiments of the methods for treating dyskinesia with a compound of Formula (IX), R⁴ is —C≡C—C₁₋₆alkyl-CO₂H, w is 1, and R³ is halogen. In some embodiments of the methods for treating dyskinesia with a compound of Formula (IX), R⁴ is —C≡C—C₁₋₆alkyl-CO₂H, w is 1, and R³ is —Cl. In some embodiments of the methods for treating dyskinesia with a compound of Formula (IX), R⁴ is —C≡C—C₁₋₆alkyl-CO₂H, w is 1, and R³ is C₁₋₆haloalkyl. In some embodiments of the methods for treating dyskinesia with a compound of Formula (IX), R⁴ is —C≡C—C₁₋₆ alkyl-CO₂H, w is 1, and R³ is —CF₃.

In some embodiments of the methods for treating dyskinesia with a compound of Formula (IX), R⁴ is —C₃₋₈cycloalkyl-CO₂H, w is 1, and R³ is selected from C₁₋₆alkyl, halogen, C₁₋₆haloalkyl, —SF₅, and —OR⁷. In some embodiments of the methods for treating dyskinesia with a compound of Formula (IX), R⁴ is —C₃₋₈cycloalkyl-CO₂H, w is 1, and R³ is selected from halogen, C₁₋₆haloalkyl, and —OR⁷. In some embodiments of the methods for treating dyskinesia with a compound of Formula (IX), R⁴ is —C₃₋₈ cycloalkyl-CO₂H, w is 1, and R³ is selected from halogen and C₁₋₆haloalkyl. In some embodiments of the methods for treating dyskinesia with a compound of Formula (IX), R⁴ is —C₃₋₈cycloalkyl-CO₂H, w is 1, and R³ is C₁₋₆alkyl. In some embodiments of the methods for treating dyskinesia with a compound of Formula (IX), R⁴ is —C₃₋₈cycloalkyl-CO₂H, w is 1, and R³ is —CH₃. In some embodiments of the methods for treating dyskinesia with a compound of Formula (IX), R⁴ is —C₃₋₈cycloalkyl-CO₂H, w is 1, and R³ is halogen. In some embodiments of the methods for treating dyskinesia with a compound of Formula (IX), R⁴ is —C₃₋₈cycloalkyl-CO₂H, w is 1, and R³ is —Cl. In some embodiments of the methods for treating dyskinesia with a compound of Formula (IX), R⁴ is —C₃₋₈cycloalkyl-CO₂H, w is 1, and R³ is C₁₋₆haloalkyl. In some embodiments of the methods for treating dyskinesia with a compound of Formula (IX), R⁴ is —C₃₋₈cycloalkyl-CO₂H, w is 1, and R³ is —CF₃.

In some embodiments of the methods for treating dyskinesia with a compound of Formula (IX), the compound is selected from:

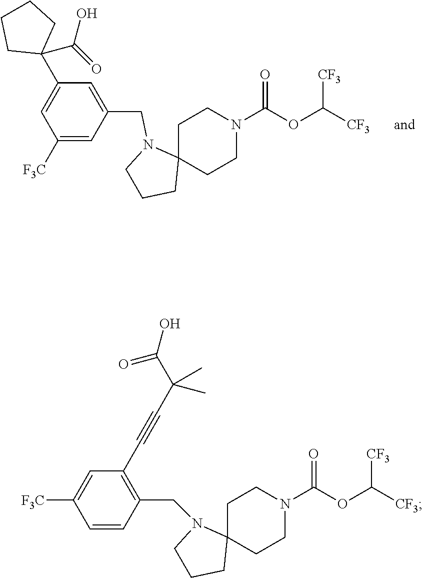 [SMILES: O=C(OC(C(F)(F)F)C(F)(F)F)N1CCC2(CCCN2Cc2cc(C(F)(F)F)cc(C3(C(=O)O)CCCC3)c2)CC1 ; CC(C)(C#Cc1cc(C(F)(F)F)ccc1CN1CCCC12CCN(C(=O)OC(C(F)(F)F)C(F)(F)F)CC2)C(=O)O]

or a pharmaceutically acceptable salt or solvate thereof.

In some embodiments is a method for treating dyskinesia in a patient in need thereof, comprising administering to the patient a therapeutically effective amount of a compound of Formula (X):

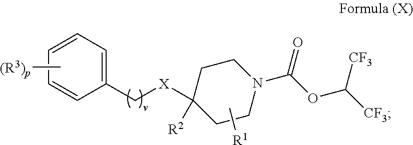

wherein:
X is —O— or —N(R¹¹)—;
R¹ is H or C₁₋₆alkyl;
R² is C₁₋₆alkyl;
each R³ is independently selected from C₁₋₆alkyl, C₂₋₆alkenyl, C₂₋₆alkynyl, —C≡C—C₁₋₆ alkyl-CO₂H, halogen, —CN, C₁₋₆haloalkyl, C₁₋₆aminoalkyl, C₃₋₈cycloalkyl, —C₁₋₆alkyl(C₂₋₉heterocycloalkyl), C₁₋₉heteroaryl, —SF₅, —NR⁵R⁶, —OR⁷, —CO₂R⁸, and —C(O)NR⁸R⁹, wherein C₃₋₈cycloalkyl, —C₁₋₆alkyl(C₂₋₉heterocycloalkyl), and C₁₋₉ heteroaryl are optionally substituted with one or two R⁴; or two adjacent R³ form a C₂₋₉heterocycloalkyl ring, wherein the C₂₋₉heterocycloalkyl ring is optionally substituted with one, two, or three R⁴;
each R⁴ is independently selected from C₁₋₆alkyl, C₃₋₈cycloalkyl, C₁₋₆haloalkyl, halogen, oxo, —CN, —CO₂R⁸, —C(O)R⁸, —C(O)NR⁸R⁹, —SO₂R⁸, —NR⁹C(O)R⁸, and —NR⁹SO₂R⁸; each R⁵ and R⁶ is independently selected from H, C₁₋₆alkyl, C₁₋₆haloalkyl, C₁₋₆ aminoalkyl, C₃₋₈cycloalkyl, —C₁₋₆alkyl(C₂₋₉heterocycloalkyl), —C₁₋₆alkyl-C(O)(C₂₋₉heterocycloalkyl), C₂₋₉heterocycloalkyl, C₆₋₁₀aryl, and C₁₋₉heteroaryl; or R⁵ and R⁶, together with the nitrogen to which they are attached, form a C₂₋₉heterocycloalkyl ring optionally substituted with one, two, or three R¹⁰;
each R⁷ is independently selected from H, C₁₋₆alkyl, C₁₋₆haloalkyl, C₁₋₆aminoalkyl, C₃₋₈cycloalkyl, —C₁₋₆alkyl(C₂₋₉heterocycloalkyl), —C₁₋₆alkyl-C(O)(C₂₋₉ heterocycloalkyl), —C₁₋₆alkyl-CO₂H, C₂₋₉heterocycloalkyl, C₆₋₁₀aryl, and C₁₋₉ heteroaryl, wherein C₂₋₉heterocycloalkyl, C₆₋₁₀aryl, and C₁₋₉heteroaryl are optionally substituted with one or two groups selected from oxo, C₁₋₆alkyl, C₁₋₆ haloalkyl, CO₂H, and CO₂NH₂;
each R⁸ and R⁹ is independently selected from H, C₁₋₆alkyl, C₁₋₆haloalkyl, C₃₋₈cycloalkyl, C₆₋₁₀aryl, and C₁₋₉heteroaryl; or R⁸ and R⁹, together with the nitrogen to which they are attached, form a C₂₋₉heterocycloalkyl ring optionally substituted with one or two groups selected from C₁₋₆alkyl, C₁₋₆haloalkyl, CO₂H, and CO₂NH₂;
each R¹⁰ is independently selected from halogen, C₁₋₆alkyl, C₁₋₆haloalkyl, C₃₋₈cycloalkyl, oxo, —CN, —CO₂R⁸, —C(O)R⁸, —C(O)NR⁸R⁹, —SO₂R⁸, —NR⁹C(O)R⁸, and —NR⁹SO₂R⁸;
R¹¹ is H, C₁₋₆alkyl, —C(O)—C₁₋₆alkyl, or —CH₂CO₂H;
p is 0, 1, 2, 3, 4, or 5; and
v is 0 or 1;
   or a pharmaceutically acceptable salt or solvate thereof.

In some embodiments is a method for treating dyskinesia in a patient in need thereof, comprising administering to the patient in need thereof a therapeutically effective amount of a compound of Formula (X), wherein the dyskinesia is levodopa-induced dyskinesia.

In some embodiments of the methods for treating dyskinesia with a compound of Formula (X), X is —O—. In some embodiments of the methods for treating dyskinesia with a compound of Formula (X), X is —N(R¹¹)—. In some embodiments of the methods for treating dyskinesia with a compound of Formula (X), X is —N(R¹¹)— and R¹¹ is H. In some embodiments of the methods for treating dyskinesia with a compound of Formula (X), X is —N(R¹¹)— and R¹¹ is C₁₋₆alkyl. In some embodiments of the methods for treating dyskinesia with a compound of Formula (X), X is —N(R¹¹)— and R¹¹ is —CH₃. In some embodiments of the methods for treating dyskinesia with a compound of Formula (X), X is —N(R¹¹)— and R¹¹ is —C(O)—C₁₋₆alkyl. In some embodiments of the methods for treating dyskinesia with a compound of Formula (X), X is —N(R¹¹)— and R¹¹ is —CH₂CO₂H.

In some embodiments of the methods for treating dyskinesia with a compound of Formula (X), R¹ is H. In some embodiments of the methods for treating dyskinesia with a compound of Formula (X), R¹ is C₁₋₆alkyl. In some embodiments of the methods for treating dyskinesia with a compound of Formula (X), R² is —CH₃. In some embodiments of the methods for treating dyskinesia with a compound of Formula (X), R¹ and R² are both —CH₃. In some embodiments of the methods for treating dyskinesia with a compound of Formula (X), R¹ is H and R² is —CH₃.

In some embodiments of the methods for treating dyskinesia with a compound of Formula (X), p is 0, 1, or 2. In some embodiments of the methods for treating dyskinesia with a compound of Formula (X), p is 0 or 1. In some embodiments of the methods for treating dyskinesia with a compound of Formula (X), p is 1 or 2. In some embodiments of the methods for treating dyskinesia with a compound of Formula (X), p is 0. In some embodiments of the methods for treating dyskinesia with a compound of Formula (X), p is 1. In some embodiments of the methods for treating dyskinesia with a compound of Formula (X), p is 2.

In some embodiments of the methods for treating dyskinesia with a compound of Formula (X), p is 2 and each R³ is independently selected from C₁₋₆alkyl, halogen, —CN, C₁₋₆haloalkyl, C₁₋₆aminoalkyl, —C₁₋₆alkyl(heterocycloalkyl), —SF₅, —NR⁵R⁶, and —OR⁷; wherein —C₁₋₆alkyl(heterocycloalkyl) is optionally substituted with one or two groups selected from halogen, C₁₋₆alkyl, C₁₋₆haloalkyl, C₃₋₈cycloalkyl, and oxo. In some embodiments of the methods for treating dyskinesia with a compound of Formula (X), p is 2 and each R³ is independently selected halogen, C₁₋₆alkyl, C₁₋₆haloalkyl, —C₁₋₆ alkyl(C₂₋₉heterocycloalkyl), —NR⁵R⁶, —OR⁷, —CO₂R⁸, and —C(O)NR⁸R⁹. In some embodiments of the methods for treating dyskinesia with a compound of Formula (X), p is 2 and each R³ is independently selected from C₁₋₆alkyl, halogen, C₁₋₆haloalkyl, —NR⁵R⁶, and —OR⁷. In some embodiments of the methods for treating dyskinesia with a compound of Formula (X), p is 2 and each R³ is independently selected from halogen, C₁₋₆haloalkyl, —NR⁵R⁶, and —OR⁷. In some embodiments of the methods for treating dyskinesia with a compound of Formula (X), p is 2 and each R³ is independently selected from halogen, —NR⁵R⁶, and C₁₋₆haloalkyl. In some embodiments of the methods for treating dyskinesia with a compound of Formula (X), p is 2, one R³ is halogen and one R³ is —NR⁵R⁶. In some embodiments of the methods for treating dyskinesia with a compound of Formula (X), p is 2, one R³ is halogen and one R³ is —NR⁵R⁶ wherein R⁵ and R⁶, together with the nitrogen to which they are attached, form a C₂₋₉ heterocycloalkyl ring optionally substituted with one, two, or three R¹⁰. In some embodiments of the methods for treating dyskinesia with a compound of Formula (X), p is 2, one R³ is halogen and one R³ is —NR⁵R⁶ wherein R⁵ and R⁶, together with the nitrogen to which they are attached, form an unsubstituted C₂₋₉heterocycloalkyl ring. In some embodiments of the methods for treating dyskinesia with a compound of Formula (X), p is 2, one R³ is halogen and one R³ is —NR⁵R⁶ wherein R⁵ and R⁶, together with the nitrogen to which they are attached, form a C₂₋₉heterocycloalkyl ring substituted with one or two R¹⁰. In some embodiments of the methods for treating dyskinesia with a compound of Formula (X), p is 2, one R³ is halogen and one R³ is —NR⁵R⁶ wherein R⁵ and R⁶, together with the nitrogen to which they are attached, form a C₂₋₉ heterocycloalkyl ring substituted with one or two R¹⁰ selected from C₁₋₆alkyl and —CO₂H. In some embodiments of the methods for treating dyskinesia with a compound of Formula (X), p is 2, one R³ is halogen and one R³ is —NR⁵R⁶ wherein R⁵ and R⁶, together with the nitrogen to which they are attached, form a C₂₋₉heterocycloalkyl ring substituted with —CO₂H. In some embodiments of the methods for treating dyskinesia with a compound of Formula (X), p is 2, one R³ is —Cl and one R³ is —NR⁵R⁶. In some embodiments of the methods for treating dyskinesia with a compound of Formula (X), p is 2, one R³ is —Cl and one R³ is —NR⁵R⁶ wherein R⁵ and R⁶, together with the nitrogen to which they are attached, form a C₂₋₉heterocycloalkyl ring optionally substituted with one, two, or three R¹⁰. In some embodiments of the methods for treating dyskinesia with a compound of Formula (X), p is 2, one R³ is —Cl and one R³ is —NR⁵R⁶ wherein R⁵ and R⁶, together with the nitrogen to which they are attached, form an unsubstituted C₂₋₉ heterocycloalkyl ring. In some embodiments of the methods for treating dyskinesia with a compound of Formula (X), p is 2, one R³ is —Cl and one R³ is —NR⁵R⁶ wherein R⁵ and R⁶, together with the nitrogen to which they are attached, form a C₂₋₉heterocycloalkyl ring substituted with one or two R¹⁰. In some embodiments of the methods for treating dyskinesia with a compound of Formula (X), p is 2, one R³ is —Cl and one R³ is —NR⁵R⁶ wherein R⁵ and R⁶, together with the nitrogen to which they are attached, form a C₂₋₉ heterocycloalkyl ring substituted with one or two R¹⁰ selected from C₁₋₆alkyl and —CO₂H. In some embodiments of the methods for treating dyskinesia with a compound of Formula (X), p is 2, one R³ is —Cl and one R³ is —NR⁵R⁶ wherein R⁵ and R⁶, together with the nitrogen to which they are attached, form a C₂₋₉heterocycloalkyl ring substituted with —CO₂H. In some embodiments of the methods for treating dyskinesia with a compound of Formula (X), p is 2, one R³ is C₁₋₆haloalkyl and one R³ is —NR⁵R⁶. In some embodiments of the methods for treating dyskinesia with a compound of Formula (X), p is 2, one R³ is C₁₋₆haloalkyl and one R³ is —NR⁵R⁶ wherein R⁵ and R⁶, together with the nitrogen to which they are attached, form a C₂₋₉heterocycloalkyl ring optionally substituted with one, two, or three R¹⁰. In some embodiments of the methods for treating dyskinesia with a compound of Formula (X), p is 2, one R³ is C₁₋₆haloalkyl and one R³ is —NR⁵R⁶ wherein R⁵ and R⁶, together with the nitrogen to which they are attached, form an unsubstituted C₂₋₉heterocycloalkyl ring. In some embodiments of the methods for treating dyskinesia with a compound of Formula (X), p is 2, one R³ is C₁₋₆haloalkyl and one R³ is —NR⁵R⁶ wherein R⁵ and R⁶, together with the nitrogen to which they are attached, form a C₂₋₉heterocycloalkyl ring substituted with one or two R¹⁰. In some embodiments of the methods for treating dyskinesia with a compound of Formula (X), p is 2, one R³ is C₁₋₆ haloalkyl and one R³ is —NR⁵R⁶ wherein R⁵ and R⁶, together with the nitrogen to which they are attached, form a C₂₋₉heterocycloalkyl ring substituted with one or two R¹⁰ selected from C₁₋₆alkyl and —CO₂H. In some embodiments of the methods for treating dyskinesia with a compound of Formula (X), p is 2, one R³ is C₁₋₆haloalkyl and one R³ is —NR⁵R⁶ wherein R⁵ and R⁶, together with the nitrogen to which they are attached, form a C₂₋₉heterocycloalkyl ring substituted with —CO₂H. In some embodiments of the methods for treating dyskinesia with a compound of Formula (X), p is 2, one R³ is —CF₃ and one R³ is —NR⁵R⁶. In some embodiments of the methods for treating dyskinesia with a compound of Formula (X), p is 2, one R³ is —CF₃ and one R³ is —NR⁵R⁶ wherein R⁵ and R⁶, together with the nitrogen to which they are attached, form a C₂₋₉heterocycloalkyl ring optionally substituted with one, two, or three R¹⁰. In some embodiments of the methods for treating dyskinesia with a compound of Formula (X), p is 2, one R³ is —CF₃ and one R³ is —NR⁵R⁶ wherein R⁵ and R⁶, together with the nitrogen to which they are attached, form an unsubstituted C₂₋₉heterocycloalkyl ring. In some embodiments of the methods for treating dyskinesia with a compound of Formula (X), p is 2, one R³ is —CF₃ and one R³ is —NR⁵R⁶ wherein R⁵ and R⁶, together with the nitrogen to which they are attached, form a C₂₋₉heterocycloalkyl ring substituted with one or two R¹⁰. In some embodiments of the methods for treating dyskinesia with a compound of Formula (X), p is 2, one R³ is —CF₃ and one R³ is —NR⁵R⁶ wherein R⁵ and R⁶, together with the nitrogen to which they are attached, form a C₂₋₉heterocycloalkyl ring substituted with one or two R¹⁰ selected from C₁₋₆alkyl and —CO₂H. In some embodiments of the methods for treating dyskinesia with a compound of Formula (X), p is 2, one R³ is —CF₃ and one R³ is —NR⁵R⁶ wherein R⁵ and R⁶, together with the nitrogen to which they are attached, form a C₂₋₉heterocycloalkyl ring substituted with —CO₂H.

In some embodiments of the methods for treating dyskinesia with a compound of Formula (X), p is 1 and R³ is selected from C₁₋₆alkyl, halogen, —CN, C₁₋₆haloalkyl, C₁₋₆aminoalkyl, —C₁₋₆alkyl(heterocycloalkyl), —SF₅, —NR⁵R⁶, and —OR⁷; wherein —C₁₋₆alkyl(heterocycloalkyl) is optionally substituted with one or two groups selected from halogen, C₁₋₆alkyl, C₁₋₆haloalkyl, C₃₋₈cycloalkyl, and oxo. In some embodiments of the methods for treating dyskinesia with a compound of Formula (X), p is 1 and R³ is selected halogen, C₁₋₆alkyl, C₁₋₆haloalkyl, —C₁₋₆alkyl(C₂₋₉heterocycloalkyl), —NR⁵R⁶, —OR⁷, —CO₂R⁸, and —C(O)NR⁸R⁹. In some embodiments of the methods for treating dyskinesia with a compound of Formula (X), p is 1 and R³ is selected from C₁₋₆ alkyl, halogen, C₁₋₆haloalkyl, —NR⁵R⁶, and —OR⁷. In some embodiments of the methods for treating dyskinesia with a compound of Formula (X), p is 1 and R³ is selected from halogen, C₁₋₆haloalkyl, —NR⁵R⁶, and —OR⁷. In some embodiments of the methods for treating dyskinesia with a compound of Formula (X), p is 1 and R³ is selected from halogen, —NR⁵R⁶, and C₁₋₆haloalkyl. In some embodiments of the methods for treating dyskinesia with a compound of Formula (X), p is 1 and R³ is C₁₋₆alkyl. In some embodiments of the methods for treating dyskinesia with a compound of Formula (X), p is 1 and R³ is halogen. In some embodiments of the methods for treating dyskinesia with a compound of Formula (X), p is 1 and R³ is C₁₋₆haloalkyl. In some embodiments of the methods for treating dyskinesia with a compound of Formula (X), p is 1 and R³ is —OR⁷. In some embodiments of the methods for treating dyskinesia with a compound of Formula (X), p is 1 and R³ is —NR⁵R⁶. In some embodiments of the methods for treating dyskinesia with a compound of Formula (X), p is 1 and R³ is —NR⁵R⁶, wherein R⁵ and R⁶, together with the nitrogen to which they are attached, form a C₂₋₉heterocycloalkyl ring optionally substituted with one, two, or three R¹⁰. In some embodiments of the methods for treating dyskinesia with a compound of Formula (X), p is 1 and R³ is —NR⁵R⁶, wherein R⁵ and R⁶, together with the nitrogen to which they are attached, form an unsubstituted C₂₋₉heterocycloalkyl ring. In some embodiments of the methods for treating dyskinesia with a compound of Formula (X), p is 1 and R³ is —NR⁵R⁶, wherein R⁵ and R⁶, together with the nitrogen to which they are attached, form a C₂₋₉ heterocycloalkyl ring substituted with one or two R¹⁰. In some embodiments of the methods for treating dyskinesia with a compound of Formula (X), p is 1 and R³ is —NR⁵R⁶, wherein R⁵ and R⁶, together with the nitrogen to which they are attached, form a C₂₋₉heterocycloalkyl ring substituted with one or two R¹⁰ selected from C₁₋₆alkyl and —CO₂H. In some embodiments of the methods for treating dyskinesia with a compound of Formula (X), p is 1 and R³ is —NR⁵R⁶, wherein R⁵ and R⁶, together with the nitrogen to which they are attached, form a C₂₋₉heterocycloalkyl ring substituted with —CO₂H.

In some embodiments of the methods for treating dyskinesia with a compound of Formula (X), R⁵ and R⁶, together with the nitrogen to which they are attached, form a C₂₋₉heterocycloalkyl ring selected from:

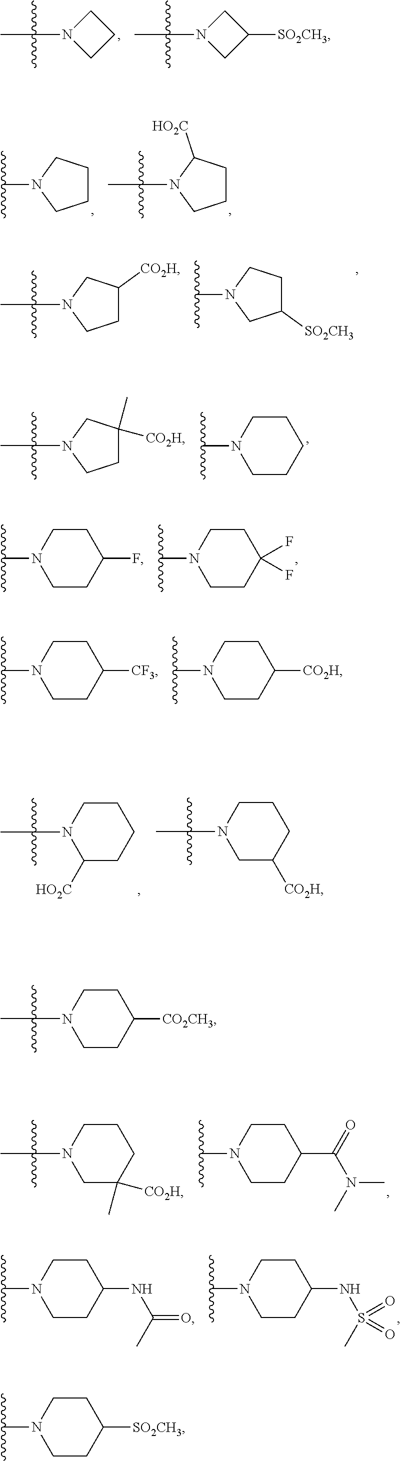

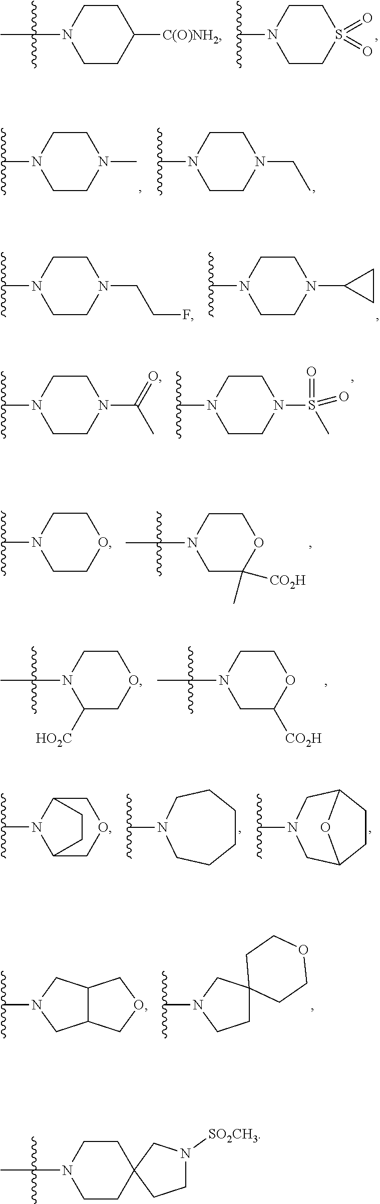

In some embodiments of the methods for treating dyskinesia with a compound of Formula (X), the compound is selected from:

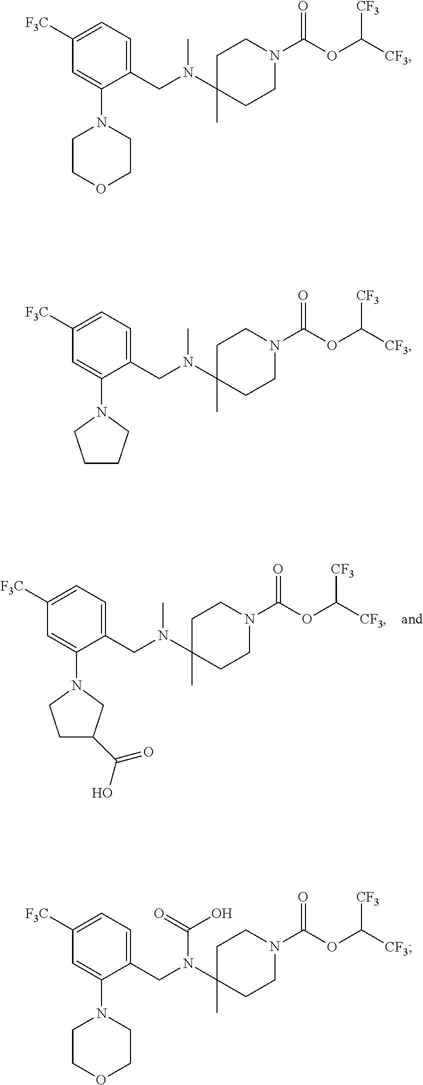 [SMILES: CN(Cc1ccc(C(F)(F)F)cc1N1CCOCC1)C1(C)CCN(C(=O)OC(C(F)(F)F)C(F)(F)F)CC1 ; CN(Cc1ccc(C(F)(F)F)cc1N1CCCC1)C1(C)CCN(C(=O)OC(C(F)(F)F)C(F)(F)F)CC1 ; CN(Cc1ccc(C(F)(F)F)cc1N1CCC(C(=O)O)C1)C1(C)CCN(C(=O)OC(C(F)(F)F)C(F)(F)F)CC1 ; CC1(N(Cc2ccc(C(F)(F)F)cc2N2CCOCC2)C(=O)O)CCN(C(=O)OC(C(F)(F)F)C(F)(F)F)CC1]

or a solvate, hydrate, tautomer, N-oxide, stereoisomer, or pharmaceutically acceptable salt thereof.

In some embodiments is a method for treating dyskinesia in a patient in need thereof, comprising administering to the patient a therapeutically effective amount of a compound of Formula (XI):

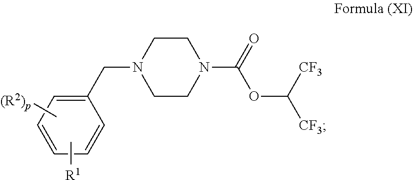

wherein:
R¹ is selected from

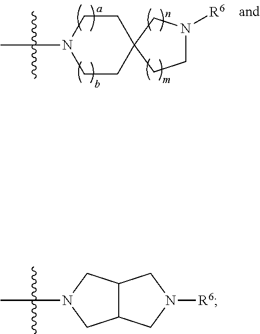

each R² is independently selected from C₁₋₆alkyl, halogen, —CN, C₁₋₆haloalkyl, C₃₋₈cycloalkyl, —SF₅, —OR³, and —C(O)NR⁴R⁵;
each R³ is independently selected from H, C₁₋₆alkyl, C₁₋₆haloalkyl, C₃₋₈cycloalkyl, and —C₁₋₆alkyl-C₃₋₈cycloalkyl;
each R⁴ and R⁵ is independently selected from H, C₁₋₆alkyl, and C₃₋₈cycloalkyl;
R⁶ is selected from C₁₋₆alkyl, —C(O)—C₁₋₆alkyl, and —S(O)₂—C₁₋₆alkyl;
a is 0 or 1;
b is 0 or 1;
m is 0, 1, or 2;
n is 0, 1, or 2; provided that when n is 0, then m is 2; and
p is 0, 1, 2, 3, or 4;
   or a pharmaceutically acceptable salt or solvate thereof.

In some embodiments is a method for treating dyskinesia in a patient in need thereof, comprising administering to the patient a therapeutically effective amount of a compound of Formula (XI), wherein the dyskinesia is levodopa-induced dyskinesia.

In some embodiments of the methods for treating dyskinesia with a compound of Formula (XI), R² is —NR⁵R⁶.

In some embodiments of the methods for treating dyskinesia with a compound of Formula (XI), p is 0, 1, or 2. In some embodiments of the methods for treating dyskinesia with a compound of Formula (XI), p is 0 or 1. In some embodiments of the methods for treating dyskinesia with a compound of Formula (XI), p is 1 or 2. In some embodiments of the methods for treating dyskinesia with a compound of Formula (XI), p is 0. In some embodiments of the methods for treating dyskinesia with a compound of Formula (XI), p is 1. In some embodiments of the methods for treating dyskinesia with a compound of Formula (XI), p is 2.

In some embodiments of the methods for treating dyskinesia with a compound of Formula (XI), R¹ is

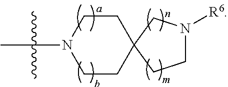

In some embodiments of the methods for treating dyskinesia with a compound of Formula (XI), R¹ is

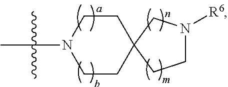

a is 1, b is 1, m is 1, and n is 1. In some embodiments of the methods for treating dyskinesia with a compound of Formula (XI), R¹ is

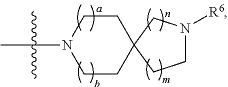

a is 1, b is 1, m is 0, and n is 1. In some embodiments of the methods for treating dyskinesia with a compound of Formula (XI), R¹ is

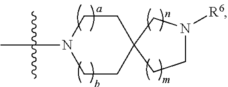

a is 1, b is 1, m is 2, and n is 0. In some embodiments of the methods for treating dyskinesia with a compound of Formula (XI), R¹ is

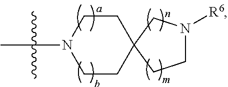

a is 0, b is 1, m is 1, and n is 1. In some embodiments of the methods for treating dyskinesia with a compound of Formula (XI), R¹ is

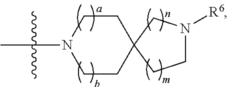

a is 0, b is 1, m is 1, and n is 2. In some embodiments of the methods for treating dyskinesia with a compound of Formula (XI), R¹ is

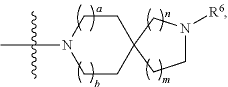

a is 0, b is 1, m is 0, and n is 1. In some embodiments of the methods for treating dyskinesia with a compound of Formula (XI), R¹ is

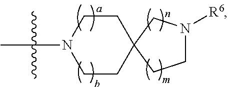

a is 0, b is 0, m is 1, and n is 1. In some embodiments of the methods for treating dyskinesia with a compound of Formula (XI), R¹ is

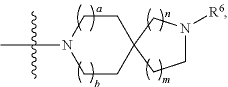

a is 0, b is 0, m is 1, and n is 2.

In some embodiments of the methods for treating dyskinesia with a compound of Formula (XI), R¹ is

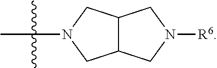

In some embodiments of the methods for treating dyskinesia with a compound of Formula (XI), R⁶ is C₁₋₆alkyl. In some embodiments of the methods for treating dyskinesia with a compound of Formula (XI), R⁶ is —CH₃. In some embodiments of the methods for treating dyskinesia with a compound of Formula (XI), R⁶ is —CH₂CH₃. In some embodiments of the methods for treating dyskinesia with a compound of Formula (XI), R⁶ is —C(O)—C₁₋₆alkyl. In some embodiments of the methods for treating dyskinesia with a compound of Formula (XI), R⁶ is —C(O)CH₃. In some embodiments of the methods for treating dyskinesia with a compound of Formula (XI), R⁶ is —S(O)₂—C₁₋₆ alkyl. In some embodiments of the methods for treating dyskinesia with a compound of Formula (XI), R⁶ is —S(O)₂CH₃.

In some embodiments of the methods for treating dyskinesia with a compound of Formula (XI), p is 1 and R³ is selected from C₁₋₆alkyl, halogen, C₁₋₆ haloalkyl, —SF₅, and —OR⁷. In some embodiments of the methods for treating dyskinesia with a compound of Formula (XI), p is 1 and R³ is selected from halogen, C₁₋₆haloalkyl, and —OR⁷. In some embodiments of the methods for treating dyskinesia with a compound of Formula (XI), p is 1 and R³ is selected from halogen and C₁₋₆haloalkyl. In some embodiments of the methods for treating dyskinesia with a compound of Formula (XI), p is 1 and R³ is C₁₋₆alkyl. In some embodiments of the methods for treating dyskinesia with a compound of Formula (XI), p is 1 and R³ is —CH₃. In some embodiments of the methods for treating dyskinesia with a compound of Formula (XI), p is 1 and R³ is halogen. In some embodiments of the methods for treating dyskinesia with a compound of Formula (XI), p is 1 and R³ is —Cl. In some embodiments of the methods for treating dyskinesia with a compound of Formula (XI), p is 1 and R³ is —CN. In some embodiments of the methods for treating dyskinesia with a compound of Formula (XI), p is 1 and R³ is C₁₋₆haloalkyl. In some embodiments of the methods for treating dyskinesia with a compound of Formula (XI), p is 1 and R³ is —CF₃. In some embodiments of the methods for treating dyskinesia with a compound of Formula (XI), p is 1 and R³ is —SF₅. In some embodiments of the methods for treating dyskinesia with a compound of Formula (XI), p is 1 and R³ is —OR⁷. In some embodiments of the methods for treating dyskinesia with a compound of Formula (XI), p is 1 and R³ is —OCH₃.

In some embodiments of the methods for treating dyskinesia with a compound of Formula (XI), the compound is selected from:

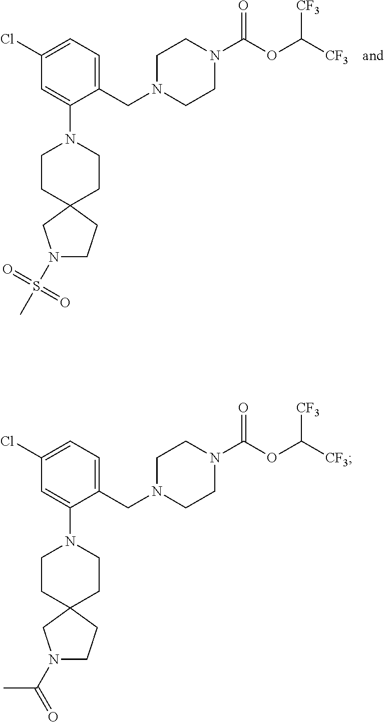 [SMILES: CS(=O)(=O)N1CCC2(CCN(c3cc(Cl)ccc3CN3CCN(C(=O)OC(C(F)(F)F)C(F)(F)F)CC3)CC2)C1 ; CC(=O)N1CCC2(CCN(c3cc(Cl)ccc3CN3CCN(C(=O)OC(C(F)(F)F)C(F)(F)F)CC3)CC2)C1]

or a pharmaceutically acceptable salt or solvate thereof.

In some embodiments is a method for treating dyskinesia in a patient in need thereof, comprising administering to the patient a therapeutically effective amount of a compound of Formula (XII):

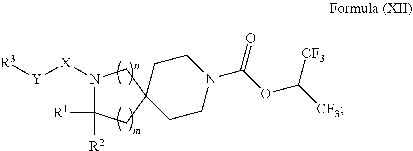

wherein:
X is —CH₂— or —C(O)—;
Y is a bond, C₁₋₆alkyl, C₁₋₆haloalkyl, or C₃₋₈cycloalkyl;
R¹ is H or C₁₋₆alkyl;
R² is H or C₁₋₆alkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, —CH₂CO₂R⁶, and —CH₂C(O)N(H)SO₂R⁸; wherein C₂₋₉heterocycloalkyl, —C₁₋₆alkyl(C₂₋₉heterocycloalkyl), phenyl, and C₁₋₉ heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl;
each R⁸ is independently selected from C₁₋₆alkyl, C₁₋₆haloalkyl, and C₃₋₈cycloalkyl;
n is 0 or 1; and
m is 1 or 2; provided that when n is 0, then m is 2; and when n is 1, then m is 1;
   or a pharmaceutically acceptable salt or solvate thereof.

In some embodiments is a method for treating dyskinesia in a patient in need thereof, comprising administering to the patient a therapeutically effective amount of a compound of Formula (XII), wherein the dyskinesia is levodopa-induced dyskinesia.

In some embodiments of the methods for treating dyskinesia with a compound of Formula (XII), n is 0 and m is 2. In some embodiments of the methods for treating dyskinesia with a compound of Formula (XII), n is 1 and m is 1.

In some embodiments of the methods for treating dyskinesia with a compound of Formula (XIII), R¹ is H. In some embodiments of the methods for treating dyskinesia with a compound of Formula (XII), R² is H. In some embodiments of the methods for treating dyskinesia with a compound of Formula (XII), R¹ is H and R² is H.

In some embodiments of the methods for treating dyskinesia with a compound of Formula (XII), X is —CH₂—. In some embodiments of the methods for treating dyskinesia with a compound of Formula (XII), X is —C(O)—.

In some embodiments of the methods for treating dyskinesia with a compound of Formula (XII), Y is a bond. In some embodiments of the methods for treating dyskinesia with a compound of Formula (XII), Y is C₁₋₆alkyl. In some embodiments of the methods for treating dyskinesia with a compound of Formula (XII), Y is —CH₂—. In some embodiments of the methods for treating dyskinesia with a compound of Formula (XII), Y is C₁₋₆haloalkyl. In some embodiments of the methods for treating dyskinesia with a compound of Formula (XII), Y is —CF₂—. In some embodiments of the methods for treating dyskinesia with a compound of Formula (XII), Y is C₃₋₈cycloalkyl. In some embodiments of the methods for treating dyskinesia with a compound of Formula (XII), Y is cyclopropyl.

In some embodiments of the methods for treating dyskinesia with a compound of Formula (XII), R³ is a 5- to 6-membered heteroaryl ring optionally substituted with one, two, or three R⁴. In some embodiments of the methods for treating dyskinesia with a compound of Formula (XII), R³ is a 5-membered heteroaryl ring optionally substituted with one, two, or three R⁴. In some embodiments of the methods for treating dyskinesia with a compound of Formula (XII), R³ is an unsubstituted 5-membered heteroaryl ring. In some embodiments of the methods for treating dyskinesia with a compound of Formula (XII), R³ is a 5-membered heteroaryl ring substituted with one, two, or three R⁴. In some embodiments of the methods for treating dyskinesia with a compound of Formula (XII), R³ is a 5-membered heteroaryl ring substituted with two or three R⁴, wherein two adjacent R⁴ form a 6-membered heterocycloalkyl ring optionally substituted with one or two R⁵. In some embodiments of the methods for treating dyskinesia with a compound of Formula (XII), R³ is a 5-membered heteroaryl ring substituted with two adjacent R⁴, wherein the two adjacent R⁴ form an unsubstituted 6-membered heterocycloalkyl ring. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is selected from C₁₋₆alkyl, C₁₋₆heteroalkyl, C₃₋₈cycloalkyl, —C₁₋₆alkyl(C₃₋₈cycloalkyl), C₂₋₉heterocycloalkyl, and —CH₂CO₂H. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is C₁₋₆alkyl. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is C₁₋₆heteroalkyl. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is C₃₋₈cycloalkyl. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is —C₁₋₆alkyl(C₃₋₈ cycloalkyl). In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is C₂₋₉heterocycloalkyl. In some embodiments of the methods for treating dyskinesia with a compound of Formula (XII), R³ is a 5-membered heteroaryl ring substituted with two adjacent R⁴, wherein the two adjacent R⁴ form a 6-membered heterocycloalkyl ring substituted with one R⁵ and R⁵ is —CH₂CO₂H. In some embodiments of the methods for treating dyskinesia with a compound of Formula (XII), R³ is a 5-membered heteroaryl ring substituted with two or three R⁴, wherein two adjacent R⁴ form a 6-membered cycloalkyl ring optionally substituted with one or two R⁵. In some embodiments of the methods for treating dyskinesia with a compound of Formula (XII), R³ is a 5-membered heteroaryl ring substituted with two adjacent R⁴, wherein the two adjacent R⁴ form an unsubstituted 6-membered cycloalkyl ring. In some embodiments of the methods for treating dyskinesia with a compound of Formula (XII), R³ is a 5-membered heteroaryl ring substituted with two adjacent R⁴, wherein the two adjacent R⁴ form a 6-membered cycloalkyl ring substituted with one R⁵. In some embodiments of the methods for treating dyskinesia with a compound of Formula (XII), R³ is selected from:

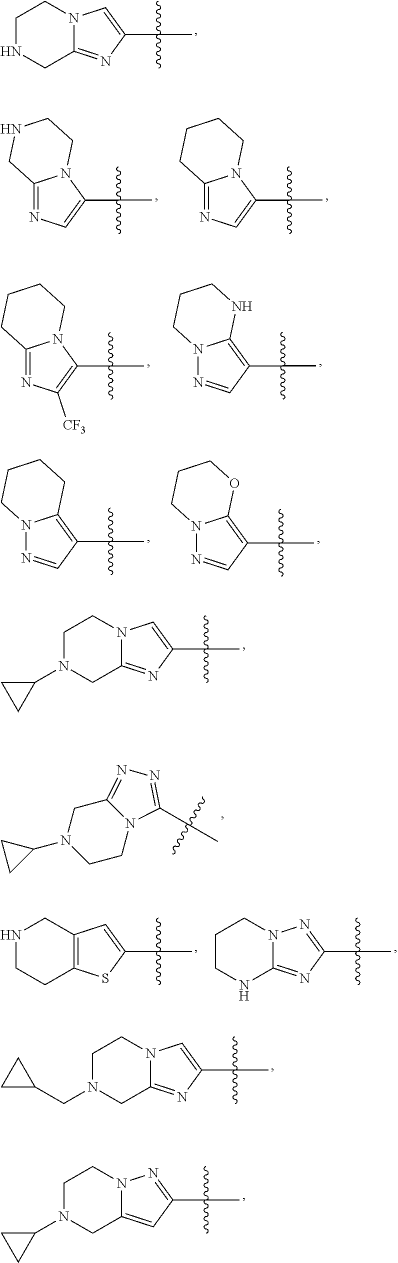

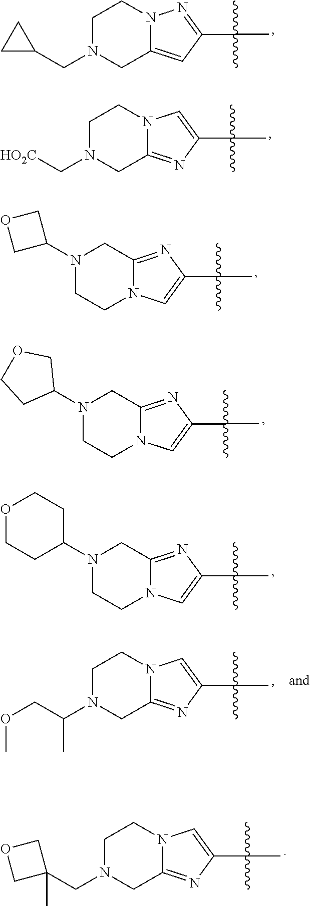

In some embodiments of the methods for treating dyskinesia with a compound of Formula (XII), the compound is:

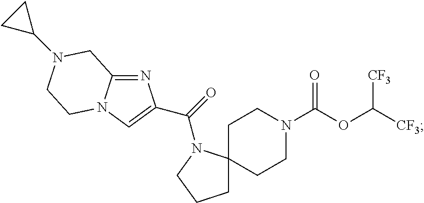 [SMILES: O=C(OC(C(F)(F)F)C(F)(F)F)N1CCC2(CCCN2C(=O)c2cn3c(n2)CN(C2CC2)CC3)CC1]

or a pharmaceutically acceptable salt or solvate thereof.

In some embodiments is a method for treating dyskinesia in a patient in need thereof, comprising administering to the patient a therapeutically effective amount of a compound of Formula (XIII):

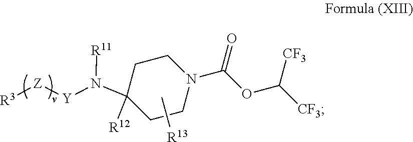

wherein:
Y is —CH₂— or —C(O)—;
Z is C₃₋₆cycloalkyl;
R³ is a 5- to 6-membered heteroaryl ring or a 9- to 10-membered bicyclic heteroaryl ring; wherein the 5- to 6-membered heteroaryl ring and the 9- to 10-membered bicyclic heteroaryl ring are optionally substituted with one, two, or three R⁴;
each R⁴ is independently selected from C₁₋₆alkyl, halogen, —CN, C₁₋₆haloalkyl, C₃₋₈cycloalkyl, C₂₋₉heterocycloalkyl, —C₁₋₆alkyl-(C₂₋₉heterocycloalkyl), phenyl, —CH₂— phenyl, C₁₋₉heteroaryl, —OR⁷, —CO₂R⁶, and —CH₂CO₂R⁶; wherein C₂₋₉heterocycloalkyl, —C₁₋₆alkyl(C₂₋₉heterocycloalkyl), phenyl, and C₁₋₉heteroaryl are optionally substituted with one or two R⁵; or two adjacent R⁴ form a 6-membered cycloalkyl or 6-membered heterocycloalkyl ring, wherein the cycloalkyl and heterocycloalkyl ring are optionally substituted with one or two R⁵;
each R⁵ is independently selected from halogen, C₁₋₆alkyl, C₁₋₆haloalkyl, C₁₋₆heteroalkyl, C₁₋₆alkoxy, C₃₋₈cycloalkyl, —C₁₋₆alkyl(C₃₋₈cycloalkyl), C₂₋₉heterocycloalkyl, —CO₂R⁶, —CH₂CO₂R⁶, and —C₁₋₆alkyl(C₂₋₉heterocycloalkyl) optionally substituted with C₁₋₆alkyl;
each R⁶ is independently selected from H and C₁₋₆alkyl;
each R⁷ is independently selected from H, C₁₋₆alkyl, C₁₋₆haloalkyl, and C₃₋₈cycloalkyl; R¹¹ is H, C₁₋₆alkyl, or —C₁₋₆alkyl-O—C₁₋₆alkyl;
R¹² is C₁₋₆alkyl;
R¹³ is H or C₁₋₆alkyl; and
v is 0 or 1;
   or a pharmaceutically acceptable salt or solvate thereof.

In some embodiments is a method for treating dyskinesia in a patient in need thereof, comprising administering to the patient in need thereof a therapeutically effective amount of a compound of Formula (XIII), wherein the dyskinesia is levodopa-induced dyskinesia.

In some embodiments of the methods for treating dyskinesia with a compound of Formula (XIII), R¹¹ is H. In some embodiments of the methods for treating dyskinesia with a compound of Formula (XIII), R¹¹ is C₁₋₆alkyl. In some embodiments of the methods for treating dyskinesia with a compound of Formula (XIII), R¹¹ is —CH₃. In some embodiments of the methods for treating dyskinesia with a compound of Formula (XIII), R¹² is —CH₃. In some embodiments of the methods for treating dyskinesia with a compound of Formula (XIII), R¹³ is H.

In some embodiments of the methods for treating dyskinesia with a compound of Formula (XIII), Y is —CH₂—. In some embodiments of the methods for treating dyskinesia with a compound of Formula (XIII), Y is —C(O)—.

In some embodiments of the methods for treating dyskinesia with a compound of Formula (XIII), v is 0. In some embodiments of the methods for treating dyskinesia with a compound of Formula (XIII), v is 1.

In some embodiments of the methods for treating dyskinesia with a compound of Formula (XIII), R³ is a 5- to 6-membered heteroaryl ring optionally substituted with one, two, or three R⁴. In some embodiments of the methods for treating dyskinesia with a compound of Formula (XIII), R³ is a 5-membered heteroaryl ring optionally substituted with one, two, or three R⁴. In some embodiments of the methods for treating dyskinesia with a compound of Formula (XIII), R³ is an unsubstituted 5-membered heteroaryl ring. In some embodiments of the methods for treating dyskinesia with a compound of Formula (XIII), R³ is a 5-membered heteroaryl ring substituted with one, two, or three R⁴. In some embodiments of the methods for treating dyskinesia with a compound of Formula (XIII), R³ is a 5-membered heteroaryl ring substituted with two or three R⁴, wherein two adjacent R⁴ form a 6-membered heterocycloalkyl ring optionally substituted with one or two R⁵. In some embodiments of the methods for treating dyskinesia with a compound of Formula (XIII), R³ is a 5-membered heteroaryl ring substituted with two adjacent R⁴, wherein the two adjacent R⁴ form an unsubstituted 6-membered heterocycloalkyl ring. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is selected from C₁₋₆alkyl, C₁₋₆heteroalkyl, C₃₋₈cycloalkyl, —C₁₋₆alkyl(C₃₋₈cycloalkyl), C₂₋₉heterocycloalkyl, and —CH₂CO₂H. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is C₁₋₆alkyl. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is C₁₋₆heteroalkyl. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is C₃₋₈cycloalkyl. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is —C₁₋₆alkyl(C₃₋₈ cycloalkyl). In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is C₂₋₉heterocycloalkyl. In some embodiments of the methods for treating dyskinesia with a compound of Formula (XIII), R³ is a 5-membered heteroaryl ring substituted with two adjacent R⁴, wherein the two adjacent R⁴ form a 6-membered heterocycloalkyl ring substituted with one R⁵ and R⁵ is —CH₂CO₂H. In some embodiments of the methods for treating dyskinesia with a compound of Formula (XIII), R³ is a 5-membered heteroaryl ring substituted with two or three R⁴, wherein two adjacent R⁴ form a 6-membered cycloalkyl ring optionally substituted with one or two R⁵. In some embodiments of the methods for treating dyskinesia with a compound of Formula (XIII), R³ is a 5-membered heteroaryl ring substituted with two adjacent R⁴, wherein the two adjacent R⁴ form an unsubstituted 6-membered cycloalkyl ring. In some embodiments of the methods for treating dyskinesia with a compound of Formula (XIII), R³ is a 5-membered heteroaryl ring substituted with two adjacent R⁴, wherein the two adjacent R⁴ form a 6-membered cycloalkyl ring substituted with one R⁵. In some embodiments of the methods for treating dyskinesia with a compound of Formula (XIII), R³ is selected from:

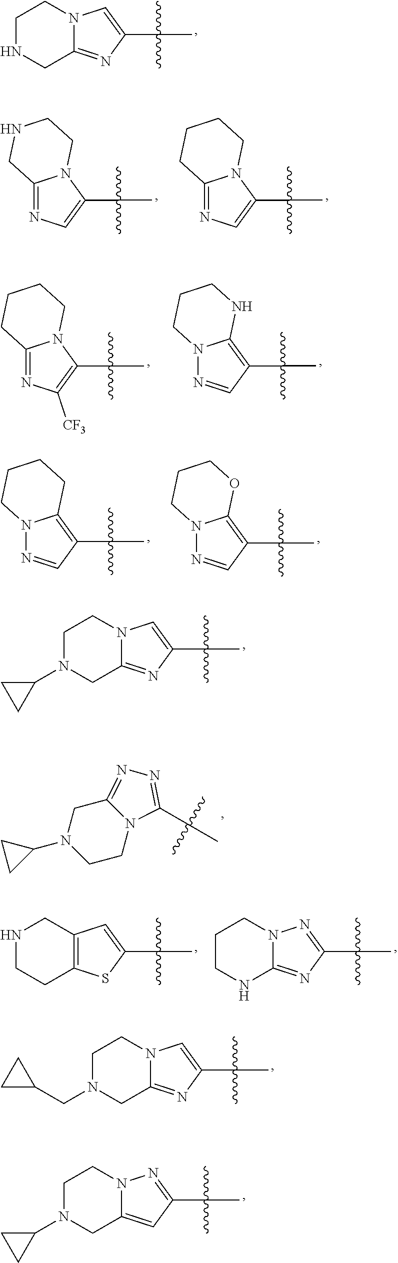

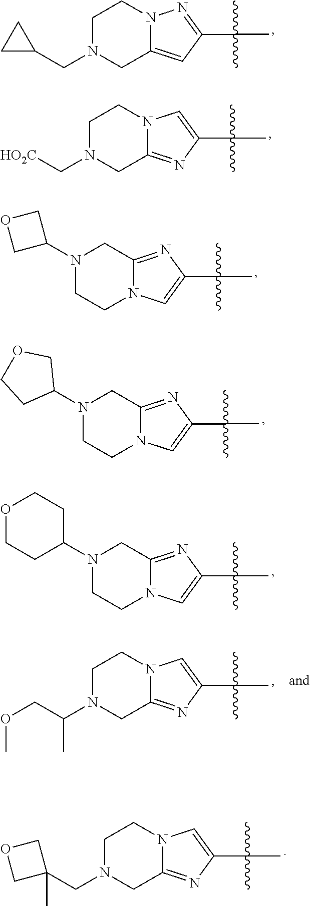

In some embodiments of the methods for treating dyskinesia with a compound of Formula (XIII), the compound is selected from:

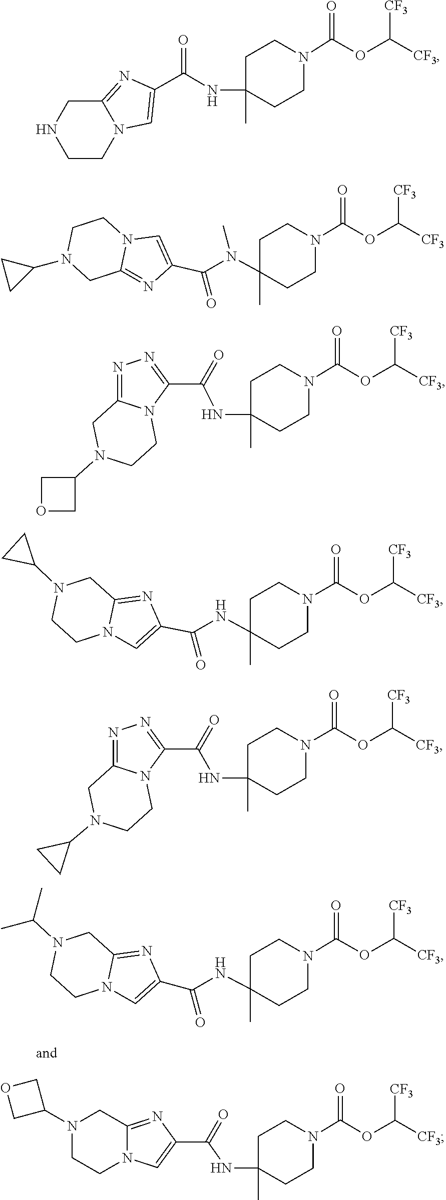

or a pharmaceutically acceptable salt or solvate thereof.

In some embodiments is a method for treating dyskinesia in a patient in need thereof, comprising administering to the patient a therapeutically effective amount of a compound of Formula (XIV):

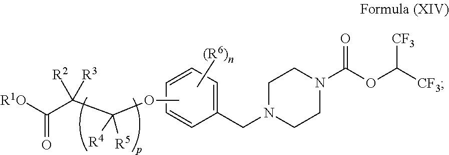

wherein:
R¹ is H or C₁₋₆alkyl;
R² is C₁₋₆alkyl;
R³ is H or C₁₋₆alkyl;
R⁴ and R⁵ are independently selected from H and C₁₋₆alkyl;
each R⁶ is independently selected from C₁₋₆alkyl, halogen, —CN, C₁₋₆haloalkyl, —OR⁷, —C(O)NR⁸R⁹, C₃₋₆cycloalkyl, C₂₋₉heterocycloalkyl, —C₁₋₆alkyl(C₂₋₉heterocycloalkyl), and C₂₋₉heteroaryl, wherein C₃₋₆cycloalkyl, C₂₋₉heterocycloalkyl, —C₁₋₆alkyl(C₂₋₉heterocycloalkyl), and C₂₋₉heteroaryl are optionally substituted with one, two, or three groups independently selected from halogen, C₁₋₆alkyl, C₁₋₆haloalkyl, and C₁₋₆alkoxy;
each R⁷ is independently selected from H, C₁₋₆alkyl, C₁₋₆haloalkyl, and C₃₋₆cycloalkyl;
each R⁸ and R⁹ is each independently selected from H, C₁₋₆alkyl, C₃₋₆cycloalkyl, aryl, and heteroaryl; or R⁸ and R⁹, together with the nitrogen to which they are attached, form a heterocycloalkyl ring optionally substituted with one, two, or three R¹⁰;
each R¹⁰ is independently selected from halogen, C₁₋₆alkyl, C₁₋₆haloalkyl, oxo, —CN, and C₃₋₆cycloalkyl;
n is 0, 1, 2, 3, or 4; and
p is 0 or 1;
   or a pharmaceutically acceptable salt or solvate thereof.

In some embodiments is a method for treating dyskinesia in a patient in need thereof, comprising administering to the patient a therapeutically effective amount of a compound of Formula (XIV), wherein the dyskinesia is levodopa-induced dyskinesia.

In some embodiments of the methods for treating dyskinesia with a compound of Formula (XIV), p is 0. In some embodiments of the methods for treating dyskinesia with a compound of Formula (XIV), p is 1. In some embodiments of the methods for treating dyskinesia with a compound of Formula (XIV), p is 1 and R⁴ and R⁵ are H. In some embodiments of the methods for treating dyskinesia with a compound of Formula (XIV), R³ is H. In some embodiments of the methods for treating dyskinesia with a compound of Formula (XIV), R³ is C₁₋₆alkyl. In some embodiments of the methods for treating dyskinesia with a compound of Formula (XIV), R³ is —CH₃. In some embodiments of the methods for treating dyskinesia with a compound of Formula (XIV), R³ is H and R² is —CH₃. In some embodiments of the methods for treating dyskinesia with a compound of Formula (XIV), R³ is C₁₋₆alkyl and R² is —CH₃. In some embodiments of the methods for treating dyskinesia with a compound of Formula (XIV), R³ is —CH₃ and R² is —CH₃. In some embodiments of the methods for treating dyskinesia with a compound of Formula (XIV), R¹ is H. In some embodiments of the methods for treating dyskinesia with a compound of Formula (XIV), R¹ is C₁₋₆alkyl. In some embodiments of the methods for treating dyskinesia with a compound of Formula (XIV), R¹ is —CH₃.

In some embodiments of the methods for treating dyskinesia with a compound of Formula (XIV), n is 0. In some embodiments of the methods for treating dyskinesia with a compound of Formula (XIV), n is 1. In some embodiments of the methods for treating dyskinesia with a compound of Formula (XIV), n is 2.

In some embodiments of the methods for treating dyskinesia with a compound of Formula (XIV), each R⁶ is independently selected from C₁₋₆alkyl, halogen, —CN, C₁₋₆haloalkyl, —OR⁷, C₃₋₆cycloalkyl, C₂₋₉heterocycloalkyl, and C₂₋₉heteroaryl, wherein C₃₋₆cycloalkyl, C₂₋₉heterocycloalkyl, and C₂₋₉heteroaryl are optionally substituted with one or two groups independently selected from halogen, C₁₋₆alkyl, C₁₋₆haloalkyl, and C₁₋₆ alkoxy. In some embodiments of the methods for treating dyskinesia with a compound of Formula (XIV), each R⁶ is independently selected from C₁₋₆alkyl, halogen, —CN, C₁₋₆ haloalkyl, —OR⁷, and C₃₋₆cycloalkyl. In some embodiments of the methods for treating dyskinesia with a compound of Formula (XIV), each R⁶ is independently selected from C₁₋₆alkyl, halogen, —CN, C₁₋₆haloalkyl, —OR⁷, and C₃₋₆cycloalkyl, wherein each R⁷ is independently selected from C₁₋₆alkyl and C₁₋₆haloalkyl. In some embodiments of the methods for treating dyskinesia with a compound of Formula (XIV), each R⁶ is independently selected from C₁₋₆alkyl, halogen, —CN, and C₁₋₆haloalkyl.

In some embodiments of the methods for treating dyskinesia with a compound of Formula (XIV), n is 1 and R⁶ is independently selected from C₁₋₆alkyl, halogen, —CN, C₁₋₆haloalkyl, —OR⁷, C₃₋₆cycloalkyl, C₂₋₉heterocycloalkyl, and C₂₋₉heteroaryl, wherein C₃₋₆cycloalkyl, C₂₋₉heterocycloalkyl, and C₂₋₉heteroaryl are optionally substituted with one or two groups independently selected from halogen, C₁₋₆alkyl, C₁₋₆haloalkyl, and C₁₋₆ alkoxy. In some embodiments of the methods for treating dyskinesia with a compound of Formula (XIV), n is 1 and R⁶ is C₁₋₆alkyl. In some embodiments of the methods for treating dyskinesia with a compound of Formula (XIV), n is 1 and R⁶ is —CH₃. In some embodiments of the methods for treating dyskinesia with a compound of Formula (XIV), n is 1 and R⁶ is halogen. In some embodiments of the methods for treating dyskinesia with a compound of Formula (XIV), n is 1 and R⁶ is —Cl. In some embodiments of the methods for treating dyskinesia with a compound of Formula (XIV), n is 1 and R⁶ is —F. In some embodiments of the methods for treating dyskinesia with a compound of Formula (XIV), n is 1 and R⁶ is —CN. In some embodiments of the methods for treating dyskinesia with a compound of Formula (XIV), n is 1 and R⁶ is C₁₋₆haloalkyl. In some embodiments of the methods for treating dyskinesia with a compound of Formula (XIV), n is 1 and R⁶ is —CF₃. In some embodiments of the methods for treating dyskinesia with a compound of Formula (XIV), n is 1 and R⁶ is —OR⁷. In some embodiments of the methods for treating dyskinesia with a compound of Formula (XIV), n is 1, R⁶ is —OR⁷, and R⁷ is selected from C₁₋₆alkyl and C₁₋₆haloalkyl. In some embodiments of the methods for treating dyskinesia with a compound of Formula (XIV), n is 1, R⁶ is —OR⁷, and R⁷ is H. In some embodiments of the methods for treating dyskinesia with a compound of Formula (XIV), n is 1, R⁶ is —OR⁷, and R⁷ is C₁₋₆alkyl. In some embodiments of the methods for treating dyskinesia with a compound of Formula (XIV), n is 1, R⁶ is —OR⁷, and R⁷ is —CH₃. In some embodiments of the methods for treating dyskinesia with a compound of Formula (XIV), n is 1, R⁶ is —OR⁷, and R⁷ is C₁₋₆ haloalkyl. In some embodiments of the methods for treating dyskinesia with a compound of Formula (XIV), n is 1, R⁶ is —OR⁷, and R⁷ is —CF₃. In some embodiments of the methods for treating dyskinesia with a compound of Formula (XIV), n is 1, R⁶ is —OR⁷, and R⁷ is C₃₋₆cycloalkyl. In some embodiments of the methods for treating dyskinesia with a compound of Formula (XIV), n is 1 and R⁶ is C₃₋₆cycloalkyl optionally substituted with one, two, or three groups independently selected from halogen, C₁₋₆alkyl, C₁₋₆haloalkyl, and C₁₋₆alkoxy. In some embodiments of the methods for treating dyskinesia with a compound of Formula (XIV), n is 1 and R⁶ is C₃₋₆cycloalkyl substituted with one or two groups independently selected from halogen, C₁₋₆alkyl, C₁₋₆ haloalkyl, and C₁₋₆alkoxy. In some embodiments of the methods for treating dyskinesia with a compound of Formula (XIV), n is 1 and R⁶ is unsubstituted C₃₋₆cycloalkyl. In some embodiments of the methods for treating dyskinesia with a compound of Formula (XIV), n is 1 and R⁶ is C₂₋₉heterocycloalkyl optionally substituted with one, two, or three groups independently selected from halogen, C₁₋₆alkyl, C₁₋₆haloalkyl, and C₁₋₆alkoxy. In some embodiments of the methods for treating dyskinesia with a compound of Formula (XIV), n is 1 and R⁶ is C₂₋₉heterocycloalkyl substituted with one or two groups independently selected from halogen, C₁₋₆alkyl, C₁₋₆haloalkyl, and C₁₋₆alkoxy. In some embodiments of the methods for treating dyskinesia with a compound of Formula (XIV), n is 1 and R⁶ is unsubstituted C₂₋₉heterocycloalkyl. In some embodiments of the methods for treating dyskinesia with a compound of Formula (XIV), n is 1 and R⁶ is C₂₋₉ heteroaryl optionally substituted with one, two, or three groups independently selected from halogen, C₁₋₆alkyl, C₁₋₆haloalkyl, and C₁₋₆alkoxy. In some embodiments of the methods for treating dyskinesia with a compound of Formula (XIV), n is 1 and R⁶ is C₂₋₉ heteroaryl substituted with one or two groups independently selected from halogen, C₁₋₆alkyl, C₁₋₆haloalkyl, and C₁₋₆alkoxy. In some embodiments of the methods for treating dyskinesia with a compound of Formula (XIV), n is 1 and R⁶ is unsubstituted C₂₋₉heteroaryl.

In some embodiments of the methods for treating dyskinesia with a compound of Formula (XIV), the compound is selected from:

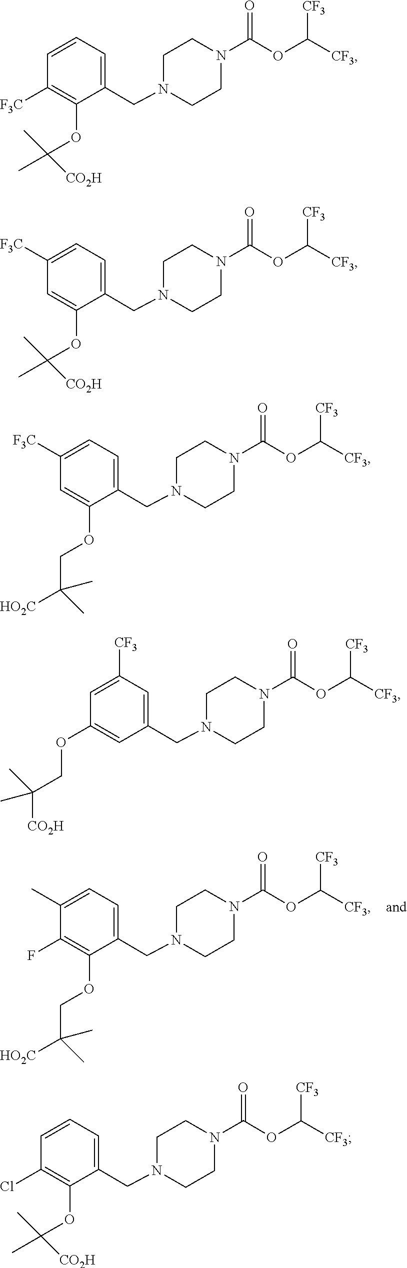

or a pharmaceutically acceptable salt or solvate thereof.

In some embodiments is a method for treating dyskinesia in a patient in need thereof, comprising administering to the patient a therapeutically effective amount of a compound of Formula (XV):

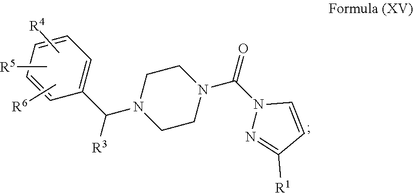

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₆alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring; and
R¹⁵ is optionally substituted C₁₋₆alkyl;
   or a pharmaceutically acceptable salt or solvate thereof.

In some embodiments is a method for treating dyskinesia in a patient in need thereof, comprising administering to the patient a therapeutically effective amount of a compound of Formula (XV), wherein the dyskinesia is levodopa-induced dyskinesia.

In some embodiments of the methods for treating dyskinesia with a compound of Formula (XV), R¹ is —N(R²)C(O)R¹⁵. In some embodiments of the methods for treating dyskinesia with a compound of Formula (XV), R¹ is —N(R²)C(O)R¹⁵ and R² is H. In some embodiments of the methods for treating dyskinesia with a compound of Formula (XV), R¹ is —N(R²)C(O)R¹⁵ and R² is C₁₋₆alkyl. In some embodiments of the methods for treating dyskinesia with a compound of Formula (XV), R¹ is —N(R²)C(O)R¹⁵ and R² is —CH₃. In some embodiments of the methods for treating dyskinesia with a compound of Formula (XV), R¹ is —N(R²)C(O)R¹⁵, R² is H, and R¹⁵ is unsubstituted C₁₋₆ alkyl. In some embodiments of the methods for treating dyskinesia with a compound of Formula (XV), R¹ is —N(R²)C(O)R¹⁵, R² is H, R¹⁵ is —CH₃. In some embodiments of the methods for treating dyskinesia with a compound of Formula (XV), R¹ is —N(R²)C(O)R¹⁵, R² is C₁₋₆alkyl, R¹⁵ is —CH₃. In some embodiments of the methods for treating dyskinesia with a compound of Formula (XV), R¹ is —N(R²)C(O)R¹⁵, R² is —CH₃, and R¹⁵ is unsubstituted C₁₋₆alkyl. In some embodiments of the methods for treating dyskinesia with a compound of Formula (XV), R¹ is —N(R²)C(O)R¹⁵, R² is —CH₃, R¹⁵ is —CH₃. In some embodiments of the methods for treating dyskinesia with a compound of Formula (XV), R¹ —N(H)SO₂R¹⁵. In some embodiments of the methods for treating dyskinesia with a compound of Formula (XV), R¹ is —N(H)SO₂R¹⁵ and R¹⁵ is unsubstituted C₁₋₆alkyl. In some embodiments of the methods for treating dyskinesia with a compound of Formula (XV), R¹ is —N(H)SO₂R¹⁵ and R¹⁵ is —CH₃. In some embodiments of the methods for treating dyskinesia with a compound of Formula (XV), R³ is H.

In some embodiments of the methods for treating dyskinesia with a compound of Formula (XV), R⁴ is H. In some embodiments of the methods for treating dyskinesia with a compound of Formula (XV), R⁴ is halogen. In some embodiments of the methods for treating dyskinesia with a compound of Formula (XV), R⁴ is —Cl. In some embodiments of the methods for treating dyskinesia with a compound of Formula (XV), R⁴ is —F. In some embodiments of the methods for treating dyskinesia with a compound of Formula (XV), R⁴ is C₁₋₆alkyl. In some embodiments of the methods for treating dyskinesia with a compound of Formula (XV), R⁴ is C₁₋₆haloalkyl. In some embodiments of the methods for treating dyskinesia with a compound of Formula (XV), R⁴ is —CF₃. In some embodiments of the methods for treating dyskinesia with a compound of Formula (XV), R⁴ is optionally substituted C₁₋₆alkyl-heterocycloalkyl. In some embodiments of the methods for treating dyskinesia with a compound of Formula (XV), R⁴ is optionally substituted heterocycloalkyl. In some embodiments of the methods for treating dyskinesia with a compound of Formula (XV), R⁴ is heterocycloalkyl optionally substituted with one or more groups selected from halogen, hydroxy, C₁₋₆alkyl, —C₁₋₆ alkyl-OH, C₁₋₆fluoroalkyl, C₃₋₆cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl. In some embodiments of the methods for treating dyskinesia with a compound of Formula (XV), R⁴ is heterocycloalkyl optionally substituted with one or two groups selected from halogen, hydroxy, C₁₋₆alkyl, —C₁₋₆alkyl-OH, C₁₋₆fluoroalkyl, C₃₋₆cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl. In some embodiments of the methods for treating dyskinesia with a compound of Formula (XV), R⁴ is optionally substituted heterocycloalkyl and the heterocycloalkyl is a 4-6 membered monocyclic heterocycloalkyl, a 8-9 membered bicyclic heterocycloalkyl, a 7-8 membered bridged heterocycloalkyl, a 5,5 fused heterocycloalkyl, or an 8-11 membered spirocyclic heterocycloalkyl. In some embodiments of the methods for treating dyskinesia with a compound of Formula (XV), R⁴ is an optionally substituted 4-6 membered monocyclic heterocycloalkyl. In some embodiments of the methods for treating dyskinesia with a compound of Formula (XV), R⁴ is an optionally substituted 8-9 membered bicyclic heterocycloalkyl. In some embodiments of the methods for treating dyskinesia with a compound of Formula (XV), R⁴ is an optionally substituted 7-8 membered bridged heterocycloalkyl. In some embodiments of the methods for treating dyskinesia with a compound of Formula (XV), R⁴ is an optionally substituted 5,5 fused heterocycloalkyl. In some embodiments of the methods for treating dyskinesia with a compound of Formula (XV), R⁴ is an optionally substituted 8-11 membered spirocyclic heterocycloalkyl. In some embodiments of the methods for treating dyskinesia with a compound of Formula (XV), R⁴ is optionally substituted heterocycloalkyl selected from

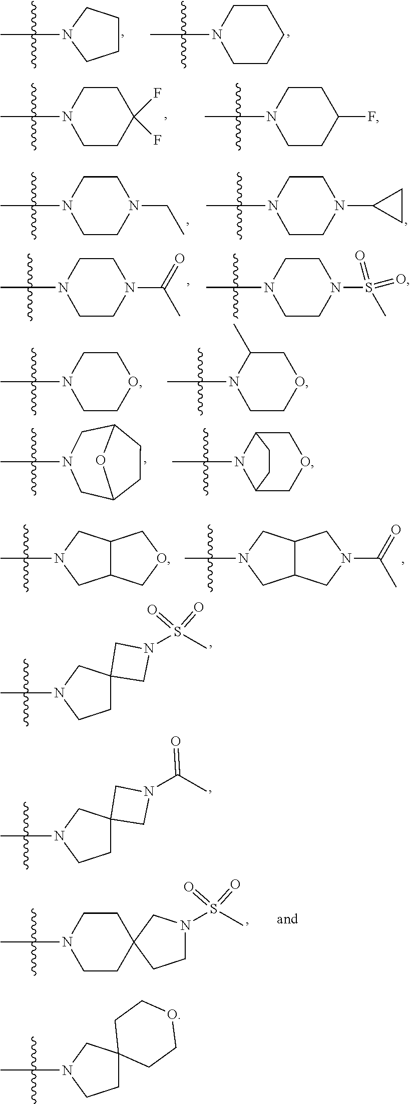

In some embodiments of the methods for treating dyskinesia with a compound of Formula (XV), R⁴ is optionally substituted heterocycloalkyl selected from

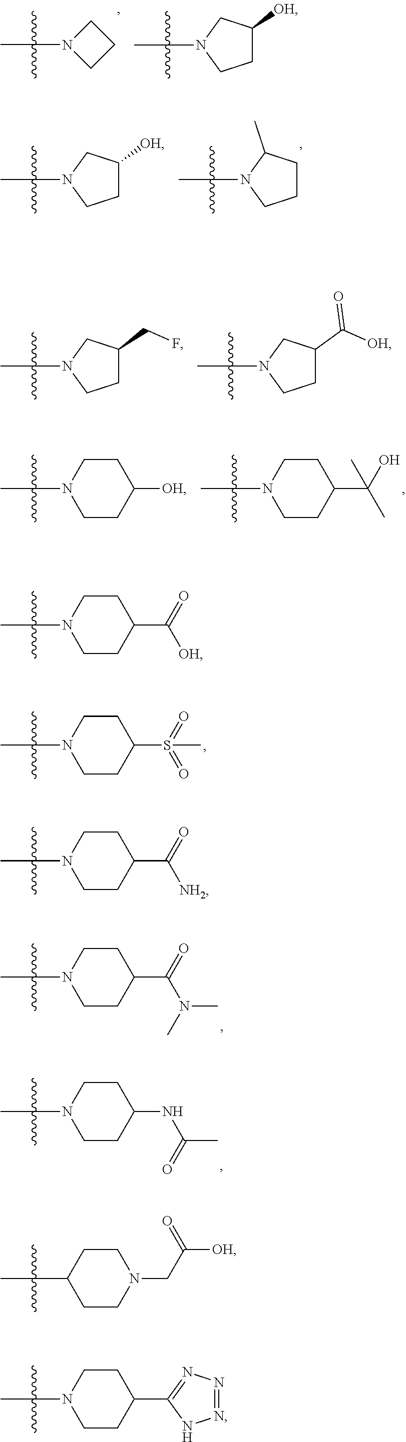

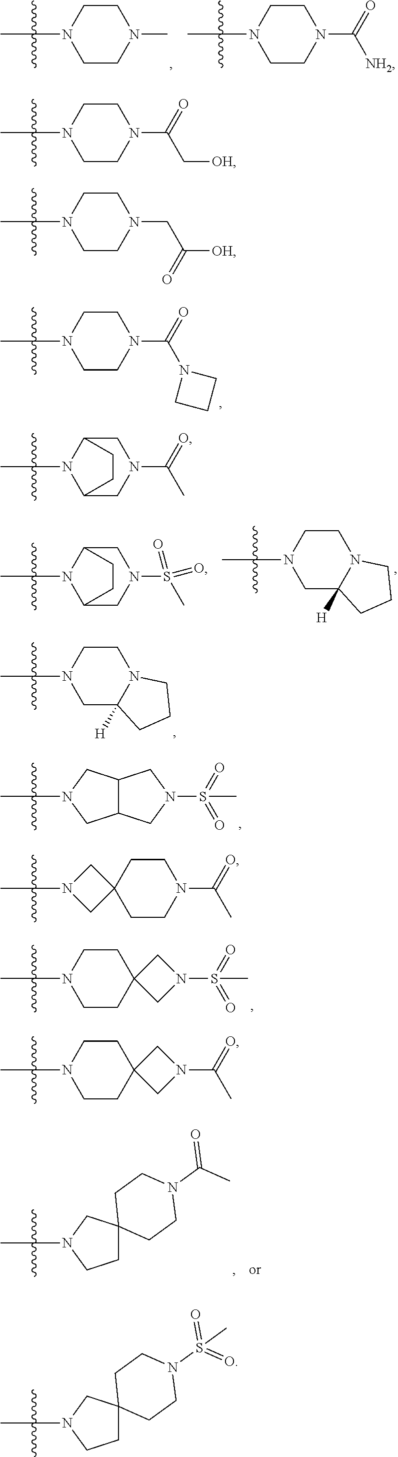

In some embodiments of the methods for treating dyskinesia with a compound of Formula (XV), R⁵ is H. In some embodiments of the methods for treating dyskinesia with a compound of Formula (XV), R⁵ is halogen. In some embodiments of the methods for treating dyskinesia with a compound of Formula (XV), R⁵ is —Cl. In some embodiments of the methods for treating dyskinesia with a compound of Formula (XV), R⁵ is —F. In some embodiments of the methods for treating dyskinesia with a compound of Formula (XV), R⁵ is C₁₋₆alkyl. In some embodiments of the methods for treating dyskinesia with a compound of Formula (XV), R⁵ is C₁₋₆haloalkyl. In some embodiments of the methods for treating dyskinesia with a compound of Formula (XV), R⁵ is —CF₃. In some embodiments of the methods for treating dyskinesia with a compound of Formula (XV), R⁵ is phenyl.

In some embodiments of the methods for treating dyskinesia with a compound of Formula (XV), R⁶ is H. In some embodiments of the methods for treating dyskinesia with a compound of Formula (XV), R⁶ is halogen. In some embodiments of the methods for treating dyskinesia with a compound of Formula (XV), R⁶ is —Cl. In some embodiments of the methods for treating dyskinesia with a compound of Formula (XV), R⁶ is —F. In some embodiments of the methods for treating dyskinesia with a compound of Formula (XV), R⁶ is C₁₋₆alkyl.

In some embodiments of the methods for treating dyskinesia with a compound of Formula (XV), the compound is selected from:

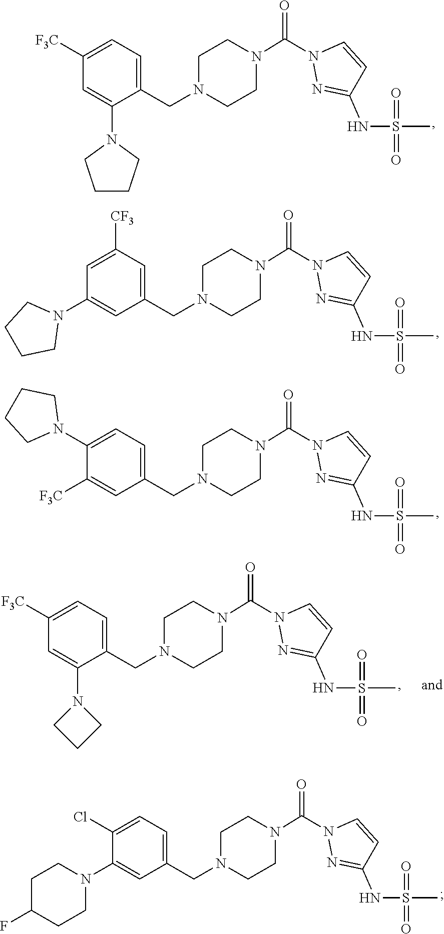

or a pharmaceutically acceptable salt or solvate thereof.

In some embodiments is a method for treating dyskinesia in a patient in need thereof, comprising administering to the patient a therapeutically effective amount of a compound of Formula (XVI):

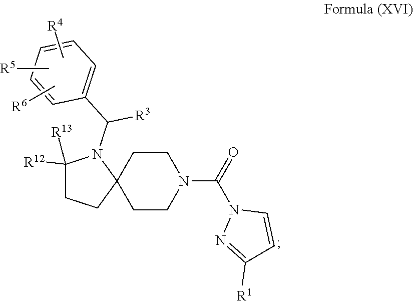

wherein:
R¹ is —N(R²)C(O)R¹⁵ or —N(H)SO₂R¹⁵;
R² is H or C₁₋₆alkyl;
R³ is H or optionally substituted phenyl;
R⁴ is H, halogen, —OR⁷, C₁₋₆alkyl, C₁₋₆haloalkyl, optionally substituted heterocycloalkyl, optionally substituted C₁₋₆alkyl-heterocycloalkyl, optionally substituted phenyl, optionally substituted heteroaryl, —CO₂H, or —C(O)NR⁸R⁹;
R⁵ is H, halogen, C₁₋₆alkyl, C₁₋₆haloalkyl, or phenyl; or
R⁴ and R⁵ are combined to form a heterocycloalkyl ring;
R⁶ is H, halogen or C₁₋₆alkyl;
R⁷ is H, C₁₋₆alkyl, optionally substituted phenyl, optionally substituted C₁₋₆alkyl-phenyl, optionally substituted heteroaryl, optionally substituted heterocycloalkyl, or —C₁₋₆alkylC(O)NR¹⁰R¹¹;
R⁸ and R⁹ are each independently H, or C₁₋₆alkyl; or R⁸ and R⁹ together with the nitrogen to which they are attached are combined to form an optionally substituted heterocycloalkyl ring;
R¹⁰ and R¹¹ are each independently H, or C₁₋₆alkyl; or R¹⁰ and R¹¹ together with the nitrogen to which they are attached are combined to form a heterocycloalkyl ring;
R¹² is H or C₁₋₆alkyl;
R¹³ is H or C₁₋₆alkyl; and
R¹⁵ is optionally substituted C₁₋₆alkyl;
   or a pharmaceutically acceptable salt or solvate thereof.

In some embodiments is a method for treating dyskinesia in a patient in need thereof, comprising administering to the patient a therapeutically effective amount of a compound of Formula (XVI), wherein the dyskinesia is levodopa-induced dyskinesia.

In some embodiments of the methods for treating dyskinesia with a compound of Formula (XVI), R¹² and R¹³ are H.

In some embodiments of the methods for treating dyskinesia with a compound of Formula (XVI), R¹ is —N(R²)C(O)R¹⁵. In some embodiments of the methods for treating dyskinesia with a compound of Formula (XVI), R¹ is —N(R²)C(O)R¹⁵ and R² is H. In some embodiments of the methods for treating dyskinesia with a compound of Formula (XVI), R¹ is —N(R²)C(O)R¹⁵ and R² is C₁₋₆alkyl. In some embodiments of the methods for treating dyskinesia with a compound of Formula (XVI), R¹ is —N(R²)C(O)R¹⁵ and R² is —CH₃. In some embodiments of the methods for treating dyskinesia with a compound of Formula (XVI), R¹ is —N(R²)C(O)R¹⁵, R² is H, and R¹⁵ is unsubstituted C₁₋₆alkyl. In some embodiments of the methods for treating dyskinesia with a compound of Formula (XVI), R¹ is —N(R²)C(O)R¹⁵, R² is H, R¹⁵ is —CH₃. In some embodiments of the methods for treating dyskinesia with a compound of Formula (XVI), R¹ is —N(R²)C(O)R¹⁵, R² is C₁₋₆alkyl, R¹⁵ is —CH₃. In some embodiments of the methods for treating dyskinesia with a compound of Formula (XVI), R¹ is —N(R²)C(O)R¹⁵, R² is —CH₃, and R¹⁵ is unsubstituted C₁₋₆alkyl. In some embodiments of the methods for treating dyskinesia with a compound of Formula (XVI), R¹ is —N(R²)C(O)R¹⁵, R² is —CH₃, R¹⁵ is —CH₃. In some embodiments of the methods for treating dyskinesia with a compound of Formula (XVI), R¹ —N(H)SO₂R¹⁵. In some embodiments of the methods for treating dyskinesia with a compound of Formula (XVI), R¹ is —N(H)SO₂R¹⁵ and R¹⁵ is unsubstituted C₁₋₆alkyl. In some embodiments of the methods for treating dyskinesia with a compound of Formula (XVI), R¹ is —N(H)SO₂R¹⁵ and R¹⁵ is —CH₃. In some embodiments of the methods for treating dyskinesia with a compound of Formula (XVI), R³ is H.

In some embodiments of the methods for treating dyskinesia with a compound of Formula (XVI), R⁴ is H. In some embodiments of the methods for treating dyskinesia with a compound of Formula (XVI), R⁴ is halogen. In some embodiments of the methods for treating dyskinesia with a compound of Formula (XVI), R⁴ is —Cl. In some embodiments of the methods for treating dyskinesia with a compound of Formula (XVI), R⁴ is —F. In some embodiments of the methods for treating dyskinesia with a compound of Formula (XVI), R⁴ is C₁₋₆alkyl. In some embodiments of the methods for treating dyskinesia with a compound of Formula (XVI), R⁴ is C₁₋₆haloalkyl. In some embodiments of the methods for treating dyskinesia with a compound of Formula (XVI), R⁴ is —CF₃. In some embodiments of the methods for treating dyskinesia with a compound of Formula (XVI), R⁴ is optionally substituted C₁₋₆alkyl-heterocycloalkyl. In some embodiments of the methods for treating dyskinesia with a compound of Formula (XVI), R⁴ is optionally substituted heterocycloalkyl. In some embodiments of the methods for treating dyskinesia with a compound of Formula (XVI), R⁴ is heterocycloalkyl optionally substituted with one or more groups selected from halogen, hydroxy, C₁₋₆alkyl, —C₁₋₆alkyl-OH, C₁₋₆fluoroalkyl, C₃₋₆cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl. In some embodiments of the methods for treating dyskinesia with a compound of Formula (XVI), R⁴ is heterocycloalkyl optionally substituted with one or two groups selected from halogen, hydroxy, C₁₋₆alkyl, —C₁₋₆alkyl-OH, C₁₋₆fluoroalkyl, C₃₋₆ cycloalkyl, heteroaryl, —CO₂H, —C₁₋₆alkyl-CO₂H, —C(O)C₁₋₆alkyl, —C(O)C₁₋₆alkyl-OH, —N(H)C(O)C₁₋₆alkyl, —C(O)NH₂, —C(O)N(H)(C₁₋₆alkyl), —C(O)N(C₁₋₆alkyl)₂, —C(O)C₂₋₇heterocycloalkyl, and —S(O)₂C₁₋₆alkyl. In some embodiments of the methods for treating dyskinesia with a compound of Formula (XVI), R⁴ is optionally substituted heterocycloalkyl and the heterocycloalkyl is a 4-6 membered monocyclic heterocycloalkyl, a 8-9 membered bicyclic heterocycloalkyl, a 7-8 membered bridged heterocycloalkyl, a 5,5 fused heterocycloalkyl, or an 8-11 membered spirocyclic heterocycloalkyl. In some embodiments of the methods for treating dyskinesia with a compound of Formula (XVI), R⁴ is an optionally substituted 4-6 membered monocyclic heterocycloalkyl. In some embodiments of the methods for treating dyskinesia with a compound of Formula (XVI), R⁴ is an optionally substituted 8-9 membered bicyclic heterocycloalkyl. In some embodiments of the methods for treating dyskinesia with a compound of Formula (XVI), R⁴ is an optionally substituted 7-8 membered bridged heterocycloalkyl. In some embodiments of the methods for treating dyskinesia with a compound of Formula (XVI), R⁴ is an optionally substituted 5,5 fused heterocycloalkyl. In some embodiments of the methods for treating dyskinesia with a compound of Formula (XVI), R⁴ is an optionally substituted 8-11 membered spirocyclic heterocycloalkyl. In some embodiments of the methods for treating dyskinesia with a compound of Formula (XVI), R⁴ is optionally substituted heterocycloalkyl selected from

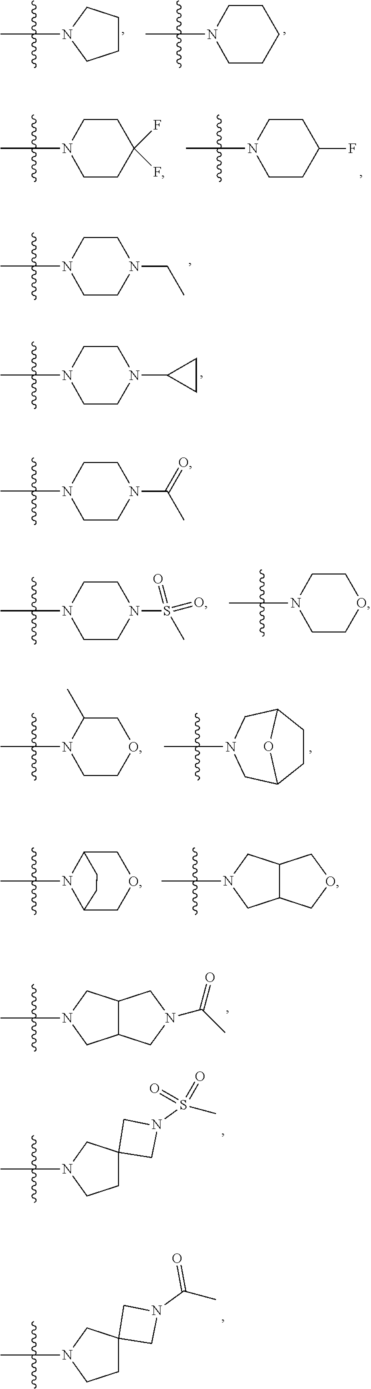

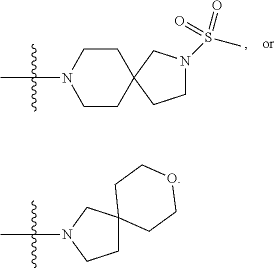 [SMILES: *N1CCC2(CC1)CCN(S(C)(=O)=O)C2 ; *N1CCC2(CCOCC2)C1]

In some embodiments of the methods for treating dyskinesia with a compound of Formula (XVI), R⁴ is optionally substituted heterocycloalkyl selected from

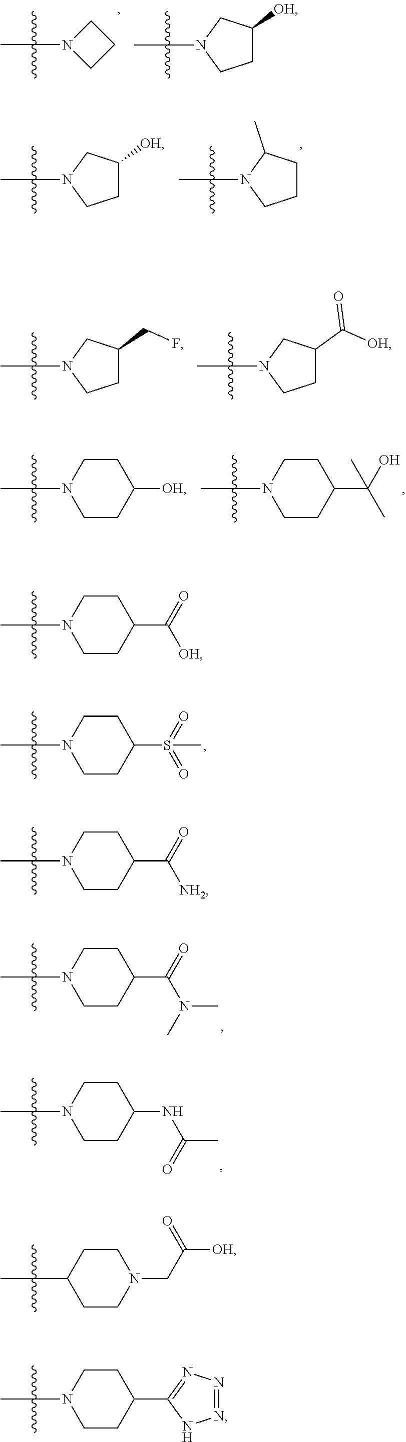

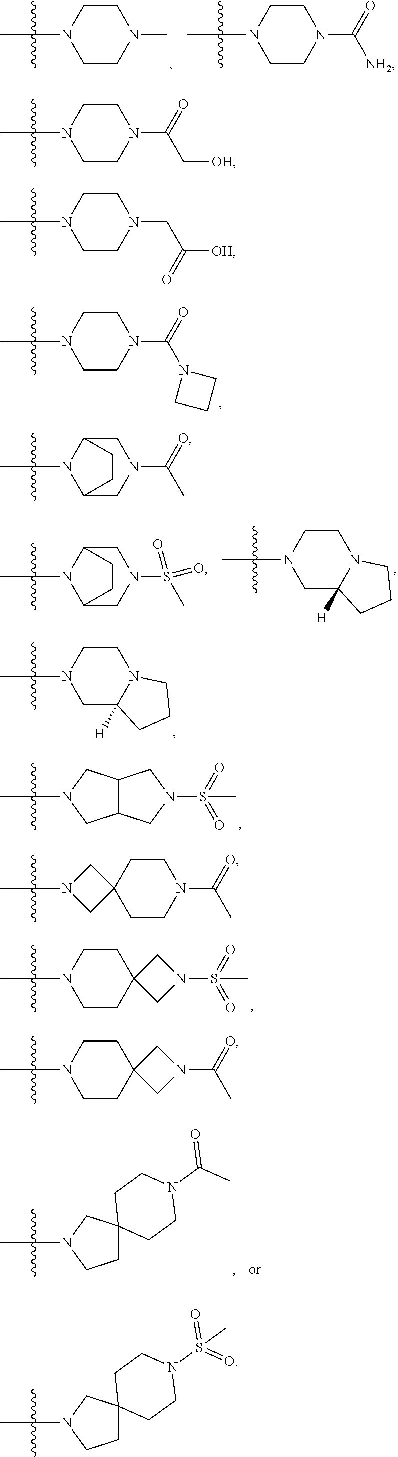

In some embodiments of the methods for treating dyskinesia with a compound of Formula (XVI), R⁵ is H. In some embodiments of the methods for treating dyskinesia with a compound of Formula (XVI), R⁵ is halogen. In some embodiments of the methods for treating dyskinesia with a compound of Formula (XVI), R⁵ is —Cl. In some embodiments of the methods for treating dyskinesia with a compound of Formula (XVI), R⁵ is —F. In some embodiments of the methods for treating dyskinesia with a compound of Formula (XVI), R⁵ is C₁₋₆alkyl. In some embodiments of the methods for treating dyskinesia with a compound of Formula (XVI), R⁵ is C₁₋₆haloalkyl. In some embodiments of the methods for treating dyskinesia with a compound of Formula (XVI), R⁵ is —CF₃. In some embodiments of the methods for treating dyskinesia with a compound of Formula (XVI), R⁵ is phenyl.

In some embodiments of the methods for treating dyskinesia with a compound of Formula (XVI), R⁶ is H. In some embodiments of the methods for treating dyskinesia with a compound of Formula (XVI), R⁶ is halogen. In some embodiments of the methods for treating dyskinesia with a compound of Formula (XVI), R⁶ is —Cl. In some embodiments of the methods for treating dyskinesia with a compound of Formula (XVI), R⁶ is —F. In some embodiments of the methods for treating dyskinesia with a compound of Formula (XVI), R⁶ is C₁₋₆alkyl.

In some embodiments of the methods for treating dyskinesia with a compound of Formula (XVI), the compound is selected from:

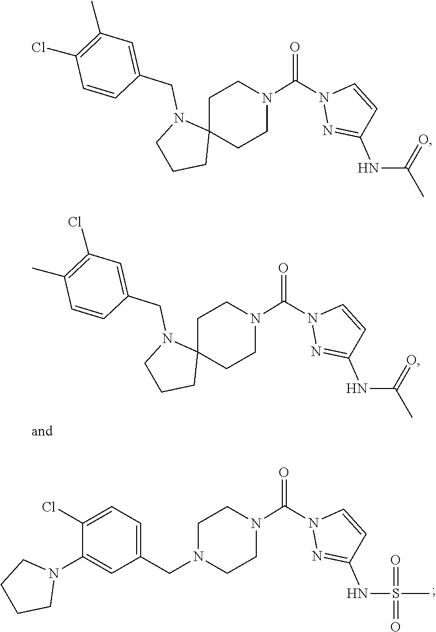

or a pharmaceutically acceptable salt or solvate thereof.

In some embodiments is a method for treating dyskinesia in a patient in need thereof, comprising administering to the patient a therapeutically effective amount of a compound of Formula XVII):

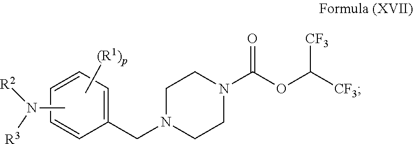

wherein:
each R¹ is independently selected from halogen, C₁₋₆alkyl, C₁₋₆haloalkyl, C₁₋₆alkoxy, C₁₋₆haloalkoxy, C₃₋₈cycloalkyl, —OH, and —CN;
R² and R³, together with the carbon to which they are attached, form
   (iii) a C₂-C₇heterocycloalkyl; or
   (iv) a C₂-C₉heteroaryl;
wherein the C₂-C₇heterocycloalkyl or the C₂-C₉heteroaryl is substituted with one R⁴ and optionally substituted with one or two additional substituents selected from halogen, C₁₋₆alkyl, C₁₋₆haloalkyl, and C₁₋₆alkoxy;
R⁴ is —CO₂H or —C₁₋₆alkyl-CO₂H; and
p is 0, 1, 2, 3, or 4;
   or a pharmaceutically acceptable salt or solvate thereof.

In some embodiments is a method for treating dyskinesia in a patient in need thereof, comprising administering to the patient a therapeutically effective amount of a compound of Formula (XVII), wherein the dyskinesia is levodopa-induced dyskinesia.

In some embodiments of the methods for treating dyskinesia with a compound of Formula (XVII), R² and R³, together with the carbon to which they are attached, form a C₂-C₇heterocycloalkyl substituted with one R⁴ and optionally substituted with one or two additional substituents selected from halogen, C₁₋₆alkyl, C₁₋₆haloalkyl, and C₁₋₆ alkoxy. In some embodiments of the methods for treating dyskinesia with a compound of Formula (XVII), R² and R³, together with the carbon to which they are attached, form a C₂-C₇heterocycloalkyl substituted with —CO₂H and optionally substituted with one or two additional substituents selected from halogen, C₁₋₆alkyl, C₁₋₆haloalkyl, and C₁₋₆ alkoxy. In some embodiments of the methods for treating dyskinesia with a compound of Formula (XVII), R² and R³, together with the carbon to which they are attached, form a C₂-C₇heterocycloalkyl substituted with —CO₂H and optionally substituted with no additional substituents. In some embodiments of the methods for treating dyskinesia with a compound of Formula (XVII), R² and R³, together with the carbon to which they are attached, form a C₂-C₇heterocycloalkyl substituted with —C₁₋₆alkyl-CO₂H and optionally substituted with one or two additional substituents selected from halogen, C₁₋₆alkyl, C₁₋₆ haloalkyl, and C₁₋₆alkoxy. In some embodiments of the methods for treating dyskinesia with a compound of Formula (XVII), R² and R³, together with the carbon to which they are attached, form a C₂-C₇heterocycloalkyl substituted with —C₁₋₆alkyl-CO₂H and optionally substituted with no additional substituents. In some embodiments of the methods for treating dyskinesia with a compound of Formula (XVII), R² and R³, together with the carbon to which they are attached, form a piperidine substituted with —CO₂H and optionally substituted with one or two additional substituents selected from halogen, C₁₋₆alkyl, C₁₋₆haloalkyl, and C₁₋₆alkoxy. In some embodiments of the methods for treating dyskinesia with a compound of Formula (XVII), R² and R³, together with the carbon to which they are attached, form a piperidine substituted with —CO₂H and optionally substituted with no additional substituents. In some embodiments of the methods for treating dyskinesia with a compound of Formula (XVII), R² and R³, together with the carbon to which they are attached, form a piperidine substituted with —C₁₋₆alkyl-CO₂H and optionally substituted with one or two additional substituents selected from halogen, C₁₋₆alkyl, C₁₋₆haloalkyl, and C₁₋₆alkoxy. In some embodiments of the methods for treating dyskinesia with a compound of Formula (XVII), R² and R³, together with the carbon to which they are attached, form a piperidine substituted with —C₁₋₆alkyl-CO₂H and optionally substituted with no additional substituents.

In some embodiments of the methods for treating dyskinesia with a compound of Formula (XVII), R² and R³, together with the carbon to which they are attached, form a C₂-C₉heteroaryl substituted with one R⁴ and optionally substituted with one or two additional substituents selected from halogen, C₁₋₆alkyl, C₁₋₆haloalkyl, and C₁₋₆alkoxy. In some embodiments of the methods for treating dyskinesia with a compound of Formula (XVII), R² and R³, together with the carbon to which they are attached, form a C₂-C₉heteroaryl substituted with —CO₂H and optionally substituted with one or two additional substituents selected from halogen, C₁₋₆alkyl, C₁₋₆haloalkyl, and C₁₋₆alkoxy. In some embodiments of the methods for treating dyskinesia with a compound of Formula (XVII), R² and R³, together with the carbon to which they are attached, form a C₂-C₉heteroaryl substituted with —CO₂H and optionally substituted with no additional substituents. In some embodiments of the methods for treating dyskinesia with a compound of Formula (XVII), R² and R³, together with the carbon to which they are attached, form a C₂-C₉heteroaryl substituted with —C₁₋₆alkyl-CO₂H and optionally substituted with one or two additional substituents selected from halogen, C₁₋₆alkyl, C₁₋₆ haloalkyl, and C₁₋₆alkoxy. In some embodiments of the methods for treating dyskinesia with a compound of Formula (XVII), R² and R³, together with the carbon to which they are attached, form a C₂-C₉heteroaryl substituted with —C₁₋₆alkyl-CO₂H and optionally substituted with no additional substituents.

In some embodiments of the methods for treating dyskinesia with a compound of Formula (XVII), each R¹ is independently selected from halogen, C₁₋₆alkyl, C₁₋₆haloalkyl, C₁₋₆alkoxy, and C₁₋₆haloalkoxy. In some embodiments of the methods for treating dyskinesia with a compound of Formula (XVII), each R¹ is independently selected from halogen, C₁₋₆alkyl, and C₁₋₆haloalkyl.

In some embodiments of the methods for treating dyskinesia with a compound of Formula (XVII), p is 0, 1, or 2. In some embodiments of the methods for treating dyskinesia with a compound of Formula (XVII), p is 2. In some embodiments of the methods for treating dyskinesia with a compound of Formula (XVII), p is 1. In some embodiments of the methods for treating dyskinesia with a compound of Formula (XVII), p is 0.

In some embodiments of the methods for treating dyskinesia with a compound of Formula (XVII), the compound is selected from:

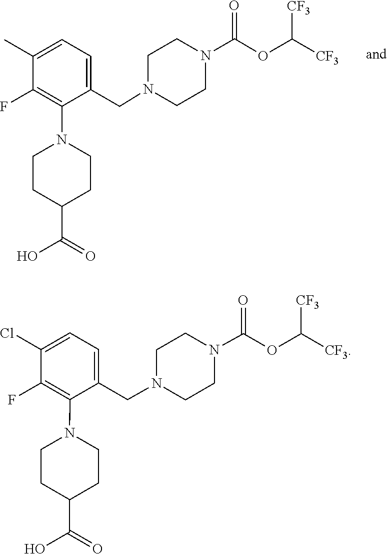 [SMILES: Cc1ccc(CN2CCN(C(=O)OC(C(F)(F)F)C(F)(F)F)CC2)c(N2CCC(C(=O)O)CC2)c1F ; O=C(O)C1CCN(c2c(CN3CCN(C(=O)OC(C(F)(F)F)C(F)(F)F)CC3)ccc(Cl)c2F)CC1]

Further embodiments provided herein include combinations of one or more of the particular embodiments set forth above.

In some embodiments is a method for treating dyskinesia in a patient in need thereof, comprising administering to the patient a therapeutically effective amount of a compound having the structure provided in Table 1. In some embodiments is a method for treating dyskinesia in a patient in need thereof, comprising administering to the patient a therapeutically effective amount of a compound having the structure provided in Table 1; wherein the dyskinesia is levodopa-induced dyskinesia.

TABLE 1

| Compound | | |
|---|---|---|
| Number | Structure | Name |
| 1 | 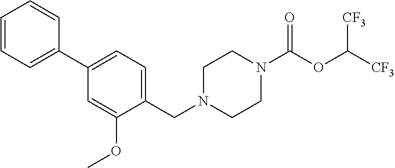 [SMILES: COc1cc(-c2ccccc2)ccc1CN1CCN(C(=O)OC(C(F)(F)F)C(F)(F)F)CC1] | 1,1,1,3,3,3-Hexafluoropropan-2-yl 4-((3- methoxy-[1,1′-biphenyl]-4- yl)methyl)piperazine-1-carboxylate |
| 2 | 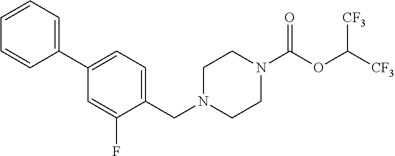 [SMILES: O=C(OC(C(F)(F)F)C(F)(F)F)N1CCN(Cc2ccc(-c3ccccc3)cc2F)CC1] | 1,1,1,3,3,3-Hexafluoropropan-2-yl 4-((3- fluoro-[1,1′-biphenyl]-4- yl)methyl)piperazine-1-carboxylate |
| 3 | 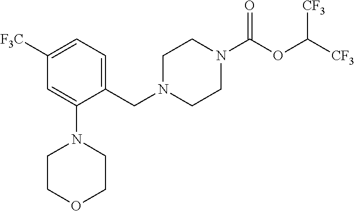 [SMILES: O=C(OC(C(F)(F)F)C(F)(F)F)N1CCN(Cc2ccc(C(F)(F)F)cc2N2CCOCC2)CC1] | 1,1,1,3,3,3-Hexafluoropropan-2-yl 4-(2- morpholino-4- (trifluoromethyl)benzyl)piperazine-1- carboxylate |
| 4 | 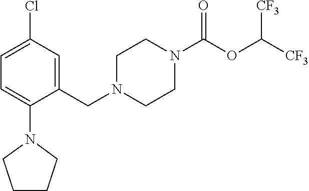 [SMILES: O=C(OC(C(F)(F)F)C(F)(F)F)N1CCN(Cc2cc(Cl)ccc2N2CCCC2)CC1] | 1,1,1,3,3,3-Hexafluoropropan-2-yl 4-(5- chloro-2-(pyrrolidin-1- yl)benzyl)piperazine-1-carboxylate |
| 5 | 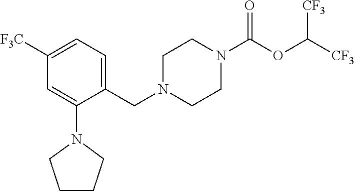 [SMILES: O=C(OC(C(F)(F)F)C(F)(F)F)N1CCN(Cc2ccc(C(F)(F)F)cc2N2CCCC2)CC1] | 1,1,1,3,3,3-Hexafluoropropan-2-yl 4-(2- (pyrrolidin-1-yl)-4- (trifluoromethyl)benzyl)piperazine-1- carboxylate |
| 6 | 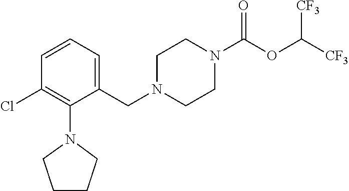 [SMILES: O=C(OC(C(F)(F)F)C(F)(F)F)N1CCN(Cc2cccc(Cl)c2N2CCCC2)CC1] | 1,1,1,3,3,3-Hexafluoropropan-2-yl 4-(3- chloro-2-(pyrrolidin-1- yl)benzyl)piperazine-1-carboxylate |
| 7 | 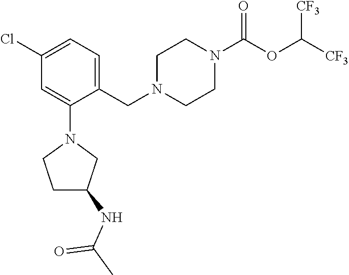 [SMILES: CC(=O)N[C@H]1CCN(c2cc(Cl)ccc2CN2CCN(C(=O)OC(C(F)(F)F)C(F)(F)F)CC2)C1] | 1,1,1,3,3,3-Hexafluoropropan-2-yl (S)-4- (2-(3-acetamidopyrrolidin-1-yl)-4- chlorobenzyl)piperazine-1-carboxylate |
| 8 | 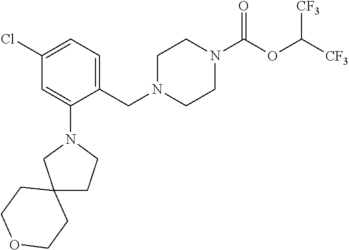 [SMILES: O=C(OC(C(F)(F)F)C(F)(F)F)N1CCN(Cc2ccc(Cl)cc2N2CCC3(CCOCC3)C2)CC1] | 1,1,1,3,3,3-Hexafluoropropan-2-yl 4-(4- chloro-2-(8-oxa-2-azaspiro[4.5]decan-2- yl)benzyl)piperazine-1-carboxylate |
| 9 | 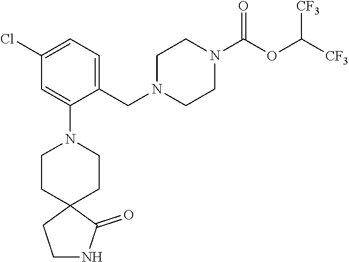 [SMILES: O=C(OC(C(F)(F)F)C(F)(F)F)N1CCN(Cc2ccc(Cl)cc2N2CCC3(CCNC3=O)CC2)CC1] | 1,1,1,3,3,3-Hexafluoropropan-2-yl 4-(4- chloro-2-(1-oxo-2,8-diazaspiro[4.5]decan- 8-yl)benzyl)piperazine-1-carboxylate |
| 10 | 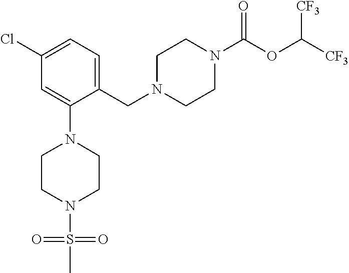 [SMILES: CS(=O)(=O)N1CCN(c2cc(Cl)ccc2CN2CCN(C(=O)OC(C(F)(F)F)C(F)(F)F)CC2)CC1] | 1,1,1,3,3,3-Hexafluoropropan-2-yl 4-(4- chloro-2-(4-(methylsulfonyl)piperazin-1- yl)benzyl)piperazine-1-carboxylate |
| 11 | 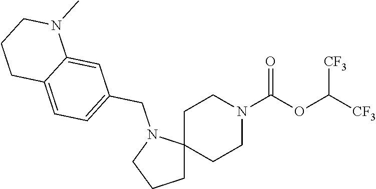 [SMILES: CN1CCCc2ccc(CN3CCCC34CCN(C(=O)OC(C(F)(F)F)C(F)(F)F)CC4)cc21] | 1,1,1,3,3,3-Hexafluoropropan-2-yl 1-((1- methyl-1,2,3,4-tetrahydroquinolin-7- yl)methyl)-1,8-diazaspiro[4.5]decane-8- carboxylate |
| 12 | 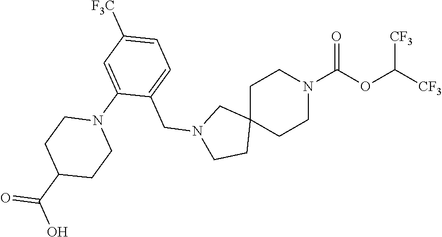 [SMILES: O=C(O)C1CCN(c2cc(C(F)(F)F)ccc2CN2CCC3(CCN(C(=O)OC(C(F)(F)F)C(F)(F)F)CC3)C2)CC1] | 1-(2-((8-(((1,1,1,3,3,3-Hexafluoropropan- 2-yl)oxy)carbonyl)-2,8- diazaspiro[4.5]decan-2-yl)methyl)-5- (trifluoromethyl)phenyl)piperidine-4- carboxylic acid |
| 13 | 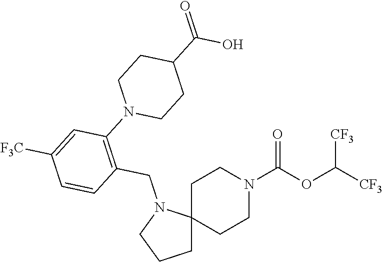 [SMILES: O=C(O)C1CCN(c2cc(C(F)(F)F)ccc2CN2CCCC23CCN(C(=O)OC(C(F)(F)F)C(F)(F)F)CC3)CC1] | 1-(2-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)piperidine-4- carboxylic acid |
| 14 | 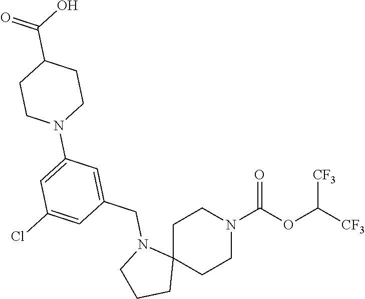 [SMILES: O=C(O)C1CCN(c2cc(Cl)cc(CN3CCCC34CCN(C(=O)OC(C(F)(F)F)C(F)(F)F)CC4)c2)CC1] | 1-(3-Chloro-5-((8-(((1,1,1,3,3,3- hexafluoropropan-2-yl)oxy)carbonyl)-1,8- diazaspiro[4.5]decan-1- yl)methyl)phenyl)piperidine-4-carboxylic acid |
| 15 | 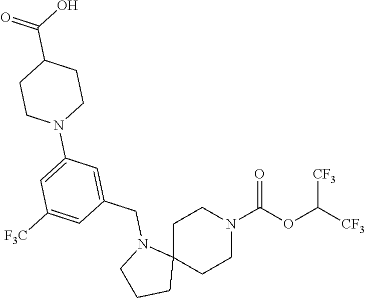 [SMILES: O=C(O)C1CCN(c2cc(CN3CCCC34CCN(C(=O)OC(C(F)(F)F)C(F)(F)F)CC4)cc(C(F)(F)F)c2)CC1] | 1-(3-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)piperidine-4- carboxylic acid |
| 16 | 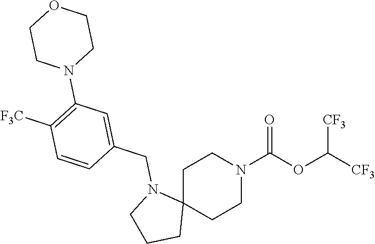 [SMILES: O=C(OC(C(F)(F)F)C(F)(F)F)N1CCC2(CCCN2Cc2ccc(C(F)(F)F)c(N3CCOCC3)c2)CC1] | 1,1,1,3,3,3-Hexafluoropropan-2-yl 1-(3- morpholino-4-(trifluoromethyl)benzyl)- 1,8-diazaspiro[4.5]decane-8-carboxylate |
| 17 | 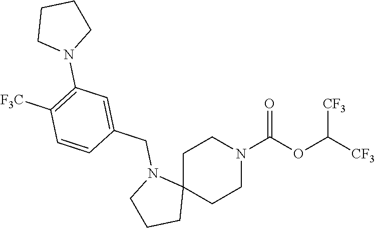 [SMILES: O=C(OC(C(F)(F)F)C(F)(F)F)N1CCC2(CCCN2Cc2ccc(C(F)(F)F)c(N3CCCC3)c2)CC1] | 1,1,1,3,3,3-Hexafluoropropan-2-yl 1-(3- (pyrrolidin-1-yl)-4- (trifluoromethyl)benzyl)-1,8- diazaspiro[4.5]decane-8-carboxylate |
| 18 | 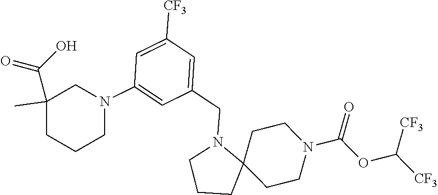 [SMILES: CC1(C(=O)O)CCCN(c2cc(CN3CCCC34CCN(C(=O)OC(C(F)(F)F)C(F)(F)F)CC4)cc(C(F)(F)F)c2)C1] | 1-(3-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)-3- methylpiperidine-3-carboxylic acid |
| 19 | 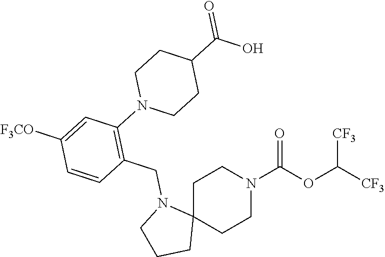 [SMILES: O=C(O)C1CCN(c2cc(OC(F)(F)F)ccc2CN2CCCC23CCN(C(=O)OC(C(F)(F)F)C(F)(F)F)CC3)CC1] | 1-(2-((8-(((1,1,1,3,3,3-Hexafluoropropan- 2-yl)oxy)carbonyl)-1,8- diazaspiro[4.5]decan-1-yl)methyl)-5- (trifluoromethoxy)phenyl)piperidine-4- carboxylic acid |
| 20 | 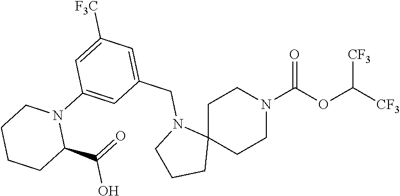 [SMILES: O=C(O)[C@H]1CCCCN1c1cc(CN2CCCC23CCN(C(=O)OC(C(F)(F)F)C(F)(F)F)CC3)cc(C(F)(F)F)c1] | (R)-1-(3-((8-(((1,1,1,3,3,3- Hexafluoropropan-2-yl)oxy)carbonyl)-1,8- diazaspiro[4.5]decan-1-yl)methyl)-5- (trifluoromethyl)phenyl)piperidine-2- carboxylic acid |
| 21 | 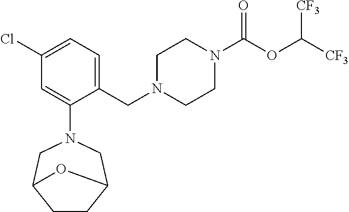 [SMILES: O=C(OC(C(F)(F)F)C(F)(F)F)N1CCN(Cc2ccc(Cl)cc2N2CC3CCC(C2)O3)CC1] | 1,1,1,3,3,3-Hexafluoropropan-2-yl 4-(2-(8- oxa-3-azabicyclo[3.2.1]octan-3-yl)-4- chlorobenzyl)piperazine-1-carboxylate |
| 22 | 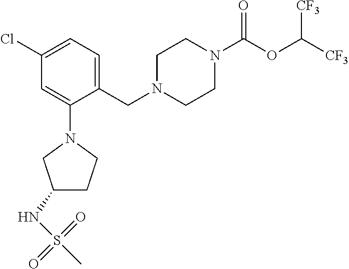 [SMILES: CS(=O)(=O)N[C@H]1CCN(c2cc(Cl)ccc2CN2CCN(C(=O)OC(C(F)(F)F)C(F)(F)F)CC2)C1] | 1,1,1,3,3,3-Hexafluoropropan-2-yl (S)-4- (4-chloro-2-(3- (methylsulfonamido)pyrrolidin-1- yl)benzyl)piperazine-1-carboxylate |
| 23 | 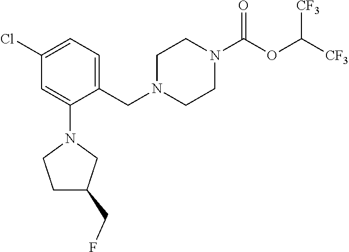 [SMILES: O=C(OC(C(F)(F)F)C(F)(F)F)N1CCN(Cc2ccc(Cl)cc2N2CC[C@H](CF)C2)CC1] | 1,1,1,3,3,3-Hexafluoropropan-2-yl (S)-4- (4-chloro-2-(3-(fluoromethyl)pyrrolidin-1- yl)benzyl)piperazine-1-carboxylate |
| 24 | 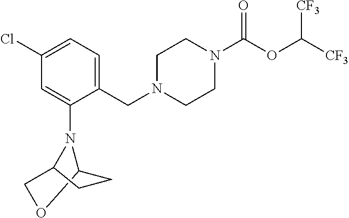 [SMILES: O=C(OC(C(F)(F)F)C(F)(F)F)N1CCN(Cc2ccc(Cl)cc2N2C3CCC2OC3)CC1] | 1,1,1,3,3,3-Hexafluoropropan-2-yl 4-(2-(3- oxa-8-azabicyclo[3.2.1]octan-8-yl)-4- chlorobenzyl)piperazine-1-carboxylate |
| 25 | 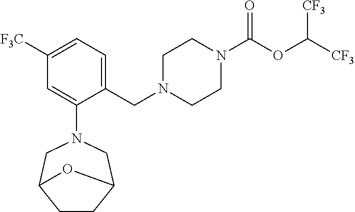 [SMILES: O=C(OC(C(F)(F)F)C(F)(F)F)N1CCN(Cc2ccc(C(F)(F)F)cc2N2CC3CCC(C2)O3)CC1] | 1,1,1,3,3,3-Hexafluoropropan-2-yl 4-(2-(8- oxa-3-azabicyclo[3.2.1]octan-3-yl)-4- (trifluoromethyl)benzyl)piperazine-1- carboxylate |
| 26 | 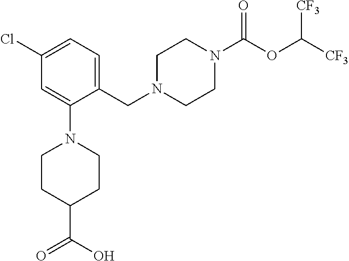 [SMILES: O=C(O)C1CCN(c2cc(Cl)ccc2CN2CCN(C(=O)OC(C(F)(F)F)C(F)(F)F)CC2)CC1] | 1-(5-Chloro-2-((4-(((1,1,1,3,3,3- hexafluoropropan-2- yl)oxy)carbonyl)piperazin-1- yl)methyl)phenyl)piperidine-4-carboxylic acid |
| 27 | 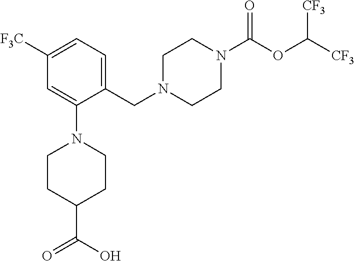 [SMILES: O=C(O)C1CCN(c2cc(C(F)(F)F)ccc2CN2CCN(C(=O)OC(C(F)(F)F)C(F)(F)F)CC2)CC1] | 1-(2-((4-(((1,1,1,3,3,3-Hexafluoropropan- 2-yl)oxy)carbonyl)piperazin-1-yl)methyl)- 5-(trifluoromethyl)phenyl)piperidine-4- carboxylic acid |
| 28 | 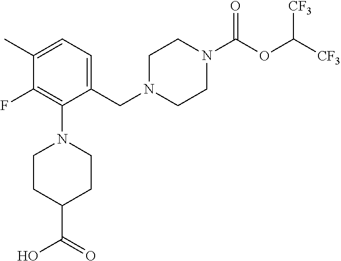 [SMILES: Cc1ccc(CN2CCN(C(=O)OC(C(F)(F)F)C(F)(F)F)CC2)c(N2CCC(C(=O)O)CC2)c1F] | 1-(2-Fluoro-6-((4-(((1,1,1,3,3,3- hexafluoropropan-2- yl)oxy)carbonyl)piperazin-1-yl)methyl)-3- methylphenyl)piperidine-4-carboxylic acid |
| 29 | 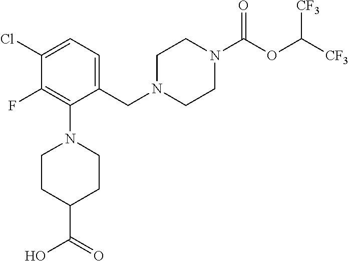 [SMILES: O=C(O)C1CCN(c2c(CN3CCN(C(=O)OC(C(F)(F)F)C(F)(F)F)CC3)ccc(Cl)c2F)CC1] | 1-(3-Chloro-2-fluoro-6-((4-(((1,1,1,3,3,3- hexafluoropropan-2- yl)oxy)carbonyl)piperazin-1- yl)methyl)phenyl)piperidine-4-carboxylic acid |
| 30 | 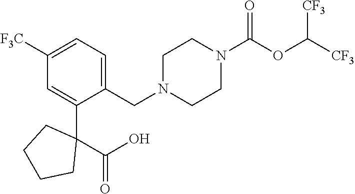 [SMILES: O=C(OC(C(F)(F)F)C(F)(F)F)N1CCN(Cc2ccc(C(F)(F)F)cc2C2(C(=O)O)CCCC2)CC1] | 1-(2-((4-(((1,1,1,3,3,3-Hexafluoropropan- 2-yl)oxy)carbonyl)piperazin-1-yl)methyl)- 5-(trifluoromethyl)phenyl)cyclopentane-1- carboxylic acid |
| 31 | 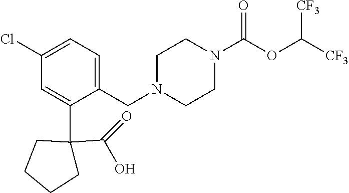 [SMILES: O=C(OC(C(F)(F)F)C(F)(F)F)N1CCN(Cc2ccc(Cl)cc2C2(C(=O)O)CCCC2)CC1] | 1-(5-Chloro-2-((4-(((1,1,1,3,3,3- hexafluoropropan-2- yl)oxy)carbonyl)piperazin-1- yl)methyl)phenyl)cyclopentane-1- carboxylic acid |
| 32 | 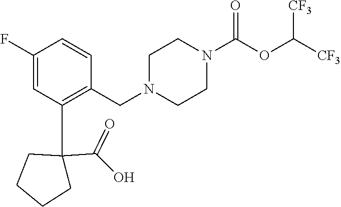 [SMILES: O=C(OC(C(F)(F)F)C(F)(F)F)N1CCN(Cc2ccc(F)cc2C2(C(=O)O)CCCC2)CC1] | 1-(5-Fluoro-2-((4-(((1,1,1,3,3,3- hexafluoropropan-2- yl)oxy)carbonyl)piperazin-1- yl)methyl)phenyl)cyclopentane-1- carboxylic acid |
| 33 | 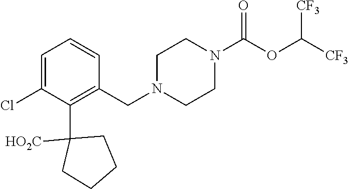 [SMILES: O=C(OC(C(F)(F)F)C(F)(F)F)N1CCN(Cc2cccc(Cl)c2C2(C(=O)O)CCCC2)CC1] | 1-(2-Chloro-6-((4-(((1,1,1,3,3,3- hexafluoropropan-2- yl)oxy)carbonyl)piperazin-1- yl)methyl)phenyl)cyclopentane-1- carboxylic acid |
| 34 | 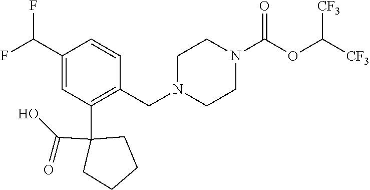 [SMILES: O=C(OC(C(F)(F)F)C(F)(F)F)N1CCN(Cc2ccc(C(F)F)cc2C2(C(=O)O)CCCC2)CC1] | 1-(5-Difluoromethyl)-2-((4-(((1,1,1,3,3,3- hexafluoropropan-2- yl)oxy)carbonyl)piperazin-1- yl)methyl)phenyl)cyclopentane-1- carboxylic acid |
| 35 | 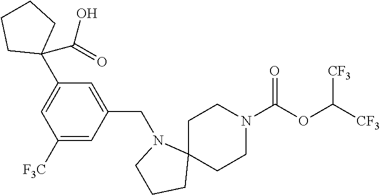 [SMILES: O=C(OC(C(F)(F)F)C(F)(F)F)N1CCC2(CCCN2Cc2cc(C(F)(F)F)cc(C3(C(=O)O)CCCC3)c2)CC1] | 1-(3-((8-(((1,1,1,3,3,3-Hexafluoropropan- 2-yl)oxy)carbonyl)-1,8- diazaspiro[4,5]decan-1-yl)methyl)-5- (trifluoromethyl)phenyl)cyclopentane-1- carboxylic acid |
| 36 | 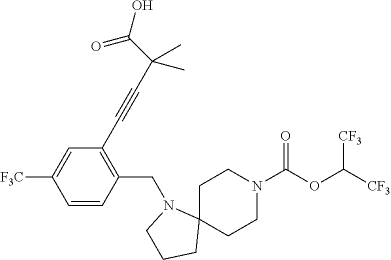 [SMILES: CC(C)(C#Cc1cc(C(F)(F)F)ccc1CN1CCCC12CCN(C(=O)OC(C(F)(F)F)C(F)(F)F)CC2)C(=O)O] | 4-(2-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)-2,2-dimethylbut- 3-ynoic acid |
| 37 | 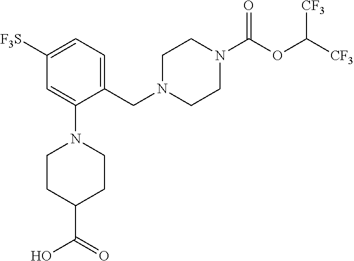 | 1-(2-((4-(((1,1,1,3,3,3-Hexafluoropropan- 2-yl)oxy)carbonyl)piperazin-1-yl)methyl)- 5-(pentafluoro-l6- sulfaneyl)phenyl)piperidine-4- carboxylic acid |
| 38 | 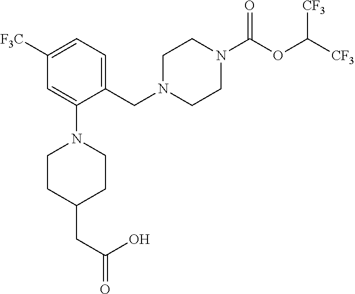 [SMILES: O=C(O)CC1CCN(c2cc(C(F)(F)F)ccc2CN2CCN(C(=O)OC(C(F)(F)F)C(F)(F)F)CC2)CC1] | 2-(1-(2-((4-(((1,1,1,3,3,3- Hexafluoropropan-2- yl)oxy)carbonyl)piperazin-1-yl)methyl)-5- (trifluoromethyl)phenyl)piperidin-4- yl)acetic acid |
| 39 | 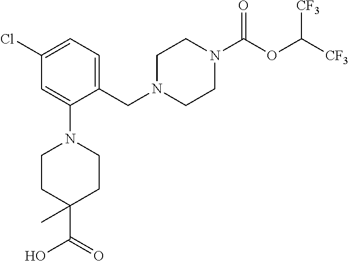 [SMILES: CC1(C(=O)O)CCN(c2cc(Cl)ccc2CN2CCN(C(=O)OC(C(F)(F)F)C(F)(F)F)CC2)CC1] | 1-(5-Chloro-2-((4-(((1,1,1,3,3,3- hexafluoropropan-2- yl)oxy)carbonyl)piperazin-1- yl)methyl)phenyl)-4-methylpiperidine-4- carboxylic acid |
| 40 | 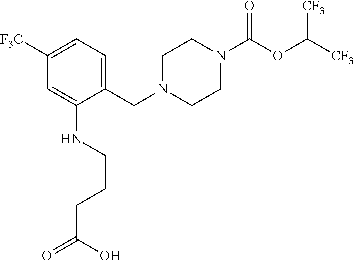 [SMILES: O=C(O)CCCNc1cc(C(F)(F)F)ccc1CN1CCN(C(=O)OC(C(F)(F)F)C(F)(F)F)CC1] | 4-((2-((4-(((1,1,1,3,3,3-Hexafluoropropan- 2-yl)oxy)carbonyl)piperazin-1-yl)methyl)- 5-(trifluoromethyl)phenyl)amino)butanoic acid |
| 41 | 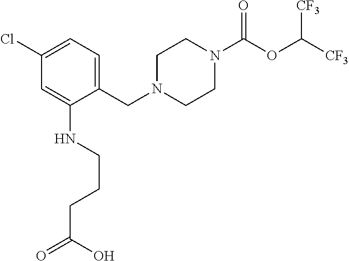 [SMILES: O=C(O)CCCNc1cc(Cl)ccc1CN1CCN(C(=O)OC(C(F)(F)F)C(F)(F)F)CC1] | 4-((5-Chloro-2-((4-(((1,1,1,3,3,3- hexafluoropropan-2- yl)oxy)carbonyl)piperazin-1- yl)methyl)phenyl)amino)butanoic acid |
| 42 | 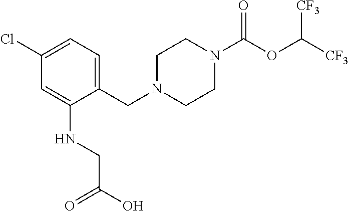 [SMILES: O=C(O)CNc1cc(Cl)ccc1CN1CCN(C(=O)OC(C(F)(F)F)C(F)(F)F)CC1] | (5-Chloro-2-((4-(((1,1,1,3,3,3- hexafluoropropan-2- yl)oxy)carbonyl)piperazin-1- yl)methyl)phenyl)glycine |
| 43 | 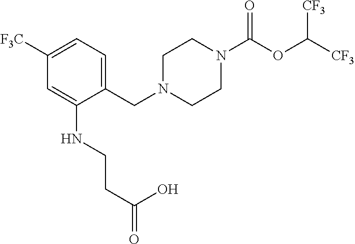 [SMILES: O=C(O)CCNc1cc(C(F)(F)F)ccc1CN1CCN(C(=O)OC(C(F)(F)F)C(F)(F)F)CC1] | 3-((2-((4-(((1,1,1,3,3,3-Hexafluoropropan- 2-yl)oxy)carbonyl)piperazin-1-yl)methyl)- 5-(trifluoromethyl)phenyl)amino) propanic acid |
| 44 | 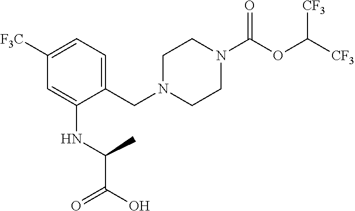 [SMILES: C[C@H](Nc1cc(C(F)(F)F)ccc1CN1CCN(C(=O)OC(C(F)(F)F)C(F)(F)F)CC1)C(=O)O] | (2-((4-(((1,1,1,3,3,3-Hexafluoropropan-2- yl)oxy)carbonyl)piperazin-1-yl)methyl)-5- (trifluoromethyl)phenyl)-L-alanine |
| 45 | 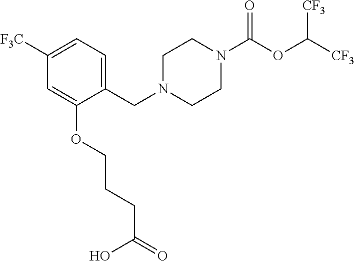 [SMILES: O=C(O)CCCOc1cc(C(F)(F)F)ccc1CN1CCN(C(=O)OC(C(F)(F)F)C(F)(F)F)CC1] | 4-(2-((4-(((1,1,1,3,3,3-Hexafluoropropan- 2-yl)oxy)carbonyl)piperazin-1-yl)methyl)- 5-(trifluoromethyl)phenoxy)butanoic acid |
| 46 | 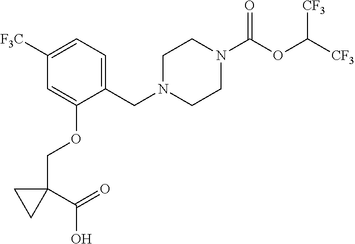 [SMILES: O=C(OC(C(F)(F)F)C(F)(F)F)N1CCN(Cc2ccc(C(F)(F)F)cc2OCC2(C(=O)O)CC2)CC1] | 1-((2-((4-(((1,1,1,3,3,3-Hexafluoropropan- 2-yl)oxy)carbonyl)piperazin-1-yl)methyl)- 5- (trifluoromethyl)phenoxy)methyl) cyclopropane-1-carboxylic acid |
| 47 | 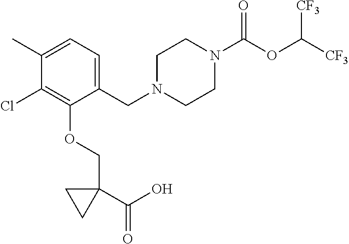 [SMILES: Cc1ccc(CN2CCN(C(=O)OC(C(F)(F)F)C(F)(F)F)CC2)c(OCC2(C(=O)O)CC2)c1Cl] | 1-((2-Chloro-6-((4-(((1,1,1,3,3,3- hexafluoropropan-2- yl)oxy)carbonyl)piperazin-1-yl)methyl)-3- methylphenoxy)methyl)cyclopropane-1- carboxylic acid |
| 48 | 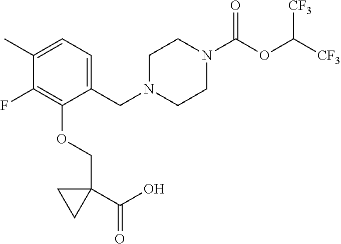 [SMILES: Cc1ccc(CN2CCN(C(=O)OC(C(F)(F)F)C(F)(F)F)CC2)c(OCC2(C(=O)O)CC2)c1F] | 1-((2-Fluoro-6-((4-(((1,1,1,3,3,3- hexafluoropropan-2- yl)oxy)carbonyl)piperazin-1-yl)methyl)-3- methylphenoxy)methyl)cyclopropane-1- carboxylic acid |
| 49 | 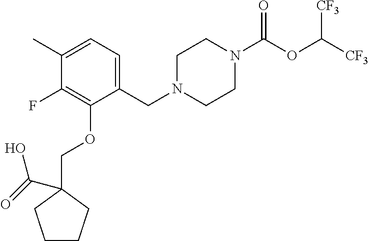 [SMILES: Cc1ccc(CN2CCN(C(=O)OC(C(F)(F)F)C(F)(F)F)CC2)c(OCC2(C(=O)O)CCCC2)c1F] | 1-((2-Fluoro-6-((4-(((1,1,1,3,3,3- hexafluoropropan-2- yl)oxy)carbonyl)piperazin-1-yl)methyl)-3- methylphenoxy)methyl)cyclopentane-1- carboxylic acid |
| 50 | 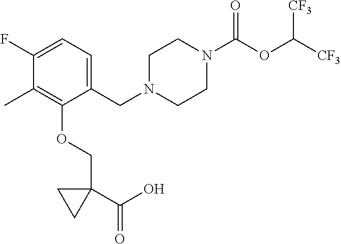 [SMILES: Cc1c(F)ccc(CN2CCN(C(=O)OC(C(F)(F)F)C(F)(F)F)CC2)c1OCC1(C(=O)O)CC1] | 1-((3-Fluoro-6-((4-(((1,1,1,3,3,3- hexafluoropropan-2- yl)oxy)carbonyl)piperazin-1-yl)methyl)-2- methylphenoxy)methyl)cyclopropane-1- carboxylic acid |
| 51 | 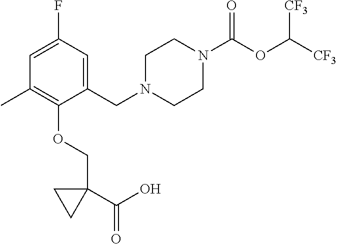 [SMILES: Cc1cc(F)cc(CN2CCN(C(=O)OC(C(F)(F)F)C(F)(F)F)CC2)c1OCC1(C(=O)O)CC1] | 1-((4-Fluoro-2-((4-(((1,1,1,3,3,3- hexafluoropropan-2- yl)oxy)carbonyl)piperazin-1-yl)methyl)-6- methylphenoxy)methyl)cyclopropane-1- carboxylic acid |
| 52 | 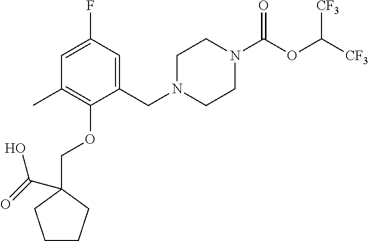 [SMILES: Cc1cc(F)cc(CN2CCN(C(=O)OC(C(F)(F)F)C(F)(F)F)CC2)c1OCC1(C(=O)O)CCCC1] | 1-((4-Fluoro-2-((4-(((1,1,1,3,3,3- hexafluoropropan-2- yl)oxy)carbonyl)piperazin-1-yl)methyl)-6- methylphenoxy)methyl)cyclopentane-1- carboxylic acid |
| 53 | 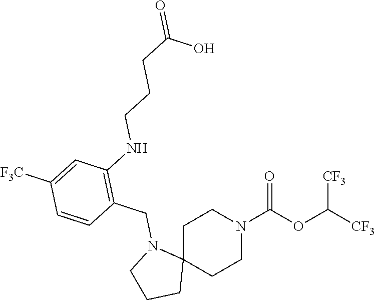 [SMILES: O=C(O)CCCNc1cc(C(F)(F)F)ccc1CN1CCCC12CCN(C(=O)OC(C(F)(F)F)C(F)(F)F)CC2] | 4-((2-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)amino) butanoic acid |
| 54 | 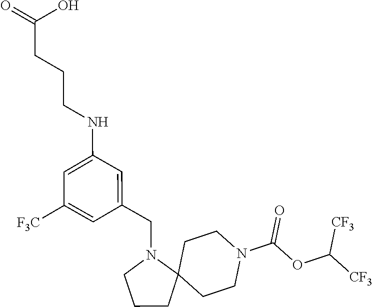 [SMILES: O=C(O)CCCNc1cc(CN2CCCC23CCN(C(=O)OC(C(F)(F)F)C(F)(F)F)CC3)cc(C(F)(F)F)c1] | 4-((3-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)amino)butanoic acid |
| 55 | 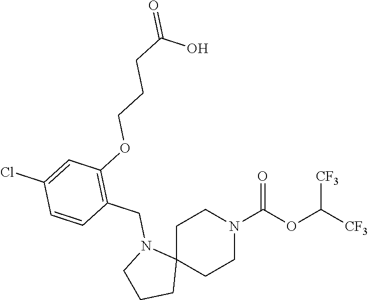 [SMILES: O=C(O)CCCOc1cc(Cl)ccc1CN1CCCC12CCN(C(=O)OC(C(F)(F)F)C(F)(F)F)CC2] | 4-(5-Chloro-2-((8-(((1,1,1,3,3,3- hexafluoropropan-2-yl)oxy)carbonyl)-1,8- diazaspiro[4.5]decan-1- yl)methyl)phenoxy)butanoic acid |
| 56 | 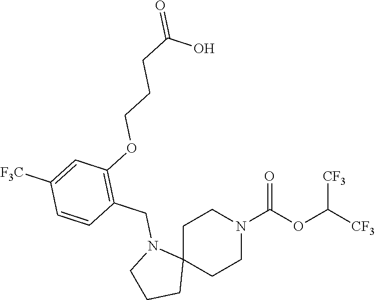 [SMILES: O=C(O)CCCOc1cc(C(F)(F)F)ccc1CN1CCCC12CCN(C(=O)OC(C(F)(F)F)C(F)(F)F)CC2] | 4-(2-((8-(((1,1,1,3,3,3-Hexafluoropropan- 2-yl)oxy)carbonyl)-1,8- diazaspiro[4.5]decan-1-yl)methyl)-5- (trifluoromethyl)phenoxy)butanoic acid |
| 57 | 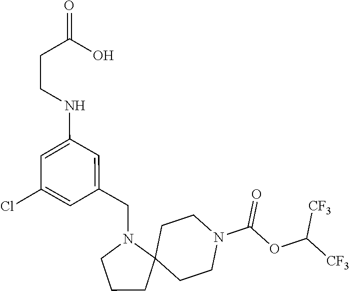 [SMILES: O=C(O)CCNc1cc(Cl)cc(CN2CCCC23CCN(C(=O)OC(C(F)(F)F)C(F)(F)F)CC3)c1] | 3-((3-Chloro-5-((8-(((1,1,1,3,3,3- hexafluoropropan-2-yl)oxy)carbonyl)-1,8- diazaspiro[4.5]decan-1- yl)methyl)phenyl)amino)propanic acid |
| 58 | 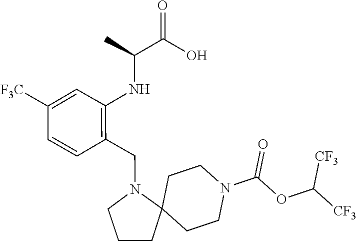 [SMILES: C[C@H](Nc1cc(C(F)(F)F)ccc1CN1CCCC12CCN(C(=O)OC(C(F)(F)F)C(F)(F)F)CC2)C(=O)O] | (2-((8-(((1,1,1,3,3,3-Hexafluoropropan-2- yl)oxy)carbonyl)-1,8-diazaspiro[4.5]decan- 1-yl)methyl)-5-(trifluoromethyl)phenyl)-L- alanine |
| 59 | 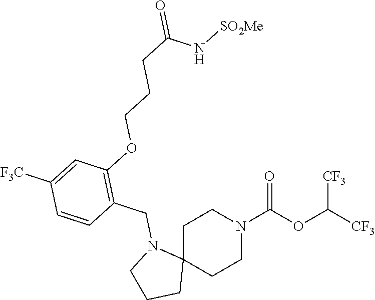 [SMILES: CS(=O)(=O)NC(=O)CCCOc1cc(C(F)(F)F)ccc1CN1CCCC12CCN(C(=O)OC(C(F)(F)F)C(F)(F)F)CC2] | 1,1,1,3,3,3-Hexafluoropropan-2-yl 1-(2-(4- (methylsulfonamido)-4-oxobutyl)-4- (trifluoromethyl)benzyl)-1,8- diazaspiro[4.5]decane-8-carboxylate |
| 60 | 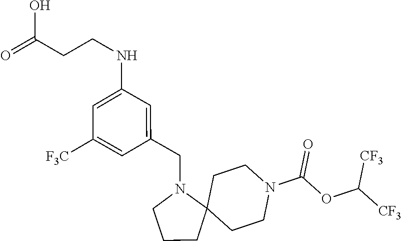 [SMILES: O=C(O)CCNc1cc(CN2CCCC23CCN(C(=O)OC(C(F)(F)F)C(F)(F)F)CC3)cc(C(F)(F)F)c1] | 3-((3-((8-(((1,1,1,3,3,3-Hexafluoropropan- 2-yl)oxy)carbonyl)-1,8- diazaspiro[4.5]decan-1-yl)methyl)-5- (trifluoromethyl)phenyl)amino)propanoic acid |
| 61 | 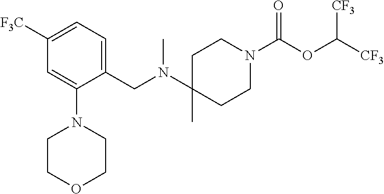 [SMILES: CN(Cc1ccc(C(F)(F)F)cc1N1CCOCC1)C1(C)CCN(C(=O)OC(C(F)(F)F)C(F)(F)F)CC1] | 1,1,1,3,3,3-Hexafluoropropan-2-yl 4- methyl-4-(methyl(2-morpholino-4- (trifluoromethyl)benzyl)amino)piperidine- 1-carboxylate |
| 62 | 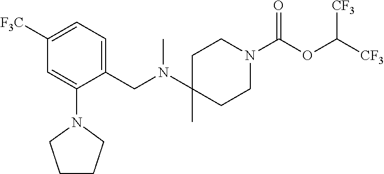 [SMILES: CN(Cc1ccc(C(F)(F)F)cc1N1CCCC1)C1(C)CCN(C(=O)OC(C(F)(F)F)C(F)(F)F)CC1] | 1,1,1,3,3,3-Hexafluoropropan-2-yl 4- methyl-4-(methyl(2-(pyrrolidin-1-yl)-4- (trifluoromethyl)benzyl)amino)piperidine- 1-carboxylate |
| 63 | 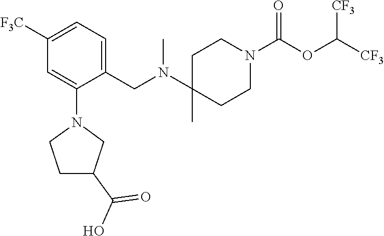 [SMILES: CN(Cc1ccc(C(F)(F)F)cc1N1CCC(C(=O)O)C1)C1(C)CCN(C(=O)OC(C(F)(F)F)C(F)(F)F)CC1] | 1-(2-(((1-(((1,1,1,3,3,3-Hexafluoropropan- 2-yl)oxy)carbonyl)-4-methylpiperidine-4- yl)(methyl)amino)methyl)-5- (trifluoromethyl)phenyl)pyrrolidine-3- carboxylic acid |
| 64 | 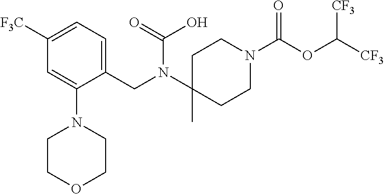 [SMILES: CC1(N(Cc2ccc(C(F)(F)F)cc2N2CCOCC2)C(=O)O)CCN(C(=O)OC(C(F)(F)F)C(F)(F)F)CC1] | (1-(((1,1,1,3,3,3-Hexafluoropropan-2- yl)oxy)carbonyl)-4-methylpiperidin-4- yl)(2-morpholino-4- (trifluoromethyl)benzyl)carbamic acid |
| 65 | 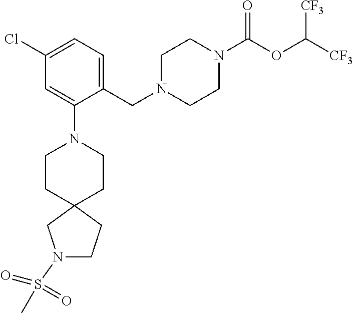 [SMILES: CS(=O)(=O)N1CCC2(CCN(c3cc(Cl)ccc3CN3CCN(C(=O)OC(C(F)(F)F)C(F)(F)F)CC3)CC2)C1] | 1,1,1,3,3,3-Hexafluoropropan-2-yl 4-(4- chloro-2-(2-(methylsulfonyl)-2,8- diazaspiro[4.5]decan-8- yl)benzyl)piperazine-1-carboxylate |
| 66 | 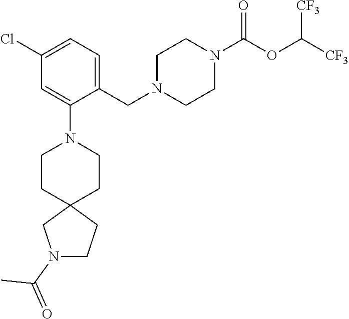 [SMILES: CC(=O)N1CCC2(CCN(c3cc(Cl)ccc3CN3CCN(C(=O)OC(C(F)(F)F)C(F)(F)F)CC3)CC2)C1] | 1,1,1,3,3,3-Hexafluoropropan-2-yl 4-(2-(2- acetyl-2,8-diazaspiro[4.5]decan-8-yl)-4- chlorobenzyl)piperazine-1-carboxylate |
| 67 | 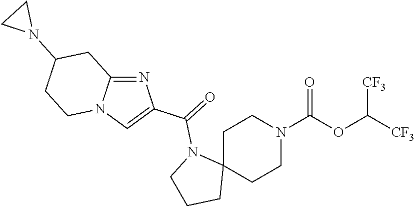 [SMILES: O=C(OC(C(F)(F)F)C(F)(F)F)N1CCC2(CCCN2C(=O)c2cn3c(n2)CC(N2CC2)CC3)CC1] | 1,1,1,3,3,3-Hexafluoropropan-2-yl 1-(7- cyclopropyl-5,6,7,8-tetrahydroimidazo [1,2-a]pyrazine-2-carbonyl)-1,8- diazaspiro[4.5]decane-8-carboxylate |
| 68 | 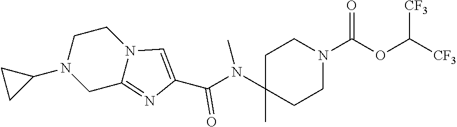 [SMILES: CN(C(=O)c1cn2c(n1)CN(C1CC1)CC2)C1(C)CCN(C(=O)OC(C(F)(F)F)C(F)(F)F)CC1] | 1,1,1,3,3,3-Hexafluoropropan-2-yl 4-(7- cyclopropyl-N-methyl-5,6,7,8- tetrahydroimidazo[1,2-a]pyrazine-2- carboxamido)-4-methylpiperidine-1- carboxylate |
| 69 | 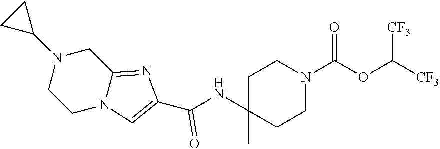 [SMILES: CC1(NC(=O)c2cn3c(n2)CN(C2CC2)CC3)CCN(C(=O)OC(C(F)(F)F)C(F)(F)F)CC1] | 1,1,1,3,3,3-Hexafluoropropan-2-yl 4-(7- cyclopropyl-5,6,7,8-tetrahydroimidazo[1,2- a]pyrazine-2-carboxamido)-4- methylpiperidine-1-carboxylate |
| 70 | 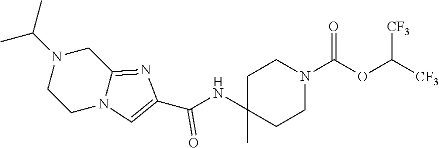 [SMILES: CC(C)N1CCn2cc(C(=O)NC3(C)CCN(C(=O)OC(C(F)(F)F)C(F)(F)F)CC3)nc2C1] | 1,1,1,3,3,3-Hexafluoropropan-2-yl 4-(7- isopropyl-5,6,7,8-tetrahydroimidazo[1,2- a]pyrazine-2-carboxamido)-4- methylpiperidine-1-carboxylate |
| 71 | 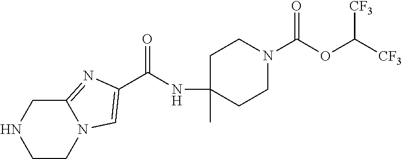 [SMILES: CC1(NC(=O)c2cn3c(n2)CNCC3)CCN(C(=O)OC(C(F)(F)F)C(F)(F)F)CC1] | 1,1,1,3,3,3-Hexafluoropropan-2-yl 4- methyl-4-(5,6,7,8-tetrahydroimidazo[1,2- a]pyrazine-2-carboxamido)piperidine-1- carboxylate |
| 72 | 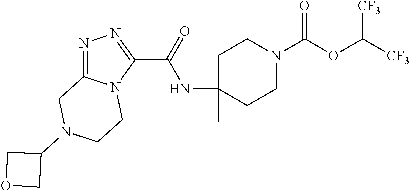 [SMILES: CC1(NC(=O)c2nnc3n2CCN(C2COC2)C3)CCN(C(=O)OC(C(F)(F)F)C(F)(F)F)CC1] | 1,1,1,3,3,3-Hexafluoropropan-2-yl 4- methyl-4-(7-(oxetan-3-yl)-5,6,7,8- tetrahydro-[1,2,4]triazolo[4,3-a]pyrazine- 3-carboxamido)piperidine-1-carboxylate |
| 73 | 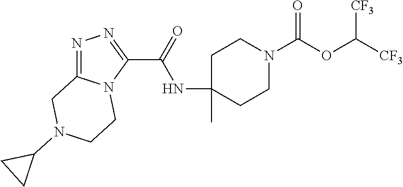 [SMILES: CC1(NC(=O)c2nnc3n2CCN(C2CC2)C3)CCN(C(=O)OC(C(F)(F)F)C(F)(F)F)CC1] | 1,1,1,3,3,3-Hexafluoropropan-2-yl 4-(7- cyclopropyl-5,6,7,8-tetrahydro- [1,2,4]triazolo[4,3-a]pyrazine-3- carboxamido)-4-methylpiperidine-1- carboxylate |
| 74 | 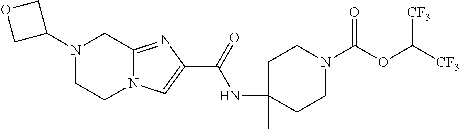 [SMILES: CC1(NC(=O)c2cn3c(n2)CN(C2COC2)CC3)CCN(C(=O)OC(C(F)(F)F)C(F)(F)F)CC1] | 1,1,1,3,3,3-Hexafluoropropan-2-yl 4- methyl-4-(7-(oxetan-3-yl)-5,6,7,8- tetrahydroimidazo[1,2-a]pyrazine-2- carboxamido)piperidine-1-carboxylate |
| 75 | 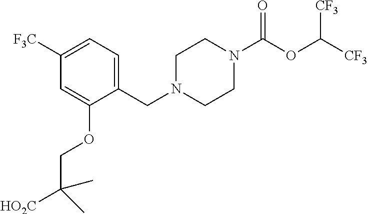 [SMILES: CC(C)(COc1cc(C(F)(F)F)ccc1CN1CCN(C(=O)OC(C(F)(F)F)C(F)(F)F)CC1)C(=O)O] | 3-(2-(4-(((1,1,1,3,3,3-Hexafluoropropan- 2-yl)oxy)carbonyl)piperazin-1-yl)methyl)- 5-(trifluoromethyl)phenoxy)-2,2- dimethylpropanoic acid |
| 76 | 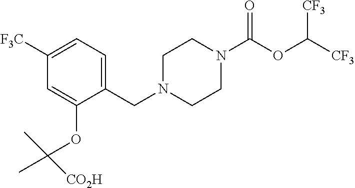 [SMILES: CC(C)(Oc1cc(C(F)(F)F)ccc1CN1CCN(C(=O)OC(C(F)(F)F)C(F)(F)F)CC1)C(=O)O] | 2-(2-(4-(((1,1,1,3,3,3-Hexafluoropropan- 2-yl)oxy)carbonyl)piperazin-1-yl)methyl)- 5-(trifluoromethyl)phenoxy)-2- methylpropanoic acid |
| 77 | 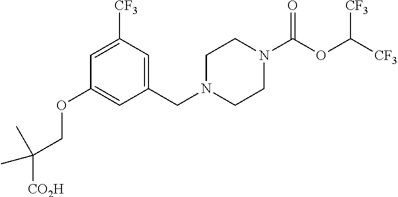 [SMILES: CC(C)(COc1cc(CN2CCN(C(=O)OC(C(F)(F)F)C(F)(F)F)CC2)cc(C(F)(F)F)c1)C(=O)O] | 3-(3-((4-(((1,1,1,3,3,3-Hexafluoropropan- 2-yl)oxy)carbonyl)piperazin-1-yl)methyl)- 5-(trifluoromethyl)phenoxy)-2,2- dimethylpropanoic acid |
| 78 | 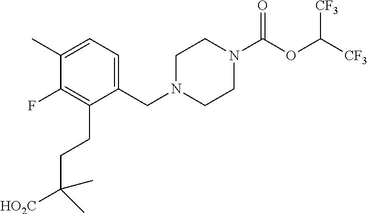 [SMILES: Cc1ccc(CN2CCN(C(=O)OC(C(F)(F)F)C(F)(F)F)CC2)c(CCC(C)(C)C(=O)O)c1F] | 3-(2-Fluoro-6-((4-(((1,1,1,3,3,3- hexafluoropropan-2- yl)oxy)carbonyl)piperazin-1-yl)methyl)-3- methylphenoxy)-2,2-dimethylpropanoic acid |
| 79 | 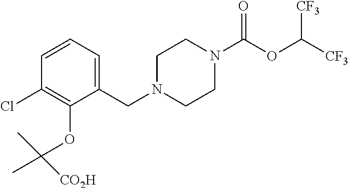 [SMILES: CC(C)(Oc1c(Cl)cccc1CN1CCN(C(=O)OC(C(F)(F)F)C(F)(F)F)CC1)C(=O)O] | 2-(2-Chloro-6-((4-(((1,1,1,3,3,3- hexafluoropropan-2- yl)oxy)carbonyl)piperazin-1- yl)methyl)phenoxy)-2-methylpropanoic acid |
| 80 | 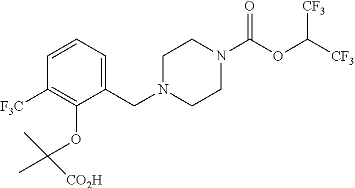 [SMILES: CC(C)(Oc1c(CN2CCN(C(=O)OC(C(F)(F)F)C(F)(F)F)CC2)cccc1C(F)(F)F)C(=O)O] | 2-(2-((4-(((1,1,1,3,3,3-Hexafluoropropan- 2-yl)oxy)carbonyl)piperazin-1-yl)methyl)- 6-(trifluoromethyl)phenoxy)-2- methylpropanoic acid |
| 81 | 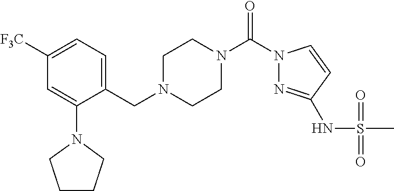 [SMILES: CS(=O)(=O)Nc1ccn(C(=O)N2CCN(Cc3ccc(C(F)(F)F)cc3N3CCCC3)CC2)n1] | N-(1-(4-(2-(Pyrrolidin-1-yl)-4- (trifluoromethyl)benzyl)piperazine-1- carbonyl)-1H-pyrazol-3- yl)methanesulfonamide |
| 82 | 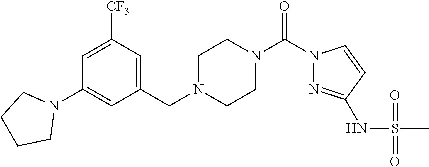 [SMILES: CS(=O)(=O)Nc1ccn(C(=O)N2CCN(Cc3cc(N4CCCC4)cc(C(F)(F)F)c3)CC2)n1] | N-(1-(4-(3-(Pyrrolidin-1-yl)-5- (trifluoromethyl)benzyl)piperazine-1- carbonyl)-1H-pyrazol-3- yl)methanesulfonamide |
| 83 | 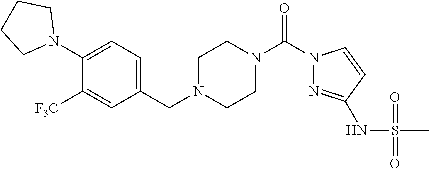 [SMILES: CS(=O)(=O)Nc1ccn(C(=O)N2CCN(Cc3ccc(N4CCCC4)c(C(F)(F)F)c3)CC2)n1] | N-(1-(4-(4-(Pyrrolidin-1-yl)-3- (trifluoromethyl)benzyl)piperazine-1- carbonyl)-1H-pyrazol-3- yl)methanesulfonamide |
| 84 | 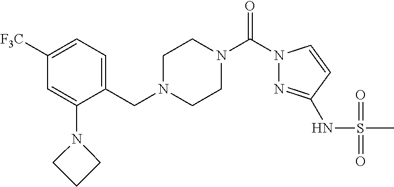 [SMILES: CS(=O)(=O)Nc1ccn(C(=O)N2CCN(Cc3ccc(C(F)(F)F)cc3N3CCC3)CC2)n1] | N-(1-(4-(2-(Azetidin-1-yl)-4- (trifluoromethyl)benzyl)piperazine-1- carbonyl)-1H-pyrazol-3- yl)methanesulfonamide |
| 85 | 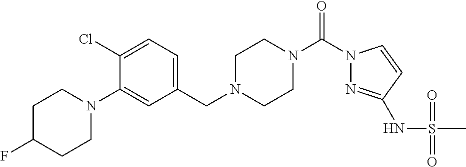 [SMILES: CS(=O)(=O)Nc1ccn(C(=O)N2CCN(Cc3ccc(Cl)c(N4CCC(F)CC4)c3)CC2)n1] | N-(1-(4-(4-Chloro-3-(4-fluoropiperidin-1- yl)benzyl)piperazine-1-carbonyl)-1H- pyrazol-3-yl)methanesulfonamide |
| 86 | 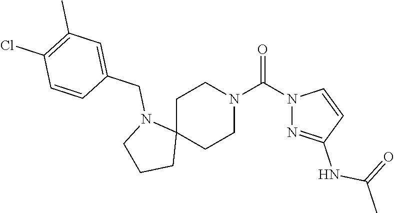 [SMILES: CC(=O)Nc1ccn(C(=O)N2CCC3(CCCN3Cc3ccc(Cl)c(C)c3)CC2)n1] | N-(1-(1-(4-Chloro-3-methylbenzyl)-1,8- diazaspiro[4.5]decane-8-carbonyl)-1H- pyrazol-3-yl)acetamide |
| 87 | 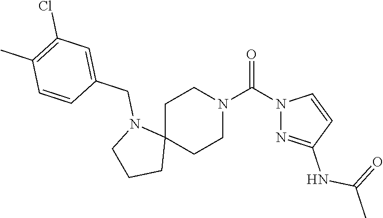 [SMILES: CC(=O)Nc1ccn(C(=O)N2CCC3(CCCN3Cc3ccc(C)c(Cl)c3)CC2)n1] | N-(1-(1-(3-Chloro-4-methylbenzyl)-1,8- diazaspiro[4.5]decane-8-carbonyl)-1H- pyrazol-3-yl)acetamide |
| 88 | 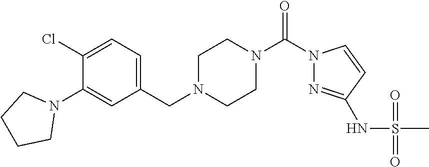 [SMILES: CS(=O)(=O)Nc1ccn(C(=O)N2CCN(Cc3ccc(Cl)c(N4CCCC4)c3)CC2)n1] | N-(1-(4-(4-Chloro-3-(pyrrolidin-1- yl)benzyl)piperazine-1-carbonyl)-1H- pyrazol-3-yl)methanesulfonamide |

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is effected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

Combination therapy also embraces the administration of the therapeutic agents as described above in further combination with other biologically active ingredients and non-drug therapies. Where the combination therapy further comprises a non-drug treatment, the non-drug treatment is conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

In some embodiments, a compound of Formula (I)-(XVII) described herein, or a pharmaceutically acceptable salt or solvate thereof, is co-administered with dopamine replacement therapy, such as levodopa or carbidopa-levodopa. In some embodiments, a compound of Formula (I)-(XVII) described herein, or a pharmaceutically acceptable salt or solvate thereof, is co-administered with levodopa. In some embodiments, a compound of Formula (I)-(XVII) described herein, or a pharmaceutically acceptable salt or solvate thereof, is co-administered with carbidopa-levodopa. In some embodiments, a compound of Formula (I)-(XVII) described herein, or a pharmaceutically acceptable salt or solvate thereof, is co-administered with amantadine.

In certain embodiments, a disclosed compound utilized by one or more of the foregoing methods is one of the generic, subgeneric, or specific compounds described herein, such as a compound of Formula (I)-(XVII).

### Preparation of the Compounds

The compounds used in the methods described herein are made according to procedures disclosed in U.S. Pat. Nos. 9,133,148; 10,030,020; 9,771,341; WO 2018/053447; U.S. Pat. Nos. 9,981,930; 10,093,635; WO 2018/093949; PCT/US18/48388; PCT/US18/48372; U.S. 62/671,985; and WO 2017/087854; which are herein incorporated by reference in their entirety. In some embodiments, compounds used in the methods described herein are made by known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, Wis., including Sigma Chemical and Fluka), Apin Chemicals Ltd. (Milton Park, UK), Ark Pharm, Inc. (Libertyville, Ill.), Avocado Research (Lancashire, U.K.), BDH Inc. (Toronto, Canada), Bionet (Cornwall, U.K.), Chemservice Inc. (West Chester, Pa.), Combi-blocks (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Spectrum Chemicals (Gardena, Calif.), Sundia Meditech, (Shanghai, China), TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²p, ³⁵S, ¹⁸F, and ³⁶Cl, respectively. Compounds described herein, and the pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Pharmaceutical Compositions

In certain embodiments, the compounds described herein are administered as a pure chemical. In other embodiments, the compounds described herein are combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising at least one compound described herein, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

In certain embodiments, the compound as described herein is substantially pure, in that it contains less than about 5%, or less than about 1%, or less than about 0.1%, of other organic small molecules, such as contaminating intermediates or by-products that are created, for example, in one or more of the steps of a synthesis method.

These formulations include those suitable for oral, rectal, topical, buccal, parenteral (e.g., subcutaneous, intramuscular, intradermal, or intravenous), vaginal, or aerosol administration.

Exemplary pharmaceutical compositions are used in the form of a pharmaceutical preparation, for example, in solid, semisolid or liquid form, which includes one or more of a disclosed compound, as an active ingredient, in a mixture with an organic or inorganic carrier or excipient suitable for external, enteral or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments for preparing solid compositions such as tablets, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, cellulose, microcrystalline cellulose, silicified microcrystalline cellulose, lactose, sucrose, glucose, mannitol, and/or silicic acid; (2) binders, such as, for example, carboxymethylcellulose, hypromellose, alginates, gelatin, polyvinyl pyrrolidone, sucrose and/or acacia; (3) humectants, such as glycerol; (4) disintegrating agents, such as crospovidone, croscarmellose sodium, sodium starch glycolate, agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, docusate sodium, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, in some embodiments, the compositions comprise buffering agents. In some embodiments, solid compositions of a similar type are also employed as fillers in soft and hard-filled gelatin capsules using such excipients as lactose or milk sugars, as well as high molecular weight polyethylene glycols and the like.

In some embodiments, a tablet is made by compression or molding, optionally with one or more accessory ingredients. In some embodiments, compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. In some embodiments, molded tablets are made by molding in a suitable machine a mixture of the subject composition moistened with an inert liquid diluent. In some embodiments, tablets, and other solid dosage forms, such as dragees, capsules, pills and granules, are scored or prepared with coatings and shells, such as enteric coatings and other coatings.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, in some embodiments, the liquid dosage forms contain inert diluents, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

In some embodiments, suspensions, in addition to the subject composition, contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. In some embodiments, the active component is mixed under sterile conditions with a pharmaceutically acceptable carrier, and with any preservatives, buffers, or propellants as required.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. In some embodiments, sprays additionally contain customary propellants, such as chlorofluorohydrocarbons and volatile unsubstituted hydrocarbons, such as butane and propane.

In some embodiments, the compounds described herein are formulated as eye drops for ophthalmic administration.

Compositions and compounds disclosed herein alternatively are administered by aerosol. This is accomplished by preparing an aqueous aerosol, liposomal preparation or solid particles containing the compound. In some embodiments, a non-aqueous (e.g., fluorocarbon propellant) suspension is used. In some embodiments, sonic nebulizers are used because they minimize exposing the agent to shear, which results in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which, in some embodiments, contain antioxidants, buffers, bacteriostats, solutes which render the formulation isotonic with the blood of the intended recipient or suspending or thickening agents.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants

Also contemplated are enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro.

The dose of the composition comprising at least one compound described herein differs, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

Oral doses typically range from about 1.0 mg to about 1000 mg, one to four times, or more, per day.

### Examples

### I. In Vitro Biological Evaluation

Compounds were tested to assess their MAGL and serine hydrolase activity using the following in vitro assays.

### In Vitro Competitive Activity-Based Protein Profiling

Proteomes (mouse brain membrane fraction or cell lysates for mouse assays; human prefrontal cortex or cell membrane fractions for human assays) (50 μL, 1.0 mg/mL total protein concentration) were preincubated with varying concentrations of inhibitors at 37° C. After 30 min, FP-Rh or HT-01 (1.0 μL, 50 μM in DMSO) was added and the mixture was incubated for another 30 min at 37° C. Reactions were quenched with SDS loading buffer (15 μL—4×) and run on SDS-PAGE. Following gel imaging, serine hydrolase activity was determined by measuring fluorescent intensity of gel bands corresponding to MAGL using ImageJ 1.43u software.

Preparation of Mouse Brain Proteomes from Inhibitor Treated Mice

Inhibitors were administered to wild-type ICR mice by oral gavage in a vehicle of 7:2:1 polyethylene glycol 400 (PEG400)/ethanol/PBS (v/v/v). Each animal was sacrificed 4 h following administration, brains were removed and brain proteomes were prepared and analyzed according to previously established methods.

The compounds shown in Table 1 demonstrated MAGL inhibitory activity with an IC₅₀ of less than 1 μM in the assays described herein.

### II. In Vivo Biological Evaluation

Compounds were tested to assess their MAGL and serine hydrolase activity using the following in vivo assay.

### MPTP-Lesioned Macaque Model of L-DOPA Induced Dyskinesia (LID)

The study utilized 8 female MPTP-lesioned cynomolgus macaques (10-15 years in age) that have received chronic repeat-treatment with L-DOPA and manifest stable and reproducible dyskinesia, of choreic and dystonic nature, in response to subsequent L-DOPA treatments.

A MGLL inhibitor, Compound 21, (3, 10 and 30 mg/kg), reference drug amantadine (10 mg/kg) and vehicle were administered by oral gavage as a single dose 2 h before a high-dose of L-DOPA (administered as Madopar™). The L-DOPA dose individualized for each animal is one that induced robust and reproducible anti-parkinsonian effects lasting ˜3-4 h but compromised by disabling dyskinesia. The animals were video-recorded for a 6 h period following L-DOPA administration and the effects of each treatment on dyskinesia, parkinsonian disability, duration and quality of anti-parkinsonian benefit (on-time) were scored blinded by a neurologist. Dyskinesia was scored using the non-human primate dyskinesia rating scale (NHPDysRS) and disability was scored using the monkey parkinsonian disability rating scale (mPDRS).

The study design was an ascending dose crossover with all animals receiving each treatment in the order of vehicle, 10 mg/kg amantadine, 3 mg/kg Compound 21, 10 mg/kg Compound 21, and 30 mg/kg Compound 21. An ascending dose design was chosen to avoid potential pharmacodynamic carryover between periods due to the long half-life of Compound 21 in MPTP-lesioned macaques (16-34 h) and the irreversible mechanism by which Compound 21 inhibits MGLL resulting pharmacodynamic effects that persist after the unbound compound is cleared from the body.

### Results

L-DOPA administration induced antiparkinsonian effects with debilitating dyskinesia in 7/8 vehicle pre-treated animals. Based on pre-determined criteria, 1 animal was excluded from subsequent analysis as it did not demonstrate the level of dyskinesia required to evaluate anti-dyskinetic effects.

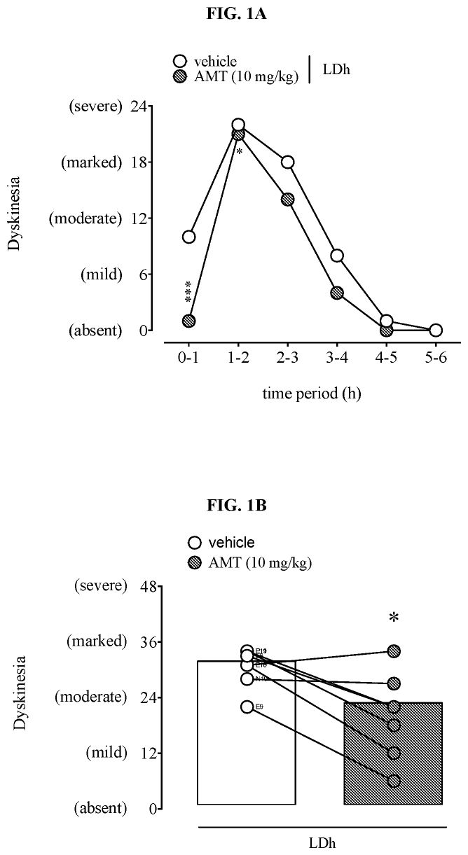

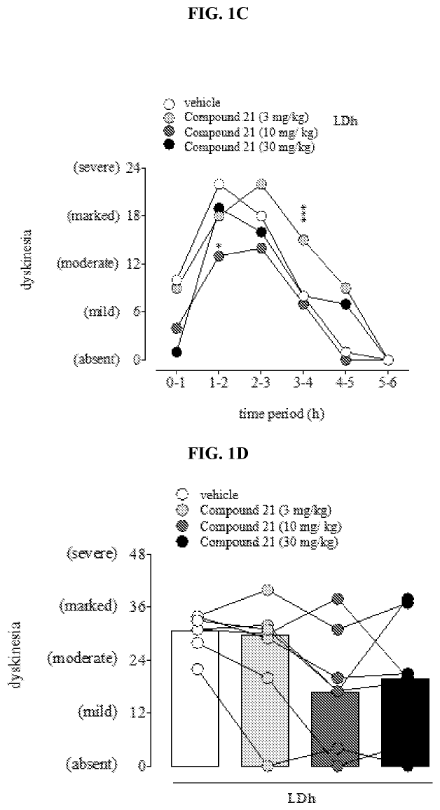

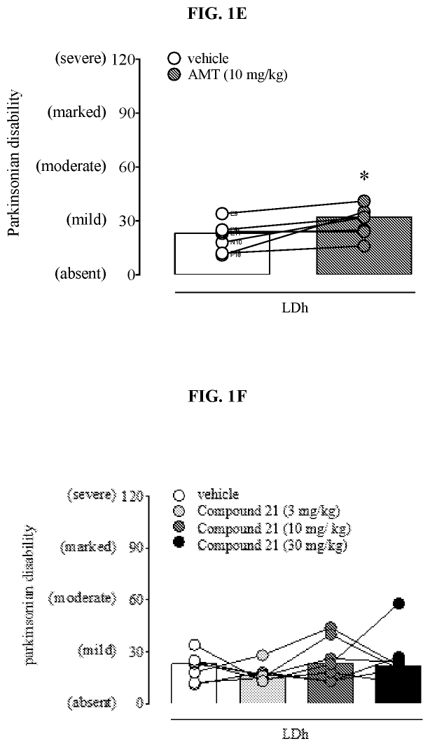

Amantadine (10 mg/kg, p.o.) was associated with a mean plasma exposure of 1,300 and 1,500 ng/mL 2 h and 8 h post-dose, respectively. The 10 mg/kg dose of amantadine produced a 29% reduction in median peak-dose dyskinesia after L-DOPA administration (P<0.05, FIGS. 1A and 1B), but was associated with a mild and statistically significant worsening of parkinsonian disability (0-2 h post L-DOPA administration totals, P<0.05, FIG. 1E).

Compound 21 (3, 10 and 30 mg/kg, p.o.) dose-dependently reduced median peak dose dyskinesia induced by L-DOPA administration (0-2 h post L-DOPA totals, FIGS. 1C and 1D). Following oral administration of 10 and 30 mg/kg Compound 21 median peak dose dyskinesia was reduced by 45% and 35%, respectively. Due to heterogeneity in animal response, the reduction in 0-2 hour total dyskinesia scores following Compound 21 administration did not reach statistical significance. However, a significant reduction in dyskinesia was observed for the 10 mg/kg group in the 1-2 h post L-DOPA interval (FIG. 1C). Although, dystonia is the predominant form of dyskinesia that presents in this model, evidence of benefit on both dystonia and chorea was observed following Compound 21 administration (data not shown). Importantly, Compound 21 (3, 10 and 30 mg/kg) did not affect the antiparkinsonian actions of L-DOPA (FIG. 1F).

Compound 21 produced robust anti-dyskinetic effects in the MPTP-lesioned macaque model of L-DOPA induced dyskinesia. The therapeutic effects of Compound 21 on median dyskinesia ratings were of greater magnitude than a therapeutically relevant dose of amantadine. Importantly, Compound 21 did not impact the antiparkinsonian effects of L-DOPA whereas amantadine was associated with a worsening of parkinsonian disability. These findings highlight a new and differentiated CNS mechanism for the treatment of L-DOPA induced dyskinesia in Parkinson's disease with a MAGL inhibitor.

III. A Randomized, Placebo-Controlled Phase II Study of a Test Compound (Compound of Formula (I)-(XVII)) in Patients with Parkinson's Disease and Dyskinesia

| | |
|---|---|
| STUDY | 1. To evaluate the efficacy of test compound in levodopa |
| OBJECTIVE | induced dyskinesia at 4 weeks compared to placebo, as |
| | measured using the change in UDysRS from baseline. |
| | 2. To evaluate the efficacy of test compound improve |
| | dyskinesia during an oral LD challenge compared to placebo |
| | measured using the change in LIDS from baseline. |
| | 3. To evaluate the efficacy of test compound on time without |
| | troublesome dyskinesia compared to placebo using PD diaries. |
| | 4. To evaluate the efficacy of test compound on motor |
| | symptoms (e.g. tremor, imbalance, freezing of gait) and |
| | NMS (e g. pain, anxiety or sleep disruption) relative to |
| | placebo, measured using the change in appropriate endpoints. |
| | 5. To evaluate the safety and tolerability of test compound in |
| | patients with PD through analysis of adverse events (AE), |
| | serious adverse events (SAE), and Suspected Unexpected |
| | Serious Adverse Reactions (SUSAR). |
| STUDY | 40 eligible patients will be randomized at 1:1 |
| POPULATION | Sequence A: Run-In Placebo, Period 1 test compound, Period 2 |
| AND NUMBER | Placebo |
| OF SUBJECTS | Sequence B: Run-In Placebo, Period 1 Placebo, Period 2 test |
| | compound |
| STUDY | Double-blind, randomized, two period, multi-center crossover |
| DESIGN | study. Eligible patients undergo a one-week single-blind |
| | placebo run-in to establish baseline symptoms. Patients continue |
| | with four weeks of double-blinded therapy, the first two weeks |
| | at a low dose of test compound/Placebo, then two weeks at a |
| | higher dose of test. compound/Placebo. There is a 1-3 week |
| | washout period with no study therapy between treatment |
| | periods. The second treatment period is a four-week treatment |
| | with the alternative study treatment. |
| MAIN | Diagnosis of PD according to the United Kingdom Parkinson's |
| INCLUSION/ | Disease Society Brain Bank criteria. |
| EXCLUSION | Inclusion: |
| CRITERIA | Men or women 30-75 years of age (30-85 years of age after |
| | DSMB agreement). |
| | LD responsive Parkinsonism |
| | Peak dose LD associated dyskinesia with a score of 1 (mild) or |
| | greater on the MDS-UPDRS question 4.2. |
| | Patients must be capable of recognizing LID |
| | Stable PD medication regimen for at least 30 days |
| | Willing to sign informed consent (and caregiver, if applicable). |
| | Exclusion: |
| | Diphasic dyskinesia |
| | Montreal Cognitive Assessment. (MoCA) ≤ 25 |
| | History of psychosis or hallucination except hallucination |
| | with past use of amantadine |
| | Current use of amantadine (patients that discontinue amantadine |
| | for 30 days and are otherwise eligible may participate) |
| | Current use of cannabis or cannabinoid medications (e.g. Sativex, |
| | dronabinol, nabilone) |
| | Dopamine receptor blockers |
| | No strong 3A4 inhibitors or inducers |
| | Significant organ dysfunction |
| DOSAGE: | Test compound or matching placebo hard gelatin capsules will |
| ROUTE AND | be administered orally in the morning with food. Study drug will |
| FORM | be administered in the morning before the LD challenge at the |
| | usual time. |
| | Each 4 week double blind treatment period is dose-escalated in |
| | double-blind fashion: |
| | Week 1-2: 20 mg test compound or Placebo daily; (2 capsules) |
| | Week 3-4: 40 mg test compound or Placebo daily; (3 capsules) |
| | If 20 mg is not tolerated, then the dose should be reduced to 10 mg |
| | If 40 mg is not tolerated, then the dose should be reduced to 30 mg. |
| | If dose reduction to 10 mg occurs during week 1-2, the dose in |
| | Week 3-4 will be 20 mg, if tolerated. |
| DURATION OF | Individual patients will participate for between 10-16 weeks. 4 |
| TREATMENT | weeks of double-blind therapy with test compound is adequate |
| | to understand its effects on dyskinesia. |
| PRIMARY | The Unified Dyskinesia Rating Seale (UDysRS) is a validated, |
| OUTCOME | FDA accepted, dyskinesia scale. Part 1 and 2 record patient |
| MEASURE(S) | perceptions of dyskinesia over the past week. Part 3 and 4 score |
| | impairment and disability from dyskinesia with 4 performance |
| | activities which are observed --communication, drinking from a |
| | cup, buttoning a lab coat, and rising from a chair, walking and |
| | returning to the chair. The objective parts of the UDysRS (Part 3 |
| | and 4) will also be separately reported. Scored by central raters |
| | from video record. |
| SECONDARY | LIDS Performance Test: The oral LD challenge paradigm |
| OUTCOME | measures ON-period dyskinesia. At 3 times after achieving full |
| MEASURE(S) | ON, the LIDS performance test is scored. Taking 5 minutes, the |
| | clinician scores 12 items (0 = none; 4 = severe) over 7 body |
| | regions, observing scoring patients at rest, and during a specified |
| | protocol of activities including speech, writing, walking, and |
| | limb movements. There are additional items to provide a global |
| | judgment of severity, impact and awareness of dyskinesia to |
| | generate a total score. In each body region, the highest severity |
| | of dyskinesia observed (even momentarily) is recorded as the |
| | ratine. Scoring is performed by central raters from video record. |
| | PD Diaries: The distribution of time affected by dyskinesia |
| | using PD diaries. Diaries are completed for 48 hours before each |
| | visit and time (30 minute intervals) is allocated into 5 options: |
| | ON without, dyskinesia, ON with dyskinesia, ON with |
| | troublesome dyskinesia, OFF, and asleep. PD diaries were a |
| | secondary endpoint for FDA review of extended release amantadine. |
| | UPDRS: The UPDRS measures general parkinsonian |
| | symptoms. Useful drugs for LID should reduce dyskinesia but. |
| | not. worsen core PD symptoms. |
| | CGI-I: Clinician's Global Impression of Change determines |
| | improvement in overall PD symptoms. It is a 7 point scale, |
| | graded by the investigator. |
| | NMSS: The Non-Motor Symptom Scale. Thirty items are |
| | scored by a clinician-rater for severity (0-3) and frequency (1-4). |
| | The NMSS will be modified to ask for symptoms over the last |
| | week instead of over the last month. The 30 items map to |
| | several domains (e.g. cardiovascular, sleep fatigue, mood and |
| | cognition, perception/hallucination, attention, memory etc). |
| | PDSS-2: Parkinson's Disease Sleep Scale consists of 15 items |
| | evaluating three domains (motor symptoms at night, PD |
| | symptoms at night, and disturbed sleep are rated by the patient |
| | using one of five categories, from 0 (never) to 4 (very |
| | frequent). Symptoms on each of 3 domains are scored 0-20 |
| | points. The questionnaire is filled out with regard to symptoms |
| | in the previous week. This scale is validated and responsive. |
| | MPQ-2: The McGill Pain Questionnaire-2 is responsive and |
| | validated in a large variety of pain conditions including |
| | muscular skeletal pain, neuropathic pain and cancer pain, and |
| | across a wide age range. It consists of 22 numeric rating scales |
| | of pain qualities (e.g. ‘burning’, ‘aching’) that the patient rates |
| | over the past week (0-10). The MPQ is considered a core |
| | endpoint in pain research. |
| | GAI: The Geriatric Anxiety Inventory consists of 20 |
| | “Agree/Disagree” items designed to assess typical common |
| | anxiety symptoms. The measurements of somatic symptoms |
| | with the instrument are limited in order to minimize confusion |
| | between symptoms common to anxiety and general medical |
| | conditions. The GAI is validated in PD. |
| | Computerized Cognition Measure: A validated computerized |
| | cognition measure (Cogstate) measures reaction time, |
| | discrimination, executive function and working memory. |

We claim:

1. A method for treating dyskinesia in a patient in need thereof, comprising administering to the patient a therapeutically effective amount of a compound of Formula (I′): 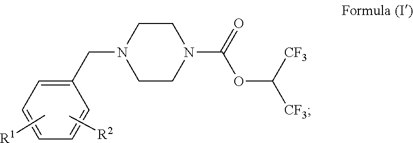
wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
(i) a 4-6 membered saturated monocyclic heterocycle; or
(ii) a 7-8 membered bridged heterocyclic ring optionally containing an additional O, N, or S;
wherein the 4-6 membered saturated monocyclic heterocycle is optionally substituted with one or two substituents independently selected from C₁₋₆ haloalkyl, —C(O)OR⁹, and —NR⁹SO₂R⁸; and the 4-6 membered saturated monocyclic heterocycle optionally contains an additional O, N, or S; and
the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
or a pharmaceutically acceptable salt or solvate thereof.

2. The method of claim 1, wherein the compound of Formula (I′) is a compound of Formula (III): 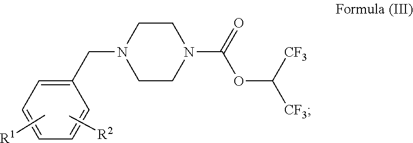
wherein:
R¹ is halogen, —OR³, —SF₅, —CN, C₁₋₆alkyl optionally substituted by halogen, or —C(O)OR⁹;
R² is —NR⁵R⁶;
R³ is selected from H, C₁₋₆alkyl, C₁₋₆haloalkyl, and C₁₋₆aminoalkyl;
R⁵ and R⁶, together with the nitrogen to which they are attached, form
(i) a 4-6 membered saturated monocyclic heterocycle; or
(ii) a 7-8 membered bridged heterocyclic ring optionally containing an additional O, N, or S;
wherein the 4-6 membered saturated monocyclic heterocycle is substituted with one or two substituents independently selected from C₁₋₆haloalkyl, —C(O)OR⁹, and —NR⁹SO₂R⁸; and the 4-6 membered saturated monocyclic heterocycle optionally contains an additional O, N, or S; and
the 7-8 membered bridged heterocyclic ring is optionally substituted with one or two substituents independently selected from halogen, oxo, and C₁₋₆alkyl;
each R⁸ is independently selected from C₁₋₆alkyl; and
each R⁹ is independently selected from H and C₁₋₆alkyl;
or a pharmaceutically acceptable salt or solvate thereof.

3. The method of claim 1 or 2, wherein R⁵ and R⁶, together with the nitrogen to which they are attached, form a 4-6 membered saturated monocyclic heterocycle, wherein
the 4-6 membered saturated monocyclic heterocycle is substituted with one substituent selected from C₁₋₆haloalkyl, —C(O)OR⁹, and —NR⁹SO₂R⁸; and
the 4-6 membered saturated monocyclic heterocycle optionally contains an additional O, N, or S.

4. The method of claim 3, wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine, piperidine, and morpholine.

5. The method of claim 4, wherein R⁵ and R⁶, together with the nitrogen to which they are attached, form a 4-6 membered saturated monocyclic heterocycle substituted with one substituent selected from C₁₋₆haloalkyl, —C(O)OR⁹, and —NR⁹SO₂R⁸, wherein the 4-6 membered saturated monocyclic heterocycle is selected from pyrrolidine and piperidine.

6. The method of claim 1, wherein R⁵ and R⁶, together with the nitrogen to which they are attached, form an unsubstituted 4-6 membered saturated monocyclic heterocycle.

7. The method of claim 6, wherein R⁵ and R⁶, together with the nitrogen to which they are attached, form an unsubstituted 4-6 membered saturated monocyclic heterocycle, wherein the 4-6 membered saturated monocyclic heterocycle is selected from pyrrolidine, piperidine, and morpholine.

8. The method of claim 1 or 2, wherein R⁵ and R⁶, together with the nitrogen to which they are attached, form a 7-8 membered bridged heterocyclic ring optionally substituted with one or two substituents independently selected from halogen, oxo, and C₁₋₆alkyl.

9. The method of claim 8, wherein R⁵ and R⁶, together with the nitrogen to which they are attached, form an unsubstituted 7-8 membered bridged heterocyclic ring.

10. The method of any one of claims 1-9, wherein R¹ is halogen, —SF₅, or optionally substituted C₁₋₆alkyl optionally substituted by halogen.

11. The method of claim any one of claims 1-10, wherein R¹ is halogen.

12. The method of claim any one of claims 1-10, wherein R¹ is C₁₋₆alkyl optionally substituted by halogen.

13. The method of claim 12, wherein R¹ is —CF₃.

14. The method of claim 1, wherein the compound is selected from: 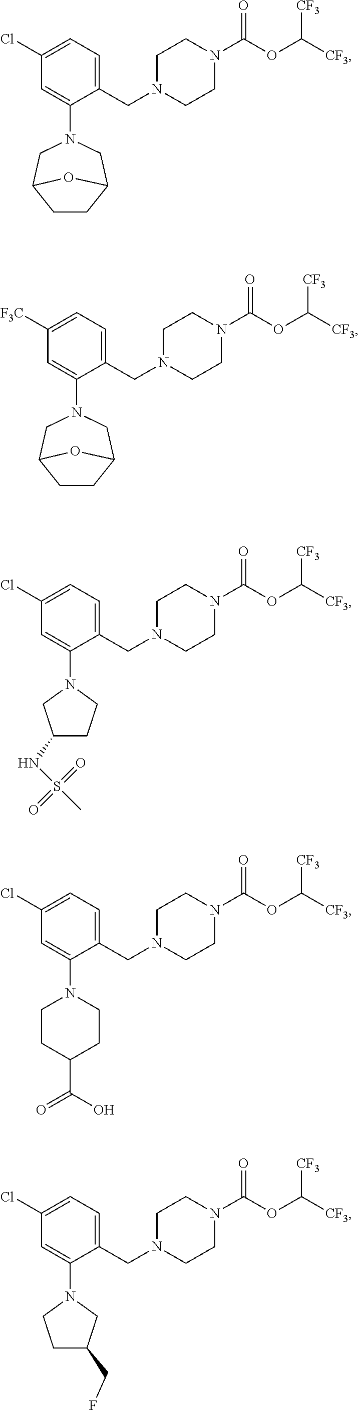 [SMILES: O=C(OC(C(F)(F)F)C(F)(F)F)N1CCN(Cc2ccc(Cl)cc2N2CC3CCC(C2)O3)CC1 ; O=C(OC(C(F)(F)F)C(F)(F)F)N1CCN(Cc2ccc(C(F)(F)F)cc2N2CC3CCC(C2)O3)CC1 ; CS(=O)(=O)N[C@H]1CCN(c2cc(Cl)ccc2CN2CCN(C(=O)OC(C(F)(F)F)C(F)(F)F)CC2)C1 ; O=C(O)C1CCN(c2cc(Cl)ccc2CN2CCN(C(=O)OC(C(F)(F)F)C(F)(F)F)CC2)CC1 ; O=C(OC(C(F)(F)F)C(F)(F)F)N1CCN(Cc2ccc(Cl)cc2N2CC[C@H](CF)C2)CC1] 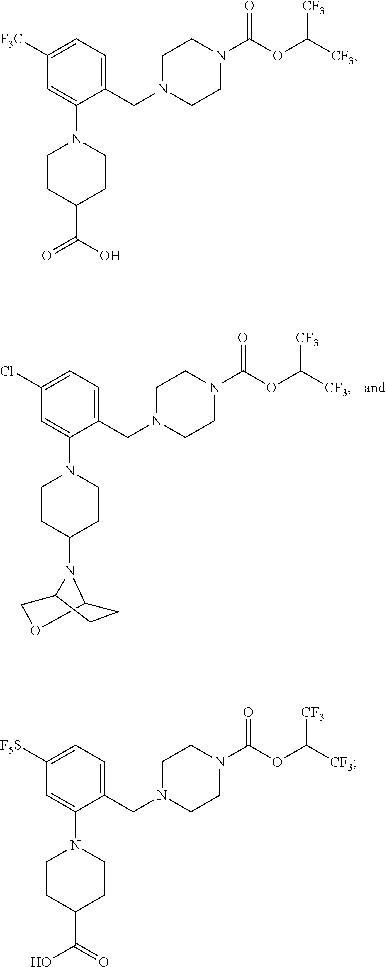
or a pharmaceutically acceptable salt or solvate thereof.

15. The method of claim 1, wherein the compound is: 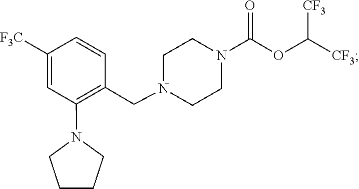 [SMILES: O=C(OC(C(F)(F)F)C(F)(F)F)N1CCN(Cc2ccc(C(F)(F)F)cc2N2CCCC2)CC1]
or a pharmaceutically acceptable salt or solvate thereof.

16. The method of any one of claims 1-15, wherein the dyskinesia is levodopa-induced dyskinesia.
